(19) United States
(12) Patent Application Publication
(10) Pub. No.: US 2026/0284108 A1
(43) Pub. Date: Sep. 24, 2026

(54) INDUCIBLE PROGRAMMED CELL DEATH-1 (PD1)-CYTOKINE CHIMERA FOR POTENTIATED IMMUNE CELL FUNCTION

(71) Applicant: Seattle Children’s Hospital d/b/a Seattle Children’s Research Institute, Seattle, WA (US)

(72) Inventors: Michael C. Jensen, Bainbridge Island, WA (US); James Rosser, Bellingham, WA (US)

(73) Assignee: Seattle Children’s Hospital d/b/a Seattle Children’s Research Institute, Seattle, WA (US)

(21) Appl. No.: 19/475,169

(22) PCT Filed: Apr. 12, 2024
(86) PCT No.: PCT/US2024/024415

§ 371 Date: Oct. 14, 2025

Related U.S. Application Data
(60) Provisional application No. 63/496,360, filed on Apr. 14, 2023.

(51) Int. Cl. A61K 35/17; A61K 38/00; A61K 40/11; A61K 40/31; A61K 40/42; A61K 48/00; A61P 35/00; C07K 14/54; C07K 14/56; C07K 14/705; C07K 14/71; C12N 5/0783; C12N 9/06; C12N 15/85

(52) U.S. Cl. CPC A61K 35/17; A61K 40/11; A61K 40/31; A61K 40/4204; A61K 40/4211; A61K 48/005; A61P 35/00; C07K 14/5434; C07K 14/5443; C07K 14/56; C07K 14/7051; C07K 14/70521; C07K 14/70578; C07K 14/71; C12N 5/0636; C12N 9/003; C12N 15/85; A61K 38/00; A61K 2239/21; A61K 2239/22; A61K 2239/31; C07K 2319/00; C12N 2510/00; C12Y 105/01003

(57) ABSTRACT

Artificial expression constructs including a PD1:cytokine chimera transgene under the control of an inducible promoter are described. The artificial expression constructs can be used to enhance the function of a recombinant receptor-immune cell (e.g., CAR-T cell). The inducible PD1:cytokine chimera transgenes disclosed herein enhance the potency of the recombinant receptor-immune cell (e.g., CAR-T cell), resulting in potentiated killing, immune cell proliferation, and/or cytokine output.

### CROSS-REFERENCE TO RELATED APPLICATIONS

This application is a U.S. National Phase Patent Application Based on International Patent Application No. PCT/US2024/024415 filed Apr. 12, 2024, which claims priority to U.S. Provisional Patent Application No. 63/496,360 filed Apr. 14, 2023, both of which are incorporated herein by reference in their entirety as if fully set forth herein.

### REFERENCE TO SEQUENCE LISTING

The Sequence Listing associated with this application is provided in XML format in lieu of a paper copy and is hereby incorporated by reference into the specification. The name of the file containing the Sequence Listing is 3121481.XML. The file is 200,704 bytes, was created on Oct. 1, 2025 and is being submitted electronically via Patent Center.

### FIELD OF THE DISCLOSURE

The current disclosure provides artificial expression constructs including a programmed cell death-1 PD1:cytokine chimera transgene under the control of an inducible promoter to potentiate the function of an immune cell (e.g., CAR-T cell). The PD1:cytokine chimera transgenes disclosed herein potentiate the function of immune cells at a tumor site by potentiating (i) the killing of targeted cells by the immune cells, (ii) proliferation of the immune cells, and/or (iii) cytokine output within the environment of the immune cells.

### BACKGROUND OF THE DISCLOSURE

According to the World Health Organization, cancer is a leading cause of death globally, and was responsible for nearly 10 million deaths in 2020. Traditional treatments such as surgery, chemotherapy, and/or radiation therapy have existed for more than a century and impact normal as well as cancerous cells. Further, surgery, chemotherapy, and radiation therapy have significant and sometimes permanent side effects.

In recent years, more targeted therapies for cancer have been developed. Targeted therapies specifically target cancer cells by identifying and exploiting specific molecular and/or immunophenotypic changes seen primarily in those cells. For example, many cancer cells preferentially express markers on their cellular surfaces which may be used as targets for antibody-based therapeutics.

Significant progress has been made in genetically engineering cells of the immune system to target and kill unwanted cell types, such as cancer cells. Many of these immune cells are T cells that have been genetically engineered to express a recombinant receptor, for example, a chimeric antigen receptor (CAR). CAR are proteins with several distinct subcomponents that allow genetically modified immune cells to recognize and kill targeted cell types. The subcomponents include at least an extracellular component and an intracellular component expressed as a single protein or in a form that assembles into a functional unit. The extracellular component includes a binding domain that specifically binds a marker (e.g., an antigen) that is preferentially present on the surface of unwanted cells such as cancerous cells. When the binding domain binds such markers, the intracellular component signals the immune cell to destroy the bound cell. CAR can additionally include a transmembrane domain that can link the extracellular component to the intracellular component.

Other subcomponents of CAR can also be used to increase CAR function. For example, spacers provide CAR with additional conformational flexibility and often increase the binding domain's ability to bind the targeted cell marker, leading to enhanced cytolytic effects. The appropriate length of a spacer within a particular CAR depends on numerous factors including how close or far a targeted marker is located from the surface of an unwanted cell's membrane.

Although CAR T cells have had substantial success in treating various cancers, challenges remain. For example, CAR T cells have seen success in treating liquid tumors but have encountered challenges when applied as a therapy against solid tumors. CAR T cells can also have suboptimal proliferation, functional anergy or exhaustion, impaired memory differentiation, and/or limited persistence limiting their ability to create or maintain remission in patients. Thus, strategies to improve CAR T cell efficacy are needed, especially for scenarios with prolonged antigen exposure that can lead to functional anergy and exhaustion.

### SUMMARY OF THE DISCLOSURE

The shortcomings of chimeric antigen receptor (CAR) T cell therapy in solid tumors necessitates the need to augment the CAR T cell with potency enhancing transgenes. Cells within solid tumors generally upregulate programmed death-ligand 1 (PD-L1), and the interaction with endogenous programmed cell death-1 (PD1) expressed on the surface of CAR T cells results in CAR T cell inhibition, loss of effector function, and cellular dysfunction. The upregulation of PD-L1, however, presents an opportunity to release a secreted CAR T cell potency enhancing molecule that can improve the efficacy of CAR T cells by, for example, promoting maintenance of the memory CAR T cell pool and proliferation and survival of memory and naive T cells as well as recruiting additional immunostimulatory cell types into the tumor microenvironment.

In particular embodiments, a secreted CAR T cell potency enhancing molecule disclosed herein includes PD1 linked to an immunostimulatory cytokine (referred to as a PD1:cytokine chimera). Because the immunostimulatory cytokine is linked to PD1 and cells within tumors upregulate PD-L1, the cytokine can be retained in a tumor microenvironment. Additionally, expression of the PD1:cytokine chimera can be regulated by a promoter that is inducible shortly after CAR activation and signaling. This feature can limit the secretion of the PD1:cytokine chimera to the area of the tumor microenvironment, further enhancing localized effects of the constructs. Particular embodiments utilize the A99L mutation in the PD1 component of the chimera to enhance PD-L1 binding.

Particular embodiments provide artificial expression constructs including a PD1:cytokine chimera transgene under the control of an inducible promoter. As indicated, the PD1:cytokine chimera can be used to enhance the function of an immune cell (e.g., T-cell or chimeric antigen receptor (CAR)-T cell), among other uses. The inducible PD1:cytokine chimera transgenes disclosed herein can enhance the potency of the immune cell (e.g., T-cell or CAR-T cell) resulting in potentiated killing, immune cell proliferation, and/or cytokine output.

In particular embodiments, the PD1:cytokine chimera transgene encodes a soluble PD1:cytokine chimera. In particular embodiments, the PD1:cytokine chimera transgene encodes a recombinant protein including (i) PD1 and (ii) interleukin 21 (IL21), interleukin 15, interleukin 15 mutein N72D (IL15(N72D)), interleukin 18 (IL18), decoy-resistant-interleukin 18 (DR-IL18 or drIL18), interleukin 12 (IL12), or interferon alpha (IFNα). PD1 refers to wildtype PD1 or a PD1(A99L) mutant.

Particular embodiments utilize inducible expression of PD1:IL21, PD1:IL15(N72D), PD1:drIL18, PD1:IL12, or PD1:IFNα to enhance immune-cell mediated cell killing. Particular embodiments utilize inducible expression of PD1:IL12, PD1:IL15(N72D), PD1:drIL18, and PD1:IL21 to enhance immune-cell mediated cell killing.

Particular embodiments utilize inducible expression of PD1:IL21, PD1:IL15(N72D), PD1:drIL18, PD1:IL12, or PD1:IFNα to enhance T cell proliferation. Particular embodiments utilize inducible expression of PD1:IFNα to enhance T cell proliferation.

Particular embodiments utilize inducible expression of PD1:IL21, PD1:IL15(N72D), PD1:drIL18, PD1:IL12, or PD1:IFNα to enhance cytokine output. Particular embodiments utilize inducible expression of PD1:IFNα to enhance cytokine output.

Particular embodiments utilize inducible expression of PD1:IFNα to enhance IFN{output.

Particular embodiments utilize inducible expression of PD1:IFNα to enhance TNFa output.

Particular embodiments utilize inducible expression of PD1:IL12 in combination with inducible expression of PD1:IL15(N72D), PD1:drIL18, and PD1:IL21. Particular embodiments utilize inducible expression of PD1:IL21 in combination with inducible expression of PD1:IL15(N72D), PD1:drIL18, PD1:IL12, and/or PD1:IFNα. Particular embodiments utilize inducible expression of PD1:IL15(N72D) in combination with inducible expression of PD1:IL21, PD1:drIL18, PD1:IL12, and/or PD1:IFNα. Particular embodiments utilize inducible expression of PD1:drIL18 in combination with inducible expression of PD1:IL21, PD1:IL15(N72D), PD1:IL12, and/or PD1:IFNα. Particular embodiments utilize inducible expression of PD1:IL12 in combination with inducible expression of PD1:IL21, PD1:IL15(N72D), PD1:drIL18, and/or PD1:IFNα. Particular embodiments utilize inducible expression of PD1:IFNα in combination with inducible expression of PD1:IL21, PD1:IL15(N72D), PD1:drIL18, and/or PD1:IL12.

In particular embodiments, PD1 and the cytokine are joined by a linker. The linker can be a flexible linker, such as a glycine-serine linker. In particular embodiments, the glycine-serine linker includes a sequence as set forth in SEQ ID NO: 128. In particular embodiments, the glycine-serine linker includes the sequence GGG.

In particular embodiments, the expression of the PD1:cytokine chimera transgene is under the control of a promoter. In particular embodiments, the promoter is an iSynPro promoter. In particular embodiments, the iSynPro promoter includes S1-61 (SEQ ID NOs: 17 or 18). In particular embodiments, the iSynPro promoter includes S1-61 (SEQ ID NO: 17). In particular embodiments, the artificial expression construct with the PD1:cytokine chimera transgene further includes or encodes a skip sequence and a control feature.

In particular embodiments, the immune cell includes a T cell. In particular embodiments, the immune cell can be engineered to express a recombinant receptor. In particular embodiments, artificial expression constructs encoding the recombinant receptor can be included in the same or a different artificial expression construct from the PD1:cytokine chimera transgene. In particular embodiments, the artificial expression construct encoding the recombinant receptor includes or encodes a promoter and a recombinant receptor. In particular embodiments, the artificial expression construct encoding the recombinant receptor includes or encodes a promoter, a recombinant receptor, a skip sequence, and a control feature. In particular embodiments, the recombinant receptor includes a CAR or engineered T cell receptor (eTCR). In particular embodiments, the recombinant receptor includes an extracellular component that binds a target antigen, a transmembrane domain, and an intracellular component.

Thus, the current disclosure provides systems to impact the killing, proliferation, and/or cytokine output of immune cells. The immune cells can include recombinant receptor-expressing immune cells which can be engineered to target different target cells (e.g., cancer cell, virus, bacteria, fungi) expressing a target antigen by engineering the extracellular domain to include a binding domain that binds the target antigen.

### BRIEF DESCRIPTION OF THE FIGURES

Some of the drawings submitted herewith may be better understood in color. Applicant considers the color versions of the drawings as part of the original submission and reserves the right to present color images of the drawings in later proceedings.

FIG. 1. Schematic of various PD1:cytokine chimera transgenes (sometimes referred to herein as “Staplekines” or where “Staplekine” replaces “PD1” in the name of a construct). The PD1:cytokine chimera utilizes the programmed cell death-1 A99L mutant (PD1(A99L)) receptor. This receptor is known to strongly interact with its ligand PDL1, a ligand that is normally upregulated on tumor derived tissue. Appended to the PD1(A99L) through small linker are five examples of immunostimulatory signals: interleukin (IL)21, IL15(N72D), decoy resistant IL18 (DR-18 or drIL18), IL12, and IFNα. By appending theses immunostimulatory signals to the PD1(A99L) receptor, the cytokine signal is retained at localized environments with high levels of PDL1 expression to augment engineered T cell potency and function.

FIG. 2. Construct architecture to facilitate T cell production and screening. PD1(A99L):cytokine chimera transgenes are driven under the regulation of a chimeric antigen receptor (CAR) inducible promoter (e.g., S1-61) allowing for the controlled release of the encoded immunomodulatory protein after CAR engagement with its target antigen. As shown in the construct design, the dual promoter construct format is adopted, featuring an inducible promoter driving a PD1:cytokine chimera transgene and a constitutively expressed promoter (e.g., EF1a(long) promoter) expressing a CAR, double mutant dihydrofolate reductase (DHFRdm), and truncated epidermal growth factor receptor (EGFRt) with a 2A skip sequence separating the CAR and DHFRdm as well as the DHFRdm and EGFRt. The inducible promoter can also drive gene expression of a cell surface-localizing polypeptide tag based on truncated human HER2 (Her2tG) which can be connected to the PD1:cytokine chimera transgene via 2A skip sequence.

FIG. 3. After cell production, effector T cell lines maintain a high level of CD19CAR positivity assessed through EGFRt detection. CD4+ T cells were examined for CAR positivity through staining for EGFRt via flow cytometry after the end of cell manufacturing using the TICLE production method. Briefly, as used herein, the TICLE method includes obtaining a CD8+ fraction, a CD4+ fraction, and a negative fraction from a sample (e.g., peripheral blood mononuclear cells); introducing the artificial expression construct to the CD8+ or CD4+ cells using electroporation or viral transduction; coculturing the transduced cells with the CD4+ or CD8+ fraction and negative fraction; and coculturing in a gas-permeable vessel in media supplemented with 7.5 U/mL IL2, 20 ng/mL IL4, 10 ng/mL IL7, and 20 ng/mL IL21. In either CD19CAR or CD19CAR+iSynPro PD1:IFNα transgene, CD4+ cells were enriched for the delivered constructs as determined by high positivity of EGFRt staining.

FIG. 4. Cell killing and cell proliferation. In a separate donor, CD4+ cells highly enriched for either CD19CAR alone or CD19CAR+iSynPro sPD1(A99L) -G3-IFNα (i.e., PD1:IFNα) were subjected to a recursive killing assay using the neuroblastoma cell line Be2-mCherry-CD19t. Effectors and targets were cocultured for 72 hours and subjected to two additional stimulations occurring at 144 and 216 hours. Effector and targets were setup at different E:T ratios. The E:T ratios of 0.25:1 for the killing assay and 1:1 for a proliferation assay are shown. A killing score (the sum of the difference in Be2 killing between CD19CAR alone and CD19CAR+iSynPro PD1:IFNα) was used to determine the difference between each group assessed. In CD4+ cells, iSynPro PD1:IFNα enhanced killing compared to CD19CAR alone suggesting PD1:IFNα can improve CAR T cell functionality. In the same assay, T cell proliferation was determined by using live cell imaging software to quantify the number of “low red” objects indicating T cells. A similar scoring system was employed to assess T cell proliferation compared to CD19CAR alone. PD1:IFNα enhanced T cell proliferation compared to CD19CAR alone.

FIG. 5. Cytokine production. Supernatant was collected 24 hours after the initiation of cocultures containing either CD4 positive CD19CAR or CD19CAR+iSynPro sPD1(A99L) -G3-IFNα (i.e., PD1:IFNα) and Be2-mCherry-CD19t targets mixed at a 1:1 E:T ratio. Cytokine levels of effector function cytokines interferon gamma (IFNg) and tumor necrosis factor alpha (TNFa) were assessed. iSynPro PD1:IFNα enhanced the production of IFNg and TNFa.

FIG. 6. Target killing by donor CD4+ cells. A library containing four different dual promoter plasmids (PD1(A99L):IL-12, PD1(A99L):IL15(N72D), PD1(A99L):drIL18, and PD1(A99L):IL-21) mixed at equimolar ratios was used to generate CD4+lines using the method referenced to herein as TICLE. Effector cells were used to conduct an Incucyte-based target killing assay using the neuroblastoma cell line, Be2-CD19t-mCherry, and was recursively stimulated with targets across multiple effector to target ratios for a total of two stimulations. Plotted is the percent of remaining Be2 signal determined at the end of the first and second stimulation. Cells harboring the library enhanced T cell killing of the target at all E:T ratios in both the first and second stimulations. Venus indicates a library of PD1:cytokine chimeras including PD1(A99L):IL-12, PD1(A99L):IL15(N72D), PD1(A99L):drIL18, and PD1(A99L):IL-21.

FIG. 7. Target killing by donor CD8+ cells. A library containing four different dual promoter plasmids (PD1(A99L):IL-12, PD1(A99L):IL15(N72D), PD1(A99L):drIL18, and PD1(A99L):IL-21) mixed at equimolar ratios was used to generate CD8+lines using the method referenced to herein as the TICLE method. Effector cells were used to conduct an Incucyte-based target killing assay using the neuroblastoma cell line Be2-CD19t-mCherry and was recursively stimulated with targets across multiple effector to target ratios for a total of two stimulations. Plotted is the percent of remaining Be2 signal determined at the end of the first and second stimulations. Cells harboring the library of PD1:cytokine chimeras enhanced T cell killing of the target at all E:T ratios in both the first and second stimulations.

FIG. 8. Sequences supporting the disclosure including sdnPD1(A99L) (SEQ ID NO: 1), IL21 (SEQ ID NO: 2), IL15(N72D) (SEQ ID NO: 3), DR-IL18 (SEQ ID NO: 4), IL12 (SEQ ID NO: 5), IFNα (SEQ ID NO: 6), PD1:IL21 (PD1(A99L) and G4S x3 linker and IL21) encoding sequence (SEQ ID NO: 7), PD1:IL15(N72D) (PD1(A99L) and G4S x3 linker and IL15(N72D)) encoding sequence (SEQ ID NO: 8), PD1:drIL18 (PD1(A99L) and G4S x3 linker and drIL18) encoding sequence (SEQ ID NO: 9), PD1:IL12 (PD1(A99L) and G4S x3 linker and IL12) encoding sequence (SEQ ID NO: 10), PD1:IFNα (PD1(A99L) and Gx3 linker and IFNα) encoding sequence (SEQ ID NO: 11), PD1:IL21 (PD1(A99L) and G4S x3 linker and IL21) amino acid sequence (SEQ ID NO: 12), PD1:IL15(N72D) (PD1(A99L) and G4S x3 linker and IL15(N72D)) amino acid sequence (SEQ ID NO: 13), PD1:drIL18 (PD1(A99L) and G4S x3 linker and drIL18) amino acid sequence (SEQ ID NO: 14), PD1:IL12 (PD1(A99L) and G4S x3 linker and IL12) amino acid sequence (SEQ ID NO: 15), PD1:IFNα (PD1(A99L) and Gx3 linker and IFNα) amino acid sequence (SEQ ID NO: 16), S1-61-version 1 (SEQ ID NO: 17), S1-61-version 2 (SEQ ID NO: 18), S1-17 (SEQ ID NO: 19), S1-37 (SEQ ID NO: 20), S1-4 (SEQ ID NO: 21), S1-1 (SEQ ID NO: 22), S1-3 (SEQ ID NO: 23), S1-42 (SEQ ID NO: 24), S1-62 (SEQ ID NO: 25), S1-15 (SEQ ID NO: 26), S1-2 (SEQ ID NO: 27), S1-27 (SEQ ID NO: 28), S1-8 (SEQ ID NO: 29), S1-30 (SEQ ID NO: 30), S1-33 (SEQ ID NO: 31), S1-41 (SEQ ID NO: 32), S1-59 (SEQ ID NO: 33), S1-66 (SEQ ID NO: 34), S1-71 (SEQ ID NO: 35), S1-56 (SEQ ID NO: 36), S1-6 (SEQ ID NO: 37), S1-60 (SEQ ID NO: 38), S1-86 (SEQ ID NO: 39), S1-32 (SEQ ID NO: 40), S1-10 (SEQ ID NO: 41), S1-18 (SEQ ID NO: 42), S1-14 (SEQ ID NO: 43), S1-16 (SEQ ID NO: 44), S1-19 (SEQ ID NO: 45), S1-26 (SEQ ID NO: 46), S1-65 (SEQ ID NO: 47), S2-n1 (SEQ ID NO: 48), S4-n1 (SEQ ID NO: 49), S6-n1 (SEQ ID NO: 50), S1-325 (SEQ ID NO: 51), S1-60 (SEQ ID NO: 52), S2-274 (SEQ ID NO: 53), S2-310 (SEQ ID NO: 54), S1-367 (SEQ ID NO: 55), S1-7 (SEQ ID NO: 56), EF1a promoter (Long; Ef1a-HTLV hybrid) (SEQ ID NO: 57), Core insulator-version 1 (SEQ ID NO: 58), Core insulator—version 2 (SEQ ID NO: 59), IgG4 hinge-A ESKYGPPCPPCP (SEQ ID NO: 60), IgG4 hinge coding sequence-A (SEQ ID NO: 61), IgG4 hinge coding sequence-B (SEQ ID NO: 62), IgG4 hinge coding sequence-C(SEQ ID NO: 63), IgG4-CH2 domain encoding sequence (SEQ ID NO: 64), IgG4-CH3 encoding sequence (SEQ ID NO: 65), CD28 Transmembrane Domain-A (SEQ ID NO: 66), CD28 Transmembrane Domain-B (SEQ ID NO: 67), CD28 Transmembrane Domain-C (SEQ ID NO: 68), CD28TM encoding sequence-A (SEQ ID NO: 69), CD28TM encoding sequence-B (SEQ ID NO: 70), CD28TM encoding sequence-C(SEQ ID NO: 71), CD28TM encoding sequence-D (SEQ ID NO: 72), 41BB costimulatory domain-A (SEQ ID NO: 73), 4-1 BB costimulatory domain-B (SEQ ID NO: 74), 4-1 BB costimulatory domain-C(SEQ ID NO: 75), 4-1BB signaling encoding sequence-A (SEQ ID NO: 76), 4-1BB signaling encoding sequence-B (SEQ ID NO: 77), 4-1BB signaling coding sequence-C(SEQ ID NO: 78), CD3ζ signaling domain-A (SEQ ID NO: 79), CD3ζ signaling domain-B (SEQ ID NO: 80), CD3ζ signaling domain-C(SEQ ID NO: 81), CD3ζ signaling domain encoding sequence-A (SEQ ID NO: 82), CD3zeta signaling domain encoding sequence-B (SEQ ID NO: 83), Thoseaasigna Virus 2A (T2A) Peptide-v1 (SEQ ID NO: 84), Thoseaasigna Virus 2A (T2A) Peptide-v2 (SEQ ID NO: 85), Thoseaasigna Virus 2A (T2A) Peptide-v3 (SEQ ID NO: 86), Porcine Teschovirus-1 2a (P2A) Peptide (SEQ ID NO: 87), Equine Rhinitis A Virus (ERAV) 2A (E2A) Peptide (SEQ ID NO: 88), Foot-And-Mouth Disease Virus 2A (F2A) Peptide (SEQ ID NO: 89), T2A encoding sequence-v1 (SEQ ID NO: 90), T2A encoding sequence-v2 (SEQ ID NO: 91), EGFRt-version 1 (SEQ ID NO: 92), EGFRt-version 2 (SEQ ID NO: 93), EGFRt encoding sequence (SEQ ID NO: 94), tCD19 coding sequence (SEQ ID NO: 95), HER2tG (without start codon) (SEQ ID NO: 96), DHFRdm (SEQ ID NO: 97), Vector s1-61-il2mp_her2tg-t2a-sdnpd1a991-g4s3-il21_rbgpolya_2x3hsa_ef1al_hucd19scfvg01s-igg4hinge-cd28tm-41bb-zeta-p2a-dhfrdm-t2a-egfrt_bghpolya_pbnp (SEQ ID NO: 98), Vector s1-61-il2mp_her2tg-t2a-sdnpd1199-g4s3-il15n72d_rbgpolya_2x3 hs-a_ef1a7-3_hucd19scfvg01s-igg4hinge-cd28tm-41bb-zeta-p2a-dhfrdm-t2a-egfrt_bghpolya_pbnp (SEQ ID NO: 99), Vector s1-61-il2mp_her2tg-t2a-sdnpd1199-g4s3-dr-il186-12_rbgpolya_2x3 hs-a_ef1a7-3_hucd19scfvg01s-igg4hinge-cd28tm-41bb-zeta-p2a-dhfrdm-t2a-egfrt_bghpolya_pbnp (SEQ ID NO: 100), Vector s1-61-il2mp_her2tg-t2a-sdnpd1a991-g4s3-il12_rbgpolya_2x3hsa_ef1al_hucd19scfvg01s-igg4hinge-cd28tm-41bb-zeta-p2a-dhfrdm-t2a-egfrt_bghpolya_pbnp (SEQ ID NO: 101), Vector s1-61-il2mp_her2tg-t2a-sdnpd1a991-g3-iFNa5878_rbgpolya_2x3hsa_ef1al_hucd19scfvg01s-igg4hinge-cd28tm-41bb-zeta-p2a-dhfrdm-t2a-egfrt_bghpolya_pbnp (SEQ ID NO: 102), and Vector s1-61-il2mp_her2tg_t2a_pd1a99-g4s3-il12_pbnp (SEQ ID NO: 103).

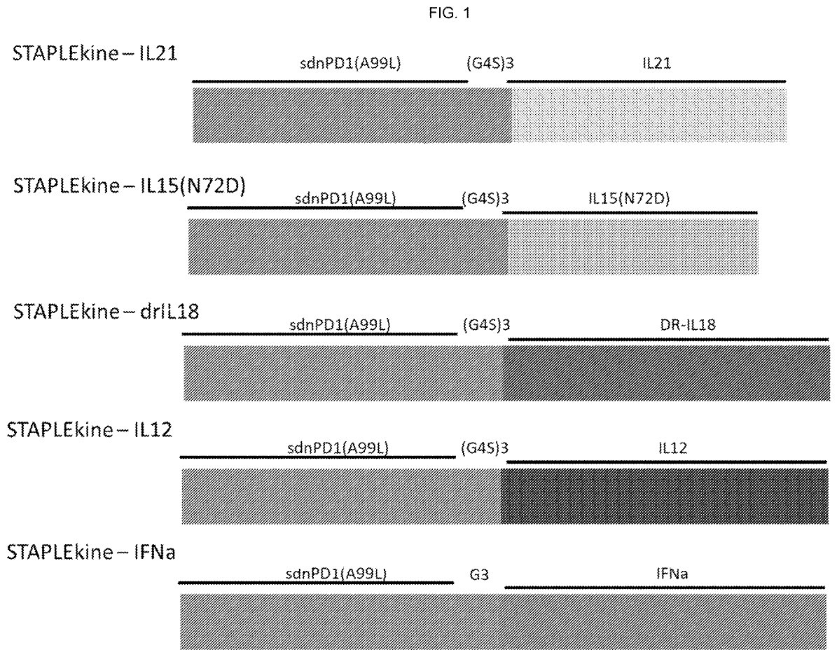

### DETAILED DESCRIPTION

A variety of therapies have been developed over the last few decades to engineer immune cells to express a recombinant receptor that binds antigens on unwanted cell types (e.g., cancer cells, virally infected cells (also referred to as targeted cells herein)). When the recombinant receptor binds the target antigen, the immune cell can be activated to destroy the bound cell type. Chimeric antigen receptor (CAR) cell therapy is one such therapy.

Chimeric antigen receptor (CAR) therapy involves engineering immune cells to express synthetic receptors designed to target cells such as cancer cells or virally infected cells. Although CAR therapies have had substantial success in treating various cancers, challenges remain. For example, CAR T cells have seen success in treating liquid tumors but have encountered challenges when applied as a therapy against solid tumors. For example, CAR T cells can have suboptimal proliferation, functional anergy or exhaustion, impaired memory differentiation, and/or limited persistence limiting their ability to create or maintain remission in patients. Thus, strategies to improve CAR T cell efficacy are needed, especially for scenarios with prolonged antigen exposure that can lead to functional anergy and exhaustion.

The shortcomings of CAR T cell therapy in solid tumors necessitates the need to augment the CAR T cell with potency enhancing transgenes. Cells within solid tumors generally upregulate programmed death-ligand 1 (PD-L1), and the interaction with endogenous PD1 expressed on the surface of CAR T cells results in CAR T cell inhibition, loss of effector function, and cellular dysfunction. The upregulation of PD-L1, however, presents an opportunity to release a secreted CAR T cell potency enhancing molecule that can improve the efficacy of CAR T cells by, for example, promoting maintenance of the memory CAR T cell pool and proliferation and survival of memory and naive T cells as well as recruiting additional immunostimulatory cell types into the tumor microenvironment.

In particular embodiments, a secreted CAR T cell potency enhancing molecule disclosed herein includes PD1 linked to an immunostimulatory cytokine (referred to as a PD1:cytokine chimera). Because the immunostimulatory cytokine is linked to PD1 and cells within tumors upregulate PD-L1, the cytokine can be retained in a tumor microenvironment. Additionally, expression of the PD1:cytokine chimera can be regulated by a promoter that is inducible shortly after CAR activation and signaling. This feature can limit the secretion of the PD1:cytokine chimera to the area of the tumor microenvironment, further enhancing localized effects of the constructs. Particular embodiments utilize the A99L mutation in the PD1 component of the chimera to enhance PD-L1 binding.

Particular embodiments provide artificial expression constructs including a programmed cell death-1 (PD1):cytokine chimera transgene under the control of an inducible promoter. As indicated, the PD1:cytokine chimera can be used to enhance the function of an immune cell (e.g., T-cell or chimeric antigen receptor (CAR)-T cell), among other uses. The inducible PD1:cytokine chimera transgenes disclosed herein can enhance the potency of the immune cell (e.g., T-cell or CAR-T cell) resulting in potentiated killing, immune cell proliferation, and/or cytokine outputs.

In particular embodiments, the PD1:cytokine chimera transgene encodes a soluble PD1:cytokine chimera transgene. In particular embodiments, the PD1:cytokine chimera transgene encodes a recombinant protein including (i) PD1 wildtype or a PD1 A99L mutant (PD1(A99L)) and (ii) interleukin 21 (IL21), interleukin 15, interleukin 15 mutein N72D (IL15(N72D)), interleukin 18 (IL18), decoy-resistant-interleukin 18 (DR-IL18 or also referred to herein as drIL18), interleukin 12 (IL12), or interferon alpha (IFNα). PD1 refers to wildtype PD1 or a PD1(A99L) mutant. In particular embodiments, PD1 is PD1 wildtype. In particular embodiments, PD1 is PD1(A99L) mutant.

Particular embodiments utilize inducible expression of PD1:IL21, PD1:IL15(N72D), PD1:drIL18, PD1:IL12, or PD1:IFNα to enhance immune-cell mediated cell killing. Particular embodiments utilize inducible expression of PD1:IL12, PD1:IL15(N72D), PD1:drIL18, and PD1:IL21 to enhance immune-cell mediated cell killing. Particular embodiments utilize inducible expression of PD1:IFNα to enhance immune-cell mediated cell killing.

Particular embodiments utilize inducible expression of PD1:IL21, PD1:IL15(N72D), PD1:drIL18, PD1:IL12, or PD1:IFNα to enhance T cell proliferation. Particular embodiments utilize inducible expression of PD1:IFNα to enhance T cell proliferation.

Particular embodiments utilize inducible expression of PD1:IL21, PD1:IL15(N72D), PD1:drIL18, PD1:IL12, or PD1:IFNα to enhance cytokine output. Particular embodiments utilize inducible expression of PD1:IFNα to enhance cytokine output. In particular embodiments, cytokine output includes IFNγ output and/or TNFa output.

Particular embodiments utilize inducible expression of PD1:IFNα to enhance IFNγ output.

Particular embodiments utilize inducible expression of PD1:IFNα to enhance TNFa output.

The terms “enhance” and “potentiate” are used synonymously herein.

Particular embodiments utilize inducible expression of PD1:IL12 in combination with inducible expression of PD1:IL15(N72D), PD1:drIL18, and PD1:IL21. Particular embodiments utilize inducible expression of PD1:IL21 in combination with inducible expression of PD1:IL15(N72D), PD1:drIL18, PD1:IL12, and/or PD1:IFNα. Particular embodiments utilize inducible expression of PD1:IL15(N72D) in combination with inducible expression of PD1:IL21, PD1:drIL18, PD1:IL12, and/or PD1:IFNα. Particular embodiments utilize inducible expression of PD1:drIL18 in combination with inducible expression of PD1:IL21, PD1:IL15(N72D), PD1:IL12, and/or PD1:IFNα. Particular embodiments utilize inducible expression of PD1:IL12 in combination with inducible expression of PD1:IL21, PD1:IL15(N72D), PD1:drIL18, and/or PD1:IFNα. Particular embodiments utilize inducible expression of PD1:IFNα in combination with inducible expression of PD1:IL21, PD1:IL15(N72D), PD1:drIL18, and/or PD1:IL12. Particular embodiments utilize inducible expression of PD1:IL15 in combination with inducible expression of PD1:IL21, PD1:drIL18, PD1:IL12, and/or PD1:IFNα.

In particular embodiments, PD1 and the stimulatory cytokine are joined by a linker. The linker can be a flexible linker, such as a glycine-serine linker. In particular embodiments, the glycine-serine linker includes a sequence as set forth in SEQ ID NO: 128. In particular embodiments, the glycine-serine linker includes the sequence GGG.

In some examples, the artificial expression construct with the PD1:cytokine chimera transgene is encoded by the sequence as set forth in SEQ ID NOs: 7, 8, 9, 10, or 11.

In particular embodiments, the artificial expression construct with the PD1:cytokine chimera transgene includes a promoter and encodes a PD1:cytokine chimera transgene. In particular embodiments, the promoter includes an iSynPro promoter and an 1L2 minimal promoter.

In particular embodiments, the artificial expression construct encoding the PD1:cytokine chimera transgene further includes or encodes a skip sequence and a control feature. In particular embodiments, the iSynPro promoter includes S1-61 (SEQ ID NO: 17).

The current disclosure also provides systems and methods for genetically modifying an immune cell to express a recombinant receptor. In some embodiments, the artificial expression construct including the PD1:cytokine chimera transgene can further include a sequence encoding the recombinant receptor. In another embodiment, the PD1:cytokine chimera transgene and the sequence encoding the recombinant receptor can be on separate artificial expression constructs.

In particular embodiments, a recombinant receptor is under the control of a second promoter. In particular embodiments, the artificial expression construct includes or encodes a promoter and a recombinant receptor. In particular embodiments, the artificial expression construct includes or encodes a promoter, a recombinant receptor, a skip sequence, and a control feature. In particular embodiments, the artificial expression construct includes or encodes a promoter, a recombinant receptor, a first skip sequence, a selection cassette (e.g., dihydrofolate reductase double mutant, DHFRdm), a second skip sequence, and a transduction marker (EGFRt). In particular embodiments, a recombinant receptor includes a CAR or an engineered T cell receptor (eTCR).

In particular embodiments, the recombinant receptor includes an extracellular component that binds a target antigen, a transmembrane domain, and an intracellular component.

In particular embodiments, the artificial expression construct with the PD1:cytokine chimera transgene and the recombinant receptor includes or encodes an inducible promoter, a PD1:cytokine chimera transgene, a 2A skip sequence, a constitutive promoter, and a recombinant receptor. In particular embodiments, the artificial expression construct with the PD1:cytokine chimera transgene and the recombinant receptor includes or encodes an inducible promoter, a PD1:cytokine chimera transgene, a first 2A skip sequence, Her2tG, a constitutive promoter, a recombinant receptor, a second 2A skip sequence, a DHFRdm, a third 2A skip sequence, and an EGFRt. In particular embodiments, the inducible promoter includes an iSynPro promoter. In particular embodiments, the inducible promoter includes an iSynPro promoter and a minimal promoter. In particular embodiments, the minimal promoter includes an IL2 minimal promoter.

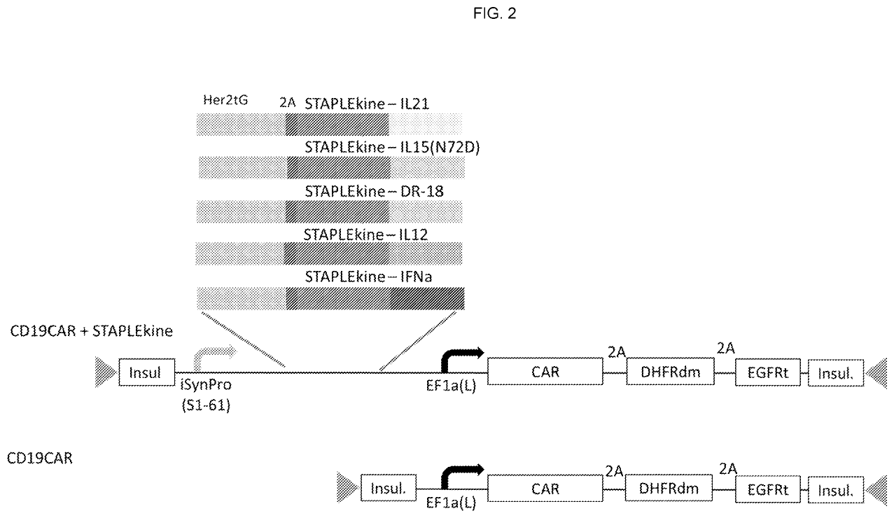

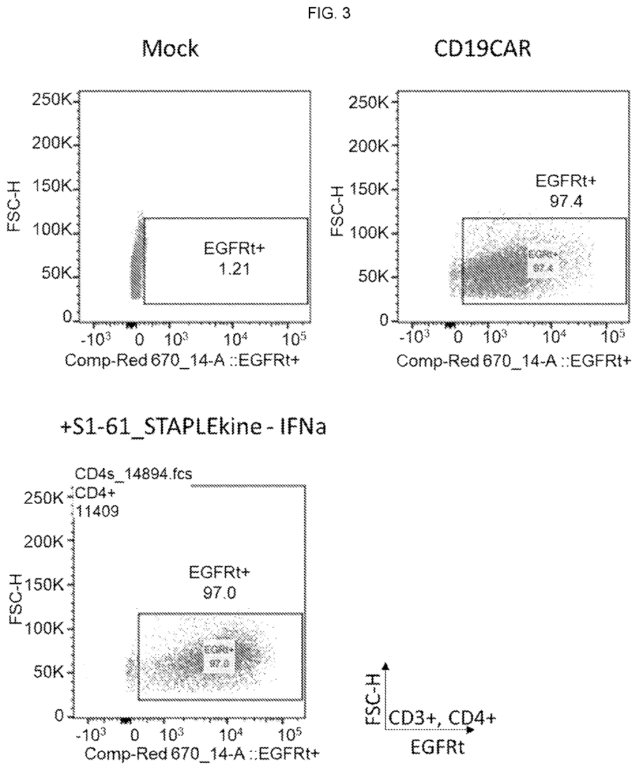

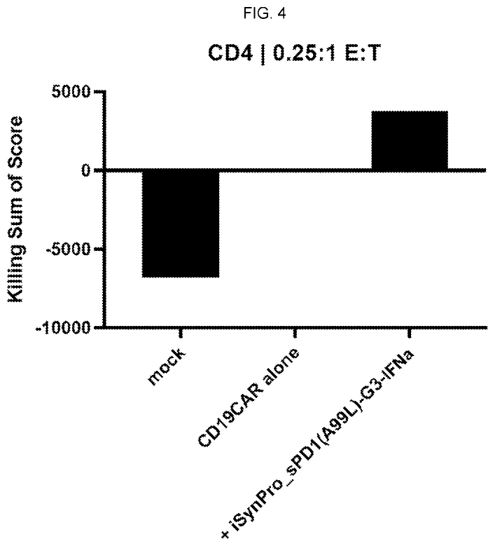

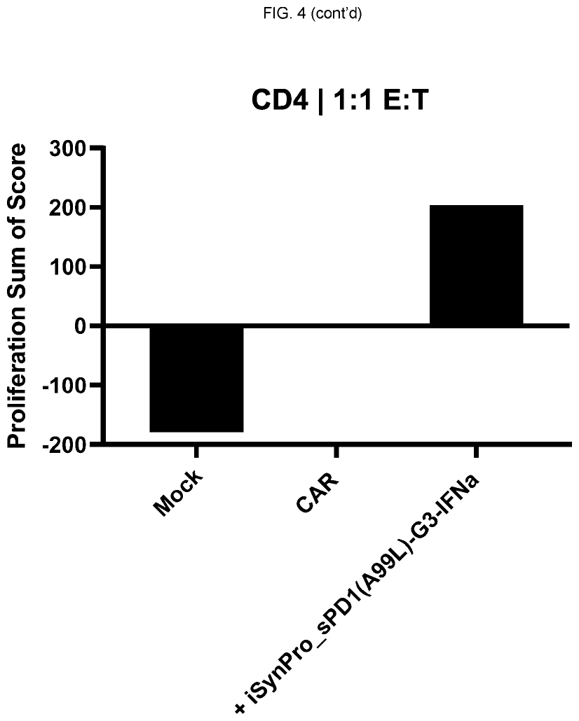

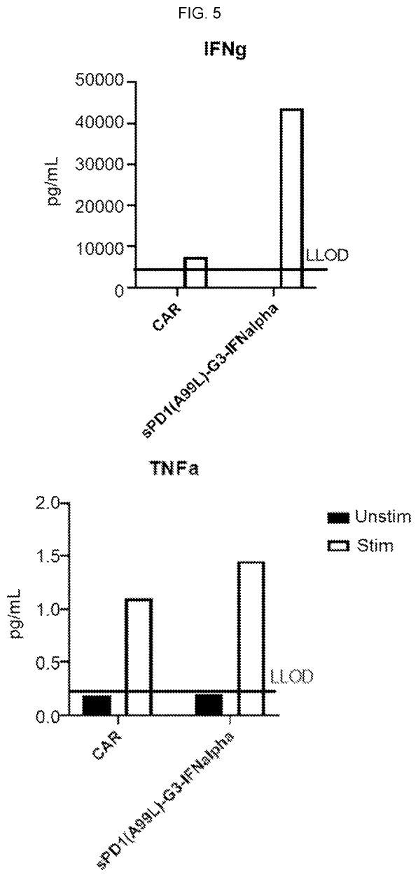

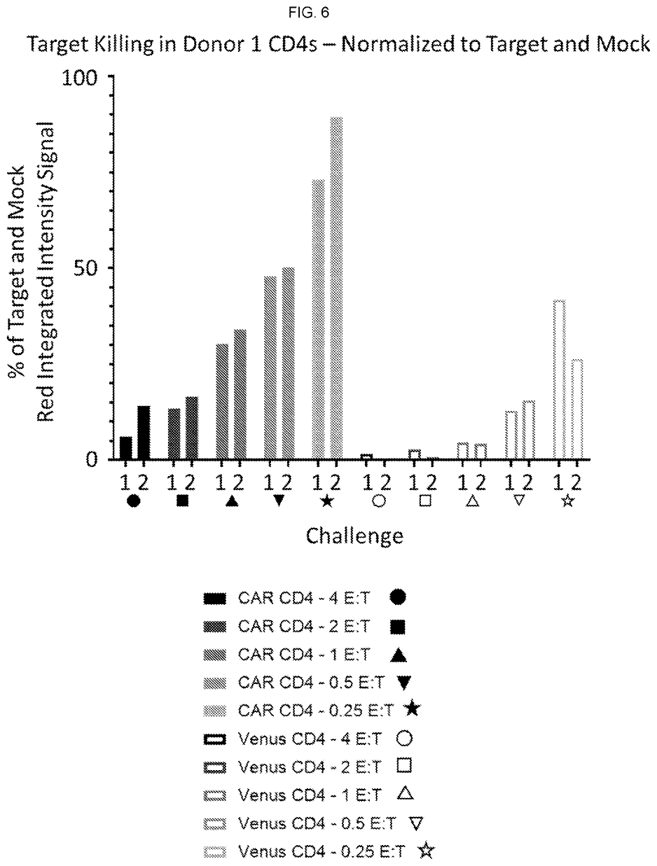

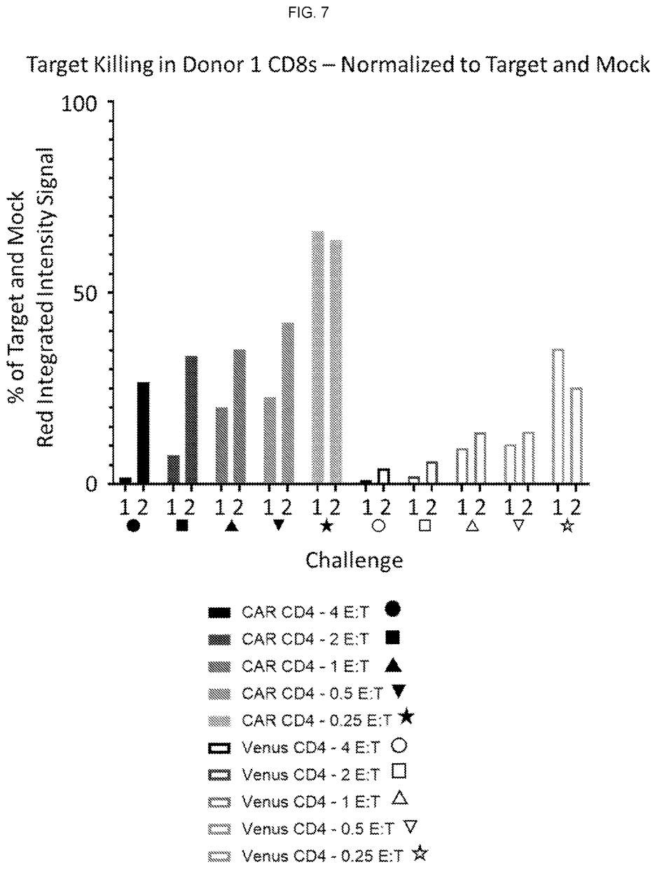

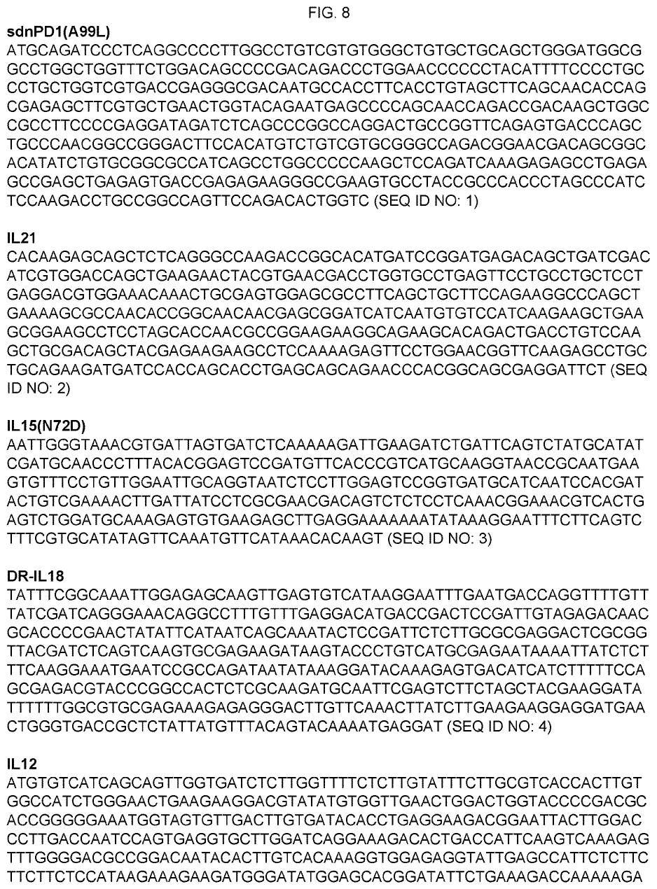

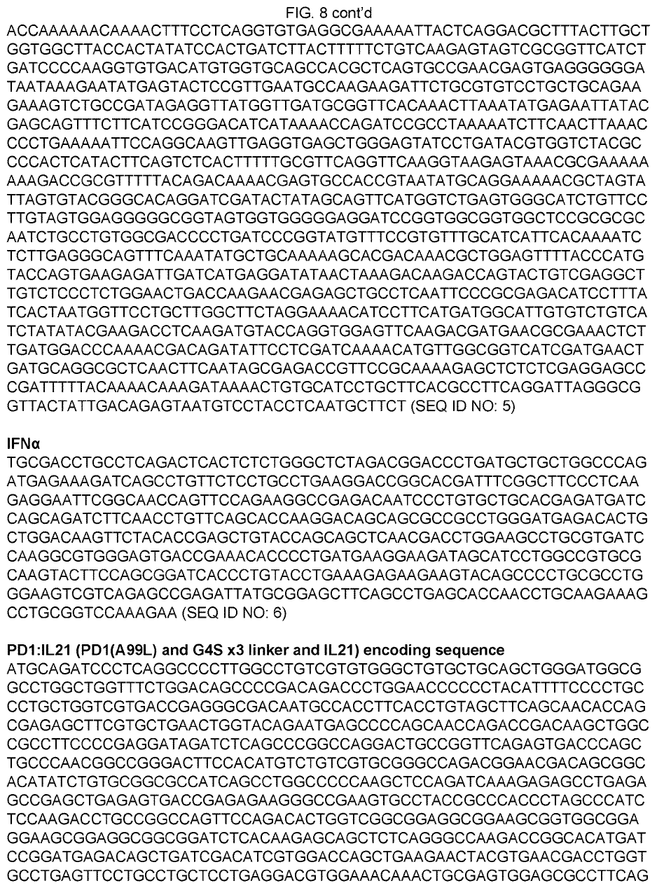

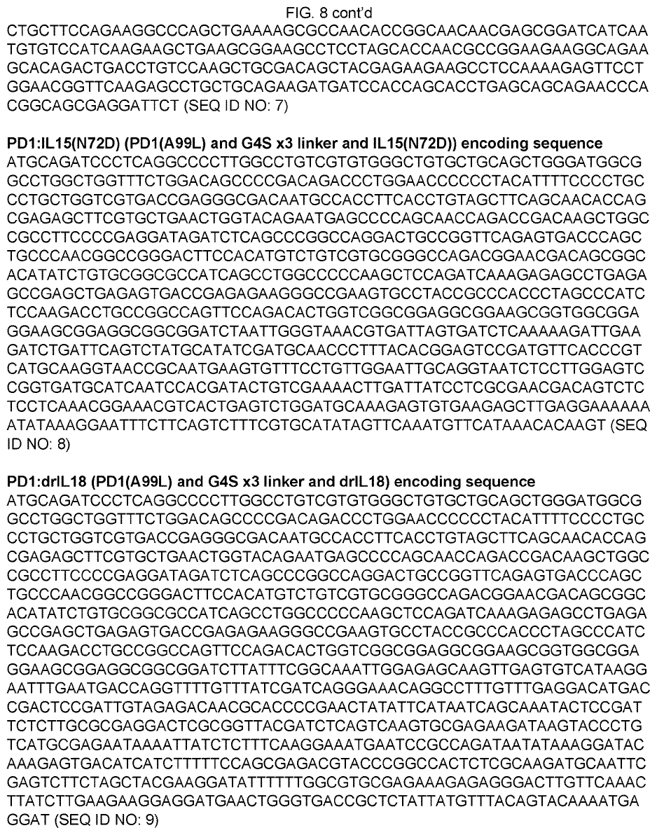

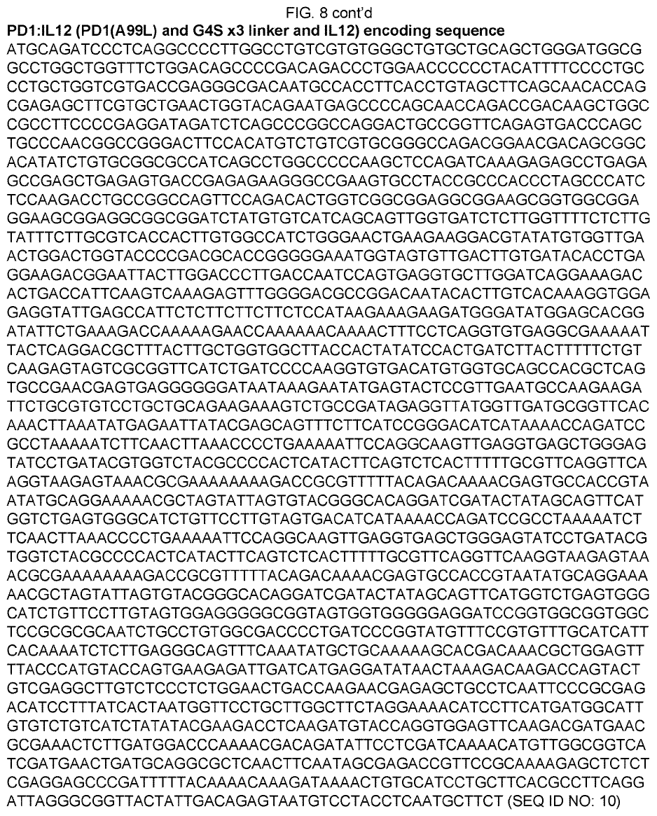

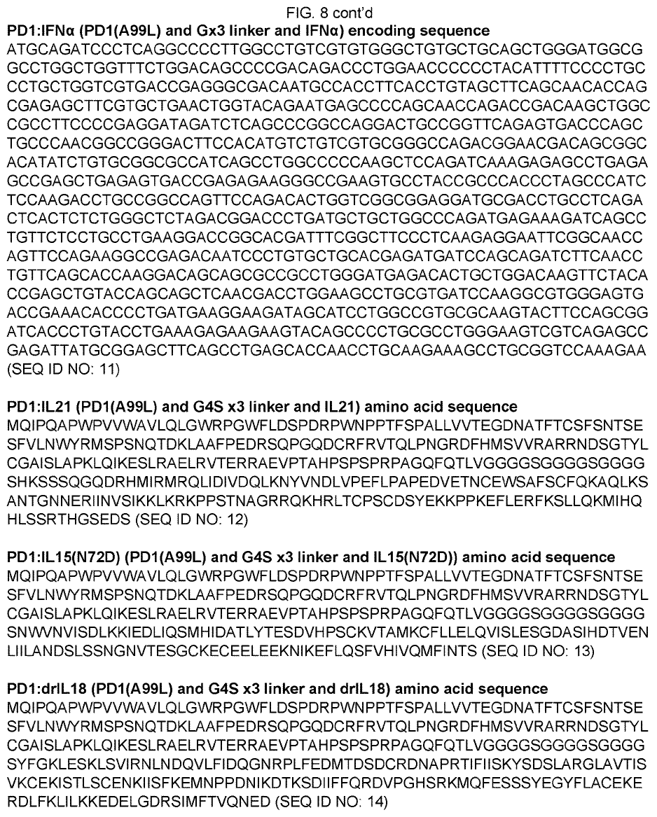

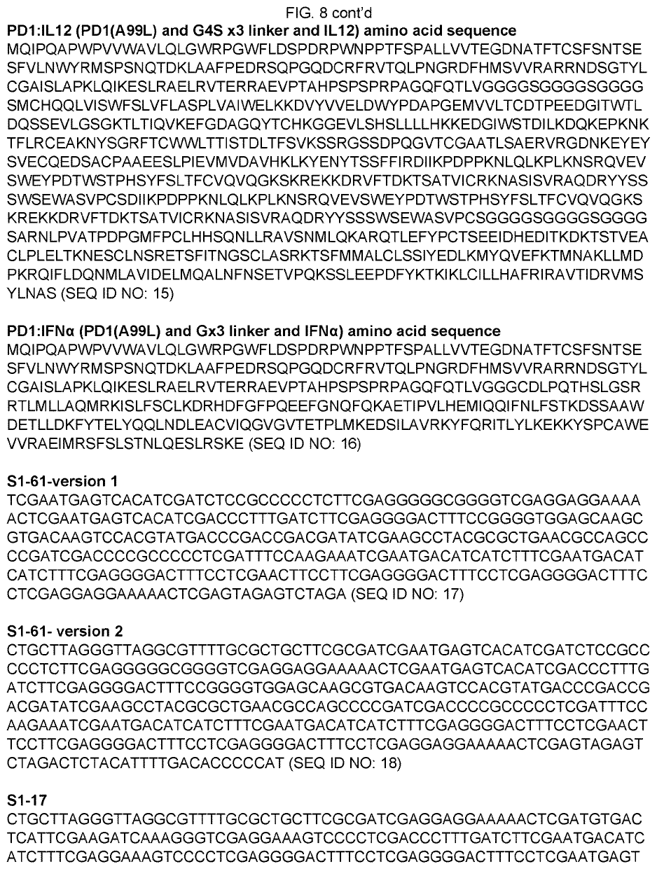

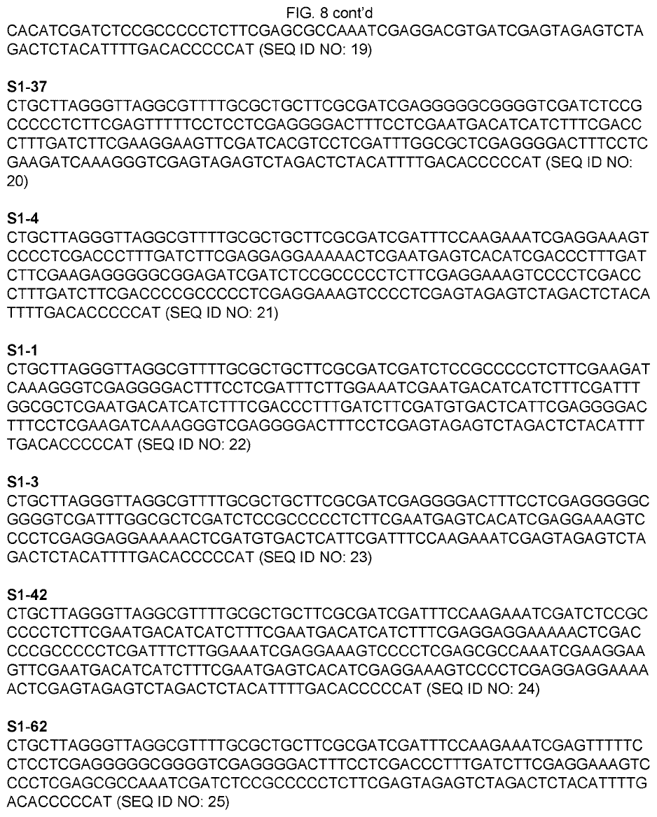

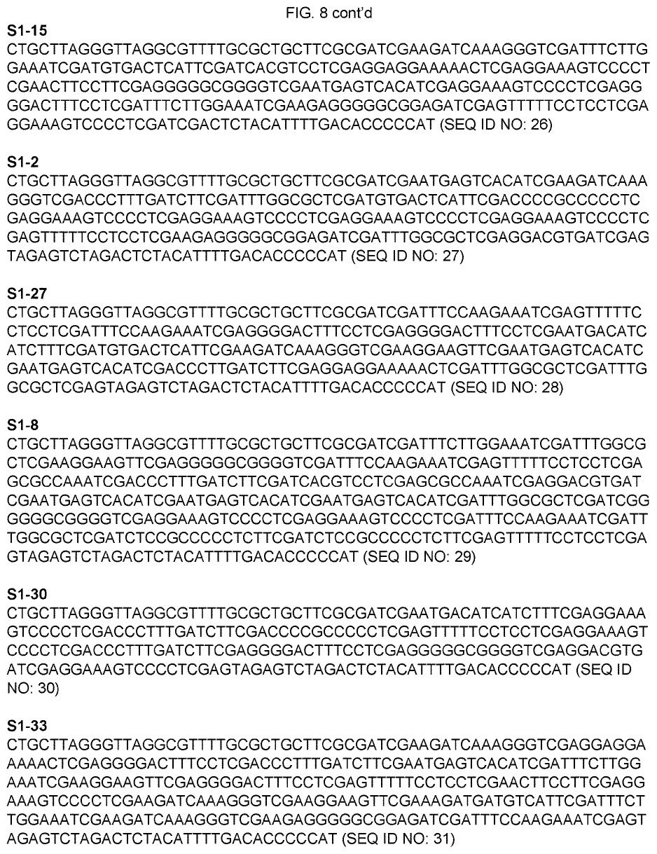

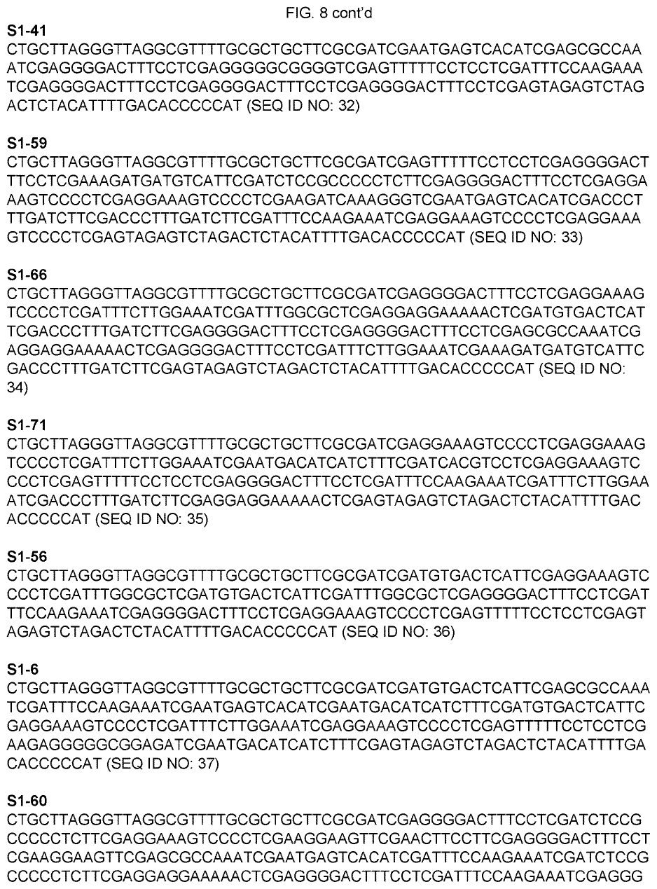

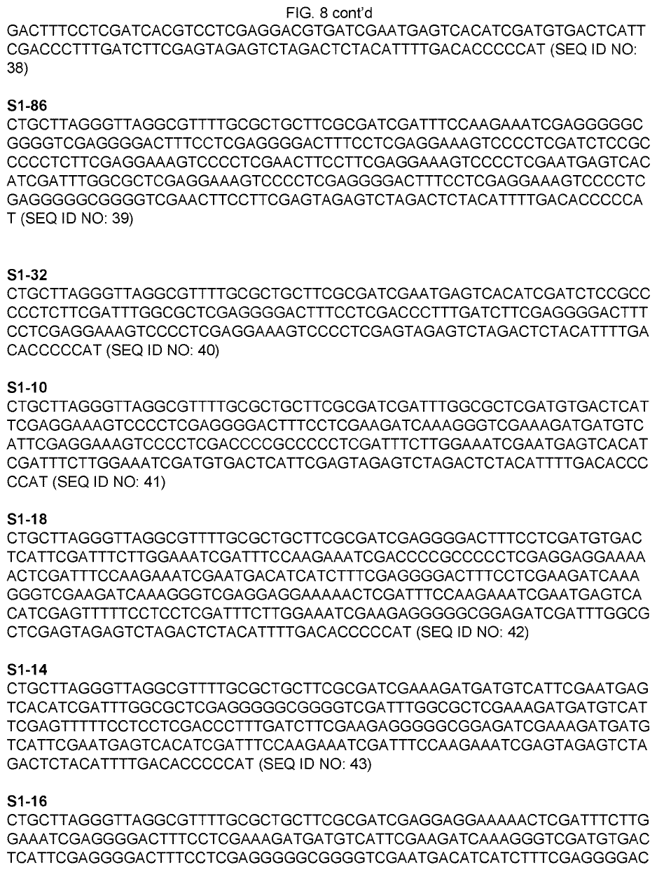

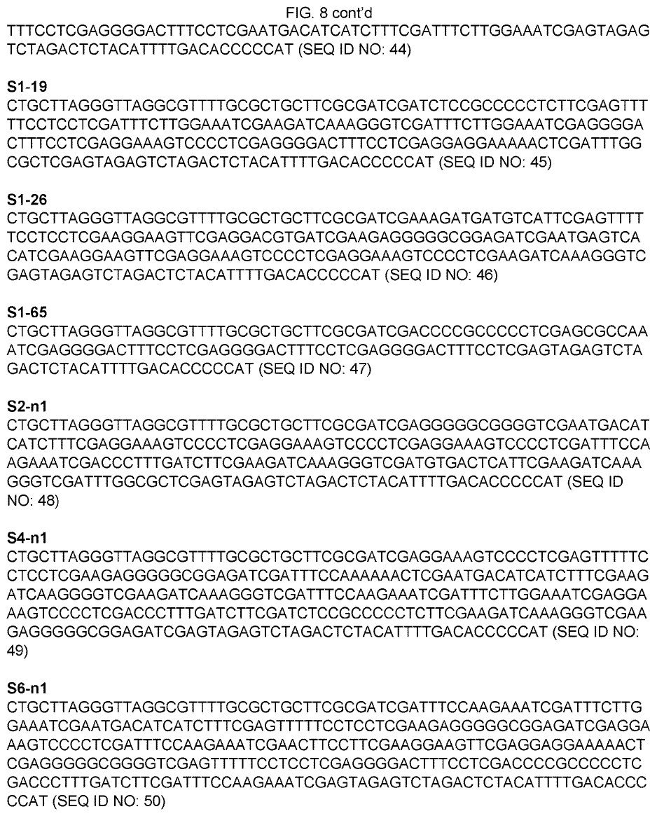

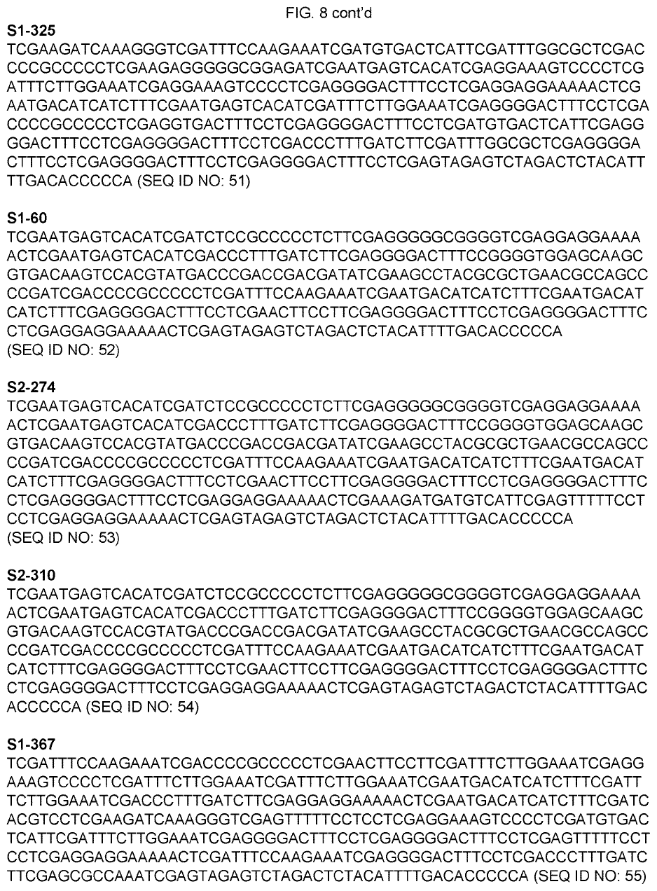

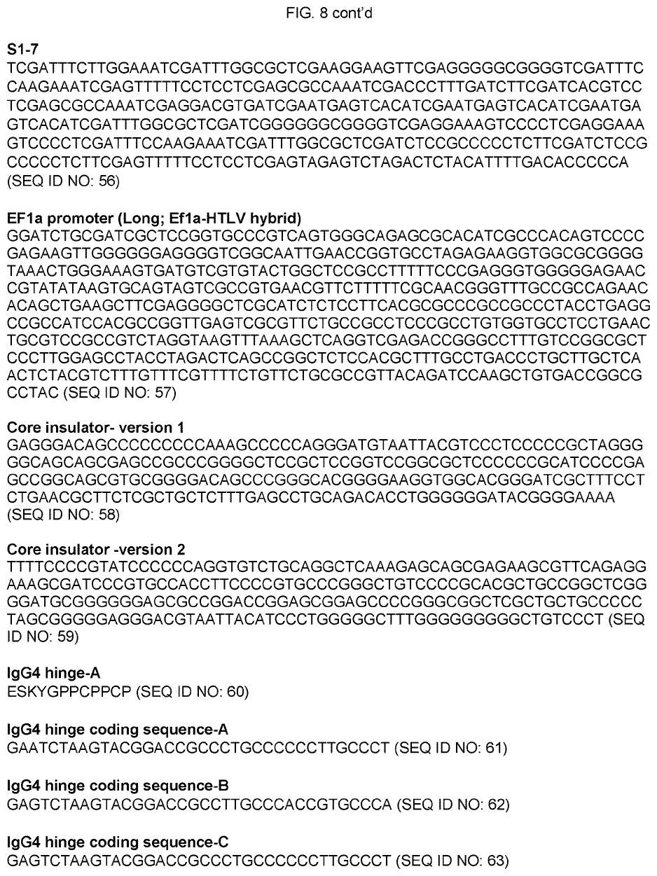

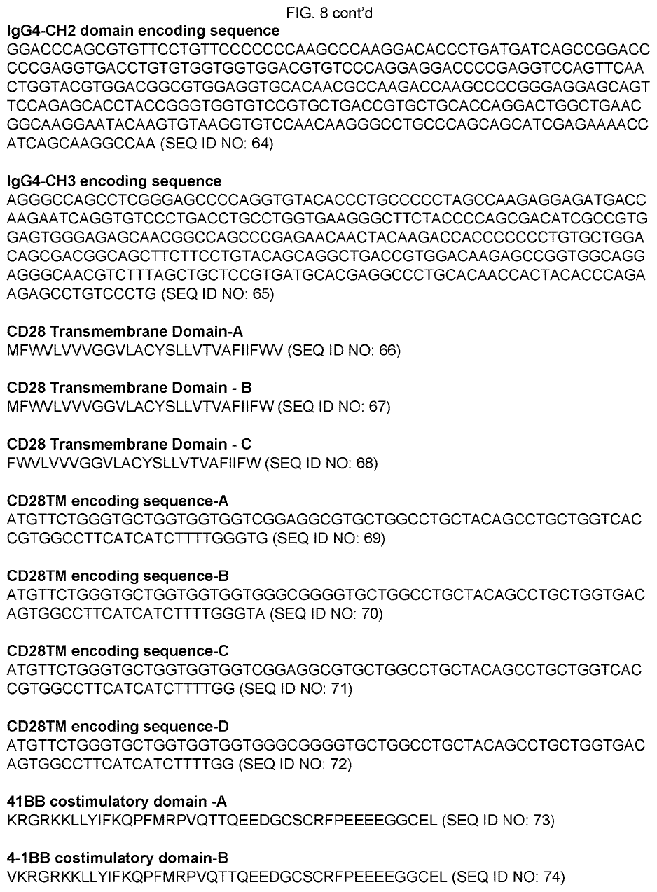

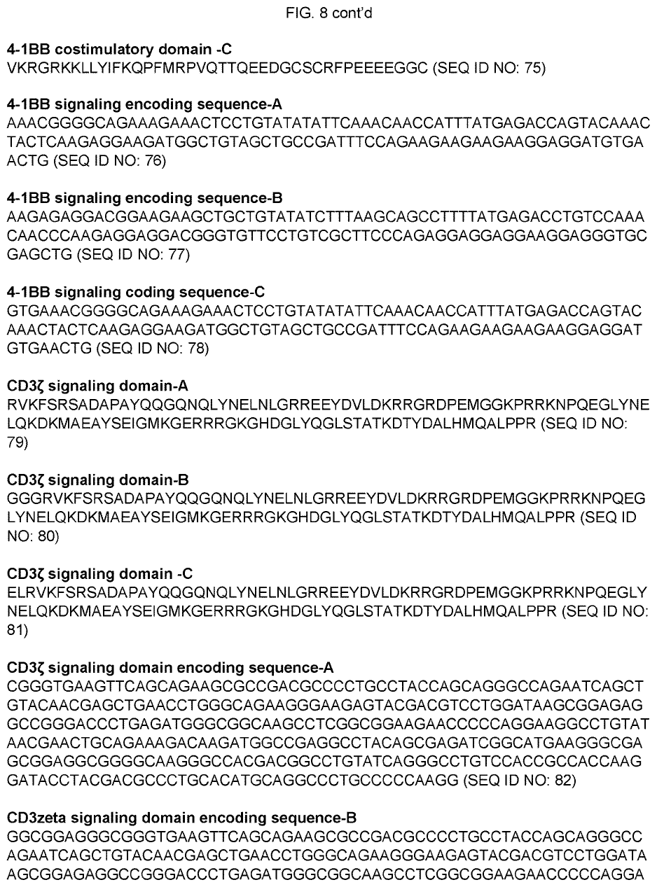

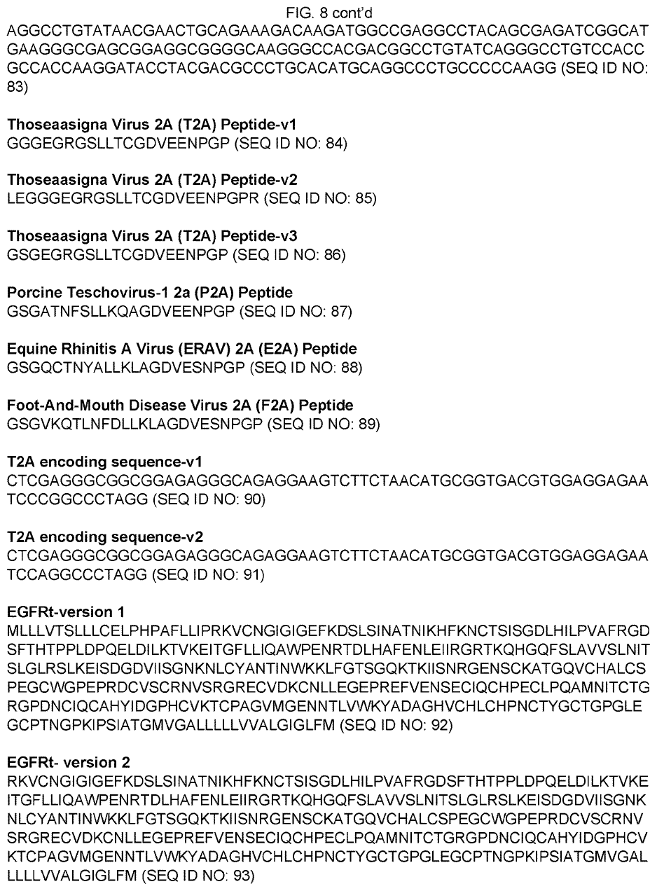

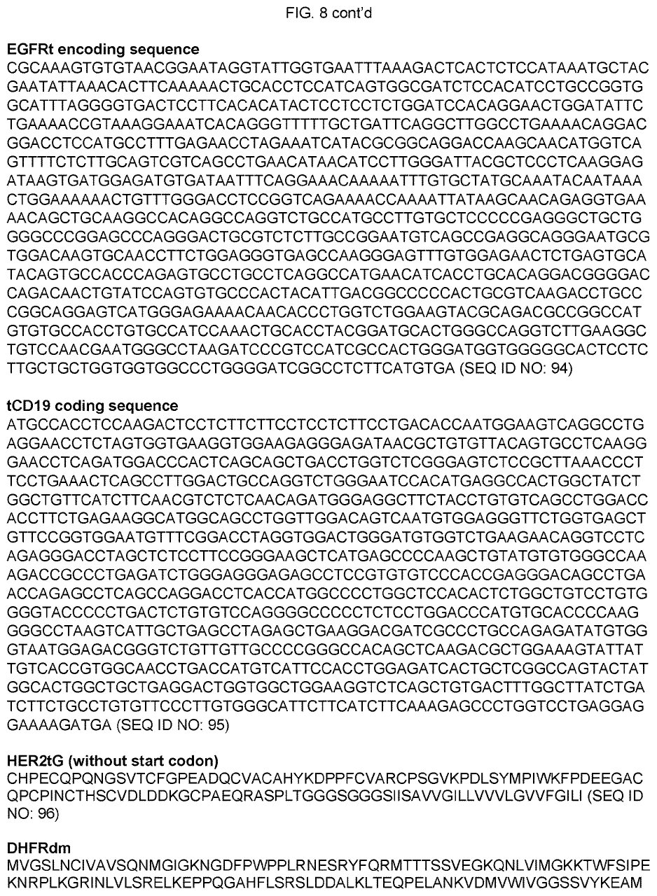

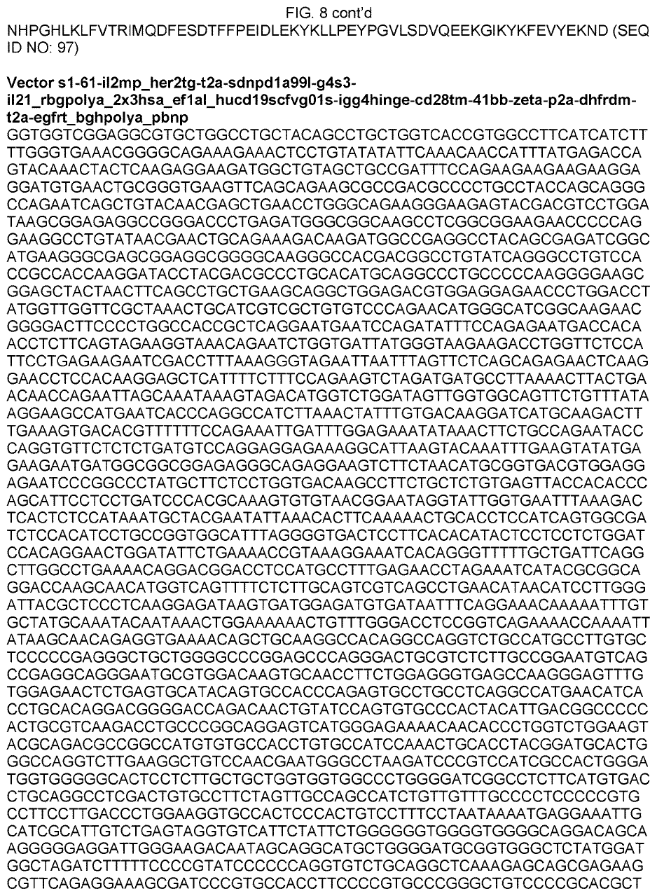

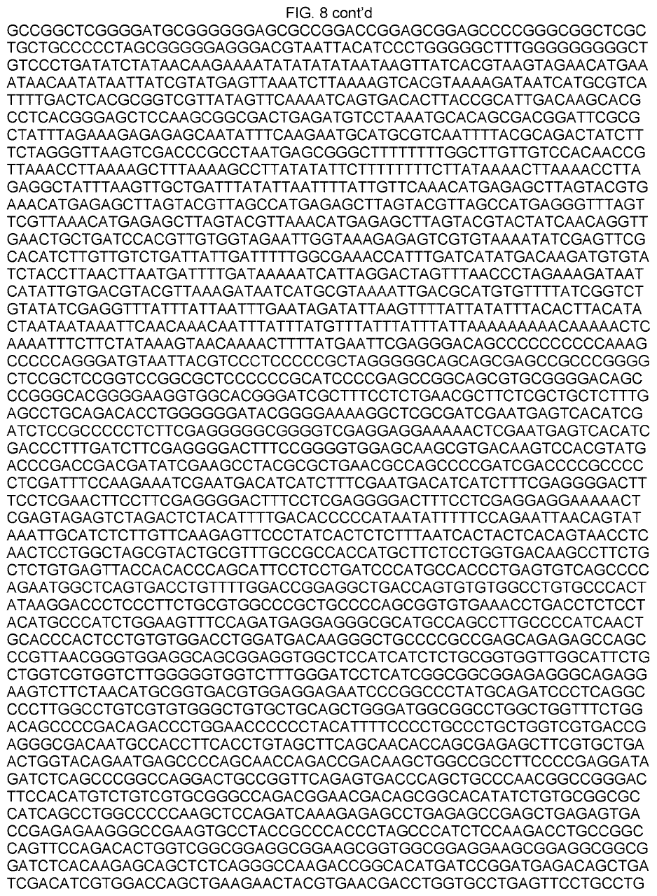

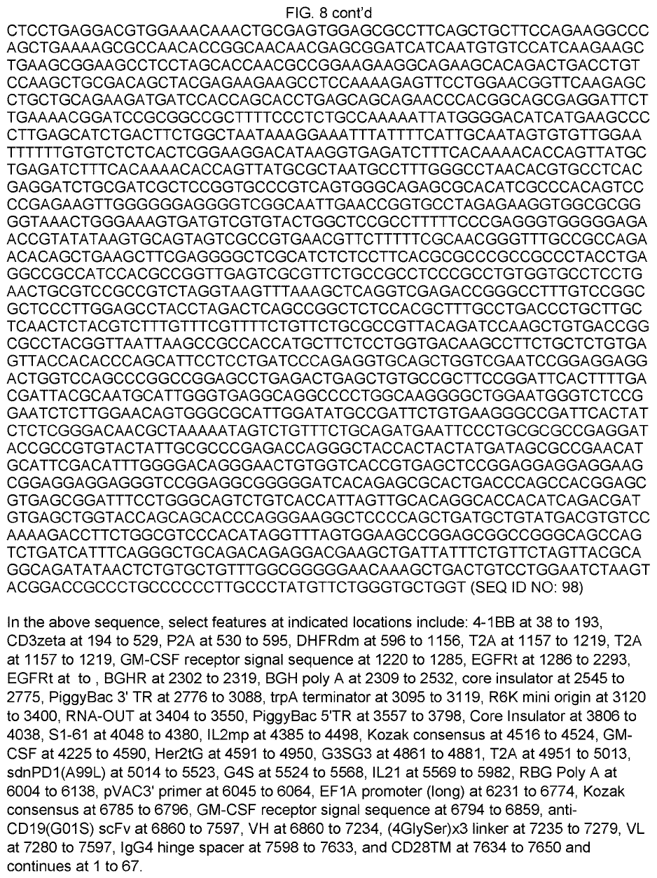

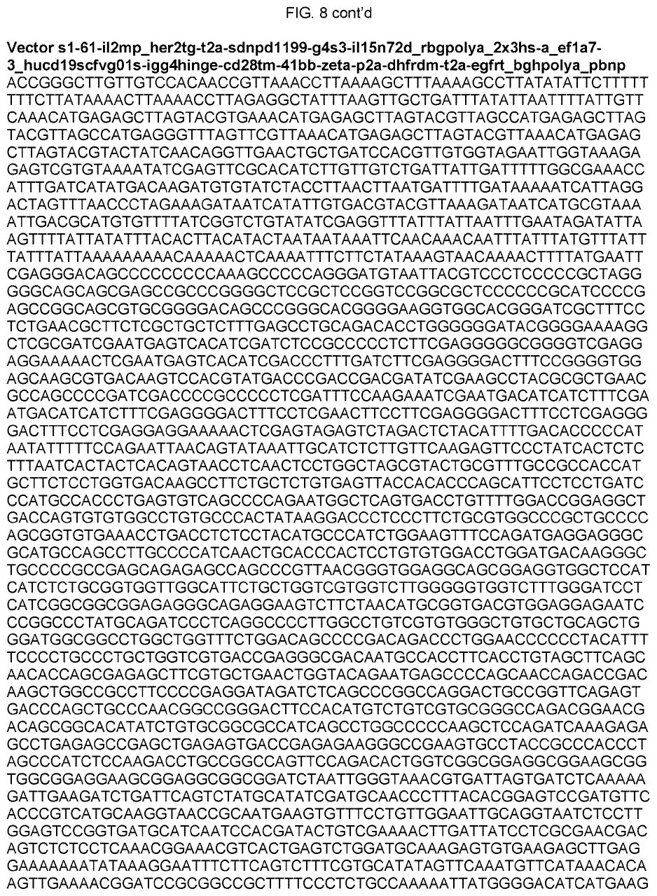

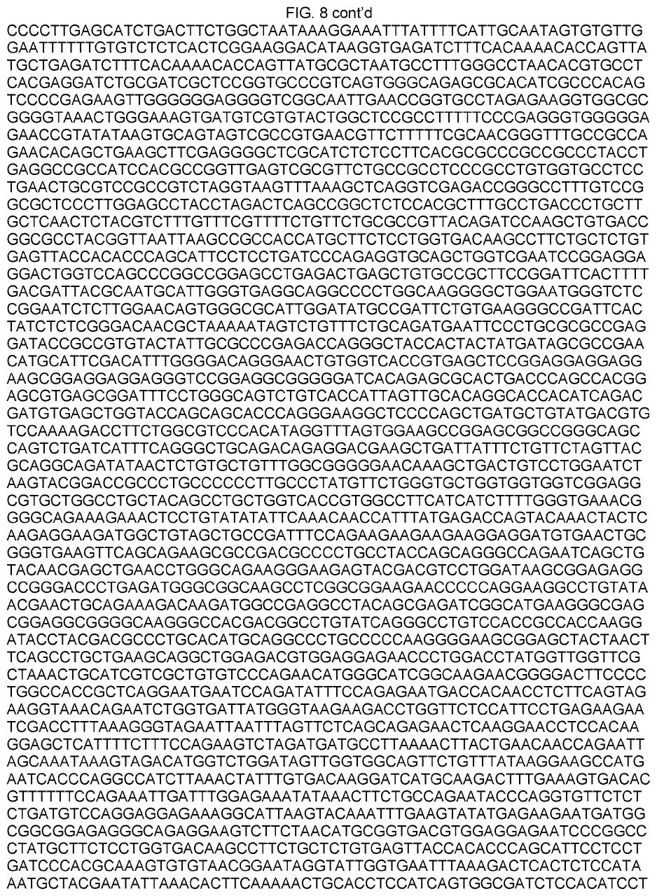

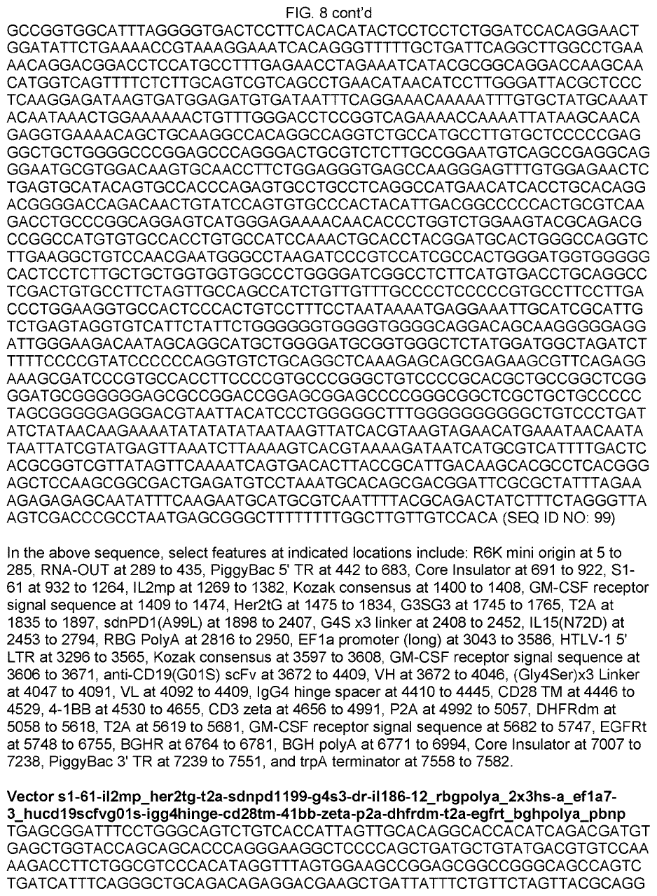

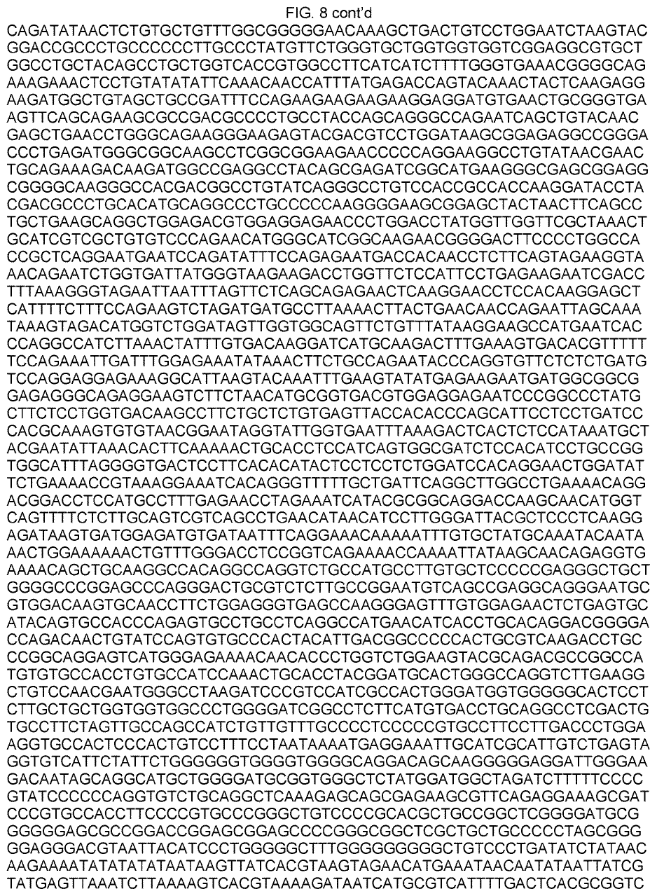

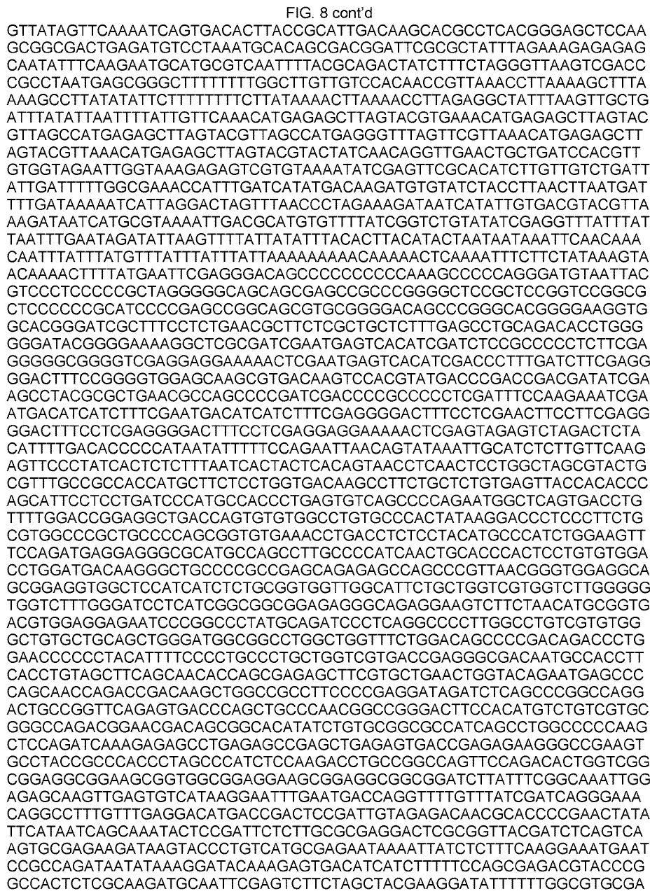

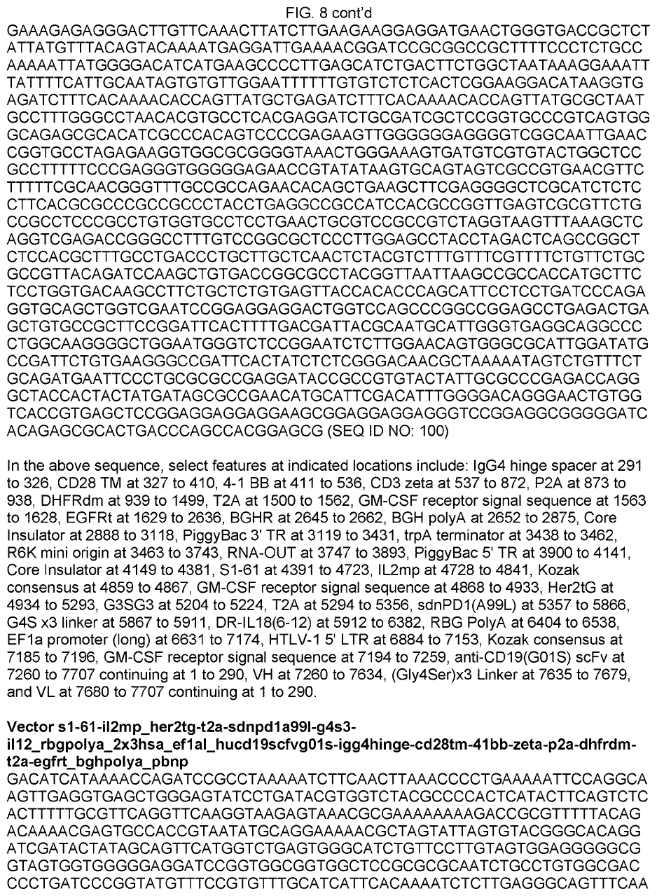

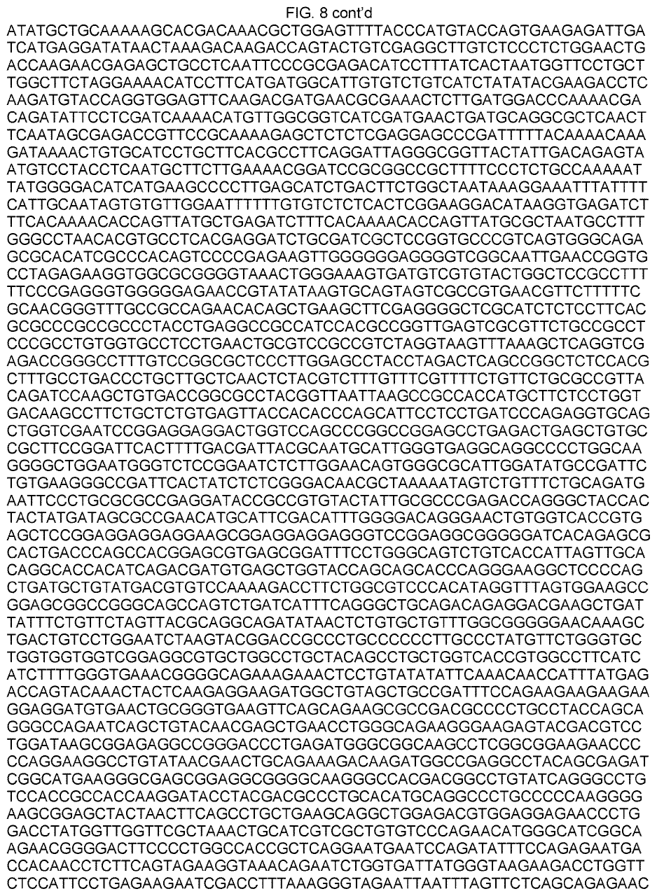

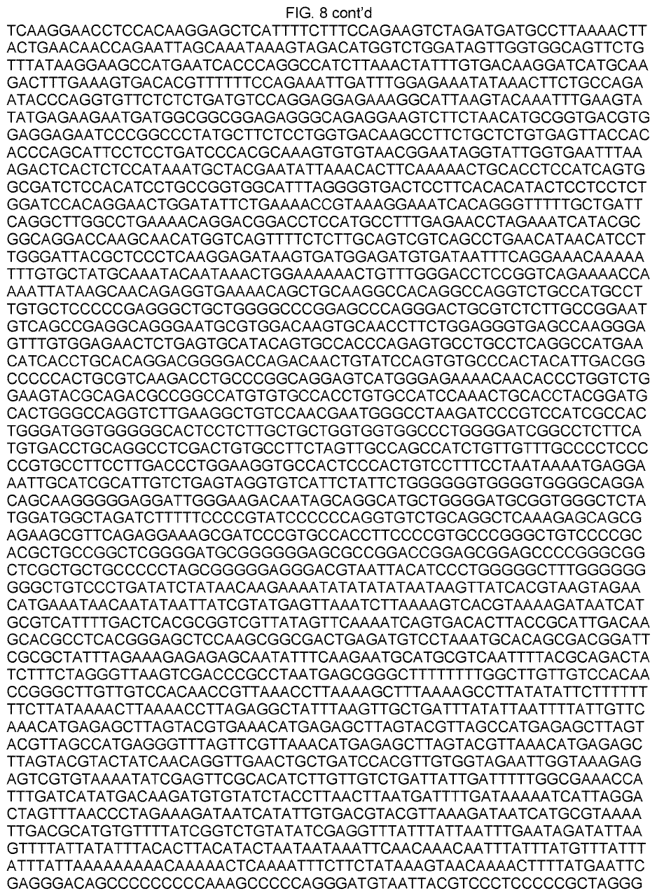

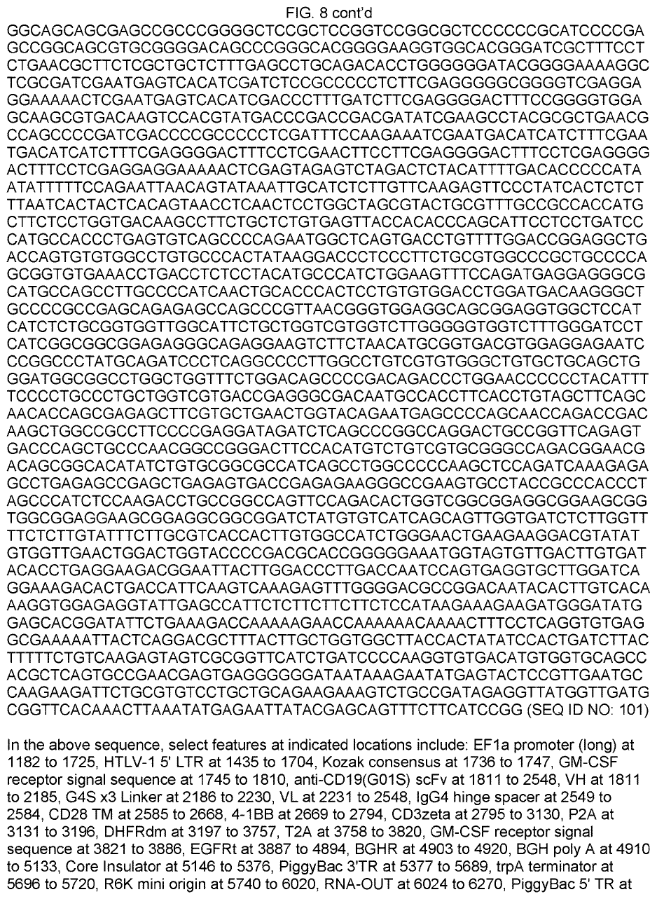

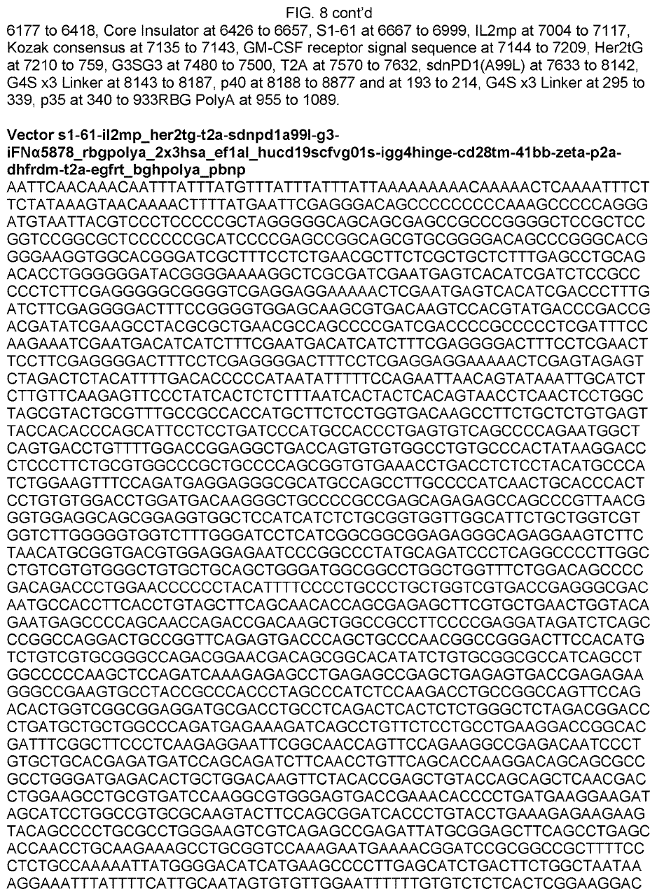

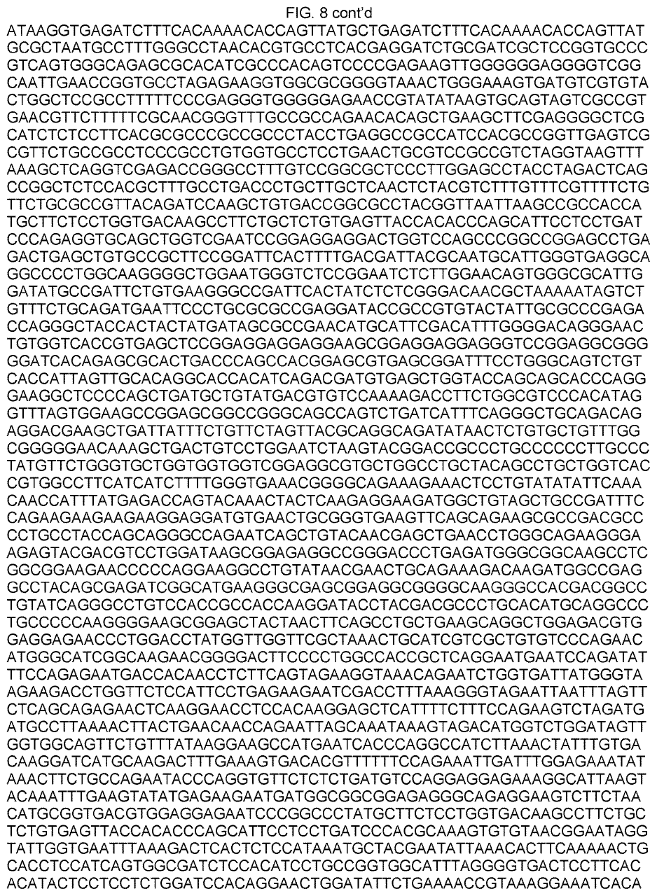

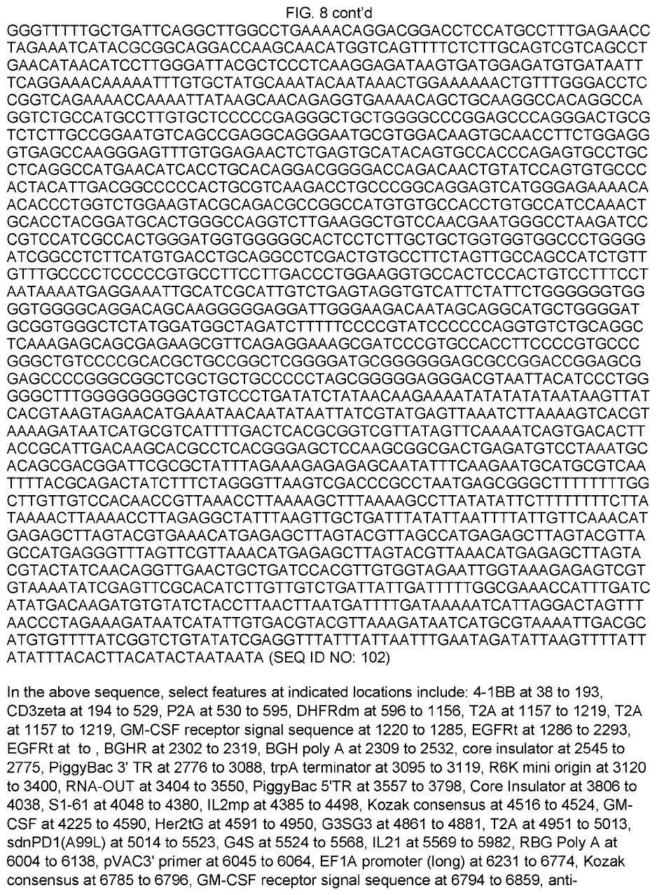

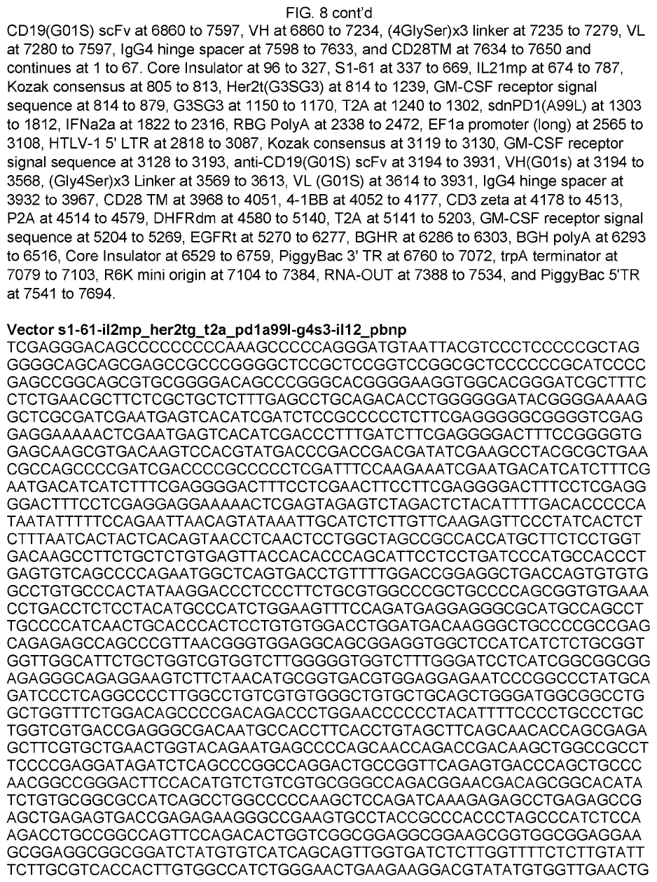

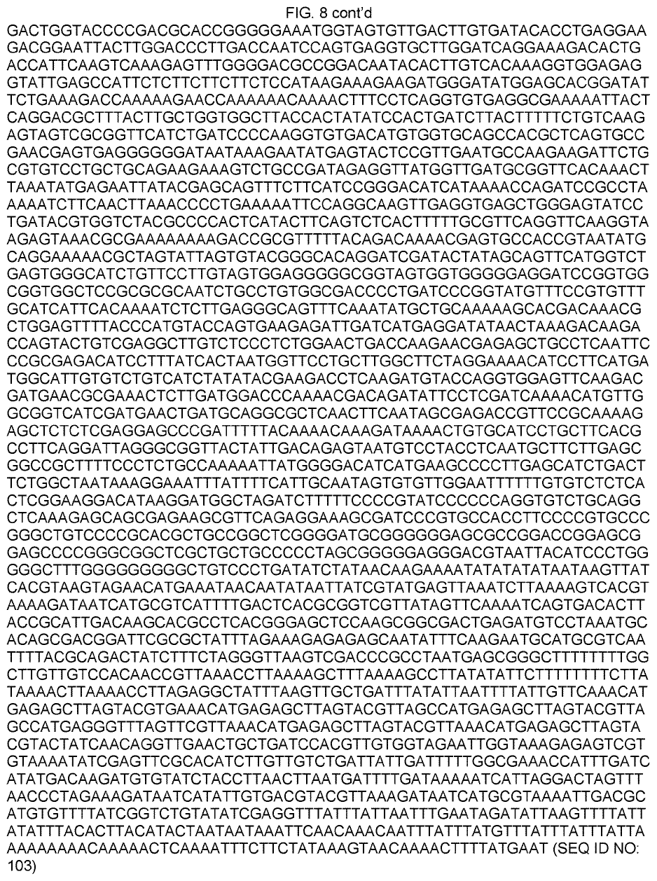

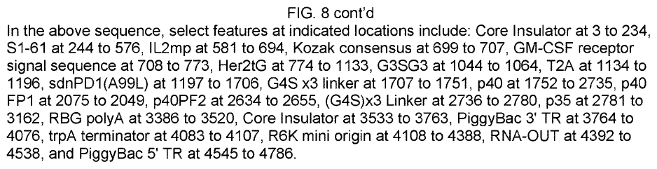

In particular embodiments, the artificial expression construct with the PD1:cytokine chimera transgene and the recombinant receptor includes or encodes an iSynPro promoter, a PD1:cytokine chimera transgene, a first 2A skip sequence, Her2tG, an EF1a(L) promoter, a recombinant receptor, a second 2A skip sequence, a DHFRdm, a third 2A skip sequence, and an EGFRt. In particular embodiments, the artificial expression construct with the PD1:cytokine chimera transgene and the recombinant receptor includes or encodes a S1-61 iSynPro promoter, a PD1:cytokine chimera transgene, a first 2A skip sequence, Her2tG, an EF1a(L) promoter, a CAR, a second 2A skip sequence, a DHFRdm, a third 2A skip sequence, and an EGFRt. In particular embodiments, the PD1:cytokine chimera transgene encodes a soluble PD1:cytokine chimera. In particular embodiments, the PD1:cytokine chimera transgene encodes PD1:IL21, PD1:IL15(N72D), PD1:drIL18, PD1:IL12, or PD1:IFNα. In particular embodiments, the CAR is an anti-CD19 CAR. See FIG. 2 for schematic of exemplary artificial expression constructs.

Thus, the current disclosure provides systems and methods to improve the killing, proliferation, and/or cytokine output of recombinant receptor-expressing immune cells. The recombinant receptor-immune cells can be engineered to target different target cells (e.g., cancer cell, virus, bacteria, fungi, parasites, or arthropods) expressing a target antigen by engineering the extracellular domain to include a binding domain that binds the target antigen.

The various constructs as described herein may be used to tailor a treatment to a specific patient or group of patients. For example, while not wishing to be bound, cytokine release syndrome generally develops when an immune system responds too aggressively. In some instances, it may therefore be helpful to administer cells treated with artificial expression constructs that generate a decreased response in comparison to existing CAR-T cell therapeutics such as CD19CAR, or that have some effect, but are not the best transcription factor transgene at potentiating the killing, proliferation, and cytokine output of recombinant receptor-immune cells.

That is, while there is an increased response over native cells, it may be a more modulated response. In other aspects, it may be helpful to administer a series or combination of cells treated with a combination of artificial expression constructs to create a graded or tiered response. For example, the stimulation dose or the number of doses during T-cell activation may be modified depending on the needs of the patient or group of patients. In other aspects more than one transcription factor transgene may be used.

Aspects of the current disclosure are now described in more supporting detail as follows: (i) Inducible Expression of PD1:Cytokine Chimera Transgenes; (ii) Immune Cells; (iii) Cell Sample Collection and Cell Enrichment; (iv) Genetic Engineering Techniques; (v) Control Features Including Tag Cassettes, Transduction Markers, Selection Cassettes, and/or Suicide Switches; (vi) Recombinant Receptors; (vi-a) Binding Domains; (vi-b) Transmembrane Domains; (vi-c) Intracellular Effector Domains; (vi-d) Linkers; (vii) Ex Vivo Manufactured Cell Formulations; (viii) Compositions for Targeted Viral Vectors & Nanoparticles for In Vivo Cell Modification; (ix) Methods of Use; (x) Kits; (xi) Exemplary Embodiments; and (xii) Closing Paragraphs. These headings are provided for organizational purposes only and do not limit the scope or interpretation of the disclosure.

### (i) Inducible Expression of Pd1:Cytokine Chimera Transgenes

In particular embodiments, a PD1:cytokine chimera includes PD1 wildtype or PD1(A99L) linked to an immunostimulatory cytokine through a linker. PD1(A99L) is a programmed cell death-1 protein having an A99L mutation. The A99L mutation in PD-1 has been shown to result in higher binding to the PD-1 receptor (Lázár-Molner et al., Proc Natl Acad Sci USA. 105(30):10483-10488 (2008)).

Cytokines are small proteins (generally 5-25 kDa) that are important in cell signaling. Cytokines are released by cells and affect the behavior of other cells, and sometimes the releasing cell itself, such as a T-cell. Cytokines can include, for example, chemokines, interferons, interleukins, lymphokines, and/or tumor necrosis factor. Cytokines can be produced by a broad range of cells, which can include, for example, immune cells like macrophages, B lymphocytes, T lymphocytes, and/or mast cells, as well as, endothelial cells, fibroblasts, and/or various stromal cells.

Cytokines can act through receptors and are important in the immune system as the cytokines can modulate the balance between humoral and cell-based immune responses, and can regulate the maturation, growth, and responsiveness of particular cell populations. Some cytokines enhance or inhibit the action of other cytokines in complex ways.

Soluble cytokines are completely translocated across the endoplasmic reticulum membrane and can be secreted from the cell that produced them. Soluble cytokines regulate inflammatory and immune events by functioning as agonists or antagonists of cytokine signaling.

Interleukin 21 (IL21) is a class I cytokine with a four-alpha-helix bundle. It has broad pleiotropic effects on both innate and adaptive immune responses. IL21 is produced by natural killer T cells, CD4(+) T cells, and Th17 cells. IL21 has roles in antitumor, antiviral, and inflammatory responses and in autoimmune and inflammatory diseases (Spolski et al., Nat Rev Drug Discov 13, 379-395 (2014)). PD1(A99L):IL21 is a PD1:cytokine chimera including a PD1(A99L) linked to IL21 with a linker.

Interleukin 15 (IL15) is an inflammatory cytokine with structural similarity to IL2. IL15 is secreted by mononuclear phagocytes following infection by a virus. Diseases associated with IL15 include Severe Combined Immunodeficiency, X-Linked and Primary Cutaneous T-Cell Non-Hodgkin Lymphoma. Among its related pathways are MIF Mediated Glucocorticoid Regulation and the TGF-Beta Pathway. IL15(N72D) is an IL15 molecule with an asparagine to aspartic acid substitution at amino acid 72. This mutation provides a 4-5 fold increase in biological activity compared to the native molecule. PD1(A99L):IL15 is a PD1:cytokine chimera including a PD1(A99L) linked to IL15 with a linker.

Decoy resistant (DR)-interleukin 18 (IL18) (DR-IL18 or drIL18) is a modified IL18 protein wherein the IL18 binds the IL18 receptor, IL18Ra and maintains IL18 function but evades IL-18 binding protein (IL-18BP) (Zhou et al., Nature 583(7817):609-614 (2020)). In other words, DR-IL18 maintains signaling potential but is impervious to inhibition by IL-18BP. IL-18 is a potent proinflammatory cytokine that induces interferon-gamma (IFN-gamma) production from Th1 cells, NK cells and activated macrophages, particularly in the presence of IL-12. IL-18 also functions in developmental regulation of T-lymphocyte helper type I cells and in Fas-mediated cytotoxicity.

Suppression of IL-18 activity is being investigated for treatment of chronic inflammatory diseases such as Crohn's disease and rheumatoid arthritis. It acts by inducing heterodimerization of the two subunits of its receptor, IL-18Ra and IL-18Rs. PD1(A99L):drIL18 is a PD1:cytokine chimera including a PD1(A99L) linked to drIL18 with a linker.

IL12 is an interleukin that is naturally produced by dendritic cells, macrophages, neutrophils, and human B-lymphoblastoid cells (NC-37) in response to antigenic stimulation. IL-12 is composed of a bundle of four alpha helices. PD1(A99L):IL12 is a PD1:cytokine chimera including a PD1(A99L) linked to IL12 with a linker.

Interferon alpha (IFNα) is a cytokine produced by cells of the innate immune system in response to viral infection and other environmental stressors. IFNα is a type I interferon which are known for modulating innate and adaptive immunity. PD1(A99L):IFNα is a PD1:cytokine chimera including a PD1(A99L) linked to interferon a (IFNα) with a linker.

PD1:cytokine chimera transgenes are placed under the control of an inducible synthetic promoter (iSynPro). The iSynPro promoter can have or be S1-17 (SEQ ID NO: 19), S1-37 (SEQ ID NO: 20), S1-4 (SEQ ID NO: 21), S1-1 (SEQ ID NO: 22), S1-3 (SEQ ID NO: 23), S1-42 (SEQ ID NO: 24), S1-61 version 1 (SEQ ID NO: 17), S1-61 version 2 (SEQ ID NO: 18), S1-62 (SEQ ID NO: 25), S1-15 (SEQ ID NO: 26), S1-2 (SEQ ID NO: 27), S1-27 (SEQ ID NO: 28), S1-8 (SEQ ID NO: 29), S1-30 (SEQ ID NO: 30), S1-33 (SEQ ID NO: 31), S1-41 (SEQ ID NO: 32), S1-59 (SEQ ID NO: 33), S1-66 (SEQ ID NO: 34), S1-71 (SEQ ID NO: 35), S1-56 (SEQ ID NO: 36), S1-6 (SEQ ID NO: 37), S1-60 (SEQ ID NO: 38), S1-86 (SEQ ID NO: 39), S1-32 (SEQ ID NO: 40), S1-10 (SEQ ID NO: 41), S1-18 (SEQ ID NO: 42), S1-14 (SEQ ID NO: 43), S1-16 (SEQ ID NO: 44), S1-19 (SEQ ID NO: 45), S1-26 (SEQ ID NO: 46), S1-65 (SEQ ID NO: 47), S2-n1 (SEQ ID NO: 48), S4-n1 (SEQ ID NO: 49), S6-n1 (SEQ ID NO: 50), S1-325 (SEQ ID NO: 51), S1-60 (SEQ ID NO: 52), S2-274 (SEQ ID NO: 53), S2-310 (SEQ ID NO: 54), S1-367 (SEQ ID NO: 55), or S1-7 (SEQ ID NO: 56). In particular embodiments, the iSynPro includes S1-61 (SEQ ID NO: 17 or SEQ ID NO: 18). In particular embodiments, the iSynPro promoter has at least 90%, 91%, 92%, 93%, 94%, 95%, 96%, 97%, 98%, or 99%, sequence identity to SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, SEQ ID NO: 30, SEQ ID NO: 31, SEQ ID NO: 32, SEQ ID NO: 33, SEQ ID NO: 34, SEQ ID NO: 35, SEQ ID NO: 36, SEQ ID NO: 37, SEQ ID NO: 38, SEQ ID NO: 39, SEQ ID NO: 40, SEQ ID NO: 41, SEQ ID NO: 42, SEQ ID NO: 43, SEQ ID NO: 44, SEQ ID NO: 45, SEQ ID NO: 46, SEQ ID NO: 47, SEQ ID NO: 48, SEQ ID NO: 49, SEQ ID NO: 50, SEQ ID NO: 51, SEQ ID NO: 52, SEQ ID NO: 53, SEQ ID NO: 54, SEQ ID NO: 55, or SEQ ID NO: 56.

In particular embodiments, the iSynPro includes S1-61 (SEQ ID NO: 17 or SEQ ID NO: 18). In particular embodiments, the iSynPro has at least 90%, 91%, 92%, 93%, 94%, 95%, 96%, 97%, 98%, or 99%, sequence identity to SEQ ID NO: 17 or SEQ ID NO: 18.

Particular embodiments combine one of the iSynPro sequences described above with a minimal promoter. In particular embodiments, the minimal promoter includes an IL2 minimal promoter. In particular embodiments, the IL2 minimal promoter includes the sequence

(SEQ ID NO: 138)
ACATTTTGACACCCCCATAATATTTTTCCAGAATTAACAGTATAAATTG
CATCTCTTGTTCAAGAGTTCCCTATCACTCTCTTTAATCACTACTCACA
GTAACCTCAACTCCTG.

Functional truncations and variants of the PD1:cytokine chimera transgenes may also be used. Functional truncations retain PD1:cytokine chimera activity while having fewer amino acid residues than a corresponding reference wild-type sequence. Functional variants retain PD1:cytokine chimera activity while having one or more amino acid mutations as compared to a corresponding reference wild-type sequence.

### (ii) IMMUNE CELLS

The present disclosure describes an immune cell genetically modified to inducibly express a PD1:cytokine chimera transgene (e.g., soluble PD1:cytokine chimera) that improves the function of an immune cell. In particular embodiments, the immune cell is also genetically modified to express a recombinant receptor, such as a CAR. The immune cell can be any cell in which the inducible promoter (e.g., iSynPro) can inducibly control expression of the PD1:cytokine chimera transgene. In particular embodiments, immune cells can include lymphocytes, monocytes/macrophages, hematopoietic stem cells (HSCs), hematopoietic progenitor cells (HPC), and/or a mixture of HSC and HPC (i.e., HSPC). In particular embodiments, immune cells include lymphocytes. In particular embodiments, lymphocytes include T-cells, B cells, natural killer (NK) cells, or NK-T cells.

Several different subsets of T-cells have been discovered, each with a distinct function. For example, a majority of T-cells have a T-cell receptor (TCR) existing as a complex of several proteins. The actual T-cell receptor is composed of two separate peptide chains, which are produced from the independent T-cell receptor alpha and beta (TCRα and TCRβ) genes and are called α- and β-TCR chains.

γδ T-cells represent a small subset of T-cells that possess a distinct T-cell receptor (TCR) on their surface. In γδ T-cells, the TCR is made up of one γ-chain and one α-chain. This group of T-cells is much less common (2% of total T-cells) than the as T-cells.

CD3 is expressed on all mature T cells. Activated T-cells express 4-1BB (CD137), CD69, and CD25. CD5 and transferrin receptor are also expressed on T-cells.

T-cells can further be classified into helper cells (CD4+ T-cells) and cytotoxic T-cells (CTLs, CD8+ T-cells), which include cytolytic T-cells. T helper cells assist other white blood cells in immunologic processes, including maturation of B cells into plasma cells and activation of cytotoxic T-cells and macrophages, among other functions. These cells are also known as CD4+ T-cells because they express the CD4 protein on their surface. Helper T-cells become activated when they are presented with peptide antigens by MHC class II molecules that are expressed on the surface of antigen presenting cells (APCs). Once activated, they divide rapidly and secrete small proteins called cytokines that regulate or assist in the active immune response.

Cytotoxic T-cells destroy virally infected cells and tumor cells and are also implicated in transplant rejection. These cells are also known as CD8+ T-cells because they express the CD8 glycoprotein on their surface. These cells recognize their targets by binding to antigen associated with MHC class I, which is present on the surface of nearly every cell of the body.

“Central memory” T-cells (or “TCM”) refers to an antigen experienced CTL that expresses CD62L or CCR7 and CD45RO on the surface thereof and does not express or has decreased expression of CD45RA as compared to naive cells. In particular embodiments, central memory cells are positive for expression of CD62L, CCR7, CD25, CD127, CD45RO, and CD95, and have decreased expression of CD45RA as compared to naive cells.

“Effector memory” T-cell (or “TEM”) refers to an antigen experienced T-cell that does not express or has decreased expression of CD62L on the surface thereof as compared to central memory cells and does not express or has decreased expression of CD45RA as compared to a naive cell. In particular embodiments, effector memory cells are negative for expression of CD62L and CCR7, compared to naive cells or central memory cells, and have variable expression of CD28 and CD45RA. Effector T-cells are positive for granzyme B and perforin as compared to memory or naive T-cells.

“Naive” T-cells refers to a non-antigen experienced T cell that expresses CD62L and CD45RA and does not express CD45RO as compared to central or effector memory cells. In particular embodiments, naive CD8+ T lymphocytes are characterized by the expression of phenotypic markers of naive T-cells including CD62L, CCR7, CD28, CD127, and CD45RA.

Natural killer cells (also known as NK cells, K cells, and killer cells) are activated in response to interferons or macrophage-derived cytokines. They serve to contain viral infections while the adaptive immune response is generating antigen-specific cytotoxic T cells that can clear the infection. NK cells express CD8, CD16 and CD56 but do not express CD3.

NK cells include NK-T cells. NK-T cells are a specialized population of T cells that express a semi invariant T cell receptor (TCR ab) and surface antigens typically associated with natural killer cells. NK-T cells contribute to antibacterial and antiviral immune responses and promote tumor-related immunosurveillance or immunosuppression. Like natural killer cells, NK-T cells can also induce perforin-, Fas-, and TNF-related cytotoxicity. Activated NK-T cells are capable of producing IFN-γ and IL-4. In particular embodiments, NK-T cells are CD3+/CD56+.

Macrophages (and their precursors, monocytes) reside in every tissue of the body (in certain instances as microglia, Kupffer cells and osteoclasts) where they engulf apoptotic cells, pathogens and other non-self-components. Monocytes/macrophages express CD11b, F4/80; CD68; CD11c; IL-4Ra; and/or CD163.

Immature dendritic cells (i.e., pre-activation) engulf antigens and other non-self-components in the periphery and subsequently, in activated form, migrate to T-cell areas of lymphoid tissues where they provide antigen presentation to T cells. Dendritic cells express CD1a, CD1b, CD1c, CD1d, CD21, CD35, CD39, CD40, CD86, CD101, CD148, CD209, and DEC-205.

Hematopoietic Stem/Progenitor Cells or HSPC refer to a combination of hematopoietic stem cells and hematopoietic progenitor cells.

Hematopoietic stem cells refer to undifferentiated hematopoietic cells that are capable of self-renewal either in vivo, essentially unlimited propagation in vitro, and capable of differentiation to all other hematopoietic cell types.

A hematopoietic progenitor cell is a cell derived from hematopoietic stem cells or fetal tissue that is capable of further differentiation into mature cell types. In certain embodiments, hematopoietic progenitor cells are CD24^{lo} Lin⁻ CD117⁺ hematopoietic progenitor cells. HPC can differentiate into (i) myeloid progenitor cells which ultimately give rise to monocytes and macrophages, neutrophils, basophils, eosinophils, erythrocytes, megakaryocytes/platelets, or dendritic cells; or (ii) lymphoid progenitor cells which ultimately give rise to T-cells, B-cells, and NK-cells.

HSPC can be positive for a specific marker expressed in increased levels on HSPC relative to other types of hematopoietic cells. For example, such markers include CD34, CD43, CD45RO, CD45RA, CD59, CD90, CD109, CD117, CD133, CD166, HLA DR, or a combination thereof. Also, the HSPC can be negative for an expressed marker relative to other types of hematopoietic cells. For example, such markers include Lin, CD38, or a combination thereof. Preferably, the HSPC are CD34⁺ cells.

A statement that a cell or population of cells is “positive” for or expressing a particular marker refers to the detectable presence on or in the cell of the particular marker. When referring to a surface marker, the term can refer to the presence of surface expression as detected by flow cytometry, for example, by staining with an antibody that specifically binds to the marker and detecting said antibody, wherein the staining is detectable by flow cytometry at a level substantially above the staining detected carrying out the same procedure with an isotype-matched control under otherwise identical conditions and/or at a level substantially similar to that for cell known to be positive for the marker, and/or at a level substantially higher than that for a cell known to be negative for the marker.

A statement that a cell or population of cells is “negative” for a particular marker or lacks expression of a marker refers to the absence of substantial detectable presence on or in the cell of a particular marker. When referring to a surface marker, the term can refer to the absence of surface expression as detected by flow cytometry, for example, by staining with an antibody that specifically binds to the marker and detecting said antibody, wherein the staining is not detected by flow cytometry at a level substantially above the staining detected carrying out the same procedure with an isotype-matched control under otherwise identical conditions, and/or at a level substantially lower than that for cell known to be positive for the marker, and/or at a level substantially similar as compared to that for a cell known to be negative for the marker.

Cells to be genetically modified according to the teachings of the current disclosure can be patient-derived cells (autologous) or allogeneic when appropriate, and can also be in vivo or ex vivo. In particular embodiments, immune cell is a lymphocyte. In particular embodiments, the lymphocyte is a T cell, B cell or NK cell. In particular embodiments, the T cell is a CD4+ T cell or CD8+ T cell.

### (iii) CELL SAMPLE COLLECTION AND CELL ENRICHMENT

Methods of sample collection and enrichment are known by those skilled in the art. In particular embodiments, cells are derived from humans, for example a patient to be treated. Cells can be derived from cell lines. The cells in some embodiments are obtained from a xenogeneic source, for example, from mouse, rat, non-human primate, or pig.

In some embodiments, T cells are derived or isolated from samples such as whole blood, peripheral blood mononuclear cells (PBMCs), leukocytes, bone marrow, thymus, tissue biopsy, tumor, lymph node, gut associated lymphoid tissue, mucosa associated lymphoid tissue, spleen, other lymphoid tissues, liver, lung, stomach, intestine, colon, kidney, pancreas, breast, bone, prostate, cervix, testes, ovaries, tonsil, or other organ, and/or cells derived therefrom. In particular embodiments, cells from the circulating blood of a subject are obtained, e.g., by apheresis or leukapheresis. The samples, in particular embodiments, contain lymphocytes, including T cells, monocytes, granulocytes, B cells, other nucleated white blood cells, HSC, HPC, HSPC, red blood cells, and/or platelets, and in some aspects contains cells other than red blood cells and platelets and further processing is necessary.

In some embodiments, blood cells collected from a subject are washed, e.g., to remove the plasma fraction and to place the cells in an appropriate buffer or media for subsequent processing steps. In particular embodiments, the cells are washed with phosphate buffered saline (PBS). In some embodiments, the wash solution lacks calcium and/or magnesium and/or many or all divalent cations. Washing can be accomplished using a semi-automated “flow-through” centrifuge (for example, the Cobe 2991 cell processor, Baxter) according to the manufacturer's instructions. Tangential flow filtration (TFF) can also be performed. In particular embodiments, cells can be re-suspended in a variety of biocompatible buffers after washing, such as, Ca++/Mg++ free PBS.

The isolation can include one or more of various cell preparation and separation steps, including separation based on one or more properties, such as size, density, sensitivity or resistance to particular reagents, and/or affinity, e.g., immunoaffinity, to antibodies or other binding partners. In particular embodiments, the isolation is carried out using the same apparatus or equipment sequentially in a single process stream and/or simultaneously. In particular embodiments, the isolation, culture, and/or engineering of the different populations is carried out from the same starting material, such as from the same sample.

In particular embodiments, a sample can be enriched for T cells by using density-based cell separation methods and related methods. For example, white blood cells can be separated from other cell types in the peripheral blood by lysing red blood cells and centrifuging the sample through a Percoll or Ficoll gradient.

In particular embodiments, a bulk T cell population can be used that has not been enriched for a particular T cell type. In particular embodiments, a selected T cell type can be enriched for and/or isolated based on cell-marker based positive and/or negative selection. In positive selection, cells having bound cellular markers are retained for further use. In negative selection, cells not bound by a capture agent, such as an antibody to a cellular marker are retained for further use. In some examples, both fractions can be retained for a further use. In particular embodiments, CD4+ and/or CD8+ T cells are enriched from PBMCs.

The separation need not result in 100% enrichment or removal of a particular cell population or cells expressing a particular marker. For example, positive selection of or enrichment for cells of a particular type refers to increasing the number or percentage of such cells but need not result in a complete absence of cells not expressing the marker. Likewise, negative selection, removal, or depletion of cells of a particular type refers to decreasing the number or percentage of such cells but need not result in a complete removal of all such cells.

In some examples, multiple rounds of separation steps are carried out, where the positively or negatively selected fraction from one step is subjected to another separation step, such as a subsequent positive or negative selection.

In some embodiments, an antibody or binding domain for a cellular marker is bound to a solid support or matrix, such as a magnetic bead or paramagnetic bead, to allow for separation of cells for positive and/or negative selection. For example, in some embodiments, the cells and cell populations are separated or isolated using immunomagnetic (or affinity magnetic) separation techniques (reviewed in Methods in Molecular Medicine, vol. 58: Metastasis Research Protocols, Vol. 2: Cell Behavior/n Vitro and/n Vivo, p 17-25 Edited by: S. A. Brooks and U. Schumacher© Humana Press Inc., Totowa, NJ); see also U.S. Pat. Nos. 4,452,773; 4,795,698; 5,200,084; and EP 452342.

In some embodiments, affinity-based selection is via magnetic-activated cell sorting (MACS) (Miltenyi Biotec, Auburn, CA). MACS systems are capable of high-purity selection of cells having magnetized particles attached thereto. In certain embodiments, MACS operates in a mode wherein the non-target and target species are sequentially eluted after the application of the external magnetic field. That is, the cells attached to magnetized particles are held in place while the unattached species are eluted. Then, after this first elution step is completed, the species that were trapped in the magnetic field and were prevented from being eluted are freed in some manner such that they can be eluted and recovered. In certain embodiments, the non-target cells are labelled and depleted from the heterogeneous population of cells.

In some embodiments, a cell population described herein is collected and enriched (or depleted) via flow cytometry, in which cells stained for multiple cell surface markers are carried in a fluidic stream. In some embodiments, a cell population described herein is collected and enriched (or depleted) via preparative scale (FACS)-sorting. In certain embodiments, a cell population described herein is collected and enriched (or depleted) by use of microelectromechanical systems (MEMS) chips in combination with a FACS-based detection system (see, e.g., WO 2010/033140, Cho et al. (2010) Lab Chip 10, 1567-1573; and Godin et al. (2008) J Biophoton. 1(5):355-376). In both cases, cells can be labeled with multiple markers, allowing for the isolation of well-defined cell subsets at high purity.

Cell-markers for different T cell subpopulations are described above. In particular embodiments, specific subpopulations of T cells, such as cells positive or expressing high levels of one or more surface markers, e.g., CCR7, CD45RO, CD8, CD27, CD28, CD62L, CD127, CD4, and/or CD45RA T cells, are isolated by positive or negative selection techniques.

CD3+, CD28+ T cells can be positively selected for and expanded using anti-CD3/anti-CD28 conjugated magnetic beads (e.g., DYNABEADS® (Life Technologies AS, Norway) M-450 CD3/CD28 T Cell Expander).

In particular embodiments, a CD8+ or CD4+ selection step is used to separate CD4+helper and CD8+ cytotoxic T cells. Such CD8+ and CD4+ populations can be further sorted into sub-populations by positive or negative selection for markers expressed or expressed to a relatively higher degree on one or more naive, memory, and/or effector T cell subpopulations.

In particular embodiments, a CD8+ and/or CD4+ selection step is used to separate CD4+ helper and CD8+ cytotoxic T cells from a negative fraction. Particular embodiments utilize the TICLE method as described in more detail elsewhere herein.

Other cell types can be enriched based on known marker profiles and techniques. For example, CD34+ HSC, HSP, and HSPC can be enriched using anti-CD34 antibodies directly or indirectly conjugated to magnetic particles in connection with a magnetic cell separator, for example, the CliniMACS® Cell Separation System (Miltenyi Biotec, Bergisch Gladbach, Germany).

### (iv) GENETIC ENGINEERING TECHNIQUES

Cell populations are genetically modified (or genetically engineered) to express a PD1:cytokine chimera and, optionally a recombinant receptor (e.g., chimeric antigen receptors, CAR) described herein. Desired genes (e.g., soluble PD1:cytokine chimera transgenes and recombinant receptors) disclosed herein can be introduced into cells by any method known in the art, including transfection, electroporation, microinjection, lipofection, calcium phosphate mediated transfection, infection with a viral or bacteriophage vector including the gene sequences, cell fusion, chromosome-mediated gene transfer, microcell-mediated gene transfer, spheroplast fusion, in vivo nanoparticle-mediated delivery, etc. Numerous techniques are known in the art for the introduction of foreign genes into cells (see e.g., Loeffler and Behr, 1993, Meth. Enzymol. 217:599-618; Cohen, et al., 1993, Meth. Enzymol. 217:618-644; Cline, 1985, Pharmac. Ther. 29:69-92) and may be used, provided that the necessary developmental and physiological functions of the recipient cells are not unduly disrupted. The technique can provide for the stable transfer of the gene to the cell, so that the gene is expressible by the cell and, in certain instances, preferably heritable and expressible by its cell progeny.

The term “gene” refers to a nucleic acid sequence (used interchangeably with polynucleotide or nucleotide sequence). The gene can include the PD1:cytokine chimera transgene or encode a recombinant receptor. This definition includes various sequence polymorphisms, mutations, and/or sequence variants wherein such alterations do not substantially affect the function of the PD1:cytokine chimera transgene or encoded recombinant receptor. The term “gene” may include not only coding sequences but also regulatory regions such as promoters, enhancers, and termination regions. The term further can include all introns and other DNA sequences spliced from an mRNA transcript, along with variants resulting from alternative splice sites. Gene sequences encoding the molecule can be DNA or RNA that directs the expression of the open reading frame(s) within the artificial expression construct. These nucleic acid sequences may be a DNA strand sequence that is transcribed into RNA or an RNA sequence that is translated into protein. The nucleic acid sequences include both the full-length nucleic acid sequences as well as non-full-length sequences derived from the full-length protein. The sequences can also include degenerate codons of the native sequence or sequences that may be introduced to provide codon preference in a specific cell type. Portions of complete gene sequences are referenced throughout the disclosure as is understood by one of ordinary skill in the art.

Gene sequences including PD1:cytokine chimera transgenes and/or encoding recombinant receptors are provided herein and can also be readily prepared by synthetic or recombinant methods from the relevant amino acid sequences and other description provided herein. In embodiments, the gene sequence encoding any of these sequences can also have one or more restriction enzyme sites at the 5 and/or 3′ ends of the coding sequence in order to provide for easy excision and replacement of the gene sequence encoding the sequence with another gene sequence encoding a different sequence. In embodiments, the gene sequence encoding the sequences can be codon optimized for expression in mammalian cells.

“Encoding” refers to the property of specific sequences of nucleotides in a gene, such as a cDNA, or an mRNA, to serve as templates for the synthesis of other macromolecules such as a defined sequence of amino acids. Thus, a gene codes for a protein if transcription and translation of mRNA corresponding to that gene produces the protein in a cell or other biological system. A “gene sequence encoding a protein” includes all nucleotide sequences that are degenerate versions of each other and that code for the same amino acid sequence or amino acid sequences of substantially similar form and function.

Polynucleotide gene sequences encoding more than one portion of an expressed artificial expression construct can be operably linked to each other and relevant regulatory sequences. For example, there can be a functional linkage between a regulatory sequence and an exogenous nucleic acid sequence resulting in expression of the latter. For another example, a first nucleic acid sequence can be operably linked with a second nucleic acid sequence when the first nucleic acid sequence is placed in a functional relationship with the second nucleic acid sequence. For instance, a promoter is operably linked to a coding sequence if the promoter affects the transcription or expression of the coding sequence. Generally, operably linked DNA sequences are contiguous and, where necessary or helpful, join coding regions, into the same reading frame.

Promoters can include general promoters, tissue-specific promoters, cell-specific promoters, and/or promoters specific for the cytoplasm. Promoters may include strong promoters, weak promoters, constitutive expression promoters (i.e., constitutive promoters), and/or inducible promoters. Constitutive promoters are promoters that allow for the continual transcription of its associated gene or genes. Inducible promoters direct expression in response to certain conditions, signals or cellular events. For example, the promoter may be an inducible promoter that requires a particular ligand, small molecule, transcription factor or hormone protein in order to effect transcription from the promoter. In particular embodiments, the inducible promoter includes the iSynPro described above and a minimal promoter. In particular embodiments, the minimal promoter includes an IL2 minimal promoter.

Particular examples of constitutive promoters include human elongation factor la promoter (EF1a, including EF1a(s) and EF1a(L)), myeloproliferative sarcoma virus (MND), cytomegalovirus (CMV), simian virus 40 (SV40) early promoter, mouse mammary tumor virus (MMTV), human immunodeficiency virus (HIV) long terminal repeat (LTR) promoter, MoMuLV promoter, an avian leukemia virus promoter, an Epstein-Barr virus immediate early promoter, a Rous sarcoma virus promoter, as well as human gene promoters such as the actin promoter, the myosin promoter, the hemoglobin promoter, and the creatine kinase promoter. In particular embodiments, the recombinant receptor is under the control of EF1a (SEQ ID NO: 57).

A “vector” is a nucleic acid molecule that is capable of transporting another nucleic acid. Vectors may be, e.g., plasmids (DNA plasmids or RNA plasmids), transposon-based systems, cosmids, bacterial artificial chromosomes, viruses, or phage. An “expression vector” is a vector that is capable of directing the expression of a protein encoded by one or more genes carried by the vector when it is present in the appropriate environment.

“Lentivirus” refers to a genus of retroviruses that are capable of infecting dividing and non-dividing cells. Several examples of lentiviruses include HIV (human immunodeficiency virus: including HIV type 1, and HIV type 2); equine infectious anemia virus; feline immunodeficiency virus (FIV); bovine immune deficiency virus (BIV); and simian immunodeficiency virus (SIV).

A lentiviral vector is a vector derived from at least a portion of a lentivirus genome, including especially a self-inactivating lentiviral vector as provided in Milone et al, Mol. Ther. 17(8): 1453-1464 (2009). Other examples of lentivirus vectors that may be used in the clinic, include: the LENTIVECTOR® gene delivery technology from Oxford BioMedica (United Kingdom), the LENTIMAX vector system from Lentigen and the like. Nonclinical types of lentiviral vectors are also available and would be known to one skilled in the art. In particular embodiments, cells are genetically engineered to express artificial expression constructs using a lentivirus or lentiviral vector.

“Retroviruses” are viruses having an RNA genome. “Gammaretrovirus” refers to a genus of the retroviridae family. Exemplary gammaretroviruses include mouse stem cell virus, murine leukemia virus, feline leukemia virus, feline sarcoma virus, and avian reticuloendotheliosis viruses.

Retroviral vectors (see Miller, et al., 1993, Meth. Enzymol. 217:581-599) can be used. In such embodiments, the gene to be expressed is cloned into the retroviral vector for its delivery into cells. In particular embodiments, a retroviral vector includes all of the cis-acting sequences necessary for the packaging and integration of the viral genome, i.e., (a) a long terminal repeat (LTR), or portions thereof, at each end of the vector; (b) primer binding sites for negative and positive strand DNA synthesis; and (c) a packaging signal, necessary for the incorporation of genomic RNA into virions. More detail about retroviral vectors can be found in Boesen, et al., 1994, Biotherapy 6:291-302; Clowes, et al., 1994, J. Clin. Invest. 93:644-651; Kiem, et al., 1994, Blood 83:1467-1473; Salmons and Gunzberg, 1993, Human Gene Therapy 4:129-141; and Grossman and Wilson, 1993, Curr. Opin. in Genetics and Devel. 3:110-114. Adenoviruses, adeno-associated viruses (AAV) and alphaviruses can also be used. See Kozarsky and Wilson, 1993, Current Opinion in Genetics and Development 3:499-503, Rosenfeld, et al., 1991, Science 252:431-434; Rosenfeld, et al., 1992, Cell 68:143-155; Mastrangeli, et al., 1993, J. Clin. Invest. 91:225-234; Walsh, et al., 1993, Proc. Soc. Exp. Bioi. Med. 204:289-300; and Lundstrom, 1999, J. Recept. Signal Transduct. Res. 19: 673-686. Other methods of gene delivery include use of mammalian artificial chromosomes (Vos, 1998, Curr. Op. Genet. Dev. 8:351-359); liposomes (Tarahovsky and Ivanitsky, 1998, Biochemistry (Mosc) 63:607-618); ribozymes (Branch and Klotman, 1998, Exp. Nephrol. 6:78-83); and triplex DNA (Chan and Glazer, 1997, J. Mol. Med. 75:267-282).

There are a large number of available viral vectors suitable within the current disclosure, including those identified for human gene therapy applications (see Pfeifer and Verma, 2001, Ann. Rev. Genomics Hum. Genet. 2:177). Methods of using retroviral and lentiviral viral vectors and packaging cells for transducing mammalian host cells with viral particles including transgenes are described in, e.g., U.S. Pat. No. 8,119,772; Walchli, et al., 2011, PLoS One 6:327930; Zhao, et al., 2005, J. Immunol. 174:4415; Engels, et al., 2003, Hum. Gene Ther. 14:1155; Frecha, et al., 2010, Mol. Ther. 18:1748; and Verhoeyen, et al., 2009, Methods Mol. Biol. 506:97. Retroviral and lentiviral vector constructs and expression systems are also commercially available.

Targeted genetic engineering approaches may also be utilized. The CRISPR (Clustered Regularly Interspaced Short Palindromic Repeats)/Cas (CRISPR-associated protein) nuclease system is an engineered nuclease system used for genetic engineering that is based on a bacterial system. Information regarding CRISPR-Cas systems and components thereof are described in, for example, U.S. Pat. Nos. 8,697,359, 8,771,945, 8,795,965, 8,865,406, 8,871,445, 8,889,356, 8,889,418, 8,895,308, 8,906,616, 8,932,814, 8,945,839, 8,993,233 and 8,999,641 and applications related thereto; and WO2014/018423, WO2014/093595, WO2014/093622, WO2014/093635, WO2014/093655, WO2014/093661, WO2014/093694, WO2014/093701, WO2014/093709, WO2014/093712, WO2014/093718, WO2014/145599, WO2014/204723, WO2014/204724, WO2014/204725, WO2014/204726, WO2014/204727, WO2014/204728, WO2014/204729, WO2015/065964, WO2015/089351, WO2015/089354, WO2015/089364, WO2015/089419, WO2015/089427, WO2015/089462, WO2015/089465, WO2015/089473 and WO2015/089486, WO2016205711, WO2017/106657, WO2017/127807 and applications related thereto.

Particular embodiments utilize zinc finger nucleases (ZFNs) as gene editing agents. ZFNs are a class of site-specific nucleases engineered to bind and cleave DNA at specific positions. For additional information regarding ZFNs and ZFNs useful within the teachings of the current disclosure, see, e.g., U.S. Pat. Nos. 6,534,261; 6,607,882; 6,746,838; 6,794,136; 6,824,978; 6,866,997; 6,933,113; 6,979,539; 7,013,219; 7,030,215; 7,220,719; 7,241,573; 7,241,574; 7,585,849; 7,595,376; 6,903,185; 6,479,626; US 2003/0232410 and US 2009/0203140 as well as Gaj et al., Nat Methods, 2012, 9(8):805-7; Ramirez et al., Nucl Acids Res, 2012, 40(12):5560-8; Kim et al., Genome Res, 2012, 22(7): 1327-33; Urnov et al., Nature Reviews Genetics, 2010, 11:636-646; Miller, et al. Nature biotechnology 25, 778-785 (2007); Bibikova, et al. Science 300, 764 (2003); Bibikova, et al. Genetics 161, 1169-1175 (2002); Wolfe, et al. Annual review of biophysics and biomolecular structure 29, 183-212 (2000); Kim, et al. Proceedings of the National Academy of Sciences of the United States of America 93, 1156-1160 (1996); and Miller, et al. The EMBO journal 4, 1609-1614 (1985).

Particular embodiments can use transcription activator like effector nucleases (TALENs) as gene editing agents. TALENs refer to fusion proteins including a transcription activator-like effector (TALE) DNA binding protein and a DNA cleavage domain. For additional information regarding TALENs, see U.S. Pat. Nos. 8,440,431; 8,440,432; 8,450,471; 8,586,363; and 8,697,853; as well as Joung and Sander, Nat Rev Mol Cell Biol, 2013, 14(1):49-55; Beurdeley et al., Nat Commun, 2013, 4: 1762; Scharenberg et al., Curr Gene Ther, 2013, 13(4):291-303; Gaj et al., Nat Methods, 2012, 9(8):805-7; Miller, et al. Nature biotechnology 29, 143-148 (2011); Christian, et al. Genetics 186, 757-761 (2010); Boch, et al. Science 326, 1509-1512 (2009); and Moscou, & Bogdanove, Science 326, 1501 (2009).

Nanoparticles that result in selective in vivo genetic modification of targeted cell types have been described and can be used within the teachings of the current disclosure. In particular embodiments, the nanoparticles can be those described in WO2014153114, WO2017181110, and WO201822672.

### (v) CONTROL FEATURES INCLUDING TAG CASSETTES, TRANSDUCTION MARKERS, SELECTION CASSETTES, AND/OR SUICIDE SWITCHES

In particular embodiments, artificial expression constructs can include or encode one or more tag cassettes and/or transduction markers. Tag cassettes and transduction markers can be used to activate, promote proliferation of, detect, enrich for, isolate, track, deplete and/or eliminate genetically modified cells in vitro, in vivo and/or ex vivo. “Tag cassette” refers to a unique synthetic peptide sequence affixed to, fused to, or that is part of an artificial expression construct, to which a cognate binding molecule (e.g., ligand, antibody, or other binding partner) is capable of specifically binding where the binding property can be used to activate, promote proliferation of, detect, enrich for, isolate, track, deplete and/or eliminate the tagged protein and/or cells expressing the tagged protein. Transduction markers can serve the same purposes but are derived from naturally occurring molecules and are often expressed using a skipping element (or skip sequence) that separates the transduction marker from the rest of the expressed molecule.

Tag cassettes that bind cognate binding molecules include, for example, His tag (HHHHHH; SEQ ID NO: 104), Flag tag (DYKDDDDK; SEQ ID NO: 105), Xpress tag (DLYDDDDK; SEQ ID NO: 106), Avi tag (GLNDIFEAQKIEWHE; SEQ ID NO: 107), Calmodulin tag (KRRWKKNFIAVSAANRFKKISSSGAL; SEQ ID NO: 108), Polyglutamate tag, HA tag (YPYDVPDYA; SEQ ID NO: 109), Myc tag (EQKLISEEDL; SEQ ID NO: 110), Strep tag (which refers the original STREP® tag (WRHPQFGG; SEQ ID NO: 111), STREP® tag II (WSHPQFEK SEQ ID NO: 112 (IBA Institut fur Bioanalytik, Germany); see, e.g., U.S. Pat. No. 7,981,632), Softag 1 (SLAELLNAGLGGS; SEQ ID NO: 113), Softag 3 (TQDPSRVG; SEQ ID NO: 114), and V5 tag (GKPIPNPLLGLDST; SEQ ID NO: 115).

Conjugate binding molecules that specifically bind tag cassette sequences disclosed herein are commercially available. For example, His tag antibodies are commercially available from suppliers including Life Technologies, Pierce Antibodies, and GenScript. Flag tag antibodies are commercially available from suppliers including Pierce Antibodies, GenScript, and Sigma-Aldrich. Xpress tag antibodies are commercially available from suppliers including Pierce Antibodies, Life Technologies and GenScript. Avi tag antibodies are commercially available from suppliers including Pierce Antibodies, IsBio, and Genecopoeia. Calmodulin tag antibodies are commercially available from suppliers including Santa Cruz Biotechnology, Abcam, and Pierce Antibodies. HA tag antibodies are commercially available from suppliers including Pierce Antibodies, Cell Signal and Abcam. Myc tag antibodies are commercially available from suppliers including Santa Cruz Biotechnology, Abcam, and Cell Signal. Strep tag antibodies are commercially available from suppliers including Abcam, Iba, and Qiagen.

Transduction markers may be selected from at least one of a truncated CD19 (tCD19; see Budde et al., Blood 122: 1660, 2013); a truncated human EGFR (tEGFR or EGFRt; see Wang et al., Blood 118: 1255, 2011); a cell surface-localizing polypeptide tag based on truncated human HER2, designated Her2tG; an ECD of human CD34; and/or RQR8 which combines target epitopes from CD34 (see Fehse et al, Mol. Therapy 1(5 Pt 1); 448-456, 2000) and CD20 antigens (see Philip et al, Blood 124: 1277-1278). In particular embodiments, cells are genetically modified to express EGFRt. In particular embodiments, cells are genetically modified to express Her2tG.

In particular embodiments, a selection cassette provides for positive selection or negative selection of a desired cell population. Negative selection is when several cell types are removed, leaving the cell type of interest. Positive selection involves targeting the desired cell population to only retain desired cells.

A selection cassette can encode proteins that (a) confer resistance to antibiotics or other toxins, (b) complement auxotrophic deficiencies, or (c) supply critical nutrients not available from complex media, e.g., the gene encoding D-alanine racemase for Bacilli. Any number of selection systems may be used to recover transformed cells. In particular embodiments, a positive selection cassette includes resistance genes to neomycin, hygromycin, ampicillin, puromycin, phleomycin, zeomycin, blasticidin, or viomycin. In particular embodiments, a selection cassette includes the DHFR (dihydrofolate reductase) gene or DHFR double mutant (DHFRdm) gene providing resistance to methotrexate (MTX), the MGMT P140K gene responsible for the resistance to O⁶BG/BCNU, the HPRT (Hypoxanthine phosphoribosyl transferase) gene responsible for the transformation of specific bases present in the HAT selection medium (aminopterin, hypoxanthine, thymidine) or other genes for detoxification with respect to some drugs. In particular embodiments, the selection agent includes neomycin, hygromycin, puromycin, phleomycin, zeomycin, blasticidin, viomycin, ampicillin, O⁶BG/BCNU, MTX, tetracycline, aminopterin, hypoxanthine, thymidine kinase, DHFR, Gln synthetase, or ADA.

In particular embodiments, the selection cassette includes DHFRdm and the selection agent includes MTX. In particular embodiments, the method does not require a selection cassette to acquire highly purified cell populations.

In particular embodiments, negative selection cassettes include a gene for transformation of a substrate present in the culture medium into a toxic substance for the cell that expresses the gene. These molecules include detoxification genes of diptheria toxin (DTA) (Yagi et al., Anal Biochem. 214(1):77-86, 1993; Yanagawa et al., Transgenic Res. 8(3):215-221, 1999), the kinase thymidine gene of the Herpes virus (HSV TK) sensitive to the presence of ganciclovir or FIAU. The HPRT gene may also be used as a negative selection by addition of 6-thioguanine (6TG) into the medium. and for all positive and negative selections, a poly A transcription termination sequence from different origins, the most classical being derived from SV40 poly A, or a eukaryotic gene poly A (bovine growth hormone, rabbit p-globin, etc.).

In particular embodiments, artificial expression constructs can include a polynucleotide or skip sequence that encodes a self-cleaving polypeptide. In particular embodiments, the polynucleotide encoding the self-cleaving polypeptide is located between the PD1:cytokine chimera transgene and the polynucleotide encoding the control feature. In particular embodiments, the polynucleotide encoding the self-cleaving polypeptide is located between the polynucleotide encoding the recombinant receptor and a polynucleotide encoding a selection cassette. In particular embodiments, the polynucleotide encoding the self-cleaving polypeptide is located between the polynucleotide encoding the selection cassette and the polynucleotide encoding the transduction marker (e.g., EGFRt). Exemplary self-cleaving polypeptides include 2A peptide from porcine teschovirus-1 (P2A), Thosea asigna virus (T2A), equine rhinitis A virus (E2A), foot-and-mouth disease virus (F2A), or variants thereof. Further exemplary nucleic acid and amino acid sequences of 2A peptides are set forth in, for example, Kim et al. (PLOS One 6:e18556 (2011). In particular embodiments, cells are genetically modified to include a self-cleaving polypeptide. In particular embodiments, the self-cleaving polypeptide includes T2A. In particular embodiments, a 2A sequence does not result in a 2A scar. In particular embodiments, a 2A sequence results in the addition of amino acids to proteins (also referred to as a 2A scar). A 2A sequence is a peptide sequence that induces ribosomal skipping during translation. Cleavage is triggered by ribosomal skipping of a peptide bond between proline and glycine within the 2A sequence. This results in the upstream protein having extra amino acids at the C terminal end, which has unknown impact on protein functionality.

Control features may be present in multiple copies in an artificial expression construct or can be expressed as distinct molecules with the use of a skip sequence. For example, an artificial expression construct can have one, two, three, four or five tag cassettes and/or one, two, three, four, or five transduction markers could also be expressed. For example, embodiments can include an artificial expression construct having two Myc tag cassettes, or a His tag and an HA tag cassette, or a HA tag and a Softag 1 tag cassette, or a Myc tag and a SBP tag cassette. Exemplary transduction markers and cognate pairs are described in U.S. Ser. No. 13/463,247.

One advantage of including at least one control feature in an artificial expression construct is that cells expressing the artificial expression construct administered to a subject can be increased or depleted using the cognate binding molecule to a tag cassette. In certain embodiments, the present disclosure provides a method for depleting a modified cell expressing an artificial expression construct by using an antibody specific for the tag cassette, using a cognate binding molecule specific for the control feature, or by using a second modified cell expressing an artificial expression construct and having specificity for the control feature. Elimination of modified cells may be accomplished using depletion agents specific for a control feature. For example, if EGFRt is used, then an anti-EGFRt binding domain (e.g., antibody, scFv) fused to or conjugated to a cell-toxic reagent (such as a toxin, radiometal) may be used, or an anti-EGFRt/anti-CD3 bispecific scFv, or an anti-EGFRt CAR T cell may be used. Similarly, if Her2tG is used, then an anti-Her2tG binding domain fused to or conjugated to a cell-toxic reagent may be used.

In particular embodiments, a polynucleotide encoding an iCaspase9 construct (iCasp9) may be inserted into an artificial expression construct as a suicide switch.

In certain embodiments, modified cells expressing an artificial expression construct may be detected or tracked in vivo by using antibodies that bind with specificity to a control feature (e.g., anti-Tag antibodies), or by other cognate binding molecules that specifically bind the control feature, which binding partners for the control feature are conjugated to a fluorescent dye, radio-tracer, iron-oxide nanoparticle or other imaging agent known in the art for detection by X-ray, CT-scan, MRI-scan, PET-scan, ultrasound, flow-cytometry, near infrared imaging systems, or other imaging modalities (see, e.g., Yu, et al., Theranostics 2:3, 2012).

Thus, modified cells expressing at least one control feature within an artificial expression construct can be, e.g., more readily identified, isolated, sorted, induced to proliferate, tracked, and/or eliminated as compared to a modified cell without a tag cassette.

### (vi) RECOMBINANT RECEPTORS

In particular embodiments, a recombinant receptor is or includes a binding domain that binds a target antigen, wherein the recombinant receptor is expressed by a cell following the artificial introduction of nucleic acids encoding the recombinant receptor into the cell. The recombinant receptor can be, e.g., a CAR, a T-cell receptor (TCR), or a CAR/TCR hybrid.

CAR, for example, include several distinct subcomponents that allow genetically modified cells (e.g., T cells) to recognize and kill target cells, such as cancer cells. The subcomponents include at least an extracellular component and an intracellular component. The extracellular component includes a binding domain that specifically binds a marker that is preferentially present on the surface of unwanted cells. When the binding domain binds such markers, the intracellular component activates the cell to destroy the bound cell. CAR can additionally include a transmembrane domain that links the extracellular component to the intracellular component, and other subcomponents that can increase the recombinant receptor's function. For example, the inclusion of a spacer and/or one or more linker sequences can allow the recombinant receptor to have additional conformational flexibility, often increasing the binding domain's ability to bind the targeted cell marker.

(vi-a) Binding Domains. In certain examples, recombinant receptors include a binding domain that binds a target antigen that is preferentially present on the surface of unwanted cells. Binding domains include any substance that binds to a cellular marker to form a complex. The choice of binding domain can depend upon the type and number of cellular markers that define the surface of a target cell. Examples of binding domains include cellular marker ligands, receptor ligands, antibodies, peptides, peptide aptamers, receptors (e.g., T cell receptors), or combinations and engineered fragments or formats thereof.

As is understood by those of ordinary skill in the art, a traditional antibody includes two heavy chains and two light chains. Each heavy chain consists of a variable region and a first, second, and third constant region, while each light chain consists of a variable region and a constant region. Mammalian heavy chains are classified as α, δ, ε, γ, and μ, and mammalian light chains are classified as λ or κ. Immunoglobulins including the α, δ, ε, γ, and μ heavy chains are classified as immunoglobulin (Ig)A, IgD, IgE, IgG, and IgM. The complete antibody forms a “Y” shape. The stem of the Y consists of the second and third constant regions (and for IgE and IgM, the fourth constant region) of two heavy chains bound together and disulfide bonds (inter-chain) are formed in the hinge. Heavy chains γ, α and δ have a constant region composed of three tandem (in a line) Ig domains, and a hinge region for added flexibility; heavy chains μ and ε have a constant region composed of four immunoglobulin domains. The second and third constant regions are referred to as “CH2 domain” and “CH3 domain”, respectively. Each arm of the Y includes the variable region and first constant region of a single heavy chain bound to the variable and constant regions of a single light chain. The variable regions of the light and heavy chains are responsible for antigen binding.

Light and heavy chain variable regions contain a “framework” region interrupted by three hypervariable regions, also called “complementarity determining regions” or “CDRs”.

CDR sets can be based on, for example, Kabat numbering (Kabat et al. (1991) “Sequences of Proteins of Immunological Interest,” 5th Ed. Public Health Service, National Institutes of Health, Bethesda, Md. (“Kabat” numbering scheme)); Chothia (AI-Lazikani et al. (1997) JMB 273:927-948 (“Chothia” numbering scheme)); Martin (Abinandan et al. (2008) Mol Immunol. 45:3832-3839 (“Martin” numbering scheme)); Gelfand (Gelfand and Kister (1995) Proc Natl Acad Sci USA. 92:10884-10888; Gelfand et al. (1998) Protein Eng. 11:1015-1025; Gelfand et al. (1996) Proc Natl Acad Sci USA. 93:3675-3678; Gelfand et al. (1998) J Comput Biol. 5:467-477 (“Gelfand” numbering scheme)); Contact (MacCallum et al. (1996) J. Mol. Biol. 262:732-745 (Contact numbering scheme)); IMGT (Lefranc et al. (2003) Dev Comp Immunol 27(1):55-77 (“IMGT” numbering scheme)); AHo (Honegger and Plückthun (2001) J Mol Biol 309(3):657-670 (“AHo” numbering scheme)); North (North et al. (2011) J Mol Biol. 406(2):228-256 (“North” numbering scheme)); or other numbering schemes. Software programs and bioinformatical tools, such as ABodyBuilder (Leem et al. (2016) MAbs 8(7):1259-1268), PIGSPro (Lepore et al. (2017) Nucleic Acids Res 45(W1):W17-W23), Kotai Antibody Builder (Yamashita et al. (2014) Bioinformatics 30(22):3279-3280), Rosetta Antibody (Weitzner et al. (2017) Nature Protocols 12:401-416), Paratome (Kunik et al. (2012) Nucleic Acids Res 40:W521-W524), Antibody i-Patch (Krawczyk et al. (2013) Protein Eng Des Sel 26(10):621-629), and proABC-2 (Ambrosetti et al. (2020) Bioinformatics 36(20):5107-5108 can also be used to determine CDR sequences.

The sequences of the framework regions of different light or heavy chains are relatively conserved within a species, such as humans. The framework region of an antibody, that is the combined framework regions of the constituent light and heavy chains, serves to position and align the CDRs in three-dimensional space. The CDRs are primarily responsible for binding to an epitope of an antigen. The CDRs of each chain are typically referred to as CDR1, CDR2, and CDR3, numbered sequentially starting from the N-terminus, and are also typically identified by the chain in which the particular CDR is located. Thus, the CDRs located in the variable domain of the heavy chain of the antibody are referred to as CDRH1, CDRH2, and CDRH3, whereas the CDRs located in the variable domain of the light chain of the antibody are referred to as CDRL1, CDRL2, and CDRL3. Antibodies with different specificities (i.e., different combining sites for different antigens) have different CDRs. Although it is the CDRs that vary from antibody to antibody, only a limited number of amino acid positions within the CDRs are directly involved in antigen binding. These positions within the CDRs are called specificity determining residues (SDRs).

References to “V_{H}” or “VH” refer to the variable region of an immunoglobulin heavy chain. References to “V_{L}” or “VL” refer to the variable region of an immunoglobulin light chain.

Antibodies that specifically bind an antigen can be prepared using methods of obtaining monoclonal antibodies, methods of phage display, methods to generate human or humanized antibodies, or methods using a transgenic animal or plant engineered to produce human antibodies. Phage display libraries of partially or fully synthetic antibodies are available and can be screened for an antibody or fragment thereof that can bind to the target antigen. Phage display libraries of human antibodies are also available. Once identified, the amino acid sequence or polynucleotide sequence coding for the antibody can be isolated and/or determined. Many relevant antibodies are also publicly known and commercially available.

In some alternatives, antibodies specifically bind to a cancer cell or virally-infected cell surface molecule and do not cross react with nonspecific components such as bovine serum albumin or other unrelated antigens.

“Antibody fragment” refers to at least one portion of an antibody, that retains the ability to specifically bind an antigen. Examples of antibody fragments include Fab, Fab′, F(ab′)₂, Fv fragments, single chain variable (scFv) antibody fragments, disulfide-linked Fvs (sdFv), a Fd fragment including VH and constant CH1 domains, linear antibodies, single domain antibodies such as sdAb (either VL or VH), camelid variable heavy only (VHH) domains, multi-specific antibodies formed from antibody fragments such as a bivalent fragment including two Fab fragments linked by a disulfide bridge at the hinge region, and an isolated CDR or other epitope binding fragments of an antibody (Harlow et al., 1999, In: Using Antibodies: A Laboratory Manual, Cold Spring Harbor Laboratory Press, NY; Harlow et al., 1989, In: Antibodies: A Laboratory Manual, Cold Spring Harbor, N.Y.; Houston et al., 1988, Proc. Natl. Acad. Sci. USA 85:5879-5883; Bird et al., 1988, Science 242:423-426). An antigen binding fragment can also be incorporated into single domain antibodies, maxibodies, minibodies, nanobodies, intrabodies, diabodies, triabodies, tetrabodies, v-NAR and bis-scFv (see, e.g., Hollinger and Hudson (2005) Nature Biotechnology 23:1126-1136).

In particular embodiments, a binding domain can include humanized forms of non-human (e.g., murine) antibodies or antigen binding fragments thereof. A humanized antibody includes an antibody in which the constant and variable framework region of one or more human immunoglobulins is fused with the binding region, e.g., the CDR, of an animal (non-human) immunoglobulin. Such humanized antibodies are designed to maintain the binding specificity of the non-human antibody from which the binding regions are derived but avoid an immune reaction against the non-human antibody. In particular embodiments, a binding domain can include a fully human antibody or antibody fragment thereof, where the whole molecule is of human origin or includes an amino acid sequence identical to a human form of the antibody or immunoglobulin. [0146]“scFv” refers to an engineered fusion protein including the VH and VL of an antibody linked via a linker and capable of being expressed as a single chain polypeptide. The scFv retains the specificity of the intact antibody from which it is derived. In particular embodiments, a linker connecting the variable regions can include glycine-serine linkers, for example, SEQ ID NOs: 123-136 or provided elsewhere herein. In particular embodiments, an scFv may have the VL and VH variable regions in either order, e.g., with respect to the N-terminal and C-terminal ends of the polypeptide, the scFv may include VL-linker-VH or may include VH-linker-VL.

Recombinant receptors also include TCR (including engineered TCR) that can be used independently or as a binding domain within a CAR (e.g., CAR/TCR hybrid). For example, the sequences of numerous TCR that bind particular antigen fragments are known and publicly available.

TCR can also be identified for use with a particular antigen by, for example, isolating T cells that bind a particular antigen/MHC complex and sequencing the TCR chains binding the complex. TCR genes encoding TCR can be readily cloned by, for example, the 5′ RACE procedure using primers corresponding to the sequences specific to the TCR α-chain gene and the TCR β-chain gene.

In particular embodiments, it may be necessary to pair TCR chains following sequencing (i.e., to perform paired chain analysis). Various methods can be utilized to pair chains, when necessary. For example, chain pairing may be assisted in silico by computer methods, such as immunology gene alignment software available from IMGT, JOINSOLVER, VDJSolver, SoDA, iHMMune-align, or other similar tools for annotating VDJ gene segments. Assays such as PairSEQ® (Adaptive Biotechnologies Corp., Seattle, WA) have also been developed.

In particular embodiments, an engineered TCR includes a single chain T cell receptor (scTCR) including Va/P and Ca/P chains (e.g., Vα-Cα, Vβ-Cβ, Vα-Vβ) or including Vα-Cα, Vβ-Cβ, Vα-Vβ pair specific for a target of interest (e.g., peptide-MHC complex).

In particular embodiments, a CAR/TCR hybrid includes components of a CAR and components of a TCR. For example, a CAR/TCR hybrid can include a TCR binding domain and intracellular signaling domains specific to CAR. This allows for antigen recognition similar to that of a TCR but use of intracellular signaling similar to a CAR.

Cancer antigens are proteins that are produced by cancer cells and viral antigens are proteins produced by virally-infected cells. Binding domains of recombinant receptors disclosed herein can be selected to bind cancer antigens or viral antigens. In some alternatives, cancer or viral antigens are selectively expressed or overexpressed on the cancerous or infected cells as compared to other cells of the same tissue type. In some alternatives, a cancer or viral antigen is a cell surface molecule that is found on cancer cells or virally-infected cells and is not substantially found on normal tissues, or restricted in its expression to non-vital normal tissues.

In particular embodiments, cancer antigens or viral antigens are preferentially expressed by cancer cells or virally-infected cells, respectively. “Preferentially expressed” means that the antigen is found on the targeted cell type at least 25%, 35%, 45%, 55%, 65%, 75%, 85%, 95%, 96%, 97%, 98%, 99%, or 100% more than on non-targeted cells.

Exemplary cancer antigens include carcinoembryonic antigen (CEA), prostate specific antigen, Prostate Stem Cell antigen (PSCA), PSMA, Her2/neu, estrogen receptor, progesterone receptor, ephrinB2, CD19, CD20, CD22, CD23, CD123, CS-1, CE7, ROR1, mesothelin, c-Met, GD-2, MAGE A3 TCR, EGFR, EGFRvIII, EphA2, IL13Ra2, L1CAM, oaGD2, GD2, B7H3, CD33, FITC, VAR2CSA, MUC16, PD-L1, ERBB2, folate receptor (FOLR), CD56; glypican-2, disialoganglioside, EpCam, L1-CAM, Lewis Y, WT-1, Tyrosinase related protein 1 (TYRP1/gp75); GD2, B-cell maturation antigen (BCMA), CD24, SV40 T, carboxy-anhydrase-IX (CAIX); and CD133. Other examples are known to those of ordinary skill in the art. Particular embodiments utilize binding domains that specifically bind CD19.

In particular embodiments, a binding domain that binds a cancer antigen includes an scFv. In particular embodiments, an scFv includes an huCD19 (G01S) scFv, a muCD19 (FMC63) scFv, a CD20 (Leu 16) scFv, a CD22 (m971) scFv, a B7H3 (hBRCA84D) scFv, an L1CAM (CE7) scFv, an EGFR scFv, an EGFRVIII (806) scFv, an EphA2 (2A4) scFv, an EpHA2 (4H5) scFv, an FITC (E2) scFv, a GD2 (hu3F8) scFv, a Her2 (Herceptin) scFv, an IL13Ra2 (hu08) VIVh scFv, an IL13Ra2 hu08 VhV1 scFv, an IL13Ra2 (hu07) VhV1 scFv, an IL13Ra2 (hu07) VhVI scFv, an oaGD2 (8B6) VIVh, a ROR1 (R12) scFv, a CD33 (h2H12) VhVI scFv, a CD33 (h2H12) VIVh scFv, a mesothelin (P4) scFv, a VAR2CSA (ID1-DBL2Xb) scFv, or an IL13Ra2 (IL13 zetakine) amino acid sequence.

In particular embodiments, the huCD19 (G01S) scFv with a GMCSFss includes the sequence:

(SEQ ID NO: 116)
MLLLVTSLLLCELPHPAFLLIPEVQLVESGGGLVQPGRSLRLSCAASGF
TFDDYAMHWVRQAPGKGLEWVSGISWNSGRIGYADSVKGRFTISRDNAK
NSLFLQMNSLRAEDTAVYYCARDQGYHYYDSAEHAFDIWGQGTVVTVSS
GGGGSGGGGSGGGGSQSALTQPRSVSGFPGQSVTISCTGTTSDDVSWYQ
QHPGKAPQLMLYDVSKRPSGVPHRFSGSRSGRAASLIISGLQTEDEADY
FCSSYAGRYNSVLFGGGTKLTVL.

In particular embodiments, a huCD19 (G01S) scFv includes the sequence

(SEQ ID NO: 117)
EVQLVESGGGLVQPGRSLRLSCAASGFTFDDYAMHWVRQAPGKGLEWVS
GISWNSGRIGYADSVKGRFTISRDNAKNSLFLQMNSLRAEDTAVYYCAR
DQGYHYYDSAEHAFDIWGQGTVVTVSSGGGGSGGGGSGGGGSQSALTQP
RSVSGFPGQSVTISCTGTTSDDVSWYQQHPGKAPQLMLYDVSKRPSGVP
HRFSGSRSGRAASLIISGLQTEDEADYFCSSYAGRYNSVLFGGGTKLTV
L.

In particular embodiments, the muCD19 (FMC63) scFv includes the sequence

(SEQ ID NO: 118)
MLLLVTSLLLCELPHPAFLLIPDIQMTQTTSSLSASLGDRVTISCRASQ
DISKYLNWYQQKPDGTVKLLIYHTSRLHSGVPSRFSGSGSGTDYSLTIS
NLEQEDIATYFCQQGNTLPYTFGGGTKLEITGSTSGSGKPGSGEGSTKG
EVKLQESGPGLVAPSQSLSVTCTVSGVSLPDYGVSWIRQPPRKGLEWLG
VIWGSETTYYNSALKSRLTIIKDNSKSQVFLKMNSLQTDDTAIYYCAKH
YYYGGSYAMDYWGQGTSVTVSS.

In particular embodiments, a CD19 scFv includes the sequence:

(SEQ ID NO: 119)
DIQMTQTTSSLSALGDRVTISCRASQDISKYLNWYQQKPDGTVKLLIYH
TSRLHSGVPSRFSGSGSGTDYSLTISNLEQEDIATYFCQQGNTLPYTFG
GGTKLEITGSTSGSGKPGSGEGSTKGEVKLQESGPGLVAPSQSLSVTCT
VSGVSLPDYGVSWIRQPPRKGLEWLGVIWGSETTYYNSALKSRLTIIKD
NSKSQVFLKMNSLQTDDTAIYYCAKHYYYGGSYAMDYWGQGTSVTVSS.

In particular embodiments the CD33 (h2H12) VhVI scFv includes the sequence:

(SEQ ID NO: 120)
QVQLVQSGAEVKKPGASVKVSCKASGYTFTNYDINWVRQAPGQGLEWIG
WIYPGDGSTKYNEKFKAKATLTADTSTSTAYMELRSLRSDDTAVYYCAS
GYEDAMDYWGQGTTVTVSSGGGGSGGGGSGGGGSDIQMTQSPSSLSASV
GDRVTINCKASQDINSYLSWFQQKPGKAPKTLIYRANRLVDGVPSRFSG
SGSGQDYTLTISSLQPEDFATYYCLQYDEFPLTFGGGTKVEIK.

In particular embodiments, the CD33 (h2H12) VIVh scFv includes the sequence:

(SEQ ID NO: 121)
DIQMTQSPSSLSASVGDRVTINCKASQDINSYLSWFQQKPGKAPKTLIY
RANRLVDGVPSRFSGSGSGQDYTLTISSLQPEDFATYYCLQYDEFPLTF
GGGTKVEIKGGGGSGGGGSGGGGSQVQLVQSGAEVKKPGASVKVSCKAS
GYTFTNYDINWVRQAPGQGLEWIGWIYPGDGSTKYNEKFKAKATLTADT
STSTAYMELRSLRSDDTAVYYCASGYEDAMDYWGQGTTVTVSS.

In particular embodiments, the CD33 scFv (e.g., CD33 (h2H12) VhVI scFv and/or CD33 (h2H12) VIVh scFv) include a granulocyte-macrophage colony stimulating factor (GM-CSF) signal sequence. In particular embodiments, the GM-CSF signal sequence includes the sequence:

(SEQ ID NO: 122)
MLLLVTSLLLCELPHPAFLLIP.

In particular embodiments, binding domains that bind CD20 can be based on the binding domains of SP32 (ab64088), EP459Y (ab78237), rIGEL/773 (ab219329), ocrelizumab, rituximab, ofatumumab, obinutuzumab), ibritumomab, or tositumomab.

Exemplary viral antigens include coronaviral antigens: the spike (S) protein; cytomegaloviral antigens: envelope glycoprotein B and CMV pp65; Epstein-Barr antigens: EBV EBNAI, EBV P18, and EBV P23; hepatitis antigens: the S, M, and L proteins of hepatitis B virus, the pre-S antigen of hepatitis B virus, HBCAG DELTA, HBV HBE, hepatitis C viral RNA, HCV NS3 and HCV NS4; herpes simplex viral antigens: immediate early proteins and glycoprotein D; HIV antigens: gene products of the gag, pol, and env genes such as HIV gp32, HIV gp41, HIV gp120, HIV gp160, HIV P17/24, HIV P24, HIV P55 GAG, HIV P66 POL, HIV TAT, HIV GP36, the Nef protein and reverse transcriptase; influenza antigens: hemagglutinin and neuraminidase; Japanese encephalitis viral antigens: proteins E, M-E, M-E-NS1, NS1, NS1-NS2A and 80% E; measles antigens: the measles virus fusion protein; rabies antigens: rabies glycoprotein and rabies nucleoprotein; respiratory syncytial viral antigens: the RSV fusion protein and the M2 protein; rotaviral antigens: VP7sc; rubella antigens: proteins E1 and E2; and varicella zoster viral antigens: gpI and gpII. See Fundamental Virology, Second Edition, eds. Fields, B. N. and Knipe, D. M. (Raven Press, New York, 1991) for additional examples of viral antigens. In particular embodiments, binding domains that bind viral antigens can be used.

In particular embodiments, a bacterial antigen includes antigens expressed by bacteria. In particular embodiments, antigens are expressed by cells associated with bacterial infections. Exemplary bacteria include anthrax; gram-negative bacilli, chlamydia, diptheria, haemophilus influenza, Helicobacter pylori, malaria, Mycobacterium tuberculosis, pertussis toxin, pneumococcus, rickettsiae, staphylococcus, streptococcus and tetanus.

As particular examples of bacterial antigens: anthrax antigens include anthrax protective antigen; gram-negative bacilli antigens include lipopolysaccharides; haemophilus influenza antigens include capsular polysaccharides; diptheria antigens include diptheria toxin; Mycobacterium tuberculosis antigens include mycolic acid, heat shock protein 65 (HSP65), the 30 kDa major secreted protein and antigen 85A; pertussis toxin antigens include hemagglutinin, pertactin, FIM2, FIM3 and adenylate cyclase; pneumococcal antigens include pneumolysin and pneumococcal capsular polysaccharides; rickettsiae antigens include rompA; streptococcal antigens include M proteins; and tetanus antigens include tetanus toxin.

In particular embodiments, a fungal antigen includes antigens expressed by fungi. In particular embodiments, antigens are expressed by cells associated with fungal infections. Exemplary fungi include candida, coccidiodes, cryptococcus, histoplasma, leishmania, plasmodium, protozoa, parasites, schistosomae, tinea, toxoplasma, and Trypanosoma cruzi.

As further particular examples of fungal antigens: coccidiodes antigens include spherule antigens; cryptococcal antigens include capsular polysaccharides; histoplasma antigens include heat shock protein 60 (HSP60); leishmania antigens include gp63 and lipophosphoglycan; Plasmodium falciparum antigens include merozoite surface antigens, sporozoite surface antigens, circumsporozoite antigens, gametocyte/gamete surface antigens, protozoal and other parasitic antigens including the blood-stage antigen pf 155/RESA; schistosomae antigens include glutathione-S-transferase and paramyosin; tinea fungal antigens include trichophytin; toxoplasma antigens include SAG-1 and p30; and Trypanosoma cruzi antigens include the 75-77 kDa antigen and the 56 kDa antigen.

In particular embodiments, an arthropod antigen includes antigens expressed by an arthropod. In particular embodiments, a parasite antigen includes antigens expressed by a parasite.

In particular embodiments, a binding domain binds an immune cell activating epitope. In particular embodiments, an immune cell activating epitope is part of an antigen. In particular embodiments, the immune cell activating epitope can be expressed by an immune cell within the negative fraction of a sample and/or can be a binding domain on a multi-specific binding molecule (also referred to as a chemical adapter). An immune cell activating epitope is a portion of a molecule (e.g., portion of a protein) that activates an immune cell when bound by a binding domain expressed by the immune cell. An immune cell activating epitope can be on anything that binds the recombinant receptor and links the bound T cell to an activating cell (e.g., PBMCs). In certain examples, a chemical adapter is an antibody.

In particular embodiments, the immune cell activating epitope can be a B-cell ligand, wherein the B-cell ligand is CD1d, CD5, CD19, CD20, CD21, CD22, CD23/Fc epsilon RII, CD24, CD25/IL-2R alphaCD27/TNFRSF7, CD32, CD34, CD35, CD38, CD40 (TNFRSF5), CD44, CD45, CD45.1, CD45.2, CD54 (ICAM-1), CD69, CD72, CD79, CD80, CD84/SLAMF5, LFA-1, CALLA, BCMA, B-cell receptor (BCR), IgMs, IgD, B220/CD45R, C1q R1/CD93, CD84/SLAMF5, BAFF R/TNFRSF13C, B220/CD45R, B7-1/CD80, B7-2/CD86, TNFSF7, TNFRSF5, ENPP-1, HVEM/TNFRSF14, BLIMP 1/PRDM1, CXCR4, DEP-1/CD148 or EMMPRIN/CD 147.

Other immune cell activating epitopes can be found on, e.g., natural killer T (NKT) cells, natural killer cells (also known as K cells and killer cells), tumor-infiltrating lymphocytes (TILs), marrow-infiltrating lymphocytes (MILs), MAIT cells, macrophages, monocytes, and/or dendritic cells. These cells and exemplary cell surface antigens are described elsewhere herein.

In particular embodiments, the immune cell activating epitope is a hapten. Haptens include any small molecule which, when combined with a larger carrier such as a protein, elicits the production of antibodies which bind specifically to it (in the free or combined state). Haptens can include peptides, other larger chemicals, and aptamers. In some embodiments, a hapten can be any hapten provided in the hapten database accessible on the World Wide Web under the URL crdd.osdd.net/raghava/haptendb/. In particular embodiments, the hapten is tethered to an activating cell. In particular embodiments, if the immune cell activating epitope is a hapten, the cell expressing the recombinant receptor includes a binding domain that binds the hapten. In particular embodiments, the binding domain that binds the hapten can be the binding domain of the recombinant receptor. In particular embodiments, the binding domain that binds the hapten can be a binding domain not on the recombinant receptor.

In particular embodiments, the immune cell activating epitope can be a binding domain of a multi-specific binding molecule (also referred to as a chemical adapter). In particular embodiments, a multi-specific binding molecule includes at least two binding domains wherein at least one binding domain is the immune cell activating epitope that binds the T cell expressing a recombinant receptor and at least one binding domain binds an immune cell within the negative fraction. Multi-specific binding molecules that are useful for T cell activation are described elsewhere herein. In particular embodiments, multi-specific binding molecules include bispecific antibodies. In particular embodiments, multi-specific binding molecules include antibodies. An antibody can be considered a multi-specific binding molecule because the antigen binding domain binds an antigen while the Fc interacts with immune cells within the negative fraction.

(vi-b) Transmembrane Domains. As indicated, transmembrane domains within a recombinant receptor serve to connect the extracellular component and intracellular component through the cell membrane. The transmembrane domain can anchor the expressed molecule in the modified cell's membrane.

The transmembrane domain can be derived either from a natural and/or a synthetic source. When the source is natural, the transmembrane domain can be derived from any membrane-bound or transmembrane protein. Transmembrane domains can include at least the transmembrane region(s) of the a, p or (chain of a T-cell receptor, CD28, CD27, CD3 epsilon, CD45, CD4, CD5, CD8, CD9, CD16, CD22; CD33, CD37, CD64, CD80, CD86, CD134, CD137 CD154, Toll-like receptor 1 (TLR1), TLR2, TLR3, TLR4, TLR5, TLR6, TLR7, TLR8, and TLR9. In particular embodiments, a transmembrane domain may include at least the transmembrane region(s) of, e.g., KIRDS2, OX40, CD2, CD27, LFA-1 (CD 11a, CD18), ICOS (CD278), 4-1BB (CD137), GITR, CD40, BAFFR, HVEM (LIGHTR), SLAMF7, NKp80 (KLRF1), NKp44, NKp30, NKp46, CD160, CD19, IL2RP, IL2Ry, IL7R a, ITGA1, VLA1, CD49a, ITGA4, IA4, CD49D, ITGA6, VLA-6, CD49f, ITGAD, CDI Id, ITGAE, CD103, ITGAL, CDI la, ITGAM, CDI lb, ITGAX, CDI Ic, ITGB1, CD29, ITGB2, CD18, ITGB7, TNFR2, DNAM1(CD226), SLAMF4 (CD244, 2B4), CD84, CD96 (Tactile), CEACAM1, CRT AM, Ly9(CD229), PSGL1, CD100 (SEMA4D), SLAMF6 (NTB-A, Ly108), SLAM (SLAMF1, CD150, IPO-3), BLAME (SLAMF8), SELPLG (CD162), LTBR, PAG/Cbp, NKG2D, or NKG2C. In particular embodiments, a variety of human hinges can be employed as well including the human Ig (immunoglobulin) hinge (e.g., an IgG4 hinge, an IgD hinge), a GS linker (e.g., a GS linker described herein), a KIR2DS2 hinge or a CD8a hinge.

In particular embodiments, a transmembrane domain has a three-dimensional structure that is thermodynamically stable in a cell membrane, and generally ranges in length from 15 to 30 amino acids. The structure of a transmembrane domain can include an a helix, a p barrel, a p sheet, a p helix, or any combination thereof.

A transmembrane domain can include one or more additional amino acids adjacent to the transmembrane region, e.g., one or more amino acid within the extracellular region of the recombinant receptor (e.g., up to 15 amino acids of the extracellular region) and/or one or more additional amino acids within the intracellular region of the recombinant receptor (e.g., up to 15 amino acids of the intracellular components). In one aspect, the transmembrane domain is from the same protein that the signaling domain, co-stimulatory domain or the hinge domain is derived from. In another aspect, the transmembrane domain is not derived from the same protein that any other domain of the recombinant receptor is derived from. In some instances, the transmembrane domain can be selected or modified by amino acid substitution to avoid binding of such domains to the transmembrane domains of the same or different surface membrane proteins to minimize interactions with other unintended members of the receptor complex. In particular embodiments, the transmembrane domain is a CD28 transmembrane domain (encoded by, for example, SEQ ID NOs: 69, 70, 71, or 72). In particular embodiments, the CD28 transmembrane domain has the sequence as set forth in SEQ ID NOs: 66, 67, or 68.

(vi-c) Intracellular Effector Domains. The intracellular effector domains of a recombinant receptor are responsible for activation of the cell in which the recombinant receptor is expressed. The term “effector domain” is thus meant to include any portion of the intracellular domain sufficient to transduce an activation signal. An effector domain can directly or indirectly promote a biological or physiological response in a cell when receiving the appropriate signal. In certain embodiments, an effector domain is part of a protein or protein complex that receives a signal when bound, or it binds directly to a target molecule, which triggers a signal from the effector domain. An effector domain may directly promote a cellular response when it contains one or more signaling domains or motifs, such as an immunoreceptor tyrosine-based activation motif (ITAM). In other embodiments, an effector domain will indirectly promote a cellular response by associating with one or more other proteins that directly promote a cellular response, such as co-stimulatory domains.

Effector domains can provide for activation of at least one function of a modified cell upon binding to the cellular marker expressed by a cancer cell. Activation of the modified cell can include one or more of differentiation, proliferation and/or activation or other effector functions. In particular embodiments, an effector domain can include an intracellular signaling component including a T cell receptor and a co-stimulatory domain which can include the cytoplasmic sequence from co-receptor or co-stimulatory molecule.

An effector domain can include one, two, three or more intracellular signaling components (e.g., receptor signaling domains, cytoplasmic signaling sequences), co-stimulatory domains, or combinations thereof. Exemplary effector domains include signaling and stimulatory domains selected from: 4-1BB (CD137), CARD11, CD3γ, CD3δ, CD3ε, CD3ζ, CD27, CD28, CD79A, CD79B, DAP10, FcRα, FcRβ (FcεR1b), FcRγ, Fyn, HVEM (LIGHTR), ICOS, LAG3, LAT, Lck, LRP, NKG2D, NOTCH1, pTa, PTCH2, OX40, ROR2, Ryk, SLAMF1, Slp76, TCRα, TCRβ, TRIM, Wnt, Zap70, or any combination thereof. In particular embodiments, exemplary effector domains include signaling and co-stimulatory domains selected from: CD86, FcγRIIa, DAP12, CD30, CD40, PD-1, lymphocyte function-associated antigen-1 (LFA-1), CD2, CD7, LIGHT, NKG2C, B7-H3, a ligand that specifically binds with CD83, CDS, ICAM-1, GITR, BAFFR, SLAMF7, NKp80 (KLRF1), CD127, CD160, CD19, CD4, CD8a, CD8P, IL2Rβ, IL2Rγ, IL7Rα, ITGA4, VLA1, CD49a, IA4, CD49D, ITGA6, VLA-6, CD49f, ITGAD, CD11d, ITGAE, CD103, ITGAL, CD11a, ITGAM, CD11b, ITGAX, CD11c, ITGB1, CD29, ITGB2, CD18, ITGB7, TNFR2, TRANCE/RANKL, DNAM1 (CD226), SLAMF4 (CD244, 2B4), CD84, CD96 (Tactile), CEACAM1, CRTAM, Ly9 (CD229), PSGL1, CD100 (SEMA4D), CD69, SLAMF6 (NTB-A, Ly108), SLAM (CD150, IPO-3), BLAME (SLAMF8), SELPLG (CD162), LTBR, GADS, PAG/Cbp, NKp44, NKp30, NKp46, TLR1, TLR2, TLR3, TLR4, TLR5, TLR6, TLR7, TLR8, or TLR9. In particular embodiments, the effector domain includes a CD3ζ signaling domain.

Intracellular signaling component sequences that act in a stimulatory manner may include iTAMs. Examples of iTAMs including primary cytoplasmic signaling sequences include those derived from CD3γ, CD3δ, CD3ε, CD3ζ, CD5, CD22, CD66d, CD79a, CD79b, and common FcRγ (FCER1G), FcγRIIa, FcRβ (Fcε Rib), DAP10, and DAP12. In particular embodiments, variants of CD3ζ retain at least one, two, three, or all ITAM regions.

In particular embodiments, an effector domain includes a cytoplasmic portion that associates with a cytoplasmic signaling protein, wherein the cytoplasmic signaling protein is a lymphocyte receptor or signaling domain thereof, a protein including a plurality of ITAMs, a co-stimulatory domain, or any combination thereof.

Additional examples of intracellular signaling components include the cytoplasmic sequences of the CD3ζ chain, and/or co-receptors that act in concert to initiate signal transduction following binding domain engagement.

A co-stimulatory domain is a domain whose activation can be required for an efficient lymphocyte response to cellular marker binding. Some molecules are interchangeable as intracellular signaling components or co-stimulatory domains. Examples of costimulatory domains include CD27, CD28, 4-1BB (CD 137), OX40, CD30, CD40, PD-1, ICOS, lymphocyte function-associated antigen-1 (LFA-1), CD2, CD7, LIGHT, NKG2C, B7-H3, and a ligand that specifically binds with CD83. For example, CD27 co-stimulation has been demonstrated to enhance proliferation, effector function, and survival of human CAR-T cells in vitro and augments human T cell persistence and anti-cancer activity in vivo (Song et al. Blood. 2012; 119(3):696-706).

Further examples of such co-stimulatory domain molecules include CDS, ICAM-1, GITR, BAFFR, HVEM (LIGHTR), SLAMF7, NKp80 (KLRF1), NKp44, NKp30, NKp46, CD160, CD19, CD4, CD8a, CD8β, IL2Rβ, IL2Rγ, IL7Rα, ITGA4, VLA1, CD49a, ITGA4, IA4, CD49D, ITGA6, VLA-6, CD49f, ITGAD, CDIld, ITGAE, CD103, ITGAL, CDIla, ITGAM, CDI lb, ITGAX, CDIlc, ITGBI, CD29, ITGB2, CD18, ITGB7, TNFR2, TRANCE/RANKL, DNAM1 (CD226), SLAMF4 (CD244, 2B4), CD84, CD96 (Tactile), NKG2D, CEACAM1, CRTAM, Ly9 (CD229), PSGL1, CD100 (SEMA4D), CD69, SLAMF6 (NTB-A, Ly108), SLAM (SLAMF1, CD150, IPO-3), BLAME (SLAMF8), SELPLG (CD162), LTBR, LAT, GADS, SLP-76, PAG/Cbp, and CD19a. In particular embodiments, the co-stimulatory domain includes a 4-1 BB signaling domain.

In particular embodiments, the intracellular signaling components include CD3ζ (encoded by, for example, SEQ ID NOs: 82 or 83) and a variant of 4-1BB (encoded by, for example, SEQ ID NOs: 76, 77, or 78). In particular embodiments, CD3ζ has the sequence as set forth in SEQ ID NOs: 79, 80, or 81. In particular embodiments, 4-1BB has the sequence as set forth in SEQ ID NOs: 73, 74, or 75.

Intracellular components may also include one or more of a protein of a Wnt signaling pathway (e.g., LRP, Ryk, or ROR2), NOTCH signaling pathway (e.g., NOTCH1, NOTCH2, NOTCH3, or NOTCH4), Hedgehog signaling pathway (e.g., PTCH or SMO), receptor tyrosine kinases (RTKs) (e.g., epidermal growth factor (EGF) receptor family, fibroblast growth factor (FGF) receptor family, hepatocyte growth factor (HGF) receptor family, insulin receptor (IR) family, platelet-derived growth factor (PDGF) receptor family, vascular endothelial growth factor (VEGF) receptor family, tropomycin receptor kinase (Trk) receptor family, ephrin (Eph) receptor family, AXL receptor family, leukocyte tyrosine kinase (LTK) receptor family, tyrosine kinase with immunoglobulin-like and EGF-like domains 1 (TIE) receptor family, receptor tyrosine kinase-like orphan (ROR) receptor family, discoidin domain (DDR) receptor family, rearranged during transfection (RET) receptor family, tyrosine-protein kinase-like (PTK7) receptor family, related to receptor tyrosine kinase (RYK) receptor family, or muscle specific kinase (MuSK) receptor family); G-protein-coupled receptors, GPCRs (Frizzled or Smoothened); serine/threonine kinase receptors (BMPR or TGFR); or cytokine receptors (IL1R, IL2R, IL7R, or IL15R).

(vi-d) Linkers. A linker within a recombinant receptor can be any portion of a recombinant receptor that serves to connect two subcomponents or domains of the recombinant receptor. In particular embodiments, linkers can provide flexibility for different components of the recombinant receptor. Linkers can also include spacer regions and junction amino acids. In certain examples, when a more rigid linker is required, proline-rich linkers can be used. Some linkers serve no purpose other than to link components while many linkers serve an additional purpose, such as a multimerization domain.

Spacers are used to create appropriate distances and/or flexibility from other recombinant receptor sub-components. As indicated, in particular embodiments, the length of a spacer is customized for binding targeted cells and mediating destruction. In particular embodiments, a spacer length can be selected based upon the location of a cellular marker epitope, affinity of a binding domain for the epitope, and/or the ability of the binding domain to mediate cell destruction following target binding.

Spacers typically include those having 10 to 250 amino acids, 10 to 200 amino acids, 10 to 150 amino acids, 10 to 100 amino acids, 10 to 50 amino acids, or 10 to 25 amino acids.

In particular embodiments, a spacer is 5 amino acids, 8 amino acids, 10 amino acids, 12 amino acids, 14 amino acids, 20 amino acids, 21 amino acids, 26 amino acids, 27 amino acids, 45 amino acids, 50 amino acids, or 75 amino acids. These lengths qualify as short spacers.

In particular embodiments, a spacer is 76 amino acids, 90 amino acids, 100 amino acids, 110 amino acids, 120 amino acids, 125 amino acids, 128 amino acids, 131 amino acids, 135 amino acids, 140 amino acids, 150 amino acids, 160 amino acids, 170 amino acids, or 179 amino acids. These lengths qualify as intermediate spacers.

In particular embodiments, a spacer is 180 amino acids, 190 amino acids, 200 amino acids, 210 amino acids, 212 amino acids, 214 amino acids, 216 amino acids, 218 amino acids, 220 amino acids, 228 amino acids, 230 amino acids, 240 amino acids, 250 amino acids, 260 amino acids, or 270 amino acids. These lengths qualify as long spacers.

Exemplary spacers include all or a portion of an immunoglobulin hinge region. An immunoglobulin hinge region may be a wild-type immunoglobulin hinge region or an altered wild-type immunoglobulin hinge region. In certain embodiments, an immunoglobulin hinge region is a human immunoglobulin hinge region. As used herein, a “wild type immunoglobulin hinge region” refers to a naturally occurring upper and middle hinge amino acid sequences interposed between and connecting the CH1 and CH2 domains (for IgG, IgA, and IgD) or interposed between and connecting the CH1 and CH3 domains (for IgE and IgM) found in the heavy chain of an antibody.

An immunoglobulin hinge region may be an IgG, IgA, IgD, IgE, or IgM hinge region. An IgG hinge region may be an IgG1, IgG2, IgG3, or IgG4 hinge region. Sequences from IgG1, IgG2, IgG3, IgG4 or IgD can be used alone or in combination with all or a portion of a CH2 region; all or a portion of a CH3 region; or all or a portion of a CH2 region and all or a portion of a CH3 region. In particular embodiments, the IgG4 hinge region includes the sequence as set forth in SEQ ID NO: 60.

Other examples of hinge regions that can be used in a recombinant receptor described herein include the hinge region present in the extracellular regions of type 1 membrane proteins, such as CD8a, CD4, CD28 and CD7, which may be wild-type or variants thereof.

In particular embodiments, a spacer includes a hinge region that includes a type II C-lectin interdomain (stalk) region or a cluster of differentiation (CD) molecule stalk region. A “stalk region” of a type II C-lectin or CD molecule refers to the portion of the extracellular domain (ECD) of the type II C-lectin or CD molecule that is located between the C-type lectin-like domain (CTLD; e.g., similar to CTLD of natural killer cell receptors) and the hydrophobic portion (transmembrane domain). For example, the ECD of human CD94 (GenBank Accession No. AAC50291.1) corresponds to amino acid residues 34-179, but the CTLD corresponds to amino acid residues 61-176, so the stalk region of the human CD94 molecule includes amino acid residues 34-60, which are located between the hydrophobic portion (transmembrane domain) and CTLD (see Boyington et al., Immunity 10:15, 1999; for descriptions of other stalk regions, see also Beavil et al., Proc. Nat'l. Acad. Sci. USA 89:153, 1992; and Figdor et al., Nat. Rev. Immunol. 2:11, 2002). These type II C-lectin or CD molecules may also have junction amino acids (described below) between the stalk region and the transmembrane region or the CTLD. In another example, the 233 amino acid human NKG2A protein (GenBank Accession No. P26715.1) has a hydrophobic portion (transmembrane domain) ranging from amino acids 71-93 and an ECD ranging from amino acids 94-233. The CTLD includes amino acids 119-231 and the stalk region includes amino acids 99-116, which may be flanked by additional junction amino acids. Other type II C-lectin or CD molecules, as well as their extracellular ligand-binding domains, stalk regions, and CTLDs are known in the art (see, e.g., GenBank Accession Nos. NP 001993.2; AAH07037.1; NP 001773.1; AAL65234.1; CAA04925.1; for the sequences of human CD23, CD69, CD72, NKG2A, and NKG2D and their descriptions, respectively).

Linkers can, for example, link VL and VH of antibody derived binding domains of scFvs and serve as junction amino acids between subcomponent portions of a recombinant receptor.

Linkers can be flexible, rigid, or semi-rigid, depending on the desired function of the linker. Linkers can include junction amino acids. For example, in particular embodiments, linkers provide flexibility and room for conformational movement between different components of the recombinant receptor. Commonly used flexible linkers include Gly-Ser linkers. In particular embodiments, the linker sequence includes sets of glycine and serine repeats such as from one to ten repeats of (GlyₓSer_{y})ₙ, wherein x and y are independently an integer from 0 to 10 provided that x and y are not both 0 and wherein n is an integer of 1, 2, 3, 4, 5, 6, 7, 8, 9 or 10). Particular examples include (Gly₄Ser)ₙ (SEQ ID NO: 123), (Gly₃Ser)ₙ(Gly₄Ser)ₙ (SEQ ID NO: 124), (Gly₃Ser)ₙ(Gly₂Ser)ₙ (SEQ ID NO: 125), or (Gly₃Ser)ₙ(Gly₄Ser)₁ (SEQ ID NO: 126). In particular embodiments, the linker is (Gly₄Ser)₄ (SEQ ID NO: 127), (Gly₄Ser)₃ (SEQ ID NO: 128), (Gly₄Ser)₂ (SEQ ID NO: 129), (Gly₄Ser)₁ (SEQ ID NO: 130), (Gly₃Ser)₂ (SEQ ID NO: 131), (Gly₃Ser)₁ (SEQ ID NO: 132), (Gly₂Ser)₂ (SEQ ID NO: 133) or (Gly₂Ser)₁, GGSGGGSGGSG (SEQ ID NO: 134), GGSGGGSGSG (SEQ ID NO: 135), or GGSGGGSG (SEQ ID NO: 136). As indicated elsewhere herein, various Gly-Ser linkers can be used to link PD1 to an immunostimulatory cytokine. In particular embodiments, the glycine-serine linker includes the sequence as set forth in SEQ ID NO: 128. In particular embodiments, the glycine-serine linker includes the sequence GGG.

In particular embodiments, a linker region is (GGGGS)n (SEQ ID NO: 123) wherein n is an integer including, 1, 2, 3, 4, 5, 6, 7, 8, 9, or more. In particular embodiments, the spacer is (EAAAK)n (SEQ ID NO: 137) wherein n is an integer including 1, 2, 3, 4, 5, 6, 7, 8, 9, or more.

In some situations, flexible linkers may be incapable of maintaining a distance or positioning of recombinant receptor needed for a particular use. In these instances, rigid or semi-rigid linkers may be useful. Examples of rigid or semi-rigid linkers include proline-rich linkers. In particular embodiments, a proline-rich linker is a peptide sequence having more proline residues than would be expected based on chance alone. In particular embodiments, a proline-rich linker is one having at least 30%, at least 35%, at least 36%, at least 39%, at least 40%, at least 48%, at least 50%, or at least 51% proline residues. Particular examples of proline-rich linkers include fragments of proline-rich salivary proteins (PRPs).

Linkers can be susceptible to cleavage (cleavable linker), such as, acid-induced cleavage, photo-induced cleavage, peptidase-induced cleavage, esterase-induced cleavage, and disulfide bond cleavage. Alternatively, linkers can be substantially resistant to cleavage (e.g., stable linker or noncleavable linker). In some aspects, the linker is a procharged linker, a hydrophilic linker, or a dicarboxylic acid-based linker.

Junction amino acids can be a linker which can be used to connect sequences when the distance provided by a spacer is not needed and/or wanted. For example, junction amino acids can be short amino acid sequences that can be used to connect co-stimulatory intracellular signaling components. In particular embodiments, junction amino acids are 9 amino acids or less (e.g., 2, 3, 4, 5, 6, 7, 8, or 9 amino acids). In particular embodiments, a glycine-serine doublet can be used as a suitable junction amino acid linker. In particular embodiments, a single amino acid, e.g., an alanine, a glycine, can be used as a suitable junction amino acid.

In particular embodiments, the recombinant receptor can optionally include a multimerization domain. Protein biological activities depend upon their tertiary and quaternary structure. The quaternary structure requires the physical and chemical interaction of different protein subunits or polypeptides. A “multimerization domain” is a domain that causes two or more proteins (monomers) to interact with each other through covalent and/or non-covalent association(s). Multimerization domains present in proteins can result in protein interactions that form dimers, trimers, tetramers, pentamers, hexamers, heptamers, etc., depending on the number of units/monomers incorporated into the multimer.

### (vii) EX VIVO MANUFACTURED CELL FORMULATIONS

In particular embodiments, genetically modified cells can be harvested from a culture medium and washed and concentrated into a carrier in a therapeutically-effective amount. Exemplary carriers include saline, buffered saline, physiological saline, water, Hanks' solution, Ringer's solution, Normosol-R (Abbott Labs), PLASMA-LYTE A® (Baxter Laboratories, Inc., Morton Grove, IL), and combinations thereof.

In particular embodiments, carriers can be supplemented with human serum albumin (HSA) or other human serum components or fetal bovine serum. In particular embodiments, a carrier for infusion includes buffered saline with 5% HSA or dextrose. Additional isotonic agents include polyhydric sugar alcohols including trihydric or higher sugar alcohols, such as glycerin, erythritol, arabitol, xylitol, sorbitol, or mannitol.

Carriers can include buffering agents, such as citrate buffers, succinate buffers, tartrate buffers, fumarate buffers, gluconate buffers, oxalate buffers, lactate buffers, acetate buffers, phosphate buffers, histidine buffers, and/or trimethylamine salts.

Stabilizers refer to a broad category of excipients which can range in function from a bulking agent to an additive which helps to prevent cell adherence to container walls. Typical stabilizers can include polyhydric sugar alcohols; amino acids, such as arginine, lysine, glycine, glutamine, asparagine, histidine, alanine, ornithine, L-leucine, 2-phenylalanine, glutamic acid, and threonine; organic sugars or sugar alcohols, such as lactose, trehalose, stachyose, mannitol, sorbitol, xylitol, ribitol, myoinisitol, galactitol, glycerol, and cyclitols, such as inositol; PEG; amino acid polymers; sulfur-containing reducing agents, such as urea, glutathione, thioctic acid, sodium thioglycolate, thioglycerol, alpha-monothioglycerol, and sodium thiosulfate; low molecular weight polypeptides (i.e., <10 residues); proteins such as HSA, bovine serum albumin, gelatin or immunoglobulins; hydrophilic polymers such as polyvinylpyrrolidone; monosaccharides such as xylose, mannose, fructose and glucose; disaccharides such as lactose, maltose and sucrose; trisaccharides such as raffinose, and polysaccharides such as dextran.

Where necessary or beneficial, formulations can include a local anesthetic such as lidocaine to ease pain at a site of injection.

Exemplary preservatives include phenol, benzyl alcohol, meta-cresol, methyl paraben, propyl paraben, octadecyldimethylbenzyl ammonium chloride, benzalkonium halides, hexamethonium chloride, alkyl parabens such as methyl or propyl paraben, catechol, resorcinol, cyclohexanol, and 3-pentanol.

Therapeutically effective amounts of cells within formulations can be greater than 10² cells, greater than 10³ cells, greater than 10⁴ cells, greater than 10⁵ cells, greater than 10⁶ cells, greater than 10⁷ cells, greater than 10⁸ cells, greater than 10⁹ cells, greater than 10¹⁰ cells, or greater than 10¹¹.

In formulations disclosed herein, cells are generally in a volume of a liter or less, 500 ml or less, 250 ml or less or 100 ml or less. Hence the density of administered cells is typically greater than 10⁴ cells/ml, 10⁷ cells/ml or 10⁸ cells/ml.

In particular embodiments, formulations can include one or more genetically modified cell types (e.g., modified T cells, NK cells, or stem cells). Formulations can include different types of genetically-modified cells (e.g., T cells, NK cells, and/or stem cells in combination).

Different types of genetically-modified cells or cell subsets (e.g., modified T cells, NK cells, and/or stem cells) can be provided in different ratios e.g., a 1:1:1 ratio, 2:1:1 ratio, 1:2:1 ratio, 1:1:2 ratio, 5:1:1 ratio, 1:5:1 ratio, 1:1:5 ratio, 10:1:1 ratio, 1:10:1 ratio, 1:1:10 ratio, 2:2:1 ratio, 1:2:2 ratio, 2:1:2 ratio, 5:5:1 ratio, 1:5:5 ratio, 5:1:5 ratio, 10:10:1 ratio, 1:10:10 ratio, 10:1:10 ratio, etc. These ratios can also apply to numbers of cells expressing the same or different PD1:cytokine chimera transgenes and/or recombinant receptors. If only two of the cell types are combined or only 2 combinations of expressed PD1:cytokine chimera transgene components are included within a formulation, the ratio can include any 2-number combination that can be created from the 3 number combinations provided above. In embodiments, the combined cell populations are tested for efficacy and/or cell proliferation in vitro, in vivo and/or ex vivo, and the ratio of cells that provides for efficacy and/or proliferation of cells is selected.

The cell-based formulations disclosed herein can be prepared for administration by, e.g., injection, infusion, perfusion, or lavage. The formulations can further be formulated for bone marrow, intravenous, intradermal, intraarterial, intranodal, intralymphatic, intraperitoneal, intralesional, intraprostatic, intravaginal, intrarectal, intrathecal, intratumoral, intramuscular, intravesicular, and/or subcutaneous injection.

### (viii) COMPOSITIONS FOR TARGETED VIRAL VECTORS & NANOPARTICLES FOR IN VIVO CELL MODIFICATION

Targeted viral vectors and/or nanoparticles can also be used to genetically-modify immune cells in vivo or ex vivo. Viral vectors that can be used to deliver artificial expression constructs to cells are described elsewhere herein, and numerous targeted (e.g., pseudotyped) viral vectors are known in the art.

Exemplary cell-targeted nanoparticles include a cell targeting ligand (e.g., CD3, CD4, CD8, CD34) on the surface of the nanoparticle wherein the cell targeting ligand results in selective uptake of the nanoparticle by a selected cell type. The nanoparticle then delivers gene modifying components that result in expression of the PD1:cytokine chimera transgene and recombinant receptor.

Exemplary nanoparticles include liposomes (microscopic vesicles including at least one concentric lipid bilayer surrounding an aqueous core), liposomal nanoparticles (a liposome structure used to encapsulate another smaller nanoparticle within its core); and lipid nanoparticles (liposome-like structures that lack the continuous lipid bilayer characteristic of liposomes). Other polymer-based nanoparticles can also be used as well as porous nanoparticles constructed from any material capable of forming a porous network. Exemplary materials include metals, transition metals and metalloids (e.g., lithium, magnesium, zinc, aluminum and silica).

For in vivo delivery and cellular uptake, nanoparticles can have a neutral or negatively-charged coating and a size of 130 nm or less. Dimensions of the nanoparticles can be determined using, e.g., conventional techniques, such as dynamic light scattering and/or electron microscopy.

In particular embodiments, the nanoparticles can be those described in WO2014153114, WO2017181110, and WO201822672.

Therapeutically effective amounts of vectors and/or nanoparticles within formulations can range from 0.1 to 5 μg/kg or from 0.5 to 1 μg/kg. In other examples, a dose can include 1 μg/kg, 30 μg/kg, 90 μg/kg, 150 μg/kg, 500 μg/kg, 750 μg/kg, 0.1 to 5 mg/kg or from 0.5 to 1 mg/kg. In other examples, a dose can include 1 mg/kg, 10 mg/kg, 30 mg/kg, 50 mg/kg, 70 mg/kg, 100 mg/kg, 300 mg/kg, 500 mg/kg, 700 mg/kg, 1000 mg/kg or more.

### (ix) METHODS OF USE

Methods disclosed herein include treating subjects (humans, non-human primates, veterinary animals (dogs, cats, reptiles, birds, etc.) livestock (horses, cattle, goats, pigs, chickens, etc.) and research animals (monkeys, rats, mice, fish, etc.)) with formulations disclosed herein. Treating subjects includes delivering therapeutically effective amounts. Therapeutically effective amounts include those that provide effective amounts, prophylactic treatments and/or therapeutic treatments.

An “effective amount” is the amount of a formulation necessary to result in a desired physiological change in the subject. For example, an effective amount can provide an enhanced immunogenic anti-cancer or anti-infection (e.g., infection with virus, bacteria, fungi, parasite, or arthropod) effect. Effective amounts are often administered for research purposes. Effective amounts disclosed herein can cause a statistically significant effect in an animal model or in vitro assay relevant to the assessment of a cancer or infection's development or progression. An immunogenic formulation can be provided in an effective amount, wherein the effective amount stimulates an immune response.

A “prophylactic treatment” includes a treatment administered to a subject who does not display signs or symptoms of a cancer or infection or displays only early signs or symptoms of a cancer or infection such that treatment is administered for the purpose of diminishing or decreasing the risk of developing the cancer or infection further. Thus, a prophylactic treatment functions as a preventative treatment against a cancer or infection. In particular embodiments, prophylactic treatments reduce, delay, or prevent metastasis from a primary cancer tumor site from occurring. In particular embodiments, prophylactic treatments reduce, delay, or prevent infection from a bacteria, virus, fungi, parasite, or arthropod.

A “therapeutic treatment” includes a treatment administered to a subject who displays symptoms or signs of a cancer or infection and is administered to the subject for the purpose of diminishing or eliminating those signs or symptoms of the cancer or infection. The therapeutic treatment can reduce, control, or eliminate the presence or activity of the cancer or infection and/or reduce control or eliminate side effects of the cancer or infection.

Function as an effective amount, prophylactic treatment or therapeutic treatment are not mutually exclusive, and in particular embodiments, administered dosages may accomplish more than one treatment type.

In particular embodiments, therapeutically effective amounts potentiate cell killing ability, proliferation, and/or cytokine output by immune cells. These effects can provide anti-cancer effects and/or anti-infection effects. Anti-cancer effects include a decrease in the number of cancer cells, decrease in the number of metastases, a decrease in tumor volume, an increase in life expectancy, induced chemo- or radiosensitivity in cancer cells, inhibited angiogenesis near cancer cells, inhibited cancer cell proliferation, inhibited tumor growth, prevented or reduced metastases, prolonged subject life, reduced cancer-associated pain, and/or reduced relapse or re-occurrence of cancer following treatment. Anti-infection effects include a decrease in the amount or level of infective pathogen, fatigue, loss of appetite, weight loss, fevers, night sweats, chills, aches and pains, diarrhea, bloating, abdominal pain, skin rashes, coughing, and/or a runny nose.

A “tumor” is a swelling or lesion formed by an abnormal growth of cells (called neoplastic cells or tumor cells). A “tumor cell” is an abnormal cell that grows by a rapid, uncontrolled cellular proliferation and continues to grow after the stimuli that initiated the new growth cease. Tumors show partial or complete lack of structural organization and functional coordination with the normal tissue, and usually form a distinct mass of tissue, which may be benign, pre-malignant or malignant.

In particular embodiments, a subject's immune cells are screened for responsiveness to inducible PD1:cytokine chimera transgene expression. Screening methods include introducing a genetic construct including a sequence encoding an inducible PD1:cytokine chimera as described elsewhere herein to a plurality of immune cells from a subject; and measuring the potentiated function of the immune cells. In particular embodiments, the potentiated function of the immune cells includes potentiated cell killing, potentiated cell proliferation, and/or cytokine output. In particular embodiments, potentiated cytokine output includes IFN{and/or TNFa output. Methods for screening a subject's immune cells for responsiveness to inducible cytokine secretion can include cell killing assays and proliferation assays such as an MTT cell proliferation assay, trypan blue assay, carboxyfluorescein succinimidyl ester (CFSE) assay, Ki67, XTT assay, BrdU assay, flow cytometry, or DNA quantitation; or cytokine output assays such as quantitative PCR or immunodetection (e.g., enzyme-linked immunosorbent assay). Methods for screening a subject's immune cells for responsiveness to inducible cytokine expression can be performed in vitro or in vivo.

In particular embodiments, inducible PD1:cytokine chimera transgenes that elicit an increased potentiated function of immune cells compared to a baseline can be selected for administration to the subject. In particular embodiments, the baseline includes immune cell function before introduction of the artificial expression construct or immune cell function after introducing a control (non-PD1:cytokine chimera expressing) artificial expression construct.

In particular embodiments, a combination of PD1:cytokine chimera transgenes can be chosen to administer to a subject based on the screening results. For example, if a first PD1:cytokine chimera increases cell killing ability and proliferation and a second PD1:cytokine chimera increases cytokine output in a first subject, then the first PD1:cytokine chimera and second PD1:cytokine chimera may be chosen for administration to the first subject. Alternatively, if a third PD1:cytokine chimera increases cell killing ability, a fourth PD1:cytokine chimera increases proliferation, and a fifth PD1:cytokine chimera increases cytokine output in a second subject, then the third PD1:cytokine chimera, the fourth PD1:cytokine chimera, and the fifth PD1:cytokine chimera may be chosen for administration to the second subject.

In particular embodiments, any combination of PD1:cytokine chimera transgenes can be administered. In particular embodiments, the combination of PD1:cytokine chimera transgenes that potentiates cell killing ability, proliferation, and/or cytokine output includes selecting at least one of PD1:IL21, PD1:IL15(N72D), PD1:drIL18, PD1:IL12, or PD1:IFNα; and at least one of PD1:IL21, PD1:IL15(N72D), PD1:drIL18, PD1:IL12, or PD1:IFNα. In particular embodiments, the combination of PD1:cytokine chimera transgenes that potentiates cell killing includes PD1:IFNα and at least one of PD1:IL21, PD1:IL15(N72D), PD1:drIL18, or PD1:IL12. In particular embodiments, the combination of PD1:cytokine chimera transgenes that potentiates cell killing includes PD1:IL21 and at least one of PD1:IL15(N72D), PD1:drIL18, PD1:IL12, or PD1:IFNα. In particular embodiments, the combination of PD1:cytokine chimera transgenes that potentiates cell killing includes PD1:IL15(N72D) and at least one of PD1:IL21, PD1:drIL18, PD1:IL12, or PD1:IFNα. In particular embodiments, the combination of PD1:cytokine chimera transgenes that potentiates cell killing includes PD1:drIL18, and at least one of PD1:IL21, PD1:IL15(N72D), PD1:IL12, or PD1:IFNα. In particular embodiments, the combination of PD1:cytokine chimera transgenes that potentiates cell killing includes PD1:IL12 and at least one of PD1:IL21, PD1:IL15(N72D), PD1:drIL18, or PD1:IFNα.

For administration, therapeutically effective amounts (also referred to herein as doses) can be initially estimated based on results from in vitro assays and/or animal model studies. Such information can be used to more accurately determine useful doses in subjects of interest. The actual dose amount administered to a particular subject can be determined by a physician, veterinarian or researcher taking into account parameters such as physical and physiological factors including target, body weight, severity of condition, type of cancer or infection, stage of cancer or infection, previous or concurrent therapeutic interventions, idiopathy of the subject and route of administration.

Therapeutically effective amounts of cell-based formulations can include 104 to 10⁹ cells/kg body weight, or 103 to 10¹¹ cells/kg body weight. Therapeutically effective amounts to administer can include greater than 10² cells, greater than 10³ cells, greater than 10⁴ cells, greater than 10⁵ cells, greater than 10⁶ cells, greater than 10⁷ cells, greater than 10⁸ cells, greater than 10⁹ cells, greater than 10¹⁰ cells, or greater than 10¹¹.

Therapeutically effective amounts of vectors and/or nanoparticles within formulations can range from 0.1 to 5 μg/kg or from 0.5 to 1 μg/kg. In other examples, a dose can include 1 μg/kg, 30 μg/kg, 90 μg/kg, 150 μg/kg, 500 μg/kg, 750 μg/kg, 0.1 to 5 mg/kg or from 0.5 to 1 mg/kg. In other examples, a dose can include 1 mg/kg, 10 mg/kg, 30 mg/kg, 50 mg/kg, 70 mg/kg, 100 mg/kg, 300 mg/kg, 500 mg/kg, 700 mg/kg, 1000 mg/kg or more.

Therapeutically effective amounts can be achieved by administering single or multiple doses during the course of a treatment regimen (e.g., daily, every other day, every 3 days, every 4 days, every 5 days, every 6 days, weekly, every 2 weeks, every 3 weeks, monthly, every 2 months, every 3 months, every 4 months, every 5 months, every 6 months, every 7 months, every 8 months, every 9 months, every 10 months, every 11 months or yearly). In particular embodiments, the treatment protocol may be dictated by a clinical trial protocol or an FDA-approved treatment protocol.

Therapeutically effective amounts can be administered by, e.g., injection, infusion, perfusion, or lavage. Routes of administration can include bolus intravenous, intradermal, intraarterial, intraparenteral, intranodal, intralymphatic, intraperitoneal, intralesional, intraprostatic, intravaginal, intrarectal, topical, intrathecal, intratumoral, intramuscular, intravesicular, and/or subcutaneous administration.

In certain embodiments, formulations and/or compositions are administered to a patient in conjunction with (e.g., before, simultaneously or following) any number of relevant treatment modalities. In particular embodiments, cells may be used in combination with chemotherapy, radiation, immunosuppressive agents, such as cyclosporin, azathioprine, methotrexate, mycophenolate, and FK506, antibodies, or other immunoablative agents such as CAM PATH, anti-CD3 antibodies or other antibody therapies, cytoxin, fludaribine, cyclosporin, FK506, rapamycin, mycoplienolic acid, steroids, FR901228, cytokines, and irradiation.

In certain embodiments, formulations and/or compositions may be administered in conjunction with any number of chemotherapeutic agents. Examples of chemotherapeutic agents include alkylating agents; alkyl sulfonates; aziridines; ethylenimines and methylamelamines; nitrogen mustards; nitrosureas; antibiotics; anti-metabolites; folic acid analogues; purine analogs; pyrimidine analogs; androgens; anti-adrenals; folic acid replenisher; platinum analogs; retinoic acid derivatives; and pharmaceutically acceptable salts, acids or derivatives of any of the above.

Also included in this definition are anti-hormonal agents that act to regulate or inhibit hormone action on tumors such as anti-estrogens and anti-androgens; and pharmaceutically acceptable salts, acids or derivatives of any of the above. Combinations of chemotherapeutic agents are also administered where appropriate, including, CHOP, i.e., Cyclophosphamide (Cytoxan®, Ingenus Pharmaceuticals, LLC, Tampa, FL), Doxorubicin (hydroxydoxorubicin), Vincristine (Oncovin), and Prednisone.

In some embodiments, the chemotherapeutic agent is administered at the same time or within one week after the administration of the formulation or composition. In other embodiments, the chemotherapeutic agent is administered from 1 to 4 weeks or from 1 week to 1 month, 1 week to 2 months, 1 week to 3 months, 1 week to 6 months, 1 week to 9 months, or 1 week to 12 months after the administration of the engineered cell or nucleic acid. In other embodiments, the chemotherapeutic agent is administered at least 1 month before administering the cell or nucleic acid. In some embodiments, the methods further include administering two or more chemotherapeutic agents.

A variety of additional therapeutic agents may be used in conjunction with the formulations described herein. For example, potentially useful additional therapeutic agents include PD-1 inhibitors such as nivolumab (Opdivo®, Bristol-Myers Squibb Company, New York, NY), pembrolizumab (Keytruda®, Merck Sharp & Dohme LLC, Rahway<NJ), pembrolizumab, pidilizumab, and atezolizumab, and CTLA-4 inhibitors, such as ipilimumab (Yervoy®, Bristol-Myers Squibb Company).

In additional embodiments, the formulations and/or compositions can be administered with an analgesic. Exemplary analgesics include acetaminophen, oxycodone, tramadol of proporxyphene hydrochloride. The formulations and/or compositions can be administered with an anti-inflammatory agent. Anti-inflammatory agents or drugs include steroids and glucocorticoids, and nonsteroidal anti-inflammatory drugs (NSAIDS). In further embodiments, the formulations and/or compositions can be administered with biologic response modifiers. Exemplary biological response modifiers include molecules directed against cell surface markers (e.g., CD4, CD5, etc.), cytokine inhibitors, such as the TNF antagonists, (e.g., etanercept (ENBREL®, Immunex Corporation, Seattle, WA), adalimumab (HUMIRA®, AbbVie Biotechnology, Ltd, North Chicago, IL) and infliximab (REMICADE®, Janssen Biotech, Inc., Horsham Township, PA), chemokine inhibitors and adhesion molecule inhibitors. In some aspects, the biological response modifiers may include monoclonal antibodies as well as recombinant forms of molecules. The formulations and/or compositions may be administered with disease-modifying antirheumatic drugs.

Exemplary disease-modifying antirheumatic drugs (DMARDs) include azathioprine, cyclophosphamide, cyclosporine, methotrexate, penicillamine, leflunomide, sulfasalazine, hydroxychloroquine, Gold (oral (auranofm) and intramuscular) and minocycline. In additional embodiments, the formulations and/or compositions can be administered with an anti-infective agent (e.g., antiviral). Exemplary anti-infective agents include ribavirin, 4′-fluorouridine, small molecule TMC353121, AVG-388, valcyclovir, acyclovir, cidofovir, foscarnet, ganciclovir, valganciclovir, penciclovir, famciclovir, idoxuridine, trifluorothymidine, vidarabine, fomivirsen, amantadine, remintadine, zanamivir, oseltamivir, lamivudine, adefovir dipivoxil, entecavir, telbivudine, clevudine, podophyllotoxin, imiquimod, interferons, pleconaril, maraviroc, enfuvirtide, zidovudine, didanosine, zalcitabine, stavudine, lamivudine, emtricitabine, tenofovir disoproxil fumarate, nevirapine, delavirdine, defavirenz, raltegravir, saquinavir, indinavir, ritonavir, nelfinavir, amprenavir, fosamprenavir, lopinavir, atazanavir, tipranavir, and darunavir.

In certain embodiments, the formulations and/or compositions are administered in conjunction with a cytokine. Cytokines that could be co-administered with the formulations and/or composition disclosed herein include, for example, interferons such as interferon-alpha, beta, and -gamma; colony stimulating factors (CSFs) such as macrophage-CSF (M-CSF); granulocyte-macrophage-CSF (GM-CSF); and granulocyte-CSF (G-CSF); interleukins (ILs) such as IL-1, IL-la, IL-2, IL-3, IL-4, IL-5, IL-6, IL-7, IL-8, IL-9, IL-10, IL-11, IL-12, scIL12, IL-15, IL18, drIL18, IL21, IL36y, a tumor necrosis factor such as TNFα or TNFβ, or TGFβ2-7m; and other polypeptide factors including LIF and kit ligand (KL). In relation to these combination treatments, the term cytokine includes proteins from natural sources or from recombinant cell culture, and biologically active equivalents of the native sequence cytokines.

### (x) KITS

The current disclosure also includes kits. Kits can include various components to practice methods disclosed herein. For example, depending on the aspect of the methods practiced, kits could include one or more of nucleic acids encoding a PD1:cytokine chimera transgene disclosed herein under the control of an iSynPro promoter; an IL2 minimal promoter; a constitutive promoter; nucleic acids encoding a recombinant receptor disclosed herein; a nucleic acid encoding Her2tg; a skip sequence; a nucleic acid encoding an scFv; a nucleic acid encoding a VL; a nucleic acid encoding a VH; a nucleic acid encoding a transmembrane domain; a nucleic acid encoding an intracellular effector domain; a nucleic acid encoding EGFRt; a nucleic acid encoding selection cassettes (e.g. DHFRdm); methotrexate; cells (e.g., immune cells, T-cells, CD4+ T cells, CD8+ T cells, B cells, natural killer (NK) cells, NK-T cells, monocytes/macrophages, lymphocytes, hematopoietic stem cells (HSCs), hematopoietic progenitor cells (HPC), and/or a mixture of HSC and HPC (i.e., HSPC), untransduced T cells, T cells transduced with artificial expression constructs describe herein); cell lines; tissue samples (e.g., peripheral blood mononuclear cells (PBMCs), leukocytes, bone marrow, thymus, tissue biopsy, tumor, lymph node, gut associated lymphoid tissue, mucosa associated lymphoid tissue, spleen, other lymphoid tissues, liver, lung, stomach, intestine, colon, kidney, pancreas, breast, bone, prostate, cervix, testes, ovaries, tonsil, or other organ, and/or cells derived therefrom); genetic expression components (e.g., genes for expression provided by vectors (e.g., lentiviral vector, retroviral vector), CRISPR components, ZFNs, TALENs, MegaTALs, targeted viral vectors and/or nanoparticles); cell formulation or activation components (e.g., saline, buffered saline, phosphate buffered saline (PBS); biocompatible buffers such as, Ca++/Mg++ free PBS; physiological saline, water, Hanks' solution, Ringer's solution, T cell stimulating epitopes (e.g., anti-CD3/anti-CD28 conjugated beads; OKT3, TGN1412), culture-initiating compositions, RPMI, non-essential amino acids, sodium pyruvate, penicillin/streptomycin, non-dividing EBV-transformed lymphoblastoid cells (LCL), IL-21, human serum albumin (HSA) or other human serum components or fetal bovine serum, dextrose, stabilizers, preservatives); combination therapy components (e.g., local anesthetics, chemotherapeutic agents, immunosuppressive agents, anti-inflammatory agents, anti-infective agents); an antibody tagged with a fluorescent molecule; PCR amplification sequences; PD1:cytokine chimera transgenes (e.g., PD1:IL21, PD1:IL15(N72D), PD1:drIL18, PD1:IL12, PD1:IFNα); culture vessels; reference levels, transgenic animals; primer pairs; GAPDH; IFN-γ enzyme-linked immunosorbent assay (ELISA); culture plates; etc.

The Exemplary Embodiments and Example below are included to demonstrate particular embodiments of the disclosure. Those of ordinary skill in the art should recognize in light of the present disclosure that many changes can be made to the specific embodiments disclosed herein and still obtain a like or similar result without departing from the spirit and scope of the disclosure.

### (xi) Exemplary Embodiments

1. An artificial expression construct including a sequence encoding a programmed cell death-1 (PD1):cytokine chimera, wherein the PD1: cytokine chimera includes a PD1 linked to a cytokine.
2. The artificial expression construct of embodiment 1, wherein the cytokine is an immunostimulatory cytokine.
3. The artificial expression construct of embodiment 2, wherein the immunostimulatory cytokine includes an interleukin (IL)21, an IL15, an N72D mutant IL15 (IL15(N72D), an IL18, a decoy resistant IL18 (drIL18), an IL12, or an interferon alpha (IFNα).
4. The artificial expression construct of any of embodiments 1-3, wherein the sequence encoding the PD1:cytokine chimera is under the regulatory control of a promoter, wherein the promoter includes a sequence having at least 95% sequence identity to any one of SEQ ID NOs: 17-56 and a minimal promoter.
5. The artificial expression construct of embodiment 4, wherein the promoter has at least 98% sequence identity to any one of SEQ ID NOs: 17-56 and a minimal promoter.
6. The artificial expression construct of embodiments 4 or 5, wherein the promoter has at least 99% sequence identity to any one of SEQ ID NOs: 17-56 and a minimal promoter.
7. The artificial expression construct of any of embodiments 4-6, wherein the promoter has the sequence as set forth in any one of SEQ ID NOs: 17-56 and a minimal promoter.
8. The artificial expression construct of any of embodiments 4-7, wherein the minimal promoter is an IL2 minimal promoter.
9. The artificial expression construct of embodiment 8, wherein the IL2 minimal promoter includes the sequence as set forth in SEQ ID NO: 138.
10. The artificial expression construct of any of embodiments 1-9, wherein the PD1:cytokine chimera includes:
   PD1 linked to interleukin (IL)21 (PD1:IL21),
   PD1 linked to IL15 (PD1:IL15),
   PD1 linked to an N72D mutant IL15 (PD1:IL15(N72D)),
   PD1 linked to IL18 (PD1:IL18),
   PD1 linked to decoy resistant IL18 (PD1:drIL18),
   PD1 linked to IL12 (PD1:IL12), or
   PD1 linked to interferon alpha (PD1:IFNα).
11. The artificial expression construct of any of embodiments 1-10, wherein the PD1 is PD1 with an A99L mutation.
12. The artificial expression construct of any of embodiments 1-10, wherein the PD1 is wildtype PD1.
13. The artificial expression construct of any of embodiments 1-12, wherein the PD1 is linked to the cytokine through a protein linker.
14. The artificial expression construct of embodiment 13, wherein the protein linker is a flexible linker.
15. The artificial expression construct of embodiment 14, wherein the flexible linker is a Gly-Ser linker.
16. The artificial expression construct of embodiment 15, wherein the Gly-Ser linker includes a sequence as set forth in SEQ ID NO: 128 or the sequence GGG.
17. The artificial expression construct of any of embodiments 1-16, wherein the PD1:cytokine chimera is encoded by the sequence as set forth in SEQ ID NO: 7, SEQ ID NO: 8, SEQ ID NO: 9, SEQ ID NO: 10, or SEQ ID NO: 11 or a sequence having at least 90% sequence identity to the sequence set forth in SEQ ID NO: 7, SEQ ID NO: 8, SEQ ID NO: 9, SEQ ID NO: 10, or SEQ ID NO: 11.
18. The artificial expression construct of any of embodiments 1-17, wherein the PD1:cytokine chimera includes the sequence as set forth in SEQ ID NO: 12, SEQ ID NO: 13, SEQ ID NO: 14, SEQ ID NO: 15, or SEQ ID NO: 16 or a sequence having at least 90% sequence identity to the sequence set forth SEQ ID NO: 12, SEQ ID NO: 13, SEQ ID NO: 14, SEQ ID NO: 15, or SEQ ID NO: 16.
19. The artificial expression construct of any of embodiments 1-18, further including a control feature.
20. The artificial expression construct of embodiment 19, wherein the control feature encodes a transduction marker.
21. The artificial expression construct of embodiment 20, wherein the transduction marker includes Her2tG.
22. The artificial expression construct of any of embodiments 1-21, further including a first skip sequence.
23. The artificial expression construct of embodiment 22, wherein the first skip sequence encodes a 2A self-cleaving polypeptide.
24. The artificial expression construct of embodiment 23, wherein the 2A self-cleaving polypeptide includes T2A, P2A, E2A, or F2A.
25. The artificial expression construct of any of embodiments 1-24, wherein the artificial expression construct includes from 5′ to 3′: a promoter including the sequence as set forth in any one of SEQ ID NOs: 17-56 and a sequence as set forth in SEQ ID NO: 138; a control feature; a skip sequence; and a sequence as set forth in any one of SEQ ID NOs: 1-11.
26. The artificial expression construct of any of embodiments 1-25, wherein the artificial expression construct includes from 5′ to 3′: a promoter including the sequence as set forth in any one of SEQ ID NOs: 17-56 and a sequence as set forth in SEQ ID NO: 138; a sequence encoding a transduction marker; a skip sequence; and a sequence as set forth in any one of SEQ ID NOs: 1-11.
27. The artificial expression construct of any of embodiments 1-26, wherein the artificial expression construct has at least 95% sequence identity to the sequence as set forth in SEQ ID NO: 103.
28. The artificial expression construct of any of embodiments 1-27, wherein the artificial expression construct has at least 98% sequence identity to the sequence as set forth in SEQ ID NO: 103.
29. The artificial expression construct of any of embodiments 1-28, wherein the artificial expression construct has at least 99% sequence identity to the sequence as set forth in SEQ ID NO: 103.
30. The artificial expression construct of any of embodiments 1-29, wherein the artificial expression construct has the sequence as set forth in SEQ ID NO: 103.
31. The artificial expression construct of any of embodiments 1-30, further including a sequence encoding a recombinant receptor, wherein the recombinant receptor includes a binding domain that binds a target antigen.
32. The artificial expression construct of embodiment 31, wherein the sequence encoding the recombinant receptor is operably linked to a second promoter.
33. The artificial expression construct of embodiment 32, wherein the second promoter includes an EF1a(L) promoter, EF1a(s) promoter, myeloproliferative sarcoma virus (MND) promoter, cytomegalovirus (CMV) promoter, simian virus 40 (SV40) early promoter, mouse mammary tumor virus (MMTV) promoter, human immunodeficiency virus (HIV) long terminal repeat (LTR) promoter, MoMuLV promoter, avian leukemia virus promoter, Epstein-Barr virus immediate early promoter, Rous sarcoma virus promoter, actin promoter, myosin promoter, hemoglobin promoter, or a creatine kinase promoter.
34. The artificial expression construct of embodiment 31, wherein the binding domain is part of an extracellular component.
35. The artificial expression construct of any of embodiments 31-34, wherein the target antigen is expressed on the surface of target cells.
36. The artificial expression construct of embodiment 35, wherein the target cells include cancer cells or cells infected with bacteria, virus, fungi, parasites, or arthropods.
37. The artificial expression construct of embodiments 35 or 36, wherein the target cells include cancer cells.
38. The artificial expression construct of any of embodiments 31-37, wherein the recombinant receptor further includes an intracellular component.
39. The artificial expression construct of embodiment 38, wherein the intracellular component includes a CD3ζ signaling domain and/or a 4-1 BB signaling domain.
40. The artificial expression construct of embodiments 38 or 39, wherein the intracellular component is linked to an extracellular component through a transmembrane domain.
41. The artificial expression construct of embodiment 40, wherein the transmembrane domain includes a CD28 transmembrane domain.
42. The artificial expression construct of any of embodiments 31-41, further including a second control feature.
43. The artificial expression construct of embodiment 42, wherein the second control feature encodes a selection cassette.
44. The artificial expression construct of embodiment 43, wherein the selection cassette includes dihydrofolate reductase double mutant (DHFRdm).
45. The artificial expression construct of any of embodiments 42-44, further including a third control feature.
46. The artificial expression construct of embodiment 45, wherein the third control feature encodes a transduction marker.
47. The artificial expression construct of embodiment 46, wherein the transduction marker includes epidermal growth factor receptor (EGFRt) or truncated CD19 (tCD19).
48. The artificial expression construct of any of embodiments 31-47, further including a second skip sequence.
49. The artificial expression construct of embodiment 48, wherein the second skip sequence encodes a 2A self-cleaving polypeptide.
50. The artificial expression construct of embodiment 49, wherein the 2A skip self-cleaving polypeptide includes T2A, P2A, E2A, or F2A.
51. The artificial expression construct of any of embodiments 31-50, wherein the artificial expression construct includes a second skip sequence at the 5′ end of the selection cassette and a third skip sequence at the 5′ end of the transduction marker.
52. The artificial expression construct of any of embodiments 1-51, wherein the artificial expression construct includes from 5′ to 3′: a first promoter, wherein the first promoter includes the sequence as set forth in any one of SEQ ID NOs: 17-56 and a sequence as set forth in SEQ ID NO: 138; a first control feature; a first skip sequence; a sequence as set forth in any one of SEQ ID NOs: 1-11; a second promoter; a sequence encoding a recombinant receptor; a second skip sequence; a second control feature; a third skip sequence; and a third control feature.
53. The artificial expression construct of any of embodiments 1-52, wherein the artificial expression construct includes from 5′ to 3′: a first promoter, wherein the first promoter includes the sequence as set forth in any one of SEQ ID NOs: 17-56 and a sequence as set forth in SEQ ID NO: 138; a sequence encoding a first transduction marker; a first skip sequence; a sequence as set forth in any one of SEQ ID NOs: 1-11; a second promoter; a sequence encoding a recombinant receptor; a second skip sequence; a sequence encoding a selection cassette; a third skip sequence; and a sequence encoding a second transduction marker.
54. The artificial expression construct of any of embodiments 1-53, wherein the artificial expression construct has at least 90% sequence identity to the sequence as set forth in SEQ ID NOs: 98, 99, 100, 101, or 102.
55. The artificial expression construct of any of embodiments 1-54, wherein the artificial expression construct has the sequence as set forth in SEQ ID NOs: 98, 99, 100, 101, or 102.
56. A system for potentiating immune cell function including: a first artificial expression construct encoding a PD1:cytokine chimera under the regulatory control of a first promoter, wherein the first promoter includes a sequence having at least 95% sequence identity to any of SEQ ID NOs: 17-56 and a minimal promoter, and wherein the PD1: chimera includes a PD1 linked to a cytokine; and a second artificial expression construct includes a sequence encoding a recombinant receptor, the recombinant receptor including a binding domain that binds an antigen expressed on the surface of target cells.
57. The system of embodiment 56, wherein the cytokine is an immunostimulatory cytokine.
58. The system of embodiment 57, wherein the immunostimulatory cytokine includes an interleukin (IL)21, an I15, an N72D mutant IL15 (IL15(N72D), an IL18, a decoy resistant IL18 (drIL18), an IL12, or an interferon alpha (IFNα).
59. The system of any of embodiments 56-58, wherein the first promoter includes a sequence having at least 98% sequence identity to any one of SEQ ID NOs: 17-56 and a minimal promoter.
60. The system of any of embodiments 56-59, wherein the first promoter includes a sequence having at least 99% sequence identity to any one of SEQ ID NOs: 17-56 and a minimal promoter.
61. The system of any of embodiments 56-60, wherein the first promoter includes the sequence as set forth in any one of SEQ ID NOs: 17-56 and a minimal promoter.
62. The system of any of embodiments 56-61, wherein the minimal promoter includes a I1L2 minimal promoter.
63. The system of embodiment 62, wherein the IL2 minimal promoter includes the sequence as set forth in SEQ ID NO: 138.
64. The system of any of embodiments 56-63, wherein the PD1:cytokine chimera includes:
   PD1 linked to interleukin (IL)21 (PD1:IL21),
   PD1 linked to IL15 (PD1:IL15),
   PD1 linked to an N72D mutant IL15 (PD1:IL15(N72D)),
   PD1 linked to IL18 (PD1:IL18),
   PD1 linked to decoy resistant IL18 (PD1:drIL18),
   PD1 linked to IL12 (PD1:IL12), or
   PD1 linked to interferon alpha (PD1:IFNα).
65. The system of any of embodiments 56-64, wherein the PD1 is PD1 with an A99L mutation.
66. The system of any of embodiments 56-64, wherein the PD1 is wildtype PD1.
67. The system of any of embodiments 56-66, wherein the PD1 is linked to the cytokine through a protein linker.
68. The system of embodiment 67, wherein the protein linker is a flexible linker.
69. The system of embodiment 68, wherein the flexible linker is a Gly-Ser linker.
70. The system of embodiment 69, wherein the Gly-Ser linker includes a sequence as set forth in SEQ ID NO: 128 or the sequence GGG.
71. The system of any of embodiments 56-70, wherein the PD1:cytokine chimera is encoded by the sequence as set forth in SEQ ID NO: 7, SEQ ID NO: 8, SEQ ID NO: 9, SEQ ID NO: 10, or SEQ ID NO: 11 or a sequence having at least 90% sequence identity to the sequence set forth in SEQ ID NO: 7, SEQ ID NO: 8, SEQ ID NO: 9, SEQ ID NO: 10, or SEQ ID NO: 11.
72. The system of any of embodiments 56-71, wherein the PD1:cytokine chimera includes the sequence as set forth in SEQ ID NO: 12, SEQ ID NO: 13, SEQ ID NO: 14, SEQ ID NO: 15, or SEQ ID NO: 16 or a sequence having at least 90% sequence identity to the sequence set forth in SEQ ID NO: 12, SEQ ID NO: 13, SEQ ID NO: 14, SEQ ID NO: 15, or SEQ ID NO: 16.
73. The system of any of embodiments 56-72, wherein the first artificial expression construct further includes a control feature.
74. The system of embodiment 73, wherein the control feature encodes a transduction marker.
75. The system of embodiment 74, wherein the transduction marker includes Her2tG.
76. The system of any of embodiments 56-75, wherein the first artificial expression construct further includes a first skip sequence.
77. The system of embodiment 76, wherein the first skip sequence encodes a 2A self-cleaving polypeptide.
78. The system of embodiment 77, wherein the 2A self-cleaving polypeptideincludes T2A, P2A, E2A, or F2A.
79. The system of any of embodiments 56-78, wherein the sequence encoding the recombinant receptor is operably linked to a second promoter.
80. The system of any of embodiments 56-79, wherein the second promoter includes an EF1a(L) promoter, EF1a(s) promoter, MND promoter, CMV promoter, SV40 early promoter, MMTV promoter, HIV LTR promoter, MoMuLV promoter, an avian leukemia virus promoter, an Epstein-Barr virus immediate early promoter, a Rous sarcoma virus promoter, an actin promoter, a myosin promoter, a hemoglobin promoter, or a creatine kinase promoter.
81. The system of any of embodiments 56-80, wherein the second artificial expression construct further includes a second control feature.
82. The system of embodiment 81, wherein the second control feature encodes a selection cassette.
83. The system of embodiment 82, wherein the selection cassette includes dihydrofolate reductase double mutant (DHFRdm).
84. The system of any of embodiments 56-83, wherein the second artificial expression construct further includes a third control feature.
85. The system of embodiment 84, wherein the third control feature encodes a transduction marker.
86. The system of embodiment 85, wherein the transduction marker includes truncated epidermal growth factor receptor (EGFRt) or tCD19.
87. The system of any of embodiments 56-86, wherein the second artificial expression construct further includes a second skip sequence.
88. The system of embodiment 87, wherein the second skip sequence encodes a 2A self-cleaving polypeptide.
89. The system of embodiment 88, wherein the 2A self-cleaving polypeptide includes T2A, P2A, E2A, or F2A.
90. The system of any of embodiments 56-89, wherein the second artificial expression construct includes a second skip sequence at the 5′ end of the selection cassette and a third skip sequence at the 5′ end of the transduction marker.
91. The system of any of embodiments 56-90, wherein the binding domain is part of an extracellular component.
92. The system of any of embodiments 56-91, wherein the target cells include cancer cells or cells infected with bacteria, virus, fungi, parasites, or arthropods.
93. The system of any of embodiments 56-92, wherein the target cells include cancer cells.
94. The system of any of embodiments 56-93, wherein the recombinant receptor further includes an intracellular component.
95. The system of embodiment 94, wherein the intracellular component includes a CD3ζ signaling domain and/or a 4-1BB signaling domain.
96. The system of embodiments 94 or 95, wherein the intracellular component is linked to an extracellular component through a transmembrane domain.
97. The system of embodiment 96, wherein the transmembrane domain includes a CD28 transmembrane domain.
98. The system of any of embodiments 56-97, wherein the first artificial expression construct and the second artificial expression construct are on the same artificial expression construct.
99. The system of any of embodiments 56-97, wherein the first artificial expression construct and the second artificial expression construct are on different artificial expression constructs.
100. The system of any of embodiments 56-99, wherein:
   (i) the first artificial expression construct includes from 5′ to 3′: a first promoter, wherein the first promoter includes the sequence as set forth in any one of SEQ ID NOs: 17-56 and a sequence as set forth in SEQ ID NO: 138; a first control feature; a first skip sequence; and a sequence as set forth in any one of SEQ ID NOs: 1-11; and
   (ii) the second artificial expression construct includes form 5′ to 3′: a second promoter; a sequence encoding a recombinant receptor; a second skip sequence; a second control feature; a third skip sequence; and a third control feature.
101. A nanoparticle encapsulating the artificial expression construct of any of embodiments 1-55 or the system of any of embodiments 56-100.
102. A cell genetically modified to express the artificial expression construct of any of embodiments 1-55 or the system of any of embodiments 56-100.
103. The cell of embodiment 102, wherein the cell is an autologous cell or an allogeneic cell in reference to a subject.
104. The cell of embodiments 102 or 103, wherein the cell is in vivo or ex vivo.
105. The cell of any of embodiments 102-104, wherein the cell is an immune cell.
106. The cell of embodiment 105, wherein the immune cell is a lymphocyte.
107. The cell of embodiment 106, wherein the lymphocyte includes a T cell, B cell, natural killer (NK) cell, or NK-T cell.
108. The cell of any of embodiments 102-107, wherein the cell is a T cell selected from a CD3+T cell, a CD4+ T cell, a CD8+ T cell, a central memory T cell, an effector memory T cell, and/or a naive T cell.
109. The cell of any of embodiments 102-108, wherein the cell is a CD8+ T cell.
110. The cell of any of embodiments 102-109, wherein the cell is a CD4+ T cell.
111. A population of cells genetically modified to express the artificial expression construct of any of embodiments 1-55 or the system of any of embodiments 56-100.
112. The population of cells of embodiment 111, wherein the population of cells includes autologous cells or allogeneic cells in reference to a subject.
113. The population of cells of embodiments 111 or 112, wherein the population is in vivo or ex vivo.
114. The population of cells of any of embodiments 111-113, wherein the cell is an immune cell.
115. The population of cells of embodiment 114, wherein the immune cell is a lymphocyte.
116. The population of cells of embodiment 115, wherein the lymphocyte includes a T cell, B cell, natural killer (NK) cell, or NK-T cell.
117. The population of cells of any of embodiments 111-116, wherein the population includes CD4+ T cells and/or CD8+ T cells.
118. A formulation including (i) cells genetically modified to express the artificial expression construct of any of embodiments 1-55 or the system of any of embodiments 56-100 and (ii) a pharmaceutically acceptable carrier.
119. A method of genetically modifying an immune cell to have potentiated function including: contacting the cell with the artificial expression construct of any of embodiments 1-55 having a sequence encoding a PD1:cytokine chimera.
120. The method of embodiment 119, wherein the potentiated function includes potentiated cell killing ability.
121. The method of embodiment 120, wherein the encoded PD1:cytokine chimera includes:
   PD1 linked to interleukin (IL)21 (PD1:IL21),
   PD1 linked to IL15 (PD1:IL15),
   PD1 linked to an N72D mutant IL15 (PD1:IL15(N72D)),
   PD1 linked to IL18 (PD1:IL18),
   PD1 linked to decoy resistant IL18 (PD1:drIL18),
   PD1 linked to IL12 (PD1:IL12), or
   PD1 linked to interferon alpha (PD1:IFNα).
122. The method of embodiment 121, wherein PD1 is PD1 with an A99L mutation.
123. The method of embodiment 121, wherein the PD1is wildtype PD1.
124. The method of any of embodiments 121-123, wherein the encoded PD1:cytokine chimera includes PD1:IFNα.
125. The method of any of embodiments 119, wherein the potentiated function includes potentiated proliferation.
126. The method of embodiment 125, wherein the encoded PD1:cytokine chimera includes:
   PD1 linked to interleukin (IL)21 (PD1:IL21),
   PD1 linked to IL15 (PD1:IL15),
   PD1 linked to an N72D mutant IL15 (PD1:IL15(N72D)),
   PD1 linked to IL18 (PD1:IL18),
   PD1 linked to decoy resistant IL18 (PD1:drIL18),
   PD1 linked to IL12 (PD1:IL12), or
   PD1 linked to interferon alpha (PD1:IFNα).
127. The method of embodiment 126, wherein the encoded PD1:cytokine chimera includes PD1:IFNα.
128. The method of embodiment 119, wherein the potentiated function includes potentiated cytokine output.
129. The method of embodiment 128, wherein the potentiated cytokine output includes IFNY.
130. The method of embodiment 129, wherein the encoded PD1:cytokine chimera includes:
   PD1 linked to interleukin (IL)21 (PD1:IL21),
   PD1 linked to IL15 (PD1:IL15),
   PD1 linked to an N72D mutant IL15 (PD1:IL15(N72D)),
   PD1 linked to IL18 (PD1:IL18),
   PD1 linked to decoy resistant IL18 (PD1:drIL18),
   PD1 linked to IL12 (PD1:IL12), or
   PD1 linked to interferon alpha (PD1:IFNα).
131. The method of embodiment 130, wherein PD1 is PD1 with an A99L mutation.
132. The method of embodiment 130, wherein the PD1 is wildtype PD1.
133. The method of any of embodiments 130-132, wherein the encoded PD1:cytokine chimera includes PD1:IFNα.
134. The method of embodiment 128, wherein the potentiated cytokine output includes TNFα output.
135. The method of embodiment 134, wherein the encoded PD1:cytokine chimera includes:
   PD1 linked to interleukin (IL)21 (PD1:IL21),
   PD1 linked to IL15 (PD1:IL15),
   PD1 linked to an N72D mutant IL15 (PD1:IL15(N72D)),
   PD1 linked to IL18 (PD1:IL18),
   PD1 linked to decoy resistant IL18 (PD1:drIL18),
   PD1 linked to IL12 (PD1:IL12), or
   PD1 linked to interferon alpha (PD1:IFNα).
136. The method of embodiment 135, wherein PD1 is PD1 with an A99L mutation.
137. The method of embodiment 135, wherein the PD1is wildtype PD1.
138. The method of any of embodiments 135-137, wherein the encoded PD1:cytokine chimera includes PD1:IFNα.
139. The method of any of embodiments 119-138, wherein the artificial expression construct further includes a sequence encoding a recombinant receptor including a binding domain that binds an antigen expressed on the surface of target cells.
140. The method of embodiment 139, wherein the sequence encoding the recombinant receptor is operably linked to a second promoter.
141. The method of embodiment 140, wherein the second promoter includes an EF1a(L) promoter, EF1a(s) promoter, myeloproliferative sarcoma virus (MND) promoter, cytomegalovirus (CMV) promoter, simian virus 40 (SV40) early promoter, mouse mammary tumor virus (MMTV) promoter, human immunodeficiency virus (HIV) long terminal repeat (LTR) promoter, MoMuLV promoter, avian leukemia virus promoter, Epstein-Barr virus immediate early promoter, Rous sarcoma virus promoter, actin promoter, myosin promoter, hemoglobin promoter, or a creatine kinase promoter.
142. The method of any of embodiments 119-141, wherein the method further includes contacting the cell with an artificial expression construct including a sequence encoding a recombinant receptor including a binding domain that binds an antigen expressed on the surface of target cells.
143. The method of embodiment 142, wherein the contacting the cell with the artificial expression construct of any of embodiments 1-55 and the artificial expression construct including the sequence encoding the recombinant receptor occurs simultaneously.
144. The method of embodiment 142, wherein the contacting the cell with the artificial expression construct of any of embodiments 1-55 and the artificial expression construct including the sequence encoding the recombinant receptor occurs at different times.
145. The method of any of embodiments 119-144, wherein the artificial expression construct includes the sequence as set forth in SEQ ID NOs: 98, 99, 100, 101, or 102 or a sequence having 90% sequence identity to a sequence as set forth in 98, 99, 100, 101, or 102.
146. A method of treating a subject in need thereof including administering a therapeutically effective amount of the artificial expression construct of any of embodiments 1-55, the system of any of embodiments 56-100, the nanoparticle of embodiment 101, or the formulation of embodiment 118 to the subject thereby treating the subject in need thereof.
147. The method of embodiment 146, wherein the subject in need thereof includes cancer or an infection.
148. The method of embodiments 146 or 147, wherein the administering a therapeutically effective amount includes administering intravesically, intravenously, intradermally, intraarterially, intraparenterally, intranodally, intralymphaticaly, intraperitoneally, intralesionally, intraprostaticaly, intravaginally, intrarectally, topically, intrathecally, intratumorally, intramuscularly, or subcutaneously.
149. A recombinant protein including programmed cell death-1 (PD1) and a cytokine.
150. The recombinant protein of embodiment 149, wherein the cytokine is an immunostimulatory cytokine.
151. The recombinant protein of embodiment 150, wherein the immunostimulatory cytokine includes an interleukin (IL)21, an I-15, an N72D mutant IL15 (IL15(N72D), an IL18, a decoy resistant IL18 (drIL18), an IL12, or an interferon alpha (IFNα).
152. The recombinant protein of embodiment 151, wherein the recombinant protein includes:
   PD1 linked to interleukin (IL)21 (PD1:IL21),
   PD1 linked to IL15 (PD1:IL15),
   PD1 linked to an N72D mutant IL15 (PD1:IL15(N72D)),
   PD1 linked to IL18 (PD1:IL18),
   PD1 linked to decoy resistant IL18 (PD1:drIL18),
   PD1 linked to IL12 (PD1:IL12),
   PD1 linked to interferon alpha (PD1:IFNα),
   PD1 with an A99L mutation (PD1(A99L)) linked to IL12 ((PD1(A99L):IL21),
   PD1(A99L) linked to IL15 (PD1(A99L):IL15),
   PD1(A99L) linked to an N72D mutant IL15 (PD1(A99L):IL15(N72D)),
   PD1(A99L) linked to IL18 (PD1(A99L):IL18),
   PD1(A99L) linked to decoy resistant IL18 (PD1(A99L):drIL18),
   PD1(A99L) linked to IL12 (PD1(A99L):IL12), or
   PD1(A99L) linked to interferon alpha (PD1(A99L):IFNα).
153. The recombinant protein of any of embodiments 149-152, wherein the PD1 is linked to the cytokine through a protein linker.
154. The recombinant protein of embodiment 153, wherein the protein linker is a flexible linker.
155. The recombinant protein of embodiment 154, wherein the flexible linker is a Gly-Ser linker.
156. The recombinant protein of embodiment 155, wherein the Gly-Ser linker includes the sequence as set forth in SEQ ID NO: 128 or the sequence GGG.
157. The recombinant protein of any of embodiments 149-156, wherein the recombinant protein is encoded by the sequence as set forth in SEQ ID NO: 7, SEQ ID NO: 8, SEQ ID NO: 9, SEQ ID NO: 10, or SEQ ID NO: 11 or a sequence having at least 90% sequence identity to the sequence set forth SEQ ID NO: 7, SEQ ID NO: 8, SEQ ID NO: 9, SEQ ID NO: 10, or SEQ ID NO: 11.
158. The recombinant protein of any of embodiments 149-157, wherein the recombinant protein includes the sequence as set forth in SEQ ID NO: 12, SEQ ID NO: 13, SEQ ID NO: 14, SEQ ID NO: 15, or SEQ ID NO: 16 or a sequence having at least 90% sequence identity to the sequence set forth in SEQ ID NO: 12, SEQ ID NO: 13, SEQ ID NO: 14, SEQ ID NO: 15, or SEQ ID NO: 16.

### (xii) CLOSING PARAGRAPHS

Recombinant protein and chimera are used interchangeably herein.

The nucleic acid and amino acid sequences provided herein are shown using letter abbreviations for nucleotide bases and amino acid residues, as defined in 37 C.F.R. § 1.831-1.839 and set forth in WIPO Standard ST.26 (implemented on Jul. 1, 2022). Only one strand of each nucleic acid sequence is shown, but the complementary strand is understood as included in embodiments where it would be appropriate.

Variants of the sequences disclosed and referenced herein are also included. Guidance in determining which amino acid residues can be substituted, inserted, or deleted without abolishing biological activity can be found using computer programs well known in the art, such as DNASTAR™ (Madison, Wisconsin) software. Preferably, amino acid changes in the protein variants disclosed herein are conservative amino acid changes, i.e., substitutions of similarly charged or uncharged amino acids. A conservative amino acid change involves substitution of one of a family of amino acids which are related in their side chains.

In a peptide or protein, suitable conservative substitutions of amino acids are known to those of skill in this art and generally can be made without altering a biological activity of a resulting molecule. Those of skill in this art recognize that, in general, single amino acid substitutions in non-essential regions of a polypeptide do not substantially alter biological activity (see, e.g., Watson et al. Molecular Biology of the Gene, 4th Edition, 1987, The Benjamin/Cummings Pub. Co., p. 224). Naturally occurring amino acids are generally divided into conservative substitution families as follows: Group 1: Alanine (Ala), Glycine (Gly), Serine (Ser), and Threonine (Thr); Group 2: (acidic): Aspartic acid (Asp), and Glutamic acid (Glu); Group 3: (acidic; also classified as polar, negatively charged residues and their amides): Asparagine (Asn), Glutamine (Gln), Asp, and Glu; Group 4: Gln and Asn; Group 5: (basic; also classified as polar, positively charged residues): Arginine (Arg), Lysine (Lys), and Histidine (His); Group 6 (large aliphatic, nonpolar residues): Isoleucine (lie), Leucine (Leu), Methionine (Met), Valine (Val) and Cysteine (Cys); Group 7 (uncharged polar): Tyrosine (Tyr), Gly, Asn, Gln, Cys, Ser, and Thr; Group 8 (large aromatic residues): Phenylalanine (Phe), Tryptophan (Trp), and Tyr; Group 9 (non-polar): Proline (Pro), Ala, Val, Leu, lie, Phe, Met, and Trp; Group 11 (aliphatic): Gly, Ala, Val, Leu, and lie; Group 10 (small aliphatic, nonpolar or slightly polar residues): Ala, Ser, Thr, Pro, and Gly; and Group 12 (sulfur-containing): Met and Cys. Additional information can be found in Creighton (1984) Proteins, W.H. Freeman and Company.

In making such changes, the hydropathic index of amino acids may be considered. The importance of the hydropathic amino acid index in conferring interactive biologic function on a protein is generally understood in the art (Kyte and Doolittle, 1982, J. Mol. Biol. 157(1), 105-32). Each amino acid has been assigned a hydropathic index on the basis of its hydrophobicity and charge characteristics (Kyte and Doolittle, 1982). These values are: Ile (+4.5); Val (+4.2); Leu (+3.8); Phe (+2.8); Cys (+2.5); Met (+1.9); Ala (+1.8); Gly (-0.4); Thr (-0.7); Ser (-0.8); Trp (-0.9); Tyr (-1.3); Pro (-1.6); His (-3.2); Glutamate (-3.5); Gln (-3.5); aspartate (-3.5); Asn (-3.5); Lys (-3.9); and Arg (-4.5).

It is known in the art that certain amino acids may be substituted by other amino acids having a similar hydropathic index or score and still result in a protein with similar biological activity, i.e., still obtain a biological functionally equivalent protein. In making such changes, the substitution of amino acids whose hydropathic indices are within ±2 is preferred, those within ±1 are particularly preferred, and those within ±0.5 are even more particularly preferred. It is also understood in the art that the substitution of like amino acids can be made effectively on the basis of hydrophilicity.

As detailed in U.S. Pat. No. 4,554,101, the following hydrophilicity values have been assigned to amino acid residues: Arg (+3.0); Lys (+3.0); aspartate (+3.0±1); glutamate (+3.0±1); Ser (+0.3); Asn (+0.2); Gln (+0.2); Gly (0); Thr (−0.4); Pro (−0.5±1); Ala (−0.5); His (−0.5); Cys (−1.0); Met (−1.3); Val (−1.5); Leu (−1.8); Ile (−1.8); Tyr (−2.3); Phe (−2.5); Trp (−3.4). It is understood that an amino acid can be substituted for another having a similar hydrophilicity value and still obtain a biologically equivalent, and in particular, an immunologically equivalent protein.

In such changes, the substitution of amino acids whose hydrophilicity values are within ±2 is preferred, those within ±1 are particularly preferred, and those within ±0.5 are even more particularly preferred.

As outlined above, amino acid substitutions may be based on the relative similarity of the amino acid side-chain substituents, for example, their hydrophobicity, hydrophilicity, charge, size, and the like. As indicated elsewhere, variants of gene sequences can include codon optimized variants, sequence polymorphisms, splice variants, and/or mutations that do not affect the function of an encoded product to a statistically-significant degree.

Variants of the protein, nucleic acid, and gene sequences disclosed herein also include sequences with at least 70% sequence identity, 80% sequence identity, 85% sequence, 90% sequence identity, 95% sequence identity, 96% sequence identity, 97% sequence identity, 98% sequence identity, or 99% sequence identity to the protein, nucleic acid, or gene sequences disclosed herein.

“% sequence identity” refers to a relationship between two or more sequences, as determined by comparing the sequences. In the art, “identity” also means the degree of sequence relatedness between protein, nucleic acid, or gene sequences as determined by the match between strings of such sequences. “Identity” (often referred to as “similarity”) can be readily calculated by known methods, including those described in: Computational Molecular Biology (Lesk, A. M., ed.) Oxford University Press, NY (1988); Biocomputing: Informatics and Genome Projects (Smith, D. W., ed.) Academic Press, NY (1994); Computer Analysis of Sequence Data, Part I (Griffin, A. M., and Griffin, H. G., eds.) Humana Press, NJ (1994); Sequence Analysis in Molecular Biology (Von Heijne, G., ed.) Academic Press (1987); and Sequence Analysis Primer (Gribskov, M. and Devereux, J., eds.) Oxford University Press, NY (1992). Methods to determine identity are designed to give the best match between the sequences tested. Methods to determine identity and similarity are codified in publicly available computer programs. Sequence alignments and percent identity calculations may be performed using the Megalign program of the LASERGENE bioinformatics computing suite (DNASTAR, Inc., Madison, Wisconsin). Multiple alignment of the sequences can also be performed using the Clustal method of alignment (Higgins and Sharp CABIOS, 5, 151-153 (1989) with default parameters (GAP PENALTY=10, GAP LENGTH PENALTY=10). Relevant programs also include the GCG suite of programs (Wisconsin Package Version 9.0, Genetics Computer Group (GCG), Madison, Wisconsin); BLASTP, BLASTN, BLASTX (Altschul, et al., J. Mol. Biol. 215:403-410 (1990); DNASTAR (DNASTAR, Inc., Madison, Wisconsin); and the FASTA program incorporating the Smith-Waterman algorithm (Pearson, Comput. Methods Genome Res., [Proc. Int. Symp.](1994), Meeting Date 1992, 111-20. Editor(s): Suhai, Sandor. Publisher: Plenum, New York, N.Y. Within the context of this disclosure it will be understood that where sequence analysis software is used for analysis, the results of the analysis are based on the “default values” of the program referenced. As used herein “default values” will mean any set of values or parameters, which originally load with the software when first initialized.

Variants also include nucleic acid molecules that hybridize under stringent hybridization conditions to a sequence disclosed herein and provide the same function as the reference sequence. Exemplary stringent hybridization conditions include an overnight incubation at 42° C. in a solution including 50% formamide, 5×SSC (750 mM NaCl, 75 mM trisodium citrate), 50 mM sodium phosphate (pH 7.6), 5×Denhardt's solution, 10% dextran sulfate, and 20 μg/ml denatured, sheared salmon sperm DNA, followed by washing the filters in 0.1×SSC at 50° C. Changes in the stringency of hybridization and signal detection are primarily accomplished through the manipulation of formamide concentration (lower percentages of formamide result in lowered stringency); salt conditions, or temperature. For example, moderately high stringency conditions include an overnight incubation at 37° C. in a solution including 6×SSPE (20×SSPE=3M NaCl; 0.2M NaH2PO4; 0.02M EDTA, pH 7.4), 0.5% SDS, 30% formamide, 100 μg/ml salmon sperm blocking DNA; followed by washes at 50° C. with 1×SSPE, 0.1% SDS. In addition, to achieve even lower stringency, washes performed following stringent hybridization can be done at higher salt concentrations (e.g., 5×SSC). Variations in the above conditions may be accomplished through the inclusion and/or substitution of alternate blocking reagents used to suppress background in hybridization experiments. Typical blocking reagents include Denhardt's reagent, BLOTTO, heparin, denatured salmon sperm DNA, and commercially available proprietary formulations. The inclusion of specific blocking reagents may require modification of the hybridization conditions described above, due to problems with compatibility.

“Binds” refers to an association of a binding domain (of, for example, a binding domain) to its cognate binding molecule with an affinity or Kₐ (i.e., an equilibrium association constant of a particular binding interaction with units of 1/M) equal to or greater than 10⁵ M⁻¹, while not significantly associating with any other molecules or components in a relevant environment sample. Binding domains may be classified as “high affinity” or “low affinity”. In particular embodiments, “high affinity” binding domains refer to those binding domains with a Kₐ of at least 10⁷ M⁻¹, at least 10⁸ M⁻¹, at least 10⁹ M⁻¹, at least 10¹⁰ M⁻¹, at least 10¹¹ M⁻¹, at least 10¹² M⁻¹, or at least 10¹³ M⁻¹. In particular embodiments, “low affinity” binding domains refer to those binding domains with a Kₐ of up to 10⁷ M⁻¹, up to 10⁶ M⁻¹, up to 10⁵ M⁻¹. Alternatively, affinity may be defined as an equilibrium dissociation constant (K_{d}) of a particular binding interaction with units of M (e.g., 10⁻⁵ M to 10⁻¹³ M). In certain embodiments, a binding domain may have “enhanced affinity,” which refers to a selected or engineered binding domains with stronger binding to a cognate binding molecule than a wild type (or parent) binding domain. For example, enhanced affinity may be due to a Kₐ (equilibrium association constant) for the cognate binding molecule that is higher than the reference binding domain or due to a K_{d} (dissociation constant) for the cognate binding molecule that is less than that of the reference binding domain, or due to an off-rate (K_{off}) for the cognate binding molecule that is less than that of the reference binding domain.

A variety of assays are known for detecting binding domains that bind a particular cognate binding molecule as well as determining binding affinities, such as Western blot, ELISA, and BIACORE® analysis (see also, e.g., Scatchard, et al., 1949, Ann. N.Y. Acad. Sci. 51:660; and U.S. Pat. Nos. 5,283,173, 5,468,614, or the equivalent).

Unless otherwise indicated, the practice of the present disclosure can employ conventional techniques of immunology, molecular biology, microbiology, cell biology and recombinant DNA. These methods are described in the following publications. See, e.g., Sambrook, et al. Molecular Cloning: A Laboratory Manual, 2nd Edition (1989); F. M. Ausubel, et al. eds., Current Protocols in Molecular Biology, (1987); the series Methods IN Enzymology (Academic Press, Inc.); M.

MacPherson, et al., PCR: A Practical Approach, IRL Press at Oxford University Press (1991); MacPherson et al., eds. PCR 2: Practical Approach, (1995); Harlow and Lane, eds. Antibodies, A Laboratory Manual, (1988); and R. I. Freshney, ed. Animal Cell Culture (1987).

As will be understood by one of ordinary skill in the art, each embodiment disclosed herein can comprise, consist essentially of or consist of its particular stated element, step, ingredient or component. Thus, the terms “include” or “including” should be interpreted to recite: “comprise, consist of, or consist essentially of.” The transition term “comprise” or “comprises” means has, but is not limited to, and allows for the inclusion of unspecified elements, steps, ingredients, or components, even in major amounts. The transitional phrase “consisting of” excludes any element, step, ingredient or component not specified. The transition phrase “consisting essentially of” limits the scope of the embodiment to the specified elements, steps, ingredients or components and to those that do not materially affect the embodiment. A material effect would cause a statistically significant reduction in recombinant receptor-immune cell killing, proliferation, and/or cytokine output, as described herein.

Unless otherwise indicated, all numbers expressing quantities of ingredients, properties such as molecular weight, reaction conditions, and so forth used in the specification and claims are to be understood as being modified in all instances by the term “about.” Accordingly, unless indicated to the contrary, the numerical parameters set forth in the specification and attached claims are approximations that may vary depending upon the desired properties sought to be obtained by the present invention. At the very least, and not as an attempt to limit the application of the doctrine of equivalents to the scope of the claims, each numerical parameter should at least be construed in light of the number of reported significant digits and by applying ordinary rounding techniques. When further clarity is required, the term “about” has the meaning reasonably ascribed to it by a person skilled in the art when used in conjunction with a stated numerical value or range, i.e. denoting somewhat more or somewhat less than the stated value or range, to within a range of ±20% of the stated value; 19% of the stated value; ±18% of the stated value; 17% of the stated value; 16% of the stated value; ±15% of the stated value; 14% of the stated value; ±13% of the stated value; 12% of the stated value; 11% of the stated value; 10% of the stated value; 9% of the stated value; 8% of the stated value; 7% of the stated value; ±6% of the stated value; 5% of the stated value; 4% of the stated value; ±3% of the stated value; 2% of the stated value; or ±1% of the stated value.

Notwithstanding that the numerical ranges and parameters setting forth the broad scope of the invention are approximations, the numerical values set forth in the specific examples are reported as precisely as possible. Any numerical value, however, inherently contains certain errors necessarily resulting from the standard deviation found in their respective testing measurements.

The terms “a,” “an,” “the” and similar referents used in the context of describing the invention (especially in the context of the following claims) are to be construed to cover both the singular and the plural, unless otherwise indicated herein or clearly contradicted by context.

Recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein is intended merely to better illuminate the invention and does not pose a limitation on the scope of the invention otherwise claimed. No language in the specification should be construed as indicating any non-claimed element essential to the practice of the invention.

Groupings of alternative elements or embodiments of the invention disclosed herein are not to be construed as limitations. Each group member may be referred to and claimed individually or in any combination with other members of the group or other elements found herein. It is anticipated that one or more members of a group may be included in, or deleted from, a group for reasons of convenience and/or patentability. When any such inclusion or deletion occurs, the specification is deemed to contain the group as modified thus fulfilling the written description of all Markush groups used in the appended claims.

Certain embodiments of this invention are described herein, including the best mode known to the inventors for carrying out the invention. Of course, variations on these described embodiments will become apparent to those of ordinary skill in the art upon reading the foregoing description. The inventor expects skilled artisans to employ such variations as appropriate, and the inventors intend for the invention to be practiced otherwise than specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

Furthermore, numerous references have been made to patents, printed publications, journal articles and other written text throughout this specification (referenced materials herein). Each of the referenced materials are individually incorporated herein by reference in their entirety for their referenced teaching.

In closing, it is to be understood that the embodiments of the invention disclosed herein are illustrative of the principles of the present invention. Other modifications that may be employed are within the scope of the invention. Thus, by way of example, but not of limitation, alternative configurations of the present invention may be utilized in accordance with the teachings herein. Accordingly, the present invention is not limited to that precisely as shown and described.

The particulars shown herein are by way of example and for purposes of illustrative discussion of the preferred embodiments of the present invention only and are presented in the cause of providing what is believed to be the most useful and readily understood description of the principles and conceptual aspects of various embodiments of the invention. In this regard, no attempt is made to show structural details of the invention in more detail than is necessary for the fundamental understanding of the invention, the description taken with the drawings and/or examples making apparent to those skilled in the art how the several forms of the invention may be embodied in practice.

Definitions and explanations used in the present disclosure are meant and intended to be controlling in any future construction unless clearly and unambiguously modified in the examples or when application of the meaning renders any construction meaningless or essentially meaningless. In cases where the construction of the term would render it meaningless or essentially meaningless, the definition should be taken from Webster's Dictionary, 3rd Edition or a dictionary known to those of ordinary skill in the art, such as the Oxford Dictionary of Biochemistry and Molecular Biology (Eds. Attwood T et al., Oxford University Press, Oxford, 2006).

What is claimed is:

1. An artificial expression construct comprising:
an inducible promoter comprising SEQ ID NO: 17 and SEQ ID NO: 138; and
a sequence encoding a programmed cell death-1 (PD1): cytokine chimera, wherein the PD1:
cytokine chimera comprises a PD1 linked to a cytokine selected from: an interferon alpha (IFNα), an interleukin (IL)21, an IL15, an N72D mutant IL15 (IL15(N72D), an IL18, a decoy resistant IL18 (drIL18), or an IL12.

2. An artificial expression construct comprising a sequence encoding a programmed cell death-1 (PD1):cytokine chimera, wherein the PD1: cytokine chimera comprises a PD1 linked to a cytokine.

3. The artificial expression construct of claim 2, wherein the cytokine is an immunostimulatory cytokine.

4. The artificial expression construct of claim 3, wherein the immunostimulatory cytokine comprises an interleukin (IL)21, an IL15, an N72D mutant IL15 (IL15(N72D), an IL18, a decoy resistant IL18 (drIL18), an IL12, or an interferon alpha (IFNα).

5. The artificial expression construct of claim 2, wherein the sequence encoding the PD1:cytokine chimera is under the regulatory control of a promoter, wherein the promoter comprises a sequence having at least 95% sequence identity to any one of SEQ ID NOs: 17-56 and a minimal promoter.

6. The artificial expression construct of claim 5, wherein the promoter has at least 98% sequence identity to any one of SEQ ID NOs: 17-56 and a minimal promoter.

7. The artificial expression construct of claim 5, wherein the promoter has at least 99% sequence identity to any one of SEQ ID NOs: 17-56 and a minimal promoter.

8. The artificial expression construct of claim 5, wherein the promoter has the sequence as set forth in any one of SEQ ID NOs: 17-56 and a minimal promoter.

9. The artificial expression construct of claim 5, wherein the minimal promoter is an IL2 minimal promoter.

10. The artificial expression construct of claim 9, wherein the IL2 minimal promoter comprises the sequence as set forth in SEQ ID NO: 138.

11. The artificial expression construct of claim 2, wherein the PD1:cytokine chimera comprises:
PD1 linked to interleukin (IL)21 (PD1:IL21),
PD1 linked to IL15 (PD1:IL15),
PD1 linked to an N72D mutant IL15 (PD1:IL15(N72D)),
PD1 linked to IL18 (PD1:IL18),
PD1 linked to decoy resistant IL18 (PD1:drIL18),
PD1 linked to IL12 (PD1:IL12), or
PD1 linked to interferon alpha (PD1:IFNα).

12. The artificial expression construct of claim 2, wherein the PD1 is PD1 with an A99L mutation.

13. The artificial expression construct of claim 2, wherein the PD1 is wildtype PD1.

14. The artificial expression construct of claim 2, wherein the PD1 is linked to the cytokine through a protein linker.

15. The artificial expression construct of claim 14, wherein the protein linker is a flexible linker.

16. The artificial expression construct of claim 15, wherein the flexible linker is a Gly-Ser linker.

17. The artificial expression construct of claim 16, wherein the Gly-Ser linker comprises a sequence as set forth in SEQ ID NO: 128 or the sequence GGG.

18. The artificial expression construct of claim 2, wherein the PD1:cytokine chimera is encoded by the sequence as set forth in SEQ ID NO: 7, SEQ ID NO: 8, SEQ ID NO: 9, SEQ ID NO: 10, or SEQ ID NO: 11 or a sequence having at least 90% sequence identity to the sequence set forth in SEQ ID NO: 7, SEQ ID NO: 8, SEQ ID NO: 9, SEQ ID NO: 10, or SEQ ID NO: 11.

19. The artificial expression construct of claim 2, wherein the PD1:cytokine chimera comprises the sequence as set forth in SEQ ID NO: 12, SEQ ID NO: 13, SEQ ID NO: 14, SEQ ID NO: 15, or SEQ ID NO: 16 or a sequence having at least 90% sequence identity to the sequence set forth SEQ ID NO: 12, SEQ ID NO: 13, SEQ ID NO: 14, SEQ ID NO: 15, or SEQ ID NO: 16.

20. The artificial expression construct of claim 2, further comprising a control feature.

21. The artificial expression construct of claim 20, wherein the control feature encodes a transduction marker.

22. The artificial expression construct of claim 21, wherein the transduction marker comprises Her2tG.

23. The artificial expression construct of claim 2, further comprising a first skip sequence.

24. The artificial expression construct of claim 23, wherein the first skip sequence encodes a 2A self-cleaving polypeptide.

25. The artificial expression construct of claim 24, wherein the 2A self-cleaving polypeptide comprises T2A, P2A, E2A, or F2A.

26. The artificial expression construct of claim 2, wherein the artificial expression construct comprises from 5′ to 3′: a promoter comprising the sequence as set forth in any one of SEQ ID NOs: 17-56 and a sequence as set forth in SEQ ID NO: 138; a control feature; a skip sequence; and a sequence as set forth in any one of SEQ ID NOs: 1-11.

27. The artificial expression construct of claim 2, wherein the artificial expression construct comprises from 5′ to 3′: a promoter comprising the sequence as set forth in any one of SEQ ID NOs: 17-56 and a sequence as set forth in SEQ ID NO: 138; a sequence encoding a transduction marker; a skip sequence; and a sequence as set forth in any one of SEQ ID NOs: 1-11.

28. The artificial expression construct of claim 2, wherein the artificial expression construct has at least 95% sequence identity to the sequence as set forth in SEQ ID NO: 103.

29. The artificial expression construct of claim 2, wherein the artificial expression construct has at least 98% sequence identity to the sequence as set forth in SEQ ID NO: 103.

30. The artificial expression construct of claim 2, wherein the artificial expression construct has at least 99% sequence identity to the sequence as set forth in SEQ ID NO: 103.

31. The artificial expression construct of claim 2, wherein the artificial expression construct has the sequence as set forth in SEQ ID NO: 103.

32. The artificial expression construct of claim 2, further comprising a sequence encoding a recombinant receptor, wherein the recombinant receptor comprises a binding domain that binds a target antigen.

33. The artificial expression construct of claim 32, wherein the sequence encoding the recombinant receptor is operably linked to a second promoter.

34. The artificial expression construct of claim 33, wherein the second promoter comprises an EF1α(L) promoter, EF1α(s) promoter, myeloproliferative sarcoma virus (MND) promoter, cytomegalovirus (CMV) promoter, simian virus 40 (SV40) early promoter, mouse mammary tumor virus (MMTV) promoter, human immunodeficiency virus (HIV) long terminal repeat (LTR) promoter, MoMuLV promoter, avian leukemia virus promoter, Epstein-Barr virus immediate early promoter, Rous sarcoma virus promoter, actin promoter, myosin promoter, hemoglobin promoter, or a creatine kinase promoter.

35. The artificial expression construct of claim 32, wherein the binding domain is part of an extracellular component.

36. The artificial expression construct of claim 32, wherein the target antigen is expressed on the surface of target cells.

37. The artificial expression construct of claim 36, wherein the target cells comprise cancer cells or cells infected with bacteria, virus, fungi, parasites, or arthropods.

38. The artificial expression construct of claim 36, wherein the target cells comprise cancer cells.

39. The artificial expression construct of claim 32, wherein the recombinant receptor further comprises an intracellular component.

40. The artificial expression construct of claim 39, wherein the intracellular component comprises a CD3ζ signaling domain and/or a 4-1BB signaling domain.

41. The artificial expression construct of claim 39, wherein the intracellular component is linked to an extracellular component through a transmembrane domain.

42. The artificial expression construct of claim 41, wherein the transmembrane domain comprises a CD28 transmembrane domain.

43. The artificial expression construct of claim 32, further comprising a second control feature.

44. The artificial expression construct of claim 43, wherein the second control feature encodes a selection cassette.

45. The artificial expression construct of claim 44, wherein the selection cassette comprises dihydrofolate reductase double mutant (DHFRdm).

46. The artificial expression construct of claim 43, further comprising a third control feature.

47. The artificial expression construct of claim 46, wherein the third control feature encodes a transduction marker.

48. The artificial expression construct of claim 47, wherein the transduction marker comprises epidermal growth factor receptor (EGFRt) or truncated CD19 (tCD19).

49. The artificial expression construct of claim 32, further comprising a second skip sequence.

50. The artificial expression construct of claim 49, wherein the second skip sequence encodes a 2A self-cleaving polypeptide.

51. The artificial expression construct of claim 50, wherein the 2A skip self-cleaving polypeptide comprises T2A, P2A, E2A, or F2A.

52. The artificial expression construct of claim 32, wherein the artificial expression construct comprises a second skip sequence at the 5′ end of the selection cassette and a third skip sequence at the 5′ end of the transduction marker.

53. The artificial expression construct of claim 2, wherein the artificial expression construct comprises from 5′ to 3′: a first promoter, wherein the first promoter comprises the sequence as set forth in any one of SEQ ID NOs: 17-56 and a sequence as set forth in SEQ ID NO: 138; a first control feature; a first skip sequence; a sequence as set forth in any one of SEQ ID NOs: 1-11; a second promoter; a sequence encoding a recombinant receptor; a second skip sequence; a second control feature; a third skip sequence; and a third control feature.

54. The artificial expression construct of claim 2, wherein the artificial expression construct comprises from 5′ to 3′: a first promoter, wherein the first promoter comprises the sequence as set forth in any one of SEQ ID NOs: 17-56 and a sequence as set forth in SEQ ID NO: 138; a sequence encoding a first transduction marker; a first skip sequence; a sequence as set forth in any one of SEQ ID NOs: 1-11; a second promoter; a sequence encoding a recombinant receptor; a second skip sequence; a sequence encoding a selection cassette; a third skip sequence; and a sequence encoding a second transduction marker.

55. The artificial expression construct of claim 2, wherein the artificial expression construct has at least 90% sequence identity to the sequence as set forth in SEQ ID NOs: 98, 99, 100, 101, or 102.

56. The artificial expression construct of claim 2, wherein the artificial expression construct has the sequence as set forth in SEQ ID NOs: 98, 99, 100, 101, or 102.

57. A system for potentiating immune cell function comprising:
a first artificial expression construct encoding a PD1:cytokine chimera under the regulatory control of a first promoter, wherein the first promoter comprises a sequence having at least 95% sequence identity to any of SEQ ID NOs: 17-56 and a minimal promoter, and wherein the PD1: chimera comprises a PD1 linked to a cytokine; and
a second artificial expression construct comprises a sequence encoding a recombinant receptor, the recombinant receptor comprising a binding domain that binds an antigen expressed on the surface of target cells.

58. The system of claim 57, wherein the cytokine is an immunostimulatory cytokine.

59. The system of claim 58, wherein the immunostimulatory cytokine comprises an interleukin (IL)21, an IL15, an N72D mutant IL15 (IL15(N72D), an IL18, a decoy resistant IL18 (drIL18), an IL12, or an interferon alpha (IFNα).

60. The system of claim 57, wherein the first promoter comprises a sequence having at least 98% sequence identity to any one of SEQ ID NOs: 17-56 and a minimal promoter.

61. The system of claim 57, wherein the first promoter comprises a sequence having at least 99% sequence identity to any one of SEQ ID NOs: 17-56 and a minimal promoter.

62. The system of claim 57, wherein the first promoter comprises the sequence as set forth in any one of SEQ ID NOs: 17-56 and a minimal promoter.

63. The system of claim 57, wherein the minimal promoter comprises a IL2 minimal promoter.

64. The system of claim 63, wherein the IL2 minimal promoter comprises the sequence as set forth in SEQ ID NO: 138.

65. The system of claim 57, wherein the PD1:cytokine chimera comprises:
PD1 linked to interleukin (IL)21 (PD1:IL21),
PD1 linked to IL15 (PD1:IL15),
PD1 linked to an N72D mutant IL15 (PD1:IL15(N72D)),
PD1 linked to IL18 (PD1:IL18),
PD1 linked to decoy resistant IL18 (PD1:drIL18),
PD1 linked to IL12 (PD1:IL12), or
PD1 linked to interferon alpha (PD1:IFNα).

66. The system of claim 57, wherein the PD1 is PD1 with an A99L mutation.

67. The system of claim 57, wherein the PD1 is wildtype PD1.

68. The system of claim 57, wherein the PD1 is linked to the cytokine through a protein linker.

69. The system of claim 68, wherein the protein linker is a flexible linker.

70. The system of claim 69, wherein the flexible linker is a Gly-Ser linker.

71. The system of claim 70, wherein the Gly-Ser linker comprises a sequence as set forth in SEQ ID NO: 128 or the sequence GGG.

72. The system of claim 57, wherein the PD1:cytokine chimera is encoded by the sequence as set forth in SEQ ID NO: 7, SEQ ID NO: 8, SEQ ID NO: 9, SEQ ID NO: 10, or SEQ ID NO: 11 or a sequence having at least 90% sequence identity to the sequence set forth in SEQ ID NO: 7, SEQ ID NO: 8, SEQ ID NO: 9, SEQ ID NO: 10, or SEQ ID NO: 11.

73. The system of claim 57, wherein the PD1:cytokine chimera comprises the sequence as set forth in SEQ ID NO: 12, SEQ ID NO: 13, SEQ ID NO: 14, SEQ ID NO: 15, or SEQ ID NO: 16 or a sequence having at least 90% sequence identity to the sequence set forth in SEQ ID NO: 12, SEQ ID NO: 13, SEQ ID NO: 14, SEQ ID NO: 15, or SEQ ID NO: 16.

74. The system of claim 57, wherein the first artificial expression construct further comprises a control feature.

75. The system of claim 74, wherein the control feature encodes a transduction marker.

76. The system of claim 75, wherein the transduction marker comprises Her2tG.

77. The system of claim 57, wherein the first artificial expression construct further comprises a first skip sequence.

78. The system of claim 77, wherein the first skip sequence encodes a 2A self-cleaving polypeptide.

79. The system of claim 78, wherein the 2A self-cleaving polypeptide comprises T2A, P2A, E2A, or F2A.

80. The system of claim 57, wherein the sequence encoding the recombinant receptor is operably linked to a second promoter.

81. The system of claim 57, wherein the second promoter comprises an EF1α(L) promoter, EF1α(s) promoter, MND promoter, CMV promoter, SV40 early promoter, MMTV promoter, HIV LTR promoter, MoMuLV promoter, an avian leukemia virus promoter, an Epstein-Barr virus immediate early promoter, a Rous sarcoma virus promoter, an actin promoter, a myosin promoter, a hemoglobin promoter, or a creatine kinase promoter.

82. The system of claim 57, wherein the second artificial expression construct further comprises a second control feature.

83. The system of claim 82, wherein the second control feature encodes a selection cassette.

84. The system of claim 83, wherein the selection cassette comprises dihydrofolate reductase double mutant (DHFRdm).

85. The system of claim 57, wherein the second artificial expression construct further comprises a third control feature.

86. The system of claim 85, wherein the third control feature encodes a transduction marker.

87. The system of claim 86, wherein the transduction marker comprises truncated epidermal growth factor receptor (EGFRt) or tCD19.

88. The system of claim 57, wherein the second artificial expression construct further comprises a second skip sequence.

89. The system of claim 88, wherein the second skip sequence encodes a 2A self-cleaving polypeptide.

90. The system of claim 89, wherein the 2A self-cleaving polypeptide comprises T2A, P2A, E2A, or F2A.

91. The system of claim 57, wherein the second artificial expression construct comprises a second skip sequence at the 5′ end of the selection cassette and a third skip sequence at the 5′ end of the transduction marker.

92. The system of claim 57, wherein the binding domain is part of an extracellular component.

93. The system of claim 57, wherein the target cells comprise cancer cells or cells infected with bacteria, virus, fungi, parasites, or arthropods.

94. The system of claim 57, wherein the target cells comprise cancer cells.

95. The system of claim 57, wherein the recombinant receptor further comprises an intracellular component.

96. The system of claim 95, wherein the intracellular component comprises a CD3ζ signaling domain and/or a 4-1BB signaling domain.

97. The system of claim 95, wherein the intracellular component is linked to an extracellular component through a transmembrane domain.

98. The system of claim 97, wherein the transmembrane domain comprises a CD28 transmembrane domain.

99. The system of claim 57, wherein the first artificial expression construct and the second artificial expression construct are on the same artificial expression construct.

100. The system of claim 57, wherein the first artificial expression construct and the second artificial expression construct are on different artificial expression constructs.

101. The system of claim 57, wherein:
(i) the first artificial expression construct comprises from 5′ to 3′: a first promoter, wherein the first promoter comprises the sequence as set forth in any one of SEQ ID NOs: 17-56 and a sequence as set forth in SEQ ID NO: 138; a first control feature; a first skip sequence; and a sequence as set forth in any one of SEQ ID NOs: 1-11; and
(ii) the second artificial expression construct comprises form 5′ to 3′: a second promoter; a sequence encoding a recombinant receptor; a second skip sequence; a second control feature; a third skip sequence; and a third control feature.

102. A nanoparticle encapsulating the artificial expression construct of claim 2 or the system of claim 57.

103. A cell genetically modified to express the artificial expression construct of claim 2 or the system of claim 57.

104. The cell of claim 103, wherein the cell is an autologous cell or an allogeneic cell in reference to a subject.

105. The cell of claim 103, wherein the cell is in vivo or ex vivo.

106. The cell of claim 103, wherein the cell is an immune cell.

107. The cell of claim 106, wherein the immune cell is a lymphocyte.

108. The cell of claim 107, wherein the lymphocyte comprises a T cell, B cell, natural killer (NK) cell, or NK-T cell.

109. The cell of claim 103, wherein the cell is a T cell selected from a CD3+ T cell, a CD4+ T cell, a CD8+ T cell, a central memory T cell, an effector memory T cell, and/or a naive T cell.

110. The cell of claim 103, wherein the cell is a CD8+ T cell.

111. The cell of claim 103, wherein the cell is a CD4+ T cell.

112. A population of cells genetically modified to express the artificial expression construct of claim 2 or the system of claim 57.

113. The population of cells of claim 112, wherein the population of cells comprises autologous cells or allogeneic cells in reference to a subject.

114. The population of cells of claim 112, wherein the population is in vivo or ex vivo.

115. The population of cells of claim 112, wherein the cell is an immune cell.

116. The population of cells of claim 115, wherein the immune cell is a lymphocyte.

117. The population of cells of claim 116, wherein the lymphocyte comprises a T cell, B cell, natural killer (NK) cell, or NK-T cell.

118. The population of cells of claim 112, wherein the population comprises CD4+ T cells and/or CD8+ T cells.

119. A formulation comprising (i) cells genetically modified to express the artificial expression construct of claim 2 or the system of claim 57 and (ii) a pharmaceutically acceptable carrier.

120. A method of genetically modifying an immune cell to have potentiated function comprising:
contacting the cell with the artificial expression construct of claim 2 having a sequence encoding a PD1:cytokine chimera.

121. The method of claim 120, wherein the potentiated function comprises potentiated cell killing ability.

122. The method of claim 121, wherein the encoded PD1:cytokine chimera comprises: PD1 linked to interleukin (IL)21 (PD1:IL21),
PD1 linked to IL15 (PD1:IL15),
PD1 linked to an N72D mutant IL15 (PD1:IL15(N72D)),
PD1 linked to IL18 (PD1:IL18),
PD1 linked to decoy resistant IL18 (PD1:drIL18),
PD1 linked to IL12 (PD1:IL12), or
PD1 linked to interferon alpha (PD1:IFNα).

123. The method of claim 122, wherein PD1 is PD1 with an A99L mutation.

124. The method of claim 122, wherein the PD1is wildtype PD1.

125. The method of claim 122, wherein the encoded PD1:cytokine chimera comprises PD1:IFNα.

126. The method of claim 120, wherein the potentiated function comprises potentiated proliferation.

127. The method of claim 126, wherein the encoded PD1:cytokine chimera comprises:
PD1 linked to interleukin (IL)21 (PD1:IL21),
PD1 linked to IL15 (PD1:IL15),
PD1 linked to an N72D mutant IL15 (PD1:IL15(N72D)),
PD1 linked to IL18 (PD1:IL18),
PD1 linked to decoy resistant IL18 (PD1:drIL18),
PD1 linked to IL12 (PD1:IL12), or
PD1 linked to interferon alpha (PD1:IFNα).

128. The method of claim 127, wherein the encoded PD1:cytokine chimera comprises PD1:IFNα.

129. The method of claim 120, wherein the potentiated function comprises potentiated cytokine output.

130. The method of claim 129, wherein the potentiated cytokine output comprises IFNY.

131. The method of claim 130, wherein the encoded PD1:cytokine chimera comprises:
PD1 linked to interleukin (IL)21 (PD1:IL21),
PD1 linked to IL15 (PD1:IL15),
PD1 linked to an N72D mutant IL15 (PD1:IL15(N72D)),
PD1 linked to IL18 (PD1:IL18),
PD1 linked to decoy resistant IL18 (PD1:drIL18),
PD1 linked to IL12 (PD1:IL12), or
PD1 linked to interferon alpha (PD1:IFNα).

132. The method of claim 131, wherein PD1 is PD1 with an A99L mutation.

133. The method of claim 131, wherein the PD1 is wildtype PD1.

134. The method of claim 131, wherein the encoded PD1:cytokine chimera comprises PD1:IFNα.

135. The method of claim 129, wherein the potentiated cytokine output comprises TNFα output.

136. The method of claim 135, wherein the encoded PD1:cytokine chimera comprises:
PD1 linked to interleukin (IL)21 (PD1:IL21),
PD1 linked to IL15 (PD1:IL15),
PD1 linked to an N72D mutant IL15 (PD1:IL15(N72D)),
PD1 linked to IL18 (PD1:IL18),
PD1 linked to decoy resistant IL18 (PD1:drIL18),
PD1 linked to IL12 (PD1:IL12), or
PD1 linked to interferon alpha (PD1:IFNα).

137. The method of claim 136, wherein PD1 is PD1 with an A99L mutation.

138. The method of claim 136, wherein the PD1is wildtype PD1.

139. The method of claim 136, wherein the encoded PD1:cytokine chimera comprises PD1:IFNα.

140. The method of claim 120, wherein the artificial expression construct further comprises a sequence encoding a recombinant receptor comprising a binding domain that binds an antigen expressed on the surface of target cells.

141. The method of claim 140, wherein the sequence encoding the recombinant receptor is operably linked to a second promoter.

142. The method of claim 141, wherein the second promoter comprises an EF1α(L) promoter, EF1α(s) promoter, myeloproliferative sarcoma virus (MND) promoter, cytomegalovirus (CMV) promoter, simian virus 40 (SV40) early promoter, mouse mammary tumor virus (MMTV) promoter, human immunodeficiency virus (HIV) long terminal repeat (LTR) promoter, MoMuLV promoter, avian leukemia virus promoter, Epstein-Barr virus immediate early promoter, Rous sarcoma virus promoter, actin promoter, myosin promoter, hemoglobin promoter, or a creatine kinase promoter.

143. The method of claim 120, wherein the method further comprises contacting the cell with an artificial expression construct comprising a sequence encoding a recombinant receptor comprising a binding domain that binds an antigen expressed on the surface of target cells.

144. The method of claim 143, wherein the contacting the cell with the artificial expression construct of claim 2 and the artificial expression construct comprising the sequence encoding the recombinant receptor occurs simultaneously.

145. The method of claim 143, wherein the contacting the cell with the artificial expression construct of claim 2 and the artificial expression construct comprising the sequence encoding the recombinant receptor occurs at different times.

146. The method of claim 120, wherein the artificial expression construct comprises the sequence as set forth in SEQ ID NOs: 98, 99, 100, 101, or 102 or a sequence having 90% sequence identity to a sequence as set forth in 98, 99, 100, 101, or 102.

147. A method of treating a subject in need thereof comprising administering a therapeutically effective amount of the artificial expression construct of claim 2, the system of claim 57, the nanoparticle of claim 102, or the formulation of claim 119 to the subject thereby treating the subject in need thereof.

148. The method of claim 147, wherein the subject in need thereof comprises cancer or an infection.

149. The method of claim 147, wherein the administering a therapeutically effective amount comprises administering intravesically, intravenously, intradermally, intraarterially, intraparenterally, intranodally, intralymphaticaly, intraperitoneally, intralesionally, intraprostaticaly, intravaginally, intrarectally, topically, intrathecally, intratumorally, intramuscularly, or subcutaneously.

150. A recombinant protein comprising programmed cell death-1 (PD1) and a cytokine.

151. The recombinant protein of claim 150, wherein the cytokine is an immunostimulatory cytokine.

152. The recombinant protein of claim 151, wherein the immunostimulatory cytokine comprises an interleukin (IL)21, an IL15, an N72D mutant IL15 (IL15(N72D), an IL18, a decoy resistant IL18 (drIL18), an IL12, or an interferon alpha (IFNα).

153. The recombinant protein of claim 150, wherein the recombinant protein comprises:
PD1 linked to interleukin (IL)21 (PD1:IL21),
PD1 linked to IL15 (PD1:IL15),
PD1 linked to an N72D mutant IL15 (PD1:IL15(N72D)),
PD1 linked to IL18 (PD1:IL18),
PD1 linked to decoy resistant IL18 (PD1:drIL18),
PD1 linked to IL12 (PD1:IL12),
PD1 linked to interferon alpha (PD1:IFNα),
PD1 with an A99L mutation (PD1(A99L)) linked to IL12 ((PD1(A99L):IL21),
PD1(A99L) linked to IL15 (PD1(A99L):IL15),
PD1(A99L) linked to an N72D mutant IL15 (PD1(A99L):IL15(N72D)),
PD1(A99L) linked to IL18 (PD1(A99L):IL18),
PD1(A99L) linked to decoy resistant IL18 (PD1(A99L):drIL18),
PD1(A99L) linked to IL12 (PD1(A99L):IL12), or
PD1(A99L) linked to interferon alpha (PD1(A99L):IFNα).

154. The recombinant protein of claim 150, wherein the PD1 is linked to the cytokine through a protein linker.

155. The recombinant protein of claim 154, wherein the protein linker is a flexible linker.

156. The recombinant protein of claim 155, wherein the flexible linker is a Gly-Ser linker.

157. The recombinant protein of claim 156, wherein the Gly-Ser linker comprises the sequence as set forth in SEQ ID NO: 128 or the sequence GGG.

158. The recombinant protein of claim 150, wherein the recombinant protein is encoded by the sequence as set forth in SEQ ID NO: 7, SEQ ID NO: 8, SEQ ID NO: 9, SEQ ID NO: 10, or SEQ ID NO: 11 or a sequence having at least 90% sequence identity to the sequence set forth SEQ ID NO: 7, SEQ ID NO: 8, SEQ ID NO: 9, SEQ ID NO: 10, or SEQ ID NO: 11.

159. The recombinant protein of claim 150, wherein the recombinant protein comprises the sequence as set forth in SEQ ID NO: 12, SEQ ID NO: 13, SEQ ID NO: 14, SEQ ID NO: 15, or SEQ ID NO: 16 or a sequence having at least 90% sequence identity to the sequence set forth in SEQ ID NO: 12, SEQ ID NO: 13, SEQ ID NO: 14, SEQ ID NO: 15, or SEQ ID NO: 16.


SEQUENCE LISTING (138 sequences)

SEQ ID NO: 1 - DNA, 510 bp, Synthetic construct
  source 1..510: mol_type = other DNA
atgcagatccctcaggccccttggcctgtcgtgtgggctgtgctgcagctgggatggcggcctggctggtttctggacagccccgacagaccctggaacccccctacattttcccctgccctgctggtcgtgaccgagggcgacaatgccaccttcacctgtagcttcagcaacaccagcgagagcttcgtgctgaactggtacagaatgagccccagcaaccagaccgacaagctggccgccttccccgaggatagatctcagcccggccaggactgccggttcagagtgacccagctgcccaacggccgggacttccacatgtctgtcgtgcgggccagacggaacgacagcggcacatatctgtgcggcgccatcagcctggcccccaagctccagatcaaagagagcctgagagccgagctgagagtgaccgagagaagggccgaagtgcctaccgcccaccctagcccatctccaagacctgccggccagttccagacactggtc

SEQ ID NO: 2 - DNA, 414 bp, Synthetic construct
  source 1..414: mol_type = other DNA
cacaagagcagctctcagggccaagaccggcacatgatccggatgagacagctgatcgacatcgtggaccagctgaagaactacgtgaacgacctggtgcctgagttcctgcctgctcctgaggacgtggaaacaaactgcgagtggagcgccttcagctgcttccagaaggcccagctgaaaagcgccaacaccggcaacaacgagcggatcatcaatgtgtccatcaagaagctgaagcggaagcctcctagcaccaacgccggaagaaggcagaagcacagactgacctgtccaagctgcgacagctacgagaagaagcctccaaaagagttcctggaacggttcaagagcctgctgcagaagatgatccaccagcacctgagcagcagaacccacggcagcgaggattct

SEQ ID NO: 3 - DNA, 342 bp, Synthetic construct
  source 1..342: mol_type = other DNA
aattgggtaaacgtgattagtgatctcaaaaagattgaagatctgattcagtctatgcatatcgatgcaaccctttacacggagtccgatgttcacccgtcatgcaaggtaaccgcaatgaagtgtttcctgttggaattgcaggtaatctccttggagtccggtgatgcatcaatccacgatactgtcgaaaacttgattatcctcgcgaacgacagtctctcctcaaacggaaacgtcactgagtctggatgcaaagagtgtgaagagcttgaggaaaaaaatataaaggaatttcttcagtctttcgtgcatatagttcaaatgttcataaacacaagt

SEQ ID NO: 4 - DNA, 471 bp, Synthetic construct
  source 1..471: mol_type = other DNA
tatttcggcaaattggagagcaagttgagtgtcataaggaatttgaatgaccaggttttgtttatcgatcagggaaacaggcctttgtttgaggacatgaccgactccgattgtagagacaacgcaccccgaactatattcataatcagcaaatactccgattctcttgcgcgaggactcgcggttacgatctcagtcaagtgcgagaagataagtaccctgtcatgcgagaataaaattatctctttcaaggaaatgaatccgccagataatataaaggatacaaagagtgacatcatctttttccagcgagacgtacccggccactctcgcaagatgcaattcgagtcttctagctacgaaggatattttttggcgtgcgagaaagagagggacttgttcaaacttatcttgaagaaggaggatgaactgggtgaccgctctattatgtttacagtacaaaatgaggat

SEQ ID NO: 5 - DNA, 1623 bp, Synthetic construct
  source 1..1623: mol_type = other DNA
atgtgtcatcagcagttggtgatctcttggttttctcttgtatttcttgcgtcaccacttgtggccatctgggaactgaagaaggacgtatatgtggttgaactggactggtaccccgacgcaccgggggaaatggtagtgttgacttgtgatacacctgaggaagacggaattacttggacccttgaccaatccagtgaggtgcttggatcaggaaagacactgaccattcaagtcaaagagtttggggacgccggacaatacacttgtcacaaaggtggagaggtattgagccattctcttcttcttctccataagaaagaagatgggatatggagcacggatattctgaaagaccaaaaagaaccaaaaaacaaaactttcctcaggtgtgaggcgaaaaattactcaggacgctttacttgctggtggcttaccactatatccactgatcttactttttctgtcaagagtagtcgcggttcatctgatccccaaggtgtgacatgtggtgcagccacgctcagtgccgaacgagtgaggggggataataaagaatatgagtactccgttgaatgccaagaagattctgcgtgtcctgctgcagaagaaagtctgccgatagaggttatggttgatgcggttcacaaacttaaatatgagaattatacgagcagtttcttcatccgggacatcataaaaccagatccgcctaaaaatcttcaacttaaacccctgaaaaattccaggcaagttgaggtgagctgggagtatcctgatacgtggtctacgccccactcatacttcagtctcactttttgcgttcaggttcaaggtaagagtaaacgcgaaaaaaaagaccgcgtttttacagacaaaacgagtgccaccgtaatatgcaggaaaaacgctagtattagtgtacgggcacaggatcgatactatagcagttcatggtctgagtgggcatctgttccttgtagtggagggggcggtagtggtgggggaggatccggtggcggtggctccgcgcgcaatctgcctgtggcgacccctgatcccggtatgtttccgtgtttgcatcattcacaaaatctcttgagggcagtttcaaatatgctgcaaaaagcacgacaaacgctggagttttacccatgtaccagtgaagagattgatcatgaggatataactaaagacaagaccagtactgtcgaggcttgtctccctctggaactgaccaagaacgagagctgcctcaattcccgcgagacatcctttatcactaatggttcctgcttggcttctaggaaaacatccttcatgatggcattgtgtctgtcatctatatacgaagacctcaagatgtaccaggtggagttcaagacgatgaacgcgaaactcttgatggacccaaaacgacagatattcctcgatcaaaacatgttggcggtcatcgatgaactgatgcaggcgctcaacttcaatagcgagaccgttccgcaaaagagctctctcgaggagcccgatttttacaaaacaaagataaaactgtgcatcctgcttcacgccttcaggattagggcggttactattgacagagtaatgtcctacctcaatgcttct

SEQ ID NO: 6 - DNA, 495 bp, Synthetic construct
  source 1..495: mol_type = other DNA
tgcgacctgcctcagactcactctctgggctctagacggaccctgatgctgctggcccagatgagaaagatcagcctgttctcctgcctgaaggaccggcacgatttcggcttccctcaagaggaattcggcaaccagttccagaaggccgagacaatccctgtgctgcacgagatgatccagcagatcttcaacctgttcagcaccaaggacagcagcgccgcctgggatgagacactgctggacaagttctacaccgagctgtaccagcagctcaacgacctggaagcctgcgtgatccaaggcgtgggagtgaccgaaacacccctgatgaaggaagatagcatcctggccgtgcgcaagtacttccagcggatcaccctgtacctgaaagagaagaagtacagcccctgcgcctgggaagtcgtcagagccgagattatgcggagcttcagcctgagcaccaacctgcaagaaagcctgcggtccaaagaa

SEQ ID NO: 7 - DNA, 969 bp, Synthetic construct
  source 1..969: mol_type = other DNA
atgcagatccctcaggccccttggcctgtcgtgtgggctgtgctgcagctgggatggcggcctggctggtttctggacagccccgacagaccctggaacccccctacattttcccctgccctgctggtcgtgaccgagggcgacaatgccaccttcacctgtagcttcagcaacaccagcgagagcttcgtgctgaactggtacagaatgagccccagcaaccagaccgacaagctggccgccttccccgaggatagatctcagcccggccaggactgccggttcagagtgacccagctgcccaacggccgggacttccacatgtctgtcgtgcgggccagacggaacgacagcggcacatatctgtgcggcgccatcagcctggcccccaagctccagatcaaagagagcctgagagccgagctgagagtgaccgagagaagggccgaagtgcctaccgcccaccctagcccatctccaagacctgccggccagttccagacactggtcggcggaggcggaagcggtggcggaggaagcggaggcggcggatctcacaagagcagctctcagggccaagaccggcacatgatccggatgagacagctgatcgacatcgtggaccagctgaagaactacgtgaacgacctggtgcctgagttcctgcctgctcctgaggacgtggaaacaaactgcgagtggagcgccttcagctgcttccagaaggcccagctgaaaagcgccaacaccggcaacaacgagcggatcatcaatgtgtccatcaagaagctgaagcggaagcctcctagcaccaacgccggaagaaggcagaagcacagactgacctgtccaagctgcgacagctacgagaagaagcctccaaaagagttcctggaacggttcaagagcctgctgcagaagatgatccaccagcacctgagcagcagaacccacggcagcgaggattct

SEQ ID NO: 8 - DNA, 897 bp, Synthetic construct
  source 1..897: mol_type = other DNA
atgcagatccctcaggccccttggcctgtcgtgtgggctgtgctgcagctgggatggcggcctggctggtttctggacagccccgacagaccctggaacccccctacattttcccctgccctgctggtcgtgaccgagggcgacaatgccaccttcacctgtagcttcagcaacaccagcgagagcttcgtgctgaactggtacagaatgagccccagcaaccagaccgacaagctggccgccttccccgaggatagatctcagcccggccaggactgccggttcagagtgacccagctgcccaacggccgggacttccacatgtctgtcgtgcgggccagacggaacgacagcggcacatatctgtgcggcgccatcagcctggcccccaagctccagatcaaagagagcctgagagccgagctgagagtgaccgagagaagggccgaagtgcctaccgcccaccctagcccatctccaagacctgccggccagttccagacactggtcggcggaggcggaagcggtggcggaggaagcggaggcggcggatctaattgggtaaacgtgattagtgatctcaaaaagattgaagatctgattcagtctatgcatatcgatgcaaccctttacacggagtccgatgttcacccgtcatgcaaggtaaccgcaatgaagtgtttcctgttggaattgcaggtaatctccttggagtccggtgatgcatcaatccacgatactgtcgaaaacttgattatcctcgcgaacgacagtctctcctcaaacggaaacgtcactgagtctggatgcaaagagtgtgaagagcttgaggaaaaaaatataaaggaatttcttcagtctttcgtgcatatagttcaaatgttcataaacacaagt

SEQ ID NO: 9 - DNA, 1026 bp, Synthetic construct
  source 1..1026: mol_type = other DNA
atgcagatccctcaggccccttggcctgtcgtgtgggctgtgctgcagctgggatggcggcctggctggtttctggacagccccgacagaccctggaacccccctacattttcccctgccctgctggtcgtgaccgagggcgacaatgccaccttcacctgtagcttcagcaacaccagcgagagcttcgtgctgaactggtacagaatgagccccagcaaccagaccgacaagctggccgccttccccgaggatagatctcagcccggccaggactgccggttcagagtgacccagctgcccaacggccgggacttccacatgtctgtcgtgcgggccagacggaacgacagcggcacatatctgtgcggcgccatcagcctggcccccaagctccagatcaaagagagcctgagagccgagctgagagtgaccgagagaagggccgaagtgcctaccgcccaccctagcccatctccaagacctgccggccagttccagacactggtcggcggaggcggaagcggtggcggaggaagcggaggcggcggatcttatttcggcaaattggagagcaagttgagtgtcataaggaatttgaatgaccaggttttgtttatcgatcagggaaacaggcctttgtttgaggacatgaccgactccgattgtagagacaacgcaccccgaactatattcataatcagcaaatactccgattctcttgcgcgaggactcgcggttacgatctcagtcaagtgcgagaagataagtaccctgtcatgcgagaataaaattatctctttcaaggaaatgaatccgccagataatataaaggatacaaagagtgacatcatctttttccagcgagacgtacccggccactctcgcaagatgcaattcgagtcttctagctacgaaggatattttttggcgtgcgagaaagagagggacttgttcaaacttatcttgaagaaggaggatgaactgggtgaccgctctattatgtttacagtacaaaatgaggat

SEQ ID NO: 10 - DNA, 2472 bp, Synthetic construct
  source 1..2472: mol_type = other DNA
atgcagatccctcaggccccttggcctgtcgtgtgggctgtgctgcagctgggatggcggcctggctggtttctggacagccccgacagaccctggaacccccctacattttcccctgccctgctggtcgtgaccgagggcgacaatgccaccttcacctgtagcttcagcaacaccagcgagagcttcgtgctgaactggtacagaatgagccccagcaaccagaccgacaagctggccgccttccccgaggatagatctcagcccggccaggactgccggttcagagtgacccagctgcccaacggccgggacttccacatgtctgtcgtgcgggccagacggaacgacagcggcacatatctgtgcggcgccatcagcctggcccccaagctccagatcaaagagagcctgagagccgagctgagagtgaccgagagaagggccgaagtgcctaccgcccaccctagcccatctccaagacctgccggccagttccagacactggtcggcggaggcggaagcggtggcggaggaagcggaggcggcggatctatgtgtcatcagcagttggtgatctcttggttttctcttgtatttcttgcgtcaccacttgtggccatctgggaactgaagaaggacgtatatgtggttgaactggactggtaccccgacgcaccgggggaaatggtagtgttgacttgtgatacacctgaggaagacggaattacttggacccttgaccaatccagtgaggtgcttggatcaggaaagacactgaccattcaagtcaaagagtttggggacgccggacaatacacttgtcacaaaggtggagaggtattgagccattctcttcttcttctccataagaaagaagatgggatatggagcacggatattctgaaagaccaaaaagaaccaaaaaacaaaactttcctcaggtgtgaggcgaaaaattactcaggacgctttacttgctggtggcttaccactatatccactgatcttactttttctgtcaagagtagtcgcggttcatctgatccccaaggtgtgacatgtggtgcagccacgctcagtgccgaacgagtgaggggggataataaagaatatgagtactccgttgaatgccaagaagattctgcgtgtcctgctgcagaagaaagtctgccgatagaggttatggttgatgcggttcacaaacttaaatatgagaattatacgagcagtttcttcatccgggacatcataaaaccagatccgcctaaaaatcttcaacttaaacccctgaaaaattccaggcaagttgaggtgagctgggagtatcctgatacgtggtctacgccccactcatacttcagtctcactttttgcgttcaggttcaaggtaagagtaaacgcgaaaaaaaagaccgcgtttttacagacaaaacgagtgccaccgtaatatgcaggaaaaacgctagtattagtgtacgggcacaggatcgatactatagcagttcatggtctgagtgggcatctgttccttgtagtgacatcataaaaccagatccgcctaaaaatcttcaacttaaacccctgaaaaattccaggcaagttgaggtgagctgggagtatcctgatacgtggtctacgccccactcatacttcagtctcactttttgcgttcaggttcaaggtaagagtaaacgcgaaaaaaaagaccgcgtttttacagacaaaacgagtgccaccgtaatatgcaggaaaaacgctagtattagtgtacgggcacaggatcgatactatagcagttcatggtctgagtgggcatctgttccttgtagtggagggggcggtagtggtgggggaggatccggtggcggtggctccgcgcgcaatctgcctgtggcgacccctgatcccggtatgtttccgtgtttgcatcattcacaaaatctcttgagggcagtttcaaatatgctgcaaaaagcacgacaaacgctggagttttacccatgtaccagtgaagagattgatcatgaggatataactaaagacaagaccagtactgtcgaggcttgtctccctctggaactgaccaagaacgagagctgcctcaattcccgcgagacatcctttatcactaatggttcctgcttggcttctaggaaaacatccttcatgatggcattgtgtctgtcatctatatacgaagacctcaagatgtaccaggtggagttcaagacgatgaacgcgaaactcttgatggacccaaaacgacagatattcctcgatcaaaacatgttggcggtcatcgatgaactgatgcaggcgctcaacttcaatagcgagaccgttccgcaaaagagctctctcgaggagcccgatttttacaaaacaaagataaaactgtgcatcctgcttcacgccttcaggattagggcggttactattgacagagtaatgtcctacctcaatgcttct

SEQ ID NO: 11 - DNA, 1014 bp, Synthetic construct
  source 1..1014: mol_type = other DNA
atgcagatccctcaggccccttggcctgtcgtgtgggctgtgctgcagctgggatggcggcctggctggtttctggacagccccgacagaccctggaacccccctacattttcccctgccctgctggtcgtgaccgagggcgacaatgccaccttcacctgtagcttcagcaacaccagcgagagcttcgtgctgaactggtacagaatgagccccagcaaccagaccgacaagctggccgccttccccgaggatagatctcagcccggccaggactgccggttcagagtgacccagctgcccaacggccgggacttccacatgtctgtcgtgcgggccagacggaacgacagcggcacatatctgtgcggcgccatcagcctggcccccaagctccagatcaaagagagcctgagagccgagctgagagtgaccgagagaagggccgaagtgcctaccgcccaccctagcccatctccaagacctgccggccagttccagacactggtcggcggaggatgcgacctgcctcagactcactctctgggctctagacggaccctgatgctgctggcccagatgagaaagatcagcctgttctcctgcctgaaggaccggcacgatttcggcttccctcaagaggaattcggcaaccagttccagaaggccgagacaatccctgtgctgcacgagatgatccagcagatcttcaacctgttcagcaccaaggacagcagcgccgcctgggatgagacactgctggacaagttctacaccgagctgtaccagcagctcaacgacctggaagcctgcgtgatccaaggcgtgggagtgaccgaaacacccctgatgaaggaagatagcatcctggccgtgcgcaagtacttccagcggatcaccctgtacctgaaagagaagaagtacagcccctgcgcctgggaagtcgtcagagccgagattatgcggagcttcagcctgagcaccaacctgcaagaaagcctgcggtccaaagaa

SEQ ID NO: 12 - protein, 323 aa, Synthetic construct
MQIPQAPWPVVWAVLQLGWRPGWFLDSPDRPWNPPTFSPALLVVTEGDNATFTCSFSNTSESFVLNWYRMSPSNQTDKLAAFPEDRSQPGQDCRFRVTQLPNGRDFHMSVVRARRNDSGTYLCGAISLAPKLQIKESLRAELRVTERRAEVPTAHPSPSPRPAGQFQTLVGGGGSGGGGSGGGGSHKSSSQGQDRHMIRMRQLIDIVDQLKNYVNDLVPEFLPAPEDVETNCEWSAFSCFQKAQLKSANTGNNERIINVSIKKLKRKPPSTNAGRRQKHRLTCPSCDSYEKKPPKEFLERFKSLLQKMIHQHLSSRTHGSEDS

SEQ ID NO: 13 - protein, 299 aa, Synthetic construct
MQIPQAPWPVVWAVLQLGWRPGWFLDSPDRPWNPPTFSPALLVVTEGDNATFTCSFSNTSESFVLNWYRMSPSNQTDKLAAFPEDRSQPGQDCRFRVTQLPNGRDFHMSVVRARRNDSGTYLCGAISLAPKLQIKESLRAELRVTERRAEVPTAHPSPSPRPAGQFQTLVGGGGSGGGGSGGGGSNWVNVISDLKKIEDLIQSMHIDATLYTESDVHPSCKVTAMKCFLLELQVISLESGDASIHDTVENLIILANDSLSSNGNVTESGCKECEELEEKNIKEFLQSFVHIVQMFINTS

SEQ ID NO: 14 - protein, 342 aa, Synthetic construct
MQIPQAPWPVVWAVLQLGWRPGWFLDSPDRPWNPPTFSPALLVVTEGDNATFTCSFSNTSESFVLNWYRMSPSNQTDKLAAFPEDRSQPGQDCRFRVTQLPNGRDFHMSVVRARRNDSGTYLCGAISLAPKLQIKESLRAELRVTERRAEVPTAHPSPSPRPAGQFQTLVGGGGSGGGGSGGGGSYFGKLESKLSVIRNLNDQVLFIDQGNRPLFEDMTDSDCRDNAPRTIFIISKYSDSLARGLAVTISVKCEKISTLSCENKIISFKEMNPPDNIKDTKSDIIFFQRDVPGHSRKMQFESSSYEGYFLACEKERDLFKLILKKEDELGDRSIMFTVQNED

SEQ ID NO: 15 - protein, 824 aa, Synthetic construct
MQIPQAPWPVVWAVLQLGWRPGWFLDSPDRPWNPPTFSPALLVVTEGDNATFTCSFSNTSESFVLNWYRMSPSNQTDKLAAFPEDRSQPGQDCRFRVTQLPNGRDFHMSVVRARRNDSGTYLCGAISLAPKLQIKESLRAELRVTERRAEVPTAHPSPSPRPAGQFQTLVGGGGSGGGGSGGGGSMCHQQLVISWFSLVFLASPLVAIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCSDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCSGGGGSGGGGSGGGGSARNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNAS

SEQ ID NO: 16 - protein, 338 aa, Synthetic construct
MQIPQAPWPVVWAVLQLGWRPGWFLDSPDRPWNPPTFSPALLVVTEGDNATFTCSFSNTSESFVLNWYRMSPSNQTDKLAAFPEDRSQPGQDCRFRVTQLPNGRDFHMSVVRARRNDSGTYLCGAISLAPKLQIKESLRAELRVTERRAEVPTAHPSPSPRPAGQFQTLVGGGCDLPQTHSLGSRRTLMLLAQMRKISLFSCLKDRHDFGFPQEEFGNQFQKAETIPVLHEMIQQIFNLFSTKDSSAAWDETLLDKFYTELYQQLNDLEACVIQGVGVTETPLMKEDSILAVRKYFQRITLYLKEKKYSPCAWEVVRAEIMRSFSLSTNLQESLRSKE

SEQ ID NO: 17 - DNA, 333 bp, Synthetic construct
  source 1..333: mol_type = other DNA
tcgaatgagtcacatcgatctccgccccctcttcgagggggcggggtcgaggaggaaaaactcgaatgagtcacatcgaccctttgatcttcgaggggactttccggggtggagcaagcgtgacaagtccacgtatgacccgaccgacgatatcgaagcctacgcgctgaacgccagccccgatcgaccccgccccctcgatttccaagaaatcgaatgacatcatctttcgaatgacatcatctttcgaggggactttcctcgaacttccttcgaggggactttcctcgaggggactttcctcgaggaggaaaaactcgagtagagtctaga

SEQ ID NO: 18 - DNA, 390 bp, Synthetic construct
  source 1..390: mol_type = other DNA
ctgcttagggttaggcgttttgcgctgcttcgcgatcgaatgagtcacatcgatctccgccccctcttcgagggggcggggtcgaggaggaaaaactcgaatgagtcacatcgaccctttgatcttcgaggggactttccggggtggagcaagcgtgacaagtccacgtatgacccgaccgacgatatcgaagcctacgcgctgaacgccagccccgatcgaccccgccccctcgatttccaagaaatcgaatgacatcatctttcgaatgacatcatctttcgaggggactttcctcgaacttccttcgaggggactttcctcgaggggactttcctcgaggaggaaaaactcgagtagagtctagactctacattttgacacccccat

SEQ ID NO: 19 - DNA, 265 bp, Synthetic construct
  source 1..265: mol_type = other DNA
ctgcttagggttaggcgttttgcgctgcttcgcgatcgaggaggaaaaactcgatgtgactcattcgaagatcaaagggtcgaggaaagtcccctcgaccctttgatcttcgaatgacatcatctttcgaggaaagtcccctcgaggggactttcctcgaggggactttcctcgaatgagtcacatcgatctccgccccctcttcgagcgccaaatcgaggacgtgatcgagtagagtctagactctacattttgacacccccat

SEQ ID NO: 20 - DNA, 232 bp, Synthetic construct
  source 1..232: mol_type = other DNA
ctgcttagggttaggcgttttgcgctgcttcgcgatcgagggggcggggtcgatctccgccccctcttcgagtttttcctcctcgaggggactttcctcgaatgacatcatctttcgaccctttgatcttcgaaggaagttcgatcacgtcctcgatttggcgctcgaggggactttcctcgaagatcaaagggtcgagtagagtctagactctacattttgacacccccat

SEQ ID NO: 21 - DNA, 257 bp, Synthetic construct
  source 1..257: mol_type = other DNA
ctgcttagggttaggcgttttgcgctgcttcgcgatcgatttccaagaaatcgaggaaagtcccctcgaccctttgatcttcgaggaggaaaaactcgaatgagtcacatcgaccctttgatcttcgaagagggggcggagatcgatctccgccccctcttcgaggaaagtcccctcgaccctttgatcttcgaccccgccccctcgaggaaagtcccctcgagtagagtctagactctacattttgacacccccat

SEQ ID NO: 22 - DNA, 256 bp, Synthetic construct
  source 1..256: mol_type = other DNA
ctgcttagggttaggcgttttgcgctgcttcgcgatcgatctccgccccctcttcgaagatcaaagggtcgaggggactttcctcgatttcttggaaatcgaatgacatcatctttcgatttggcgctcgaatgacatcatctttcgaccctttgatcttcgatgtgactcattcgaggggactttcctcgaagatcaaagggtcgaggggactttcctcgagtagagtctagactctacattttgacacccccat

SEQ ID NO: 23 - DNA, 205 bp, Synthetic construct
  source 1..205: mol_type = other DNA
ctgcttagggttaggcgttttgcgctgcttcgcgatcgaggggactttcctcgagggggcggggtcgatttggcgctcgatctccgccccctcttcgaatgagtcacatcgaggaaagtcccctcgaggaggaaaaactcgatgtgactcattcgatttccaagaaatcgagtagagtctagactctacattttgacacccccat

SEQ ID NO: 24 - DNA, 283 bp, Synthetic construct
  source 1..283: mol_type = other DNA
ctgcttagggttaggcgttttgcgctgcttcgcgatcgatttccaagaaatcgatctccgccccctcttcgaatgacatcatctttcgaatgacatcatctttcgaggaggaaaaactcgaccccgccccctcgatttcttggaaatcgaggaaagtcccctcgagcgccaaatcgaaggaagttcgaatgacatcatctttcgaatgagtcacatcgaggaaagtcccctcgaggaggaaaaactcgagtagagtctagactctacattttgacacccccat

SEQ ID NO: 25 - DNA, 192 bp, Synthetic construct
  source 1..192: mol_type = other DNA
ctgcttagggttaggcgttttgcgctgcttcgcgatcgatttccaagaaatcgagtttttcctcctcgagggggcggggtcgaggggactttcctcgaccctttgatcttcgaggaaagtcccctcgagcgccaaatcgatctccgccccctcttcgagtagagtctagactctacattttgacacccccat

SEQ ID NO: 26 - DNA, 283 bp, Synthetic construct
  source 1..283: mol_type = other DNA
ctgcttagggttaggcgttttgcgctgcttcgcgatcgaagatcaaagggtcgatttcttggaaatcgatgtgactcattcgatcacgtcctcgaggaggaaaaactcgaggaaagtcccctcgaacttccttcgagggggcggggtcgaatgagtcacatcgaggaaagtcccctcgaggggactttcctcgatttcttggaaatcgaagagggggcggagatcgagtttttcctcctcgaggaaagtcccctcgatcgactctacattttgacacccccat

SEQ ID NO: 27 - DNA, 274 bp, Synthetic construct
  source 1..274: mol_type = other DNA
ctgcttagggttaggcgttttgcgctgcttcgcgatcgaatgagtcacatcgaagatcaaagggtcgaccctttgatcttcgatttggcgctcgatgtgactcattcgaccccgccccctcgaggaaagtcccctcgaggaaagtcccctcgaggaaagtcccctcgaggaaagtcccctcgagtttttcctcctcgaagagggggcggagatcgatttggcgctcgaggacgtgatcgagtagagtctagactctacattttgacacccccat

SEQ ID NO: 28 - DNA, 286 bp, Synthetic construct
  source 1..286: mol_type = other DNA
ctgcttagggttaggcgttttgcgctgcttcgcgatcgatttccaagaaatcgagtttttcctcctcgatttccaagaaatcgaggggactttcctcgaggggactttcctcgaatgacatcatctttcgatgtgactcattcgaagatcaaagggtcgaaggaagttcgaatgagtcacatcgaatgagtcacatcgacccttgatcttcgaggaggaaaaactcgatttggcgctcgatttggcgctcgagtagagtctagactctacattttgacacccccat

SEQ ID NO: 29 - DNA, 397 bp, Synthetic construct
  source 1..397: mol_type = other DNA
ctgcttagggttaggcgttttgcgctgcttcgcgatcgatttcttggaaatcgatttggcgctcgaaggaagttcgagggggcggggtcgatttccaagaaatcgagtttttcctcctcgagcgccaaatcgaccctttgatcttcgatcacgtcctcgagcgccaaatcgaggacgtgatcgaatgagtcacatcgaatgagtcacatcgaatgagtcacatcgatttggcgctcgatcggggggcggggtcgaggaaagtcccctcgaggaaagtcccctcgatttccaagaaatcgatttggcgctcgatctccgccccctcttcgatctccgccccctcttcgagtttttcctcctcgagtagagtctagactctacattttgacacccccat

SEQ ID NO: 30 - DNA, 235 bp, Synthetic construct
  source 1..235: mol_type = other DNA
ctgcttagggttaggcgttttgcgctgcttcgcgatcgaatgacatcatctttcgaggaaagtcccctcgaccctttgatcttcgaccccgccccctcgagtttttcctcctcgaggaaagtcccctcgaccctttgatcttcgaggggactttcctcgagggggcggggtcgaggacgtgatcgaggaaagtcccctcgagtagagtctagactctacattttgacacccccat

SEQ ID NO: 31 - DNA, 335 bp, Synthetic construct
  source 1..335: mol_type = other DNA
ctgcttagggttaggcgttttgcgctgcttcgcgatcgaagatcaaagggtcgaggaggaaaaactcgaggggactttcctcgaccctttgatcttcgaatgagtcacatcgatttcttggaaatcgaaggaagttcgaggggactttcctcgagtttttcctcctcgaacttccttcgaggaaagtcccctcgaagatcaaagggtcgaaggaagttcgaaagatgatgtcattcgatttcttggaaatcgaagatcaaagggtcgaagagggggcggagatcgatttccaagaaatcgagtagagtctagactctacattttgacacccccat

SEQ ID NO: 32 - DNA, 203 bp, Synthetic construct
  source 1..203: mol_type = other DNA
ctgcttagggttaggcgttttgcgctgcttcgcgatcgaatgagtcacatcgagcgccaaatcgaggggactttcctcgagggggcggggtcgagtttttcctcctcgatttccaagaaatcgaggggactttcctcgaggggactttcctcgaggggactttcctcgagtagagtctagactctacattttgacacccccat

SEQ ID NO: 33 - DNA, 287 bp, Synthetic construct
  source 1..287: mol_type = other DNA
ctgcttagggttaggcgttttgcgctgcttcgcgatcgagtttttcctcctcgaggggactttcctcgaaagatgatgtcattcgatctccgccccctcttcgaggggactttcctcgaggaaagtcccctcgaggaaagtcccctcgaagatcaaagggtcgaatgagtcacatcgaccctttgatcttcgaccctttgatcttcgatttccaagaaatcgaggaaagtcccctcgaggaaagtcccctcgagtagagtctagactctacattttgacacccccat

SEQ ID NO: 34 - DNA, 293 bp, Synthetic construct
  source 1..293: mol_type = other DNA
ctgcttagggttaggcgttttgcgctgcttcgcgatcgaggggactttcctcgaggaaagtcccctcgatttcttggaaatcgatttggcgctcgaggaggaaaaactcgatgtgactcattcgaccctttgatcttcgaggggactttcctcgaggggactttcctcgagcgccaaatcgaggaggaaaaactcgaggggactttcctcgatttcttggaaatcgaaagatgatgtcattcgaccctttgatcttcgagtagagtctagactctacattttgacacccccat

SEQ ID NO: 35 - DNA, 252 bp, Synthetic construct
  source 1..252: mol_type = other DNA
ctgcttagggttaggcgttttgcgctgcttcgcgatcgaggaaagtcccctcgaggaaagtcccctcgatttcttggaaatcgaatgacatcatctttcgatcacgtcctcgaggaaagtcccctcgagtttttcctcctcgaggggactttcctcgatttccaagaaatcgatttcttggaaatcgaccctttgatcttcgaggaggaaaaactcgagtagagtctagactctacattttgacacccccat

SEQ ID NO: 36 - DNA, 215 bp, Synthetic construct
  source 1..215: mol_type = other DNA
ctgcttagggttaggcgttttgcgctgcttcgcgatcgatgtgactcattcgaggaaagtcccctcgatttggcgctcgatgtgactcattcgatttggcgctcgaggggactttcctcgatttccaagaaatcgaggggactttcctcgaggaaagtcccctcgagtttttcctcctcgagtagagtctagactctacattttgacacccccat

SEQ ID NO: 37 - DNA, 254 bp, Synthetic construct
  source 1..254: mol_type = other DNA
ctgcttagggttaggcgttttgcgctgcttcgcgatcgatgtgactcattcgagcgccaaatcgatttccaagaaatcgaatgagtcacatcgaatgacatcatctttcgatgtgactcattcgaggaaagtcccctcgatttcttggaaatcgaggaaagtcccctcgagtttttcctcctcgaagagggggcggagatcgaatgacatcatctttcgagtagagtctagactctacattttgacacccccat

SEQ ID NO: 38 - DNA, 355 bp, Synthetic construct
  source 1..355: mol_type = other DNA
ctgcttagggttaggcgttttgcgctgcttcgcgatcgaggggactttcctcgatctccgccccctcttcgaggaaagtcccctcgaaggaagttcgaacttccttcgaggggactttcctcgaaggaagttcgagcgccaaatcgaatgagtcacatcgatttccaagaaatcgatctccgccccctcttcgaggaggaaaaactcgaggggactttcctcgatttccaagaaatcgaggggactttcctcgatcacgtcctcgaggacgtgatcgaatgagtcacatcgatgtgactcattcgaccctttgatcttcgagtagagtctagactctacattttgacacccccat

SEQ ID NO: 39 - DNA, 302 bp, Synthetic construct
  source 1..302: mol_type = other DNA
ctgcttagggttaggcgttttgcgctgcttcgcgatcgatttccaagaaatcgagggggcggggtcgaggggactttcctcgaggggactttcctcgaggaaagtcccctcgatctccgccccctcttcgaggaaagtcccctcgaacttccttcgaggaaagtcccctcgaatgagtcacatcgatttggcgctcgaggaaagtcccctcgaggggactttcctcgaggaaagtcccctcgagggggcggggtcgaacttccttcgagtagagtctagactctacattttgacacccccat

SEQ ID NO: 40 - DNA, 192 bp, Synthetic construct
  source 1..192: mol_type = other DNA
ctgcttagggttaggcgttttgcgctgcttcgcgatcgaatgagtcacatcgatctccgccccctcttcgatttggcgctcgaggggactttcctcgaccctttgatcttcgaggggactttcctcgaggaaagtcccctcgaggaaagtcccctcgagtagagtctagactctacattttgacacccccat

SEQ ID NO: 41 - DNA, 248 bp, Synthetic construct
  source 1..248: mol_type = other DNA
ctgcttagggttaggcgttttgcgctgcttcgcgatcgatttggcgctcgatgtgactcattcgaggaaagtcccctcgaggggactttcctcgaagatcaaagggtcgaaagatgatgtcattcgaggaaagtcccctcgaccccgccccctcgatttcttggaaatcgaatgagtcacatcgatttcttggaaatcgatgtgactcattcgagtagagtctagactctacattttgacacccccat

SEQ ID NO: 42 - DNA, 342 bp, Synthetic construct
  source 1..342: mol_type = other DNA
ctgcttagggttaggcgttttgcgctgcttcgcgatcgaggggactttcctcgatgtgactcattcgatttcttggaaatcgatttccaagaaatcgaccccgccccctcgaggaggaaaaactcgatttccaagaaatcgaatgacatcatctttcgaggggactttcctcgaagatcaaagggtcgaagatcaaagggtcgaggaggaaaaactcgatttccaagaaatcgaatgagtcacatcgagtttttcctcctcgatttcttggaaatcgaagagggggcggagatcgatttggcgctcgagtagagtctagactctacattttgacacccccat

SEQ ID NO: 43 - DNA, 268 bp, Synthetic construct
  source 1..268: mol_type = other DNA
ctgcttagggttaggcgttttgcgctgcttcgcgatcgaaagatgatgtcattcgaatgagtcacatcgatttggcgctcgagggggcggggtcgatttggcgctcgaaagatgatgtcattcgagtttttcctcctcgaccctttgatcttcgaagagggggcggagatcgaaagatgatgtcattcgaatgagtcacatcgatttccaagaaatcgatttccaagaaatcgagtagagtctagactctacattttgacacccccat

SEQ ID NO: 44 - DNA, 272 bp, Synthetic construct
  source 1..272: mol_type = other DNA
ctgcttagggttaggcgttttgcgctgcttcgcgatcgaggaggaaaaactcgatttcttggaaatcgaggggactttcctcgaaagatgatgtcattcgaagatcaaagggtcgatgtgactcattcgaggggactttcctcgagggggcggggtcgaatgacatcatctttcgaggggactttcctcgaggggactttcctcgaatgacatcatctttcgatttcttggaaatcgagtagagtctagactctacattttgacacccccat

SEQ ID NO: 45 - DNA, 223 bp, Synthetic construct
  source 1..223: mol_type = other DNA
ctgcttagggttaggcgttttgcgctgcttcgcgatcgatctccgccccctcttcgagtttttcctcctcgatttcttggaaatcgaagatcaaagggtcgatttcttggaaatcgaggggactttcctcgaggaaagtcccctcgaggggactttcctcgaggaggaaaaactcgatttggcgctcgagtagagtctagactctacattttgacacccccat

SEQ ID NO: 46 - DNA, 216 bp, Synthetic construct
  source 1..216: mol_type = other DNA
ctgcttagggttaggcgttttgcgctgcttcgcgatcgaaagatgatgtcattcgagtttttcctcctcgaaggaagttcgaggacgtgatcgaagagggggcggagatcgaatgagtcacatcgaaggaagttcgaggaaagtcccctcgaggaaagtcccctcgaagatcaaagggtcgagtagagtctagactctacattttgacacccccat

SEQ ID NO: 47 - DNA, 144 bp, Synthetic construct
  source 1..144: mol_type = other DNA
ctgcttagggttaggcgttttgcgctgcttcgcgatcgaccccgccccctcgagcgccaaatcgaggggactttcctcgaggggactttcctcgaggggactttcctcgagtagagtctagactctacattttgacacccccat

SEQ ID NO: 48 - DNA, 235 bp, Synthetic construct
  source 1..235: mol_type = other DNA
ctgcttagggttaggcgttttgcgctgcttcgcgatcgagggggcggggtcgaatgacatcatctttcgaggaaagtcccctcgaggaaagtcccctcgaggaaagtcccctcgatttccaagaaatcgaccctttgatcttcgaagatcaaagggtcgatgtgactcattcgaagatcaaagggtcgatttggcgctcgagtagagtctagactctacattttgacacccccat

SEQ ID NO: 49 - DNA, 295 bp, Synthetic construct
  source 1..295: mol_type = other DNA
ctgcttagggttaggcgttttgcgctgcttcgcgatcgaggaaagtcccctcgagtttttcctcctcgaagagggggcggagatcgatttccaaaaaactcgaatgacatcatctttcgaagatcaaggggtcgaagatcaaagggtcgatttccaagaaatcgatttcttggaaatcgaggaaagtcccctcgaccctttgatcttcgatctccgccccctcttcgaagatcaaagggtcgaagagggggcggagatcgagtagagtctagactctacattttgacacccccat

SEQ ID NO: 50 - DNA, 308 bp, Synthetic construct
  source 1..308: mol_type = other DNA
ctgcttagggttaggcgttttgcgctgcttcgcgatcgatttccaagaaatcgatttcttggaaatcgaatgacatcatctttcgagtttttcctcctcgaagagggggcggagatcgaggaaagtcccctcgatttccaagaaatcgaacttccttcgaaggaagttcgaggaggaaaaactcgagggggcggggtcgagtttttcctcctcgaggggactttcctcgaccccgccccctcgaccctttgatcttcgatttccaagaaatcgagtagagtctagactctacattttgacacccccat

SEQ ID NO: 51 - DNA, 435 bp, Synthetic construct
  source 1..435: mol_type = other DNA
tcgaagatcaaagggtcgatttccaagaaatcgatgtgactcattcgatttggcgctcgaccccgccccctcgaagagggggcggagatcgaatgagtcacatcgaggaaagtcccctcgatttcttggaaatcgaggaaagtcccctcgaggggactttcctcgaggaggaaaaactcgaatgacatcatctttcgaatgagtcacatcgatttcttggaaatcgaggggactttcctcgaccccgccccctcgaggtgactttcctcgaggggactttcctcgatgtgactcattcgaggggactttcctcgaggggactttcctcgaccctttgatcttcgatttggcgctcgaggggactttcctcgaggggactttcctcgaggggactttcctcgagtagagtctagactctacattttgacaccccca

SEQ ID NO: 52 - DNA, 354 bp, Synthetic construct
  source 1..354: mol_type = other DNA
tcgaatgagtcacatcgatctccgccccctcttcgagggggcggggtcgaggaggaaaaactcgaatgagtcacatcgaccctttgatcttcgaggggactttccggggtggagcaagcgtgacaagtccacgtatgacccgaccgacgatatcgaagcctacgcgctgaacgccagccccgatcgaccccgccccctcgatttccaagaaatcgaatgacatcatctttcgaatgacatcatctttcgaggggactttcctcgaacttccttcgaggggactttcctcgaggggactttcctcgaggaggaaaaactcgagtagagtctagactctacattttgacaccccca

SEQ ID NO: 53 - DNA, 416 bp, Synthetic construct
  source 1..416: mol_type = other DNA
tcgaatgagtcacatcgatctccgccccctcttcgagggggcggggtcgaggaggaaaaactcgaatgagtcacatcgaccctttgatcttcgaggggactttccggggtggagcaagcgtgacaagtccacgtatgacccgaccgacgatatcgaagcctacgcgctgaacgccagccccgatcgaccccgccccctcgatttccaagaaatcgaatgacatcatctttcgaatgacatcatctttcgaggggactttcctcgaacttccttcgaggggactttcctcgaggggactttcctcgaggggactttcctcgaggaggaaaaactcgaaagatgatgtcattcgagtttttcctcctcgaggaggaaaaactcgagtagagtctagactctacattttgacaccccca

SEQ ID NO: 54 - DNA, 369 bp, Synthetic construct
  source 1..369: mol_type = other DNA
tcgaatgagtcacatcgatctccgccccctcttcgagggggcggggtcgaggaggaaaaactcgaatgagtcacatcgaccctttgatcttcgaggggactttccggggtggagcaagcgtgacaagtccacgtatgacccgaccgacgatatcgaagcctacgcgctgaacgccagccccgatcgaccccgccccctcgatttccaagaaatcgaatgacatcatctttcgaatgacatcatctttcgaggggactttcctcgaacttccttcgaggggactttcctcgaggggactttcctcgaggggactttcctcgaggaggaaaaactcgagtagagtctagactctacattttgacaccccca

SEQ ID NO: 55 - DNA, 419 bp, Synthetic construct
  source 1..419: mol_type = other DNA
tcgatttccaagaaatcgaccccgccccctcgaacttccttcgatttcttggaaatcgaggaaagtcccctcgatttcttggaaatcgatttcttggaaatcgaatgacatcatctttcgatttcttggaaatcgaccctttgatcttcgaggaggaaaaactcgaatgacatcatctttcgatcacgtcctcgaagatcaaagggtcgagtttttcctcctcgaggaaagtcccctcgatgtgactcattcgatttcttggaaatcgaggggactttcctcgaggggactttcctcgagtttttcctcctcgaggaggaaaaactcgatttccaagaaatcgaggggactttcctcgaccctttgatcttcgagcgccaaatcgagtagagtctagactctacattttgacaccccca

SEQ ID NO: 56 - DNA, 361 bp, Synthetic construct
  source 1..361: mol_type = other DNA
tcgatttcttggaaatcgatttggcgctcgaaggaagttcgagggggcggggtcgatttccaagaaatcgagtttttcctcctcgagcgccaaatcgaccctttgatcttcgatcacgtcctcgagcgccaaatcgaggacgtgatcgaatgagtcacatcgaatgagtcacatcgaatgagtcacatcgatttggcgctcgatcggggggcggggtcgaggaaagtcccctcgaggaaagtcccctcgatttccaagaaatcgatttggcgctcgatctccgccccctcttcgatctccgccccctcttcgagtttttcctcctcgagtagagtctagactctacattttgacaccccca

SEQ ID NO: 57 - DNA, 544 bp, Synthetic construct
  source 1..544: mol_type = other DNA
ggatctgcgatcgctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacagctgaagcttcgaggggctcgcatctctccttcacgcgcccgccgccctacctgaggccgccatccacgccggttgagtcgcgttctgccgcctcccgcctgtggtgcctcctgaactgcgtccgccgtctaggtaagtttaaagctcaggtcgagaccgggcctttgtccggcgctcccttggagcctacctagactcagccggctctccacgctttgcctgaccctgcttgctcaactctacgtctttgtttcgttttctgttctgcgccgttacagatccaagctgtgaccggcgcctac

SEQ ID NO: 58 - DNA, 232 bp, Synthetic construct
  source 1..232: mol_type = other DNA
gagggacagcccccccccaaagcccccagggatgtaattacgtccctcccccgctagggggcagcagcgagccgcccggggctccgctccggtccggcgctccccccgcatccccgagccggcagcgtgcggggacagcccgggcacggggaaggtggcacgggatcgctttcctctgaacgcttctcgctgctctttgagcctgcagacacctggggggatacggggaaaa

SEQ ID NO: 59 - DNA, 231 bp, Synthetic construct
  source 1..231: mol_type = other DNA
ttttccccgtatccccccaggtgtctgcaggctcaaagagcagcgagaagcgttcagaggaaagcgatcccgtgccaccttccccgtgcccgggctgtccccgcacgctgccggctcggggatgcggggggagcgccggaccggagcggagccccgggcggctcgctgctgccccctagcgggggagggacgtaattacatccctgggggctttgggggggggctgtccct

SEQ ID NO: 60 - protein, 12 aa, synthetic construct
ESKYGPPCPPCP

SEQ ID NO: 61 - DNA, 36 bp, Synthetic construct
  source 1..36: mol_type = other DNA
gaatctaagtacggaccgccctgccccccttgccct

SEQ ID NO: 62 - DNA, 36 bp, Synthetic construct
  source 1..36: mol_type = other DNA
gagtctaagtacggaccgccttgcccaccgtgccca

SEQ ID NO: 63 - DNA, 36 bp, Synthetic construct
  source 1..36: mol_type = other DNA
gagtctaagtacggaccgccctgccccccttgccct

SEQ ID NO: 64 - DNA, 311 bp, Synthetic construct
  source 1..311: mol_type = other DNA
ggacccagcgtgttcctgttcccccccaagcccaaggacaccctgatgatcagccggacccccgaggtgacctgtgtggtggtggacgtgtcccaggaggaccccgaggtccagttcaactggtacgtggacggcgtggaggtgcacaacgccaagaccaagccccgggaggagcagttccagagcacctaccgggtggtgtccgtgctgaccgtgctgcaccaggactggctgaacggcaaggaatacaagtgtaaggtgtccaacaagggcctgcccagcagcatcgagaaaaccatcagcaaggccaa

SEQ ID NO: 65 - DNA, 311 bp, Synthetic construct
  source 1..311: mol_type = other DNA
agggccagcctcgggagccccaggtgtacaccctgccccctagccaagaggagatgaccaagaatcaggtgtccctgacctgcctggtgaagggcttctaccccagcgacatcgccgtggagtgggagagcaacggccagcccgagaacaactacaagaccaccccccctgtgctggacagcgacggcagcttcttcctgtacagcaggctgaccgtggacaagagccggtggcaggagggcaacgtctttagctgctccgtgatgcacgaggccctgcacaaccactacacccagaagagcctgtccctg

SEQ ID NO: 66 - protein, 28 aa, Synthetic construct
MFWVLVVVGGVLACYSLLVTVAFIIFWV

SEQ ID NO: 67 - protein, 27 aa, Synthetic construct
MFWVLVVVGGVLACYSLLVTVAFIIFW

SEQ ID NO: 68 - protein, 26 aa, Synthetic construct
FWVLVVVGGVLACYSLLVTVAFIIFW

SEQ ID NO: 69 - DNA, 84 bp, Synthetic construct
  source 1..84: mol_type = other DNA
atgttctgggtgctggtggtggtcggaggcgtgctggcctgctacagcctgctggtcaccgtggccttcatcatcttttgggtg

SEQ ID NO: 70 - DNA, 84 bp, Synthetic construct
  source 1..84: mol_type = other DNA
atgttctgggtgctggtggtggtgggcggggtgctggcctgctacagcctgctggtgacagtggccttcatcatcttttgggta

SEQ ID NO: 71 - DNA, 81 bp, Synthetic construct
  source 1..81: mol_type = other DNA
atgttctgggtgctggtggtggtcggaggcgtgctggcctgctacagcctgctggtcaccgtggccttcatcatcttttgg

SEQ ID NO: 72 - DNA, 81 bp, Synthetic construct
  source 1..81: mol_type = other DNA
atgttctgggtgctggtggtggtgggcggggtgctggcctgctacagcctgctggtgacagtggccttcatcatcttttgg

SEQ ID NO: 73 - protein, 42 aa, Synthetic construct
KRGRKKLLYIFKQPFMRPVQTTQEEDGCSCRFPEEEEGGCEL

SEQ ID NO: 74 - protein, 43 aa, Synthetic construct
VKRGRKKLLYIFKQPFMRPVQTTQEEDGCSCRFPEEEEGGCEL

SEQ ID NO: 75 - protein, 41 aa, Synthetic construct
VKRGRKKLLYIFKQPFMRPVQTTQEEDGCSCRFPEEEEGGC

SEQ ID NO: 76 - DNA, 126 bp, Synthetic construct
  source 1..126: mol_type = other DNA
aaacggggcagaaagaaactcctgtatatattcaaacaaccatttatgagaccagtacaaactactcaagaggaagatggctgtagctgccgatttccagaagaagaagaaggaggatgtgaactg

SEQ ID NO: 77 - DNA, 126 bp, Synthetic construct
  source 1..126: mol_type = other DNA
aagagaggacggaagaagctgctgtatatctttaagcagccttttatgagacctgtccaaacaacccaagaggaggacgggtgttcctgtcgcttcccagaggaggaggaaggagggtgcgagctg

SEQ ID NO: 78 - DNA, 129 bp, Synthetic construct
  source 1..129: mol_type = other DNA
gtgaaacggggcagaaagaaactcctgtatatattcaaacaaccatttatgagaccagtacaaactactcaagaggaagatggctgtagctgccgatttccagaagaagaagaaggaggatgtgaactg

SEQ ID NO: 79 - protein, 112 aa, Synthetic construct
RVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 80 - protein, 115 aa, Synthetic construct
GGGRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 81 - protein, 114 aa, Synthetic construct
ELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 82 - DNA, 336 bp, Synthetic construct
  source 1..336: mol_type = other DNA
cgggtgaagttcagcagaagcgccgacgcccctgcctaccagcagggccagaatcagctgtacaacgagctgaacctgggcagaagggaagagtacgacgtcctggataagcggagaggccgggaccctgagatgggcggcaagcctcggcggaagaacccccaggaaggcctgtataacgaactgcagaaagacaagatggccgaggcctacagcgagatcggcatgaagggcgagcggaggcggggcaagggccacgacggcctgtatcagggcctgtccaccgccaccaaggatacctacgacgccctgcacatgcaggccctgcccccaagg

SEQ ID NO: 83 - DNA, 345 bp, Synthetic construct
  source 1..345: mol_type = other DNA
ggcggagggcgggtgaagttcagcagaagcgccgacgcccctgcctaccagcagggccagaatcagctgtacaacgagctgaacctgggcagaagggaagagtacgacgtcctggataagcggagaggccgggaccctgagatgggcggcaagcctcggcggaagaacccccaggaaggcctgtataacgaactgcagaaagacaagatggccgaggcctacagcgagatcggcatgaagggcgagcggaggcggggcaagggccacgacggcctgtatcagggcctgtccaccgccaccaaggatacctacgacgccctgcacatgcaggccctgcccccaagg

SEQ ID NO: 84 - protein, 21 aa, Synthetic construct
GGGEGRGSLLTCGDVEENPGP

SEQ ID NO: 85 - protein, 24 aa, Synthetic construct
LEGGGEGRGSLLTCGDVEENPGPR

SEQ ID NO: 86 - protein, 21 aa, Synthetic construct
GSGEGRGSLLTCGDVEENPGP

SEQ ID NO: 87 - protein, 22 aa, Synthetic construct
GSGATNFSLLKQAGDVEENPGP

SEQ ID NO: 88 - protein, 23 aa, Synthetic construct
GSGQCTNYALLKLAGDVESNPGP

SEQ ID NO: 89 - protein, 25 aa, Synthetic construct
GSGVKQTLNFDLLKLAGDVESNPGP

SEQ ID NO: 90 - DNA, 72 bp, Synthetic construct
  source 1..72: mol_type = other DNA
ctcgagggcggcggagagggcagaggaagtcttctaacatgcggtgacgtggaggagaatcccggccctagg

SEQ ID NO: 91 - DNA, 72 bp, Synthetic construct
  source 1..72: mol_type = other DNA
ctcgagggcggcggagagggcagaggaagtcttctaacatgcggtgacgtggaggagaatccaggccctagg

SEQ ID NO: 92 - protein, 357 aa, Synthetic construct
MLLLVTSLLLCELPHPAFLLIPRKVCNGIGIGEFKDSLSINATNIKHFKNCTSISGDLHILPVAFRGDSFTHTPPLDPQELDILKTVKEITGFLLIQAWPENRTDLHAFENLEIIRGRTKQHGQFSLAVVSLNITSLGLRSLKEISDGDVIISGNKNLCYANTINWKKLFGTSGQKTKIISNRGENSCKATGQVCHALCSPEGCWGPEPRDCVSCRNVSRGRECVDKCNLLEGEPREFVENSECIQCHPECLPQAMNITCTGRGPDNCIQCAHYIDGPHCVKTCPAGVMGENNTLVWKYADAGHVCHLCHPNCTYGCTGPGLEGCPTNGPKIPSIATGMVGALLLLLVVALGIGLFM

SEQ ID NO: 93 - protein, 335 aa, Synthetic construct
RKVCNGIGIGEFKDSLSINATNIKHFKNCTSISGDLHILPVAFRGDSFTHTPPLDPQELDILKTVKEITGFLLIQAWPENRTDLHAFENLEIIRGRTKQHGQFSLAVVSLNITSLGLRSLKEISDGDVIISGNKNLCYANTINWKKLFGTSGQKTKIISNRGENSCKATGQVCHALCSPEGCWGPEPRDCVSCRNVSRGRECVDKCNLLEGEPREFVENSECIQCHPECLPQAMNITCTGRGPDNCIQCAHYIDGPHCVKTCPAGVMGENNTLVWKYADAGHVCHLCHPNCTYGCTGPGLEGCPTNGPKIPSIATGMVGALLLLLVVALGIGLFM

SEQ ID NO: 94 - DNA, 1008 bp, Synthetic construct
  source 1..1008: mol_type = other DNA
cgcaaagtgtgtaacggaataggtattggtgaatttaaagactcactctccataaatgctacgaatattaaacacttcaaaaactgcacctccatcagtggcgatctccacatcctgccggtggcatttaggggtgactccttcacacatactcctcctctggatccacaggaactggatattctgaaaaccgtaaaggaaatcacagggtttttgctgattcaggcttggcctgaaaacaggacggacctccatgcctttgagaacctagaaatcatacgcggcaggaccaagcaacatggtcagttttctcttgcagtcgtcagcctgaacataacatccttgggattacgctccctcaaggagataagtgatggagatgtgataatttcaggaaacaaaaatttgtgctatgcaaatacaataaactggaaaaaactgtttgggacctccggtcagaaaaccaaaattataagcaacagaggtgaaaacagctgcaaggccacaggccaggtctgccatgccttgtgctcccccgagggctgctggggcccggagcccagggactgcgtctcttgccggaatgtcagccgaggcagggaatgcgtggacaagtgcaaccttctggagggtgagccaagggagtttgtggagaactctgagtgcatacagtgccacccagagtgcctgcctcaggccatgaacatcacctgcacaggacggggaccagacaactgtatccagtgtgcccactacattgacggcccccactgcgtcaagacctgcccggcaggagtcatgggagaaaacaacaccctggtctggaagtacgcagacgccggccatgtgtgccacctgtgccatccaaactgcacctacggatgcactgggccaggtcttgaaggctgtccaacgaatgggcctaagatcccgtccatcgccactgggatggtgggggcactcctcttgctgctggtggtggccctggggatcggcctcttcatgtga

SEQ ID NO: 95 - DNA, 972 bp, Synthetic construct
  source 1..972: mol_type = other DNA
atgccacctccaagactcctcttcttcctcctcttcctgacaccaatggaagtcaggcctgaggaacctctagtggtgaaggtggaagagggagataacgctgtgttacagtgcctcaagggaacctcagatggacccactcagcagctgacctggtctcgggagtctccgcttaaacccttcctgaaactcagccttggactgccaggtctgggaatccacatgaggccactggctatctggctgttcatcttcaacgtctctcaacagatgggaggcttctacctgtgtcagcctggaccaccttctgagaaggcatggcagcctggttggacagtcaatgtggagggttctggtgagctgttccggtggaatgtttcggacctaggtggactgggatgtggtctgaagaacaggtcctcagagggacctagctctccttccgggaagctcatgagccccaagctgtatgtgtgggccaaagaccgccctgagatctgggagggagagcctccgtgtgtcccaccgagggacagcctgaaccagagcctcagccaggacctcaccatggcccctggctccacactctggctgtcctgtggggtaccccctgactctgtgtccaggggccccctctcctggacccatgtgcaccccaaggggcctaagtcattgctgagcctagagctgaaggacgatcgccctgccagagatatgtgggtaatggagacgggtctgttgttgccccgggccacagctcaagacgctggaaagtattattgtcaccgtggcaacctgaccatgtcattccacctggagatcactgctcggccagtactatggcactggctgctgaggactggtggctggaaggtctcagctgtgactttggcttatctgatcttctgcctgtgttcccttgtgggcattcttcatcttcaaagagccctggtcctgaggaggaaaagatga

SEQ ID NO: 96 - protein, 120 aa, Synthetic construct
CHPECQPQNGSVTCFGPEADQCVACAHYKDPPFCVARCPSGVKPDLSYMPIWKFPDEEGACQPCPINCTHSCVDLDDKGCPAEQRASPLTGGGSGGGSIISAVVGILLVVVLGVVFGILI

SEQ ID NO: 97 - protein, 187 aa, Synthetic construct
MVGSLNCIVAVSQNMGIGKNGDFPWPPLRNESRYFQRMTTTSSVEGKQNLVIMGKKTWFSIPEKNRPLKGRINLVLSRELKEPPQGAHFLSRSLDDALKLTEQPELANKVDMVWIVGGSSVYKEAMNHPGHLKLFVTRIMQDFESDTFFPEIDLEKYKLLPEYPGVLSDVQEEKGIKYKFEVYEKND

SEQ ID NO: 98 - DNA, 7650 bp, Synthetic construct
  source 1..7650: mol_type = other DNA
ggtggtcggaggcgtgctggcctgctacagcctgctggtcaccgtggccttcatcatcttttgggtgaaacggggcagaaagaaactcctgtatatattcaaacaaccatttatgagaccagtacaaactactcaagaggaagatggctgtagctgccgatttccagaagaagaagaaggaggatgtgaactgcgggtgaagttcagcagaagcgccgacgcccctgcctaccagcagggccagaatcagctgtacaacgagctgaacctgggcagaagggaagagtacgacgtcctggataagcggagaggccgggaccctgagatgggcggcaagcctcggcggaagaacccccaggaaggcctgtataacgaactgcagaaagacaagatggccgaggcctacagcgagatcggcatgaagggcgagcggaggcggggcaagggccacgacggcctgtatcagggcctgtccaccgccaccaaggatacctacgacgccctgcacatgcaggccctgcccccaaggggaagcggagctactaacttcagcctgctgaagcaggctggagacgtggaggagaaccctggacctatggttggttcgctaaactgcatcgtcgctgtgtcccagaacatgggcatcggcaagaacggggacttcccctggccaccgctcaggaatgaatccagatatttccagagaatgaccacaacctcttcagtagaaggtaaacagaatctggtgattatgggtaagaagacctggttctccattcctgagaagaatcgacctttaaagggtagaattaatttagttctcagcagagaactcaaggaacctccacaaggagctcattttctttccagaagtctagatgatgccttaaaacttactgaacaaccagaattagcaaataaagtagacatggtctggatagttggtggcagttctgtttataaggaagccatgaatcacccaggccatcttaaactatttgtgacaaggatcatgcaagactttgaaagtgacacgttttttccagaaattgatttggagaaatataaacttctgccagaatacccaggtgttctctctgatgtccaggaggagaaaggcattaagtacaaatttgaagtatatgagaagaatgatggcggcggagagggcagaggaagtcttctaacatgcggtgacgtggaggagaatcccggccctatgcttctcctggtgacaagccttctgctctgtgagttaccacacccagcattcctcctgatcccacgcaaagtgtgtaacggaataggtattggtgaatttaaagactcactctccataaatgctacgaatattaaacacttcaaaaactgcacctccatcagtggcgatctccacatcctgccggtggcatttaggggtgactccttcacacatactcctcctctggatccacaggaactggatattctgaaaaccgtaaaggaaatcacagggtttttgctgattcaggcttggcctgaaaacaggacggacctccatgcctttgagaacctagaaatcatacgcggcaggaccaagcaacatggtcagttttctcttgcagtcgtcagcctgaacataacatccttgggattacgctccctcaaggagataagtgatggagatgtgataatttcaggaaacaaaaatttgtgctatgcaaatacaataaactggaaaaaactgtttgggacctccggtcagaaaaccaaaattataagcaacagaggtgaaaacagctgcaaggccacaggccaggtctgccatgccttgtgctcccccgagggctgctggggcccggagcccagggactgcgtctcttgccggaatgtcagccgaggcagggaatgcgtggacaagtgcaaccttctggagggtgagccaagggagtttgtggagaactctgagtgcatacagtgccacccagagtgcctgcctcaggccatgaacatcacctgcacaggacggggaccagacaactgtatccagtgtgcccactacattgacggcccccactgcgtcaagacctgcccggcaggagtcatgggagaaaacaacaccctggtctggaagtacgcagacgccggccatgtgtgccacctgtgccatccaaactgcacctacggatgcactgggccaggtcttgaaggctgtccaacgaatgggcctaagatcccgtccatcgccactgggatggtgggggcactcctcttgctgctggtggtggccctggggatcggcctcttcatgtgacctgcaggcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggatggctagatctttttccccgtatccccccaggtgtctgcaggctcaaagagcagcgagaagcgttcagaggaaagcgatcccgtgccaccttccccgtgcccgggctgtccccgcacgctgccggctcggggatgcggggggagcgccggaccggagcggagccccgggcggctcgctgctgccccctagcgggggagggacgtaattacatccctgggggctttgggggggggctgtccctgatatctataacaagaaaatatatatataataagttatcacgtaagtagaacatgaaataacaatataattatcgtatgagttaaatcttaaaagtcacgtaaaagataatcatgcgtcattttgactcacgcggtcgttatagttcaaaatcagtgacacttaccgcattgacaagcacgcctcacgggagctccaagcggcgactgagatgtcctaaatgcacagcgacggattcgcgctatttagaaagagagagcaatatttcaagaatgcatgcgtcaattttacgcagactatctttctagggttaagtcgacccgcctaatgagcgggcttttttttggcttgttgtccacaaccgttaaaccttaaaagctttaaaagccttatatattcttttttttcttataaaacttaaaaccttagaggctatttaagttgctgatttatattaattttattgttcaaacatgagagcttagtacgtgaaacatgagagcttagtacgttagccatgagagcttagtacgttagccatgagggtttagttcgttaaacatgagagcttagtacgttaaacatgagagcttagtacgtactatcaacaggttgaactgctgatccacgttgtggtagaattggtaaagagagtcgtgtaaaatatcgagttcgcacatcttgttgtctgattattgatttttggcgaaaccatttgatcatatgacaagatgtgtatctaccttaacttaatgattttgataaaaatcattaggactagtttaaccctagaaagataatcatattgtgacgtacgttaaagataatcatgcgtaaaattgacgcatgtgttttatcggtctgtatatcgaggtttatttattaatttgaatagatattaagttttattatatttacacttacatactaataataaattcaacaaacaatttatttatgtttatttatttattaaaaaaaaacaaaaactcaaaatttcttctataaagtaacaaaacttttatgaattcgagggacagccccccccccaaagcccccagggatgtaattacgtccctcccccgctagggggcagcagcgagccgcccggggctccgctccggtccggcgctccccccgcatccccgagccggcagcgtgcggggacagcccgggcacggggaaggtggcacgggatcgctttcctctgaacgcttctcgctgctctttgagcctgcagacacctggggggatacggggaaaaggctcgcgatcgaatgagtcacatcgatctccgccccctcttcgagggggcggggtcgaggaggaaaaactcgaatgagtcacatcgaccctttgatcttcgaggggactttccggggtggagcaagcgtgacaagtccacgtatgacccgaccgacgatatcgaagcctacgcgctgaacgccagccccgatcgaccccgccccctcgatttccaagaaatcgaatgacatcatctttcgaatgacatcatctttcgaggggactttcctcgaacttccttcgaggggactttcctcgaggggactttcctcgaggaggaaaaactcgagtagagtctagactctacattttgacacccccataatatttttccagaattaacagtataaattgcatctcttgttcaagagttccctatcactctctttaatcactactcacagtaacctcaactcctggctagcgtactgcgtttgccgccaccatgcttctcctggtgacaagccttctgctctgtgagttaccacacccagcattcctcctgatcccatgccaccctgagtgtcagccccagaatggctcagtgacctgttttggaccggaggctgaccagtgtgtggcctgtgcccactataaggaccctcccttctgcgtggcccgctgccccagcggtgtgaaacctgacctctcctacatgcccatctggaagtttccagatgaggagggcgcatgccagccttgccccatcaactgcacccactcctgtgtggacctggatgacaagggctgccccgccgagcagagagccagcccgttaacgggtggaggcagcggaggtggctccatcatctctgcggtggttggcattctgctggtcgtggtcttgggggtggtctttgggatcctcatcggcggcggagagggcagaggaagtcttctaacatgcggtgacgtggaggagaatcccggccctatgcagatccctcaggccccttggcctgtcgtgtgggctgtgctgcagctgggatggcggcctggctggtttctggacagccccgacagaccctggaacccccctacattttcccctgccctgctggtcgtgaccgagggcgacaatgccaccttcacctgtagcttcagcaacaccagcgagagcttcgtgctgaactggtacagaatgagccccagcaaccagaccgacaagctggccgccttccccgaggatagatctcagcccggccaggactgccggttcagagtgacccagctgcccaacggccgggacttccacatgtctgtcgtgcgggccagacggaacgacagcggcacatatctgtgcggcgccatcagcctggcccccaagctccagatcaaagagagcctgagagccgagctgagagtgaccgagagaagggccgaagtgcctaccgcccaccctagcccatctccaagacctgccggccagttccagacactggtcggcggaggcggaagcggtggcggaggaagcggaggcggcggatctcacaagagcagctctcagggccaagaccggcacatgatccggatgagacagctgatcgacatcgtggaccagctgaagaactacgtgaacgacctggtgcctgagttcctgcctgctcctgaggacgtggaaacaaactgcgagtggagcgccttcagctgcttccagaaggcccagctgaaaagcgccaacaccggcaacaacgagcggatcatcaatgtgtccatcaagaagctgaagcggaagcctcctagcaccaacgccggaagaaggcagaagcacagactgacctgtccaagctgcgacagctacgagaagaagcctccaaaagagttcctggaacggttcaagagcctgctgcagaagatgatccaccagcacctgagcagcagaacccacggcagcgaggattcttgaaaacggatccgcggccgcttttccctctgccaaaaattatggggacatcatgaagccccttgagcatctgacttctggctaataaaggaaatttattttcattgcaatagtgtgttggaattttttgtgtctctcactcggaaggacataaggtgagatctttcacaaaacaccagttatgctgagatctttcacaaaacaccagttatgcgctaatgcctttgggcctaacacgtgcctcacgaggatctgcgatcgctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacagctgaagcttcgaggggctcgcatctctccttcacgcgcccgccgccctacctgaggccgccatccacgccggttgagtcgcgttctgccgcctcccgcctgtggtgcctcctgaactgcgtccgccgtctaggtaagtttaaagctcaggtcgagaccgggcctttgtccggcgctcccttggagcctacctagactcagccggctctccacgctttgcctgaccctgcttgctcaactctacgtctttgtttcgttttctgttctgcgccgttacagatccaagctgtgaccggcgcctacggttaattaagccgccaccatgcttctcctggtgacaagccttctgctctgtgagttaccacacccagcattcctcctgatcccagaggtgcagctggtcgaatccggaggaggactggtccagcccggccggagcctgagactgagctgtgccgcttccggattcacttttgacgattacgcaatgcattgggtgaggcaggcccctggcaaggggctggaatgggtctccggaatctcttggaacagtgggcgcattggatatgccgattctgtgaagggccgattcactatctctcgggacaacgctaaaaatagtctgtttctgcagatgaattccctgcgcgccgaggataccgccgtgtactattgcgcccgagaccagggctaccactactatgatagcgccgaacatgcattcgacatttggggacagggaactgtggtcaccgtgagctccggaggaggaggaagcggaggaggagggtccggaggcgggggatcacagagcgcactgacccagccacggagcgtgagcggatttcctgggcagtctgtcaccattagttgcacaggcaccacatcagacgatgtgagctggtaccagcagcacccagggaaggctccccagctgatgctgtatgacgtgtccaaaagaccttctggcgtcccacataggtttagtggaagccggagcggccgggcagccagtctgatcatttcagggctgcagacagaggacgaagctgattatttctgttctagttacgcaggcagatataactctgtgctgtttggcgggggaacaaagctgactgtcctggaatctaagtacggaccgccctgccccccttgccctatgttctgggtgctggt

SEQ ID NO: 99 - DNA, 7597 bp, Synthetic construct
  source 1..7597: mol_type = other DNA
accgggcttgttgtccacaaccgttaaaccttaaaagctttaaaagccttatatattcttttttttcttataaaacttaaaaccttagaggctatttaagttgctgatttatattaattttattgttcaaacatgagagcttagtacgtgaaacatgagagcttagtacgttagccatgagagcttagtacgttagccatgagggtttagttcgttaaacatgagagcttagtacgttaaacatgagagcttagtacgtactatcaacaggttgaactgctgatccacgttgtggtagaattggtaaagagagtcgtgtaaaatatcgagttcgcacatcttgttgtctgattattgatttttggcgaaaccatttgatcatatgacaagatgtgtatctaccttaacttaatgattttgataaaaatcattaggactagtttaaccctagaaagataatcatattgtgacgtacgttaaagataatcatgcgtaaaattgacgcatgtgttttatcggtctgtatatcgaggtttatttattaatttgaatagatattaagttttattatatttacacttacatactaataataaattcaacaaacaatttatttatgtttatttatttattaaaaaaaaacaaaaactcaaaatttcttctataaagtaacaaaacttttatgaattcgagggacagcccccccccaaagcccccagggatgtaattacgtccctcccccgctagggggcagcagcgagccgcccggggctccgctccggtccggcgctccccccgcatccccgagccggcagcgtgcggggacagcccgggcacggggaaggtggcacgggatcgctttcctctgaacgcttctcgctgctctttgagcctgcagacacctggggggatacggggaaaaggctcgcgatcgaatgagtcacatcgatctccgccccctcttcgagggggcggggtcgaggaggaaaaactcgaatgagtcacatcgaccctttgatcttcgaggggactttccggggtggagcaagcgtgacaagtccacgtatgacccgaccgacgatatcgaagcctacgcgctgaacgccagccccgatcgaccccgccccctcgatttccaagaaatcgaatgacatcatctttcgaatgacatcatctttcgaggggactttcctcgaacttccttcgaggggactttcctcgaggggactttcctcgaggaggaaaaactcgagtagagtctagactctacattttgacacccccataatatttttccagaattaacagtataaattgcatctcttgttcaagagttccctatcactctctttaatcactactcacagtaacctcaactcctggctagcgtactgcgtttgccgccaccatgcttctcctggtgacaagccttctgctctgtgagttaccacacccagcattcctcctgatcccatgccaccctgagtgtcagccccagaatggctcagtgacctgttttggaccggaggctgaccagtgtgtggcctgtgcccactataaggaccctcccttctgcgtggcccgctgccccagcggtgtgaaacctgacctctcctacatgcccatctggaagtttccagatgaggagggcgcatgccagccttgccccatcaactgcacccactcctgtgtggacctggatgacaagggctgccccgccgagcagagagccagcccgttaacgggtggaggcagcggaggtggctccatcatctctgcggtggttggcattctgctggtcgtggtcttgggggtggtctttgggatcctcatcggcggcggagagggcagaggaagtcttctaacatgcggtgacgtggaggagaatcccggccctatgcagatccctcaggccccttggcctgtcgtgtgggctgtgctgcagctgggatggcggcctggctggtttctggacagccccgacagaccctggaacccccctacattttcccctgccctgctggtcgtgaccgagggcgacaatgccaccttcacctgtagcttcagcaacaccagcgagagcttcgtgctgaactggtacagaatgagccccagcaaccagaccgacaagctggccgccttccccgaggatagatctcagcccggccaggactgccggttcagagtgacccagctgcccaacggccgggacttccacatgtctgtcgtgcgggccagacggaacgacagcggcacatatctgtgcggcgccatcagcctggcccccaagctccagatcaaagagagcctgagagccgagctgagagtgaccgagagaagggccgaagtgcctaccgcccaccctagcccatctccaagacctgccggccagttccagacactggtcggcggaggcggaagcggtggcggaggaagcggaggcggcggatctaattgggtaaacgtgattagtgatctcaaaaagattgaagatctgattcagtctatgcatatcgatgcaaccctttacacggagtccgatgttcacccgtcatgcaaggtaaccgcaatgaagtgtttcctgttggaattgcaggtaatctccttggagtccggtgatgcatcaatccacgatactgtcgaaaacttgattatcctcgcgaacgacagtctctcctcaaacggaaacgtcactgagtctggatgcaaagagtgtgaagagcttgaggaaaaaaatataaaggaatttcttcagtctttcgtgcatatagttcaaatgttcataaacacaagttgaaaacggatccgcggccgcttttccctctgccaaaaattatggggacatcatgaagccccttgagcatctgacttctggctaataaaggaaatttattttcattgcaatagtgtgttggaattttttgtgtctctcactcggaaggacataaggtgagatctttcacaaaacaccagttatgctgagatctttcacaaaacaccagttatgcgctaatgcctttgggcctaacacgtgcctcacgaggatctgcgatcgctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacagctgaagcttcgaggggctcgcatctctccttcacgcgcccgccgccctacctgaggccgccatccacgccggttgagtcgcgttctgccgcctcccgcctgtggtgcctcctgaactgcgtccgccgtctaggtaagtttaaagctcaggtcgagaccgggcctttgtccggcgctcccttggagcctacctagactcagccggctctccacgctttgcctgaccctgcttgctcaactctacgtctttgtttcgttttctgttctgcgccgttacagatccaagctgtgaccggcgcctacggttaattaagccgccaccatgcttctcctggtgacaagccttctgctctgtgagttaccacacccagcattcctcctgatcccagaggtgcagctggtcgaatccggaggaggactggtccagcccggccggagcctgagactgagctgtgccgcttccggattcacttttgacgattacgcaatgcattgggtgaggcaggcccctggcaaggggctggaatgggtctccggaatctcttggaacagtgggcgcattggatatgccgattctgtgaagggccgattcactatctctcgggacaacgctaaaaatagtctgtttctgcagatgaattccctgcgcgccgaggataccgccgtgtactattgcgcccgagaccagggctaccactactatgatagcgccgaacatgcattcgacatttggggacagggaactgtggtcaccgtgagctccggaggaggaggaagcggaggaggagggtccggaggcgggggatcacagagcgcactgacccagccacggagcgtgagcggatttcctgggcagtctgtcaccattagttgcacaggcaccacatcagacgatgtgagctggtaccagcagcacccagggaaggctccccagctgatgctgtatgacgtgtccaaaagaccttctggcgtcccacataggtttagtggaagccggagcggccgggcagccagtctgatcatttcagggctgcagacagaggacgaagctgattatttctgttctagttacgcaggcagatataactctgtgctgtttggcgggggaacaaagctgactgtcctggaatctaagtacggaccgccctgccccccttgccctatgttctgggtgctggtggtggtcggaggcgtgctggcctgctacagcctgctggtcaccgtggccttcatcatcttttgggtgaaacggggcagaaagaaactcctgtatatattcaaacaaccatttatgagaccagtacaaactactcaagaggaagatggctgtagctgccgatttccagaagaagaagaaggaggatgtgaactgcgggtgaagttcagcagaagcgccgacgcccctgcctaccagcagggccagaatcagctgtacaacgagctgaacctgggcagaagggaagagtacgacgtcctggataagcggagaggccgggaccctgagatgggcggcaagcctcggcggaagaacccccaggaaggcctgtataacgaactgcagaaagacaagatggccgaggcctacagcgagatcggcatgaagggcgagcggaggcggggcaagggccacgacggcctgtatcagggcctgtccaccgccaccaaggatacctacgacgccctgcacatgcaggccctgcccccaaggggaagcggagctactaacttcagcctgctgaagcaggctggagacgtggaggagaaccctggacctatggttggttcgctaaactgcatcgtcgctgtgtcccagaacatgggcatcggcaagaacggggacttcccctggccaccgctcaggaatgaatccagatatttccagagaatgaccacaacctcttcagtagaaggtaaacagaatctggtgattatgggtaagaagacctggttctccattcctgagaagaatcgacctttaaagggtagaattaatttagttctcagcagagaactcaaggaacctccacaaggagctcattttctttccagaagtctagatgatgccttaaaacttactgaacaaccagaattagcaaataaagtagacatggtctggatagttggtggcagttctgtttataaggaagccatgaatcacccaggccatcttaaactatttgtgacaaggatcatgcaagactttgaaagtgacacgttttttccagaaattgatttggagaaatataaacttctgccagaatacccaggtgttctctctgatgtccaggaggagaaaggcattaagtacaaatttgaagtatatgagaagaatgatggcggcggagagggcagaggaagtcttctaacatgcggtgacgtggaggagaatcccggccctatgcttctcctggtgacaagccttctgctctgtgagttaccacacccagcattcctcctgatcccacgcaaagtgtgtaacggaataggtattggtgaatttaaagactcactctccataaatgctacgaatattaaacacttcaaaaactgcacctccatcagtggcgatctccacatcctgccggtggcatttaggggtgactccttcacacatactcctcctctggatccacaggaactggatattctgaaaaccgtaaaggaaatcacagggtttttgctgattcaggcttggcctgaaaacaggacggacctccatgcctttgagaacctagaaatcatacgcggcaggaccaagcaacatggtcagttttctcttgcagtcgtcagcctgaacataacatccttgggattacgctccctcaaggagataagtgatggagatgtgataatttcaggaaacaaaaatttgtgctatgcaaatacaataaactggaaaaaactgtttgggacctccggtcagaaaaccaaaattataagcaacagaggtgaaaacagctgcaaggccacaggccaggtctgccatgccttgtgctcccccgagggctgctggggcccggagcccagggactgcgtctcttgccggaatgtcagccgaggcagggaatgcgtggacaagtgcaaccttctggagggtgagccaagggagtttgtggagaactctgagtgcatacagtgccacccagagtgcctgcctcaggccatgaacatcacctgcacaggacggggaccagacaactgtatccagtgtgcccactacattgacggcccccactgcgtcaagacctgcccggcaggagtcatgggagaaaacaacaccctggtctggaagtacgcagacgccggccatgtgtgccacctgtgccatccaaactgcacctacggatgcactgggccaggtcttgaaggctgtccaacgaatgggcctaagatcccgtccatcgccactgggatggtgggggcactcctcttgctgctggtggtggccctggggatcggcctcttcatgtgacctgcaggcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggatggctagatctttttccccgtatccccccaggtgtctgcaggctcaaagagcagcgagaagcgttcagaggaaagcgatcccgtgccaccttccccgtgcccgggctgtccccgcacgctgccggctcggggatgcggggggagcgccggaccggagcggagccccgggcggctcgctgctgccccctagcgggggagggacgtaattacatccctgggggctttggggggggggctgtccctgatatctataacaagaaaatatatatataataagttatcacgtaagtagaacatgaaataacaatataattatcgtatgagttaaatcttaaaagtcacgtaaaagataatcatgcgtcattttgactcacgcggtcgttatagttcaaaatcagtgacacttaccgcattgacaagcacgcctcacgggagctccaagcggcgactgagatgtcctaaatgcacagcgacggattcgcgctatttagaaagagagagcaatatttcaagaatgcatgcgtcaattttacgcagactatctttctagggttaagtcgacccgcctaatgagcgggcttttttttggcttgttgtccaca

SEQ ID NO: 100 - DNA, 7707 bp, Synthetic construct
  source 1..7707: mol_type = other DNA
tgagcggatttcctgggcagtctgtcaccattagttgcacaggcaccacatcagacgatgtgagctggtaccagcagcacccagggaaggctccccagctgatgctgtatgacgtgtccaaaagaccttctggcgtcccacataggtttagtggaagccggagcggccgggcagccagtctgatcatttcagggctgcagacagaggacgaagctgattatttctgttctagttacgcaggcagatataactctgtgctgtttggcgggggaacaaagctgactgtcctggaatctaagtacggaccgccctgccccccttgccctatgttctgggtgctggtggtggtcggaggcgtgctggcctgctacagcctgctggtcaccgtggccttcatcatcttttgggtgaaacggggcagaaagaaactcctgtatatattcaaacaaccatttatgagaccagtacaaactactcaagaggaagatggctgtagctgccgatttccagaagaagaagaaggaggatgtgaactgcgggtgaagttcagcagaagcgccgacgcccctgcctaccagcagggccagaatcagctgtacaacgagctgaacctgggcagaagggaagagtacgacgtcctggataagcggagaggccgggaccctgagatgggcggcaagcctcggcggaagaacccccaggaaggcctgtataacgaactgcagaaagacaagatggccgaggcctacagcgagatcggcatgaagggcgagcggaggcggggcaagggccacgacggcctgtatcagggcctgtccaccgccaccaaggatacctacgacgccctgcacatgcaggccctgcccccaaggggaagcggagctactaacttcagcctgctgaagcaggctggagacgtggaggagaaccctggacctatggttggttcgctaaactgcatcgtcgctgtgtcccagaacatgggcatcggcaagaacggggacttcccctggccaccgctcaggaatgaatccagatatttccagagaatgaccacaacctcttcagtagaaggtaaacagaatctggtgattatgggtaagaagacctggttctccattcctgagaagaatcgacctttaaagggtagaattaatttagttctcagcagagaactcaaggaacctccacaaggagctcattttctttccagaagtctagatgatgccttaaaacttactgaacaaccagaattagcaaataaagtagacatggtctggatagttggtggcagttctgtttataaggaagccatgaatcacccaggccatcttaaactatttgtgacaaggatcatgcaagactttgaaagtgacacgttttttccagaaattgatttggagaaatataaacttctgccagaatacccaggtgttctctctgatgtccaggaggagaaaggcattaagtacaaatttgaagtatatgagaagaatgatggcggcggagagggcagaggaagtcttctaacatgcggtgacgtggaggagaatcccggccctatgcttctcctggtgacaagccttctgctctgtgagttaccacacccagcattcctcctgatcccacgcaaagtgtgtaacggaataggtattggtgaatttaaagactcactctccataaatgctacgaatattaaacacttcaaaaactgcacctccatcagtggcgatctccacatcctgccggtggcatttaggggtgactccttcacacatactcctcctctggatccacaggaactggatattctgaaaaccgtaaaggaaatcacagggtttttgctgattcaggcttggcctgaaaacaggacggacctccatgcctttgagaacctagaaatcatacgcggcaggaccaagcaacatggtcagttttctcttgcagtcgtcagcctgaacataacatccttgggattacgctccctcaaggagataagtgatggagatgtgataatttcaggaaacaaaaatttgtgctatgcaaatacaataaactggaaaaaactgtttgggacctccggtcagaaaaccaaaattataagcaacagaggtgaaaacagctgcaaggccacaggccaggtctgccatgccttgtgctcccccgagggctgctggggcccggagcccagggactgcgtctcttgccggaatgtcagccgaggcagggaatgcgtggacaagtgcaaccttctggagggtgagccaagggagtttgtggagaactctgagtgcatacagtgccacccagagtgcctgcctcaggccatgaacatcacctgcacaggacggggaccagacaactgtatccagtgtgcccactacattgacggcccccactgcgtcaagacctgcccggcaggagtcatgggagaaaacaacaccctggtctggaagtacgcagacgccggccatgtgtgccacctgtgccatccaaactgcacctacggatgcactgggccaggtcttgaaggctgtccaacgaatgggcctaagatcccgtccatcgccactgggatggtgggggcactcctcttgctgctggtggtggccctggggatcggcctcttcatgtgacctgcaggcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggatggctagatctttttccccgtatccccccaggtgtctgcaggctcaaagagcagcgagaagcgttcagaggaaagcgatcccgtgccaccttccccgtgcccgggctgtccccgcacgctgccggctcggggatgcggggggagcgccggaccggagcggagccccgggcggctcgctgctgccccctagcgggggagggacgtaattacatccctgggggctttgggggggggctgtccctgatatctataacaagaaaatatatatataataagttatcacgtaagtagaacatgaaataacaatataattatcgtatgagttaaatcttaaaagtcacgtaaaagataatcatgcgtcattttgactcacgcggtcgttatagttcaaaatcagtgacacttaccgcattgacaagcacgcctcacgggagctccaagcggcgactgagatgtcctaaatgcacagcgacggattcgcgctatttagaaagagagagcaatatttcaagaatgcatgcgtcaattttacgcagactatctttctagggttaagtcgacccgcctaatgagcgggcttttttttggcttgttgtccacaaccgttaaaccttaaaagctttaaaagccttatatattcttttttttcttataaaacttaaaaccttagaggctatttaagttgctgatttatattaattttattgttcaaacatgagagcttagtacgtgaaacatgagagcttagtacgttagccatgagagcttagtacgttagccatgagggtttagttcgttaaacatgagagcttagtacgttaaacatgagagcttagtacgtactatcaacaggttgaactgctgatccacgttgtggtagaattggtaaagagagtcgtgtaaaatatcgagttcgcacatcttgttgtctgattattgatttttggcgaaaccatttgatcatatgacaagatgtgtatctaccttaacttaatgattttgataaaaatcattaggactagtttaaccctagaaagataatcatattgtgacgtacgttaaagataatcatgcgtaaaattgacgcatgtgttttatcggtctgtatatcgaggtttatttattaatttgaatagatattaagttttattatatttacacttacatactaataataaattcaacaaacaatttatttatgtttatttatttattaaaaaaaaacaaaaactcaaaatttcttctataaagtaacaaaacttttatgaattcgagggacagccccccccccaaagcccccagggatgtaattacgtccctcccccgctagggggcagcagcgagccgcccggggctccgctccggtccggcgctccccccgcatccccgagccggcagcgtgcggggacagcccgggcacggggaaggtggcacgggatcgctttcctctgaacgcttctcgctgctctttgagcctgcagacacctggggggatacggggaaaaggctcgcgatcgaatgagtcacatcgatctccgccccctcttcgagggggcggggtcgaggaggaaaaactcgaatgagtcacatcgaccctttgatcttcgaggggactttccggggtggagcaagcgtgacaagtccacgtatgacccgaccgacgatatcgaagcctacgcgctgaacgccagccccgatcgaccccgccccctcgatttccaagaaatcgaatgacatcatctttcgaatgacatcatctttcgaggggactttcctcgaacttccttcgaggggactttcctcgaggggactttcctcgaggaggaaaaactcgagtagagtctagactctacattttgacacccccataatatttttccagaattaacagtataaattgcatctcttgttcaagagttccctatcactctctttaatcactactcacagtaacctcaactcctggctagcgtactgcgtttgccgccaccatgcttctcctggtgacaagccttctgctctgtgagttaccacacccagcattcctcctgatcccatgccaccctgagtgtcagccccagaatggctcagtgacctgttttggaccggaggctgaccagtgtgtggcctgtgcccactataaggaccctcccttctgcgtggcccgctgccccagcggtgtgaaacctgacctctcctacatgcccatctggaagtttccagatgaggagggcgcatgccagccttgccccatcaactgcacccactcctgtgtggacctggatgacaagggctgccccgccgagcagagagccagcccgttaacgggtggaggcagcggaggtggctccatcatctctgcggtggttggcattctgctggtcgtggtcttgggggtggtctttgggatcctcatcggcggcggagagggcagaggaagtcttctaacatgcggtgacgtggaggagaatcccggccctatgcagatccctcaggccccttggcctgtcgtgtgggctgtgctgcagctgggatggcggcctggctggtttctggacagccccgacagaccctggaacccccctacattttcccctgccctgctggtcgtgaccgagggcgacaatgccaccttcacctgtagcttcagcaacaccagcgagagcttcgtgctgaactggtacagaatgagccccagcaaccagaccgacaagctggccgccttccccgaggatagatctcagcccggccaggactgccggttcagagtgacccagctgcccaacggccgggacttccacatgtctgtcgtgcgggccagacggaacgacagcggcacatatctgtgcggcgccatcagcctggcccccaagctccagatcaaagagagcctgagagccgagctgagagtgaccgagagaagggccgaagtgcctaccgcccaccctagcccatctccaagacctgccggccagttccagacactggtcggcggaggcggaagcggtggcggaggaagcggaggcggcggatcttatttcggcaaattggagagcaagttgagtgtcataaggaatttgaatgaccaggttttgtttatcgatcagggaaacaggcctttgtttgaggacatgaccgactccgattgtagagacaacgcaccccgaactatattcataatcagcaaatactccgattctcttgcgcgaggactcgcggttacgatctcagtcaagtgcgagaagataagtaccctgtcatgcgagaataaaattatctctttcaaggaaatgaatccgccagataatataaaggatacaaagagtgacatcatctttttccagcgagacgtacccggccactctcgcaagatgcaattcgagtcttctagctacgaaggatattttttggcgtgcgagaaagagagggacttgttcaaacttatcttgaagaaggaggatgaactgggtgaccgctctattatgtttacagtacaaaatgaggattgaaaacggatccgcggccgcttttccctctgccaaaaattatggggacatcatgaagccccttgagcatctgacttctggctaataaaggaaatttattttcattgcaatagtgtgttggaattttttgtgtctctcactcggaaggacataaggtgagatctttcacaaaacaccagttatgctgagatctttcacaaaacaccagttatgcgctaatgcctttgggcctaacacgtgcctcacgaggatctgcgatcgctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacagctgaagcttcgaggggctcgcatctctccttcacgcgcccgccgccctacctgaggccgccatccacgccggttgagtcgcgttctgccgcctcccgcctgtggtgcctcctgaactgcgtccgccgtctaggtaagtttaaagctcaggtcgagaccgggcctttgtccggcgctcccttggagcctacctagactcagccggctctccacgctttgcctgaccctgcttgctcaactctacgtctttgtttcgttttctgttctgcgccgttacagatccaagctgtgaccggcgcctacggttaattaagccgccaccatgcttctcctggtgacaagccttctgctctgtgagttaccacacccagcattcctcctgatcccagaggtgcagctggtcgaatccggaggaggactggtccagcccggccggagcctgagactgagctgtgccgcttccggattcacttttgacgattacgcaatgcattgggtgaggcaggcccctggcaaggggctggaatgggtctccggaatctcttggaacagtgggcgcattggatatgccgattctgtgaagggccgattcactatctctcgggacaacgctaaaaatagtctgtttctgcagatgaattccctgcgcgccgaggataccgccgtgtactattgcgcccgagaccagggctaccactactatgatagcgccgaacatgcattcgacatttggggacagggaactgtggtcaccgtgagctccggaggaggaggaagcggaggaggagggtccggaggcgggggatcacagagcgcactgacccagccacggagcg

SEQ ID NO: 101 - DNA, 8877 bp, Synthetic construct
  source 1..8877: mol_type = other DNA
gacatcataaaaccagatccgcctaaaaatcttcaacttaaacccctgaaaaattccaggcaagttgaggtgagctgggagtatcctgatacgtggtctacgccccactcatacttcagtctcactttttgcgttcaggttcaaggtaagagtaaacgcgaaaaaaaagaccgcgtttttacagacaaaacgagtgccaccgtaatatgcaggaaaaacgctagtattagtgtacgggcacaggatcgatactatagcagttcatggtctgagtgggcatctgttccttgtagtggagggggcggtagtggtgggggaggatccggtggcggtggctccgcgcgcaatctgcctgtggcgacccctgatcccggtatgtttccgtgtttgcatcattcacaaaatctcttgagggcagtttcaaatatgctgcaaaaagcacgacaaacgctggagttttacccatgtaccagtgaagagattgatcatgaggatataactaaagacaagaccagtactgtcgaggcttgtctccctctggaactgaccaagaacgagagctgcctcaattcccgcgagacatcctttatcactaatggttcctgcttggcttctaggaaaacatccttcatgatggcattgtgtctgtcatctatatacgaagacctcaagatgtaccaggtggagttcaagacgatgaacgcgaaactcttgatggacccaaaacgacagatattcctcgatcaaaacatgttggcggtcatcgatgaactgatgcaggcgctcaacttcaatagcgagaccgttccgcaaaagagctctctcgaggagcccgatttttacaaaacaaagataaaactgtgcatcctgcttcacgccttcaggattagggcggttactattgacagagtaatgtcctacctcaatgcttcttgaaaacggatccgcggccgcttttccctctgccaaaaattatggggacatcatgaagccccttgagcatctgacttctggctaataaaggaaatttattttcattgcaatagtgtgttggaattttttgtgtctctcactcggaaggacataaggtgagatctttcacaaaacaccagttatgctgagatctttcacaaaacaccagttatgcgctaatgcctttgggcctaacacgtgcctcacgaggatctgcgatcgctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacagctgaagcttcgaggggctcgcatctctccttcacgcgcccgccgccctacctgaggccgccatccacgccggttgagtcgcgttctgccgcctcccgcctgtggtgcctcctgaactgcgtccgccgtctaggtaagtttaaagctcaggtcgagaccgggcctttgtccggcgctcccttggagcctacctagactcagccggctctccacgctttgcctgaccctgcttgctcaactctacgtctttgtttcgttttctgttctgcgccgttacagatccaagctgtgaccggcgcctacggttaattaagccgccaccatgcttctcctggtgacaagccttctgctctgtgagttaccacacccagcattcctcctgatcccagaggtgcagctggtcgaatccggaggaggactggtccagcccggccggagcctgagactgagctgtgccgcttccggattcacttttgacgattacgcaatgcattgggtgaggcaggcccctggcaaggggctggaatgggtctccggaatctcttggaacagtgggcgcattggatatgccgattctgtgaagggccgattcactatctctcgggacaacgctaaaaatagtctgtttctgcagatgaattccctgcgcgccgaggataccgccgtgtactattgcgcccgagaccagggctaccactactatgatagcgccgaacatgcattcgacatttggggacagggaactgtggtcaccgtgagctccggaggaggaggaagcggaggaggagggtccggaggcgggggatcacagagcgcactgacccagccacggagcgtgagcggatttcctgggcagtctgtcaccattagttgcacaggcaccacatcagacgatgtgagctggtaccagcagcacccagggaaggctccccagctgatgctgtatgacgtgtccaaaagaccttctggcgtcccacataggtttagtggaagccggagcggccgggcagccagtctgatcatttcagggctgcagacagaggacgaagctgattatttctgttctagttacgcaggcagatataactctgtgctgtttggcgggggaacaaagctgactgtcctggaatctaagtacggaccgccctgccccccttgccctatgttctgggtgctggtggtggtcggaggcgtgctggcctgctacagcctgctggtcaccgtggccttcatcatcttttgggtgaaacggggcagaaagaaactcctgtatatattcaaacaaccatttatgagaccagtacaaactactcaagaggaagatggctgtagctgccgatttccagaagaagaagaaggaggatgtgaactgcgggtgaagttcagcagaagcgccgacgcccctgcctaccagcagggccagaatcagctgtacaacgagctgaacctgggcagaagggaagagtacgacgtcctggataagcggagaggccgggaccctgagatgggcggcaagcctcggcggaagaacccccaggaaggcctgtataacgaactgcagaaagacaagatggccgaggcctacagcgagatcggcatgaagggcgagcggaggcggggcaagggccacgacggcctgtatcagggcctgtccaccgccaccaaggatacctacgacgccctgcacatgcaggccctgcccccaaggggaagcggagctactaacttcagcctgctgaagcaggctggagacgtggaggagaaccctggacctatggttggttcgctaaactgcatcgtcgctgtgtcccagaacatgggcatcggcaagaacggggacttcccctggccaccgctcaggaatgaatccagatatttccagagaatgaccacaacctcttcagtagaaggtaaacagaatctggtgattatgggtaagaagacctggttctccattcctgagaagaatcgacctttaaagggtagaattaatttagttctcagcagagaactcaaggaacctccacaaggagctcattttctttccagaagtctagatgatgccttaaaacttactgaacaaccagaattagcaaataaagtagacatggtctggatagttggtggcagttctgtttataaggaagccatgaatcacccaggccatcttaaactatttgtgacaaggatcatgcaagactttgaaagtgacacgttttttccagaaattgatttggagaaatataaacttctgccagaatacccaggtgttctctctgatgtccaggaggagaaaggcattaagtacaaatttgaagtatatgagaagaatgatggcggcggagagggcagaggaagtcttctaacatgcggtgacgtggaggagaatcccggccctatgcttctcctggtgacaagccttctgctctgtgagttaccacacccagcattcctcctgatcccacgcaaagtgtgtaacggaataggtattggtgaatttaaagactcactctccataaatgctacgaatattaaacacttcaaaaactgcacctccatcagtggcgatctccacatcctgccggtggcatttaggggtgactccttcacacatactcctcctctggatccacaggaactggatattctgaaaaccgtaaaggaaatcacagggtttttgctgattcaggcttggcctgaaaacaggacggacctccatgcctttgagaacctagaaatcatacgcggcaggaccaagcaacatggtcagttttctcttgcagtcgtcagcctgaacataacatccttgggattacgctccctcaaggagataagtgatggagatgtgataatttcaggaaacaaaaatttgtgctatgcaaatacaataaactggaaaaaactgtttgggacctccggtcagaaaaccaaaattataagcaacagaggtgaaaacagctgcaaggccacaggccaggtctgccatgccttgtgctcccccgagggctgctggggcccggagcccagggactgcgtctcttgccggaatgtcagccgaggcagggaatgcgtggacaagtgcaaccttctggagggtgagccaagggagtttgtggagaactctgagtgcatacagtgccacccagagtgcctgcctcaggccatgaacatcacctgcacaggacggggaccagacaactgtatccagtgtgcccactacattgacggcccccactgcgtcaagacctgcccggcaggagtcatgggagaaaacaacaccctggtctggaagtacgcagacgccggccatgtgtgccacctgtgccatccaaactgcacctacggatgcactgggccaggtcttgaaggctgtccaacgaatgggcctaagatcccgtccatcgccactgggatggtgggggcactcctcttgctgctggtggtggccctggggatcggcctcttcatgtgacctgcaggcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggatggctagatctttttccccgtatccccccaggtgtctgcaggctcaaagagcagcgagaagcgttcagaggaaagcgatcccgtgccaccttccccgtgcccgggctgtccccgcacgctgccggctcggggatgcggggggagcgccggaccggagcggagccccgggcggctcgctgctgccccctagcgggggagggacgtaattacatccctgggggctttgggggggggctgtccctgatatctataacaagaaaatatatatataataagttatcacgtaagtagaacatgaaataacaatataattatcgtatgagttaaatcttaaaagtcacgtaaaagataatcatgcgtcattttgactcacgcggtcgttatagttcaaaatcagtgacacttaccgcattgacaagcacgcctcacgggagctccaagcggcgactgagatgtcctaaatgcacagcgacggattcgcgctatttagaaagagagagcaatatttcaagaatgcatgcgtcaattttacgcagactatctttctagggttaagtcgacccgcctaatgagcgggcttttttttggcttgttgtccacaaccgggcttgttgtccacaaccgttaaaccttaaaagctttaaaagccttatatattcttttttttcttataaaacttaaaaccttagaggctatttaagttgctgatttatattaattttattgttcaaacatgagagcttagtacgtgaaacatgagagcttagtacgttagccatgagagcttagtacgttagccatgagggtttagttcgttaaacatgagagcttagtacgttaaacatgagagcttagtacgtactatcaacaggttgaactgctgatccacgttgtggtagaattggtaaagagagtcgtgtaaaatatcgagttcgcacatcttgttgtctgattattgatttttggcgaaaccatttgatcatatgacaagatgtgtatctaccttaacttaatgattttgataaaaatcattaggactagtttaaccctagaaagataatcatattgtgacgtacgttaaagataatcatgcgtaaaattgacgcatgtgttttatcggtctgtatatcgaggtttatttattaatttgaatagatattaagttttattatatttacacttacatactaataataaattcaacaaacaatttatttatgtttatttatttattaaaaaaaaacaaaaactcaaaatttcttctataaagtaacaaaacttttatgaattcgagggacagcccccccccaaagcccccagggatgtaattacgtccctcccccgctagggggcagcagcgagccgcccggggctccgctccggtccggcgctccccccgcatccccgagccggcagcgtgcggggacagcccgggcacggggaaggtggcacgggatcgctttcctctgaacgcttctcgctgctctttgagcctgcagacacctggggggatacggggaaaaggctcgcgatcgaatgagtcacatcgatctccgccccctcttcgagggggcggggtcgaggaggaaaaactcgaatgagtcacatcgaccctttgatcttcgaggggactttccggggtggagcaagcgtgacaagtccacgtatgacccgaccgacgatatcgaagcctacgcgctgaacgccagccccgatcgaccccgccccctcgatttccaagaaatcgaatgacatcatctttcgaatgacatcatctttcgaggggactttcctcgaacttccttcgaggggactttcctcgaggggactttcctcgaggaggaaaaactcgagtagagtctagactctacattttgacacccccataatatttttccagaattaacagtataaattgcatctcttgttcaagagttccctatcactctctttaatcactactcacagtaacctcaactcctggctagcgtactgcgtttgccgccaccatgcttctcctggtgacaagccttctgctctgtgagttaccacacccagcattcctcctgatcccatgccaccctgagtgtcagccccagaatggctcagtgacctgttttggaccggaggctgaccagtgtgtggcctgtgcccactataaggaccctcccttctgcgtggcccgctgccccagcggtgtgaaacctgacctctcctacatgcccatctggaagtttccagatgaggagggcgcatgccagccttgccccatcaactgcacccactcctgtgtggacctggatgacaagggctgccccgccgagcagagagccagcccgttaacgggtggaggcagcggaggtggctccatcatctctgcggtggttggcattctgctggtcgtggtcttgggggtggtctttgggatcctcatcggcggcggagagggcagaggaagtcttctaacatgcggtgacgtggaggagaatcccggccctatgcagatccctcaggccccttggcctgtcgtgtgggctgtgctgcagctgggatggcggcctggctggtttctggacagccccgacagaccctggaacccccctacattttcccctgccctgctggtcgtgaccgagggcgacaatgccaccttcacctgtagcttcagcaacaccagcgagagcttcgtgctgaactggtacagaatgagccccagcaaccagaccgacaagctggccgccttccccgaggatagatctcagcccggccaggactgccggttcagagtgacccagctgcccaacggccgggacttccacatgtctgtcgtgcgggccagacggaacgacagcggcacatatctgtgcggcgccatcagcctggcccccaagctccagatcaaagagagcctgagagccgagctgagagtgaccgagagaagggccgaagtgcctaccgcccaccctagcccatctccaagacctgccggccagttccagacactggtcggcggaggcggaagcggtggcggaggaagcggaggcggcggatctatgtgtcatcagcagttggtgatctcttggttttctcttgtatttcttgcgtcaccacttgtggccatctgggaactgaagaaggacgtatatgtggttgaactggactggtaccccgacgcaccgggggaaatggtagtgttgacttgtgatacacctgaggaagacggaattacttggacccttgaccaatccagtgaggtgcttggatcaggaaagacactgaccattcaagtcaaagagtttggggacgccggacaatacacttgtcacaaaggtggagaggtattgagccattctcttcttcttctccataagaaagaagatgggatatggagcacggatattctgaaagaccaaaaagaaccaaaaaacaaaactttcctcaggtgtgaggcgaaaaattactcaggacgctttacttgctggtggcttaccactatatccactgatcttactttttctgtcaagagtagtcgcggttcatctgatccccaaggtgtgacatgtggtgcagccacgctcagtgccgaacgagtgaggggggataataaagaatatgagtactccgttgaatgccaagaagattctgcgtgtcctgctgcagaagaaagtctgccgatagaggttatggttgatgcggttcacaaacttaaatatgagaattatacgagcagtttcttcatccgg

SEQ ID NO: 102 - DNA, 7694 bp, Synthetic construct
  source 1..7694: mol_type = other DNA
aattcaacaaacaatttatttatgtttatttatttattaaaaaaaaacaaaaactcaaaatttcttctataaagtaacaaaacttttatgaattcgagggacagcccccccccaaagcccccagggatgtaattacgtccctcccccgctagggggcagcagcgagccgcccggggctccgctccggtccggcgctccccccgcatccccgagccggcagcgtgcggggacagcccgggcacggggaaggtggcacgggatcgctttcctctgaacgcttctcgctgctctttgagcctgcagacacctggggggatacggggaaaaggctcgcgatcgaatgagtcacatcgatctccgccccctcttcgagggggcggggtcgaggaggaaaaactcgaatgagtcacatcgaccctttgatcttcgaggggactttccggggtggagcaagcgtgacaagtccacgtatgacccgaccgacgatatcgaagcctacgcgctgaacgccagccccgatcgaccccgccccctcgatttccaagaaatcgaatgacatcatctttcgaatgacatcatctttcgaggggactttcctcgaacttccttcgaggggactttcctcgaggggactttcctcgaggaggaaaaactcgagtagagtctagactctacattttgacacccccataatatttttccagaattaacagtataaattgcatctcttgttcaagagttccctatcactctctttaatcactactcacagtaacctcaactcctggctagcgtactgcgtttgccgccaccatgcttctcctggtgacaagccttctgctctgtgagttaccacacccagcattcctcctgatcccatgccaccctgagtgtcagccccagaatggctcagtgacctgttttggaccggaggctgaccagtgtgtggcctgtgcccactataaggaccctcccttctgcgtggcccgctgccccagcggtgtgaaacctgacctctcctacatgcccatctggaagtttccagatgaggagggcgcatgccagccttgccccatcaactgcacccactcctgtgtggacctggatgacaagggctgccccgccgagcagagagccagcccgttaacgggtggaggcagcggaggtggctccatcatctctgcggtggttggcattctgctggtcgtggtcttgggggtggtctttgggatcctcatcggcggcggagagggcagaggaagtcttctaacatgcggtgacgtggaggagaatcccggccctatgcagatccctcaggccccttggcctgtcgtgtgggctgtgctgcagctgggatggcggcctggctggtttctggacagccccgacagaccctggaacccccctacattttcccctgccctgctggtcgtgaccgagggcgacaatgccaccttcacctgtagcttcagcaacaccagcgagagcttcgtgctgaactggtacagaatgagccccagcaaccagaccgacaagctggccgccttccccgaggatagatctcagcccggccaggactgccggttcagagtgacccagctgcccaacggccgggacttccacatgtctgtcgtgcgggccagacggaacgacagcggcacatatctgtgcggcgccatcagcctggcccccaagctccagatcaaagagagcctgagagccgagctgagagtgaccgagagaagggccgaagtgcctaccgcccaccctagcccatctccaagacctgccggccagttccagacactggtcggcggaggatgcgacctgcctcagactcactctctgggctctagacggaccctgatgctgctggcccagatgagaaagatcagcctgttctcctgcctgaaggaccggcacgatttcggcttccctcaagaggaattcggcaaccagttccagaaggccgagacaatccctgtgctgcacgagatgatccagcagatcttcaacctgttcagcaccaaggacagcagcgccgcctgggatgagacactgctggacaagttctacaccgagctgtaccagcagctcaacgacctggaagcctgcgtgatccaaggcgtgggagtgaccgaaacacccctgatgaaggaagatagcatcctggccgtgcgcaagtacttccagcggatcaccctgtacctgaaagagaagaagtacagcccctgcgcctgggaagtcgtcagagccgagattatgcggagcttcagcctgagcaccaacctgcaagaaagcctgcggtccaaagaatgaaaacggatccgcggccgcttttccctctgccaaaaattatggggacatcatgaagccccttgagcatctgacttctggctaataaaggaaatttattttcattgcaatagtgtgttggaattttttgtgtctctcactcggaaggacataaggtgagatctttcacaaaacaccagttatgctgagatctttcacaaaacaccagttatgcgctaatgcctttgggcctaacacgtgcctcacgaggatctgcgatcgctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacagctgaagcttcgaggggctcgcatctctccttcacgcgcccgccgccctacctgaggccgccatccacgccggttgagtcgcgttctgccgcctcccgcctgtggtgcctcctgaactgcgtccgccgtctaggtaagtttaaagctcaggtcgagaccgggcctttgtccggcgctcccttggagcctacctagactcagccggctctccacgctttgcctgaccctgcttgctcaactctacgtctttgtttcgttttctgttctgcgccgttacagatccaagctgtgaccggcgcctacggttaattaagccgccaccatgcttctcctggtgacaagccttctgctctgtgagttaccacacccagcattcctcctgatcccagaggtgcagctggtcgaatccggaggaggactggtccagcccggccggagcctgagactgagctgtgccgcttccggattcacttttgacgattacgcaatgcattgggtgaggcaggcccctggcaaggggctggaatgggtctccggaatctcttggaacagtgggcgcattggatatgccgattctgtgaagggccgattcactatctctcgggacaacgctaaaaatagtctgtttctgcagatgaattccctgcgcgccgaggataccgccgtgtactattgcgcccgagaccagggctaccactactatgatagcgccgaacatgcattcgacatttggggacagggaactgtggtcaccgtgagctccggaggaggaggaagcggaggaggagggtccggaggcgggggatcacagagcgcactgacccagccacggagcgtgagcggatttcctgggcagtctgtcaccattagttgcacaggcaccacatcagacgatgtgagctggtaccagcagcacccagggaaggctccccagctgatgctgtatgacgtgtccaaaagaccttctggcgtcccacataggtttagtggaagccggagcggccgggcagccagtctgatcatttcagggctgcagacagaggacgaagctgattatttctgttctagttacgcaggcagatataactctgtgctgtttggcgggggaacaaagctgactgtcctggaatctaagtacggaccgccctgccccccttgccctatgttctgggtgctggtggtggtcggaggcgtgctggcctgctacagcctgctggtcaccgtggccttcatcatcttttgggtgaaacggggcagaaagaaactcctgtatatattcaaacaaccatttatgagaccagtacaaactactcaagaggaagatggctgtagctgccgatttccagaagaagaagaaggaggatgtgaactgcgggtgaagttcagcagaagcgccgacgcccctgcctaccagcagggccagaatcagctgtacaacgagctgaacctgggcagaagggaagagtacgacgtcctggataagcggagaggccgggaccctgagatgggcggcaagcctcggcggaagaacccccaggaaggcctgtataacgaactgcagaaagacaagatggccgaggcctacagcgagatcggcatgaagggcgagcggaggcggggcaagggccacgacggcctgtatcagggcctgtccaccgccaccaaggatacctacgacgccctgcacatgcaggccctgcccccaaggggaagcggagctactaacttcagcctgctgaagcaggctggagacgtggaggagaaccctggacctatggttggttcgctaaactgcatcgtcgctgtgtcccagaacatgggcatcggcaagaacggggacttcccctggccaccgctcaggaatgaatccagatatttccagagaatgaccacaacctcttcagtagaaggtaaacagaatctggtgattatgggtaagaagacctggttctccattcctgagaagaatcgacctttaaagggtagaattaatttagttctcagcagagaactcaaggaacctccacaaggagctcattttctttccagaagtctagatgatgccttaaaacttactgaacaaccagaattagcaaataaagtagacatggtctggatagttggtggcagttctgtttataaggaagccatgaatcacccaggccatcttaaactatttgtgacaaggatcatgcaagactttgaaagtgacacgttttttccagaaattgatttggagaaatataaacttctgccagaatacccaggtgttctctctgatgtccaggaggagaaaggcattaagtacaaatttgaagtatatgagaagaatgatggcggcggagagggcagaggaagtcttctaacatgcggtgacgtggaggagaatcccggccctatgcttctcctggtgacaagccttctgctctgtgagttaccacacccagcattcctcctgatcccacgcaaagtgtgtaacggaataggtattggtgaatttaaagactcactctccataaatgctacgaatattaaacacttcaaaaactgcacctccatcagtggcgatctccacatcctgccggtggcatttaggggtgactccttcacacatactcctcctctggatccacaggaactggatattctgaaaaccgtaaaggaaatcacagggtttttgctgattcaggcttggcctgaaaacaggacggacctccatgcctttgagaacctagaaatcatacgcggcaggaccaagcaacatggtcagttttctcttgcagtcgtcagcctgaacataacatccttgggattacgctccctcaaggagataagtgatggagatgtgataatttcaggaaacaaaaatttgtgctatgcaaatacaataaactggaaaaaactgtttgggacctccggtcagaaaaccaaaattataagcaacagaggtgaaaacagctgcaaggccacaggccaggtctgccatgccttgtgctcccccgagggctgctggggcccggagcccagggactgcgtctcttgccggaatgtcagccgaggcagggaatgcgtggacaagtgcaaccttctggagggtgagccaagggagtttgtggagaactctgagtgcatacagtgccacccagagtgcctgcctcaggccatgaacatcacctgcacaggacggggaccagacaactgtatccagtgtgcccactacattgacggcccccactgcgtcaagacctgcccggcaggagtcatgggagaaaacaacaccctggtctggaagtacgcagacgccggccatgtgtgccacctgtgccatccaaactgcacctacggatgcactgggccaggtcttgaaggctgtccaacgaatgggcctaagatcccgtccatcgccactgggatggtgggggcactcctcttgctgctggtggtggccctggggatcggcctcttcatgtgacctgcaggcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggatggctagatctttttccccgtatccccccaggtgtctgcaggctcaaagagcagcgagaagcgttcagaggaaagcgatcccgtgccaccttccccgtgcccgggctgtccccgcacgctgccggctcggggatgcggggggagcgccggaccggagcggagccccgggcggctcgctgctgccccctagcgggggagggacgtaattacatccctgggggctttgggggggggctgtccctgatatctataacaagaaaatatatatataataagttatcacgtaagtagaacatgaaataacaatataattatcgtatgagttaaatcttaaaagtcacgtaaaagataatcatgcgtcattttgactcacgcggtcgttatagttcaaaatcagtgacacttaccgcattgacaagcacgcctcacgggagctccaagcggcgactgagatgtcctaaatgcacagcgacggattcgcgctatttagaaagagagagcaatatttcaagaatgcatgcgtcaattttacgcagactatctttctagggttaagtcgacccgcctaatgagcgggcttttttttggcttgttgtccacaaccgttaaaccttaaaagctttaaaagccttatatattcttttttttcttataaaacttaaaaccttagaggctatttaagttgctgatttatattaattttattgttcaaacatgagagcttagtacgtgaaacatgagagcttagtacgttagccatgagagcttagtacgttagccatgagggtttagttcgttaaacatgagagcttagtacgttaaacatgagagcttagtacgtactatcaacaggttgaactgctgatccacgttgtggtagaattggtaaagagagtcgtgtaaaatatcgagttcgcacatcttgttgtctgattattgatttttggcgaaaccatttgatcatatgacaagatgtgtatctaccttaacttaatgattttgataaaaatcattaggactagtttaaccctagaaagataatcatattgtgacgtacgttaaagataatcatgcgtaaaattgacgcatgtgttttatcggtctgtatatcgaggtttatttattaatttgaatagatattaagttttattatatttacacttacatactaataata

SEQ ID NO: 103 - DNA, 4791 bp, Synthetic construct
  source 1..4791: mol_type = other DNA
tcgagggacagcccccccccaaagcccccagggatgtaattacgtccctcccccgctagggggcagcagcgagccgcccggggctccgctccggtccggcgctccccccgcatccccgagccggcagcgtgcggggacagcccgggcacggggaaggtggcacgggatcgctttcctctgaacgcttctcgctgctctttgagcctgcagacacctggggggatacggggaaaaggctcgcgatcgaatgagtcacatcgatctccgccccctcttcgagggggcggggtcgaggaggaaaaactcgaatgagtcacatcgaccctttgatcttcgaggggactttccggggtggagcaagcgtgacaagtccacgtatgacccgaccgacgatatcgaagcctacgcgctgaacgccagccccgatcgaccccgccccctcgatttccaagaaatcgaatgacatcatctttcgaatgacatcatctttcgaggggactttcctcgaacttccttcgaggggactttcctcgaggggactttcctcgaggaggaaaaactcgagtagagtctagactctacattttgacacccccataatatttttccagaattaacagtataaattgcatctcttgttcaagagttccctatcactctctttaatcactactcacagtaacctcaactcctggctagccgccaccatgcttctcctggtgacaagccttctgctctgtgagttaccacacccagcattcctcctgatcccatgccaccctgagtgtcagccccagaatggctcagtgacctgttttggaccggaggctgaccagtgtgtggcctgtgcccactataaggaccctcccttctgcgtggcccgctgccccagcggtgtgaaacctgacctctcctacatgcccatctggaagtttccagatgaggagggcgcatgccagccttgccccatcaactgcacccactcctgtgtggacctggatgacaagggctgccccgccgagcagagagccagcccgttaacgggtggaggcagcggaggtggctccatcatctctgcggtggttggcattctgctggtcgtggtcttgggggtggtctttgggatcctcatcggcggcggagagggcagaggaagtcttctaacatgcggtgacgtggaggagaatcccggccctatgcagatccctcaggccccttggcctgtcgtgtgggctgtgctgcagctgggatggcggcctggctggtttctggacagccccgacagaccctggaacccccctacattttcccctgccctgctggtcgtgaccgagggcgacaatgccaccttcacctgtagcttcagcaacaccagcgagagcttcgtgctgaactggtacagaatgagccccagcaaccagaccgacaagctggccgccttccccgaggatagatctcagcccggccaggactgccggttcagagtgacccagctgcccaacggccgggacttccacatgtctgtcgtgcgggccagacggaacgacagcggcacatatctgtgcggcgccatcagcctggcccccaagctccagatcaaagagagcctgagagccgagctgagagtgaccgagagaagggccgaagtgcctaccgcccaccctagcccatctccaagacctgccggccagttccagacactggtcggcggaggcggaagcggtggcggaggaagcggaggcggcggatctatgtgtcatcagcagttggtgatctcttggttttctcttgtatttcttgcgtcaccacttgtggccatctgggaactgaagaaggacgtatatgtggttgaactggactggtaccccgacgcaccgggggaaatggtagtgttgacttgtgatacacctgaggaagacggaattacttggacccttgaccaatccagtgaggtgcttggatcaggaaagacactgaccattcaagtcaaagagtttggggacgccggacaatacacttgtcacaaaggtggagaggtattgagccattctcttcttcttctccataagaaagaagatgggatatggagcacggatattctgaaagaccaaaaagaaccaaaaaacaaaactttcctcaggtgtgaggcgaaaaattactcaggacgctttacttgctggtggcttaccactatatccactgatcttactttttctgtcaagagtagtcgcggttcatctgatccccaaggtgtgacatgtggtgcagccacgctcagtgccgaacgagtgaggggggataataaagaatatgagtactccgttgaatgccaagaagattctgcgtgtcctgctgcagaagaaagtctgccgatagaggttatggttgatgcggttcacaaacttaaatatgagaattatacgagcagtttcttcatccgggacatcataaaaccagatccgcctaaaaatcttcaacttaaacccctgaaaaattccaggcaagttgaggtgagctgggagtatcctgatacgtggtctacgccccactcatacttcagtctcactttttgcgttcaggttcaaggtaagagtaaacgcgaaaaaaaagaccgcgtttttacagacaaaacgagtgccaccgtaatatgcaggaaaaacgctagtattagtgtacgggcacaggatcgatactatagcagttcatggtctgagtgggcatctgttccttgtagtggagggggcggtagtggtgggggaggatccggtggcggtggctccgcgcgcaatctgcctgtggcgacccctgatcccggtatgtttccgtgtttgcatcattcacaaaatctcttgagggcagtttcaaatatgctgcaaaaagcacgacaaacgctggagttttacccatgtaccagtgaagagattgatcatgaggatataactaaagacaagaccagtactgtcgaggcttgtctccctctggaactgaccaagaacgagagctgcctcaattcccgcgagacatcctttatcactaatggttcctgcttggcttctaggaaaacatccttcatgatggcattgtgtctgtcatctatatacgaagacctcaagatgtaccaggtggagttcaagacgatgaacgcgaaactcttgatggacccaaaacgacagatattcctcgatcaaaacatgttggcggtcatcgatgaactgatgcaggcgctcaacttcaatagcgagaccgttccgcaaaagagctctctcgaggagcccgatttttacaaaacaaagataaaactgtgcatcctgcttcacgccttcaggattagggcggttactattgacagagtaatgtcctacctcaatgcttcttgagcggccgcttttccctctgccaaaaattatggggacatcatgaagccccttgagcatctgacttctggctaataaaggaaatttattttcattgcaatagtgtgttggaattttttgtgtctctcactcggaaggacataaggatggctagatctttttccccgtatccccccaggtgtctgcaggctcaaagagcagcgagaagcgttcagaggaaagcgatcccgtgccaccttccccgtgcccgggctgtccccgcacgctgccggctcggggatgcggggggagcgccggaccggagcggagccccgggcggctcgctgctgccccctagcgggggagggacgtaattacatccctgggggctttgggggggggctgtccctgatatctataacaagaaaatatatatataataagttatcacgtaagtagaacatgaaataacaatataattatcgtatgagttaaatcttaaaagtcacgtaaaagataatcatgcgtcattttgactcacgcggtcgttatagttcaaaatcagtgacacttaccgcattgacaagcacgcctcacgggagctccaagcggcgactgagatgtcctaaatgcacagcgacggattcgcgctatttagaaagagagagcaatatttcaagaatgcatgcgtcaattttacgcagactatctttctagggttaagtcgacccgcctaatgagcgggcttttttttggcttgttgtccacaaccgttaaaccttaaaagctttaaaagccttatatattcttttttttcttataaaacttaaaaccttagaggctatttaagttgctgatttatattaattttattgttcaaacatgagagcttagtacgtgaaacatgagagcttagtacgttagccatgagagcttagtacgttagccatgagggtttagttcgttaaacatgagagcttagtacgttaaacatgagagcttagtacgtactatcaacaggttgaactgctgatccacgttgtggtagaattggtaaagagagtcgtgtaaaatatcgagttcgcacatcttgttgtctgattattgatttttggcgaaaccatttgatcatatgacaagatgtgtatctaccttaacttaatgattttgataaaaatcattaggactagtttaaccctagaaagataatcatattgtgacgtacgttaaagataatcatgcgtaaaattgacgcatgtgttttatcggtctgtatatcgaggtttatttattaatttgaatagatattaagttttattatatttacacttacatactaataataaattcaacaaacaatttatttatgtttatttatttattaaaaaaaaacaaaaactcaaaatttcttctataaagtaacaaaacttttatgaat

SEQ ID NO: 104 - protein, 6 aa, Synthetic construct
HHHHHH

SEQ ID NO: 105 - protein, 8 aa, Synthetic construct
DYKDDDDK

SEQ ID NO: 106 - protein, 8 aa, Synthetic construct
DLYDDDDK

SEQ ID NO: 107 - protein, 15 aa, Synthetic construct
GLNDIFEAQKIEWHE

SEQ ID NO: 108 - protein, 26 aa, Synthetic construct
KRRWKKNFIAVSAANRFKKISSSGAL

SEQ ID NO: 109 - protein, 9 aa, Synthetic construct
YPYDVPDYA

SEQ ID NO: 110 - protein, 10 aa, Synthetic construct
EQKLISEEDL

SEQ ID NO: 111 - protein, 8 aa, Synthetic construct
WRHPQFGG

SEQ ID NO: 112 - protein, 8 aa, Synthetic construct
WSHPQFEK

SEQ ID NO: 113 - protein, 13 aa, Synthetic construct
SLAELLNAGLGGS

SEQ ID NO: 114 - protein, 8 aa, Synthetic construct
TQDPSRVG

SEQ ID NO: 115 - protein, 14 aa, Synthetic construct
GKPIPNPLLGLDST

SEQ ID NO: 116 - protein, 268 aa, Synthetic construct
MLLLVTSLLLCELPHPAFLLIPEVQLVESGGGLVQPGRSLRLSCAASGFTFDDYAMHWVRQAPGKGLEWVSGISWNSGRIGYADSVKGRFTISRDNAKNSLFLQMNSLRAEDTAVYYCARDQGYHYYDSAEHAFDIWGQGTVVTVSSGGGGSGGGGSGGGGSQSALTQPRSVSGFPGQSVTISCTGTTSDDVSWYQQHPGKAPQLMLYDVSKRPSGVPHRFSGSRSGRAASLIISGLQTEDEADYFCSSYAGRYNSVLFGGGTKLTVL

SEQ ID NO: 117 - protein, 246 aa, Synthetic construct
EVQLVESGGGLVQPGRSLRLSCAASGFTFDDYAMHWVRQAPGKGLEWVSGISWNSGRIGYADSVKGRFTISRDNAKNSLFLQMNSLRAEDTAVYYCARDQGYHYYDSAEHAFDIWGQGTVVTVSSGGGGSGGGGSGGGGSQSALTQPRSVSGFPGQSVTISCTGTTSDDVSWYQQHPGKAPQLMLYDVSKRPSGVPHRFSGSRSGRAASLIISGLQTEDEADYFCSSYAGRYNSVLFGGGTKLTVL

SEQ ID NO: 118 - protein, 267 aa, Synthetic construct
MLLLVTSLLLCELPHPAFLLIPDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGSTSGSGKPGSGEGSTKGEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSS

SEQ ID NO: 119 - protein, 244 aa, Synthetic construct
DIQMTQTTSSLSALGDRVTISCRASQDISKYLNWYQQKPDGTVKLLIYHTSRLHSGVPSRFSGSGSGTDYSLTISNLEQEDIATYFCQQGNTLPYTFGGGTKLEITGSTSGSGKPGSGEGSTKGEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSS

SEQ ID NO: 120 - protein, 239 aa, Synthetic construct
QVQLVQSGAEVKKPGASVKVSCKASGYTFTNYDINWVRQAPGQGLEWIGWIYPGDGSTKYNEKFKAKATLTADTSTSTAYMELRSLRSDDTAVYYCASGYEDAMDYWGQGTTVTVSSGGGGSGGGGSGGGGSDIQMTQSPSSLSASVGDRVTINCKASQDINSYLSWFQQKPGKAPKTLIYRANRLVDGVPSRFSGSGSGQDYTLTISSLQPEDFATYYCLQYDEFPLTFGGGTKVEIK

SEQ ID NO: 121 - protein, 239 aa, Synthetic construct
DIQMTQSPSSLSASVGDRVTINCKASQDINSYLSWFQQKPGKAPKTLIYRANRLVDGVPSRFSGSGSGQDYTLTISSLQPEDFATYYCLQYDEFPLTFGGGTKVEIKGGGGSGGGGSGGGGSQVQLVQSGAEVKKPGASVKVSCKASGYTFTNYDINWVRQAPGQGLEWIGWIYPGDGSTKYNEKFKAKATLTADTSTSTAYMELRSLRSDDTAVYYCASGYEDAMDYWGQGTTVTVSS

SEQ ID NO: 122 - protein, 22 aa, Synthetic construct
MLLLVTSLLLCELPHPAFLLIP

SEQ ID NO: 123 - protein, 5 aa, Synthetic construct
  REPEAT 1..5: note = (GGGGS) can be repeated n times, wherein n is an integer of 1, 2, 3, 4, 5, 6, 7, 8, 9 or 10
GGGGS

SEQ ID NO: 124 - protein, 9 aa, Synthetic construct
  REPEAT 1..4: note = (GGGS) can be repeated n times, wherein n is an integer of 1, 2, 3, 4, 5, 6, 7, 8, 9 or 10
  REPEAT 5..9: note = (GGGGS) can be repeated n times, wherein n is an integer of 1, 2, 3, 4, 5, 6, 7, 8, 9 or 10
GGGSGGGGS

SEQ ID NO: 125 - protein, 7 aa, Synthetic construct
  REPEAT 1..4: note = (GGGS) can be repeated n times, wherein n is an integer of 1, 2, 3, 4, 5, 6, 7, 8, 9 or 10
  REPEAT 5..7: note = (GGS) can be repeated n times, wherein n is an integer of 1, 2, 3, 4, 5, 6, 7, 8, 9 or 10
GGGSGGS

SEQ ID NO: 126 - protein, 9 aa, Synthetic construct
  REPEAT 1..4: note = (GGGS) can be repeated n times, wherein n is an integer of 1, 2, 3, 4, 5, 6, 7, 8, 9 or 10
GGGSGGGGS

SEQ ID NO: 127 - protein, 20 aa, Synthetic construct
GGGGSGGGGSGGGGSGGGGS

SEQ ID NO: 128 - protein, 15 aa, Synthetic construct
GGGGSGGGGSGGGGS

SEQ ID NO: 129 - protein, 10 aa, Synthetic construct
GGGGSGGGGS

SEQ ID NO: 130 - protein, 5 aa, Synthetic construct
GGGGS

SEQ ID NO: 131 - protein, 8 aa, Synthetic construct
GGGSGGGS

SEQ ID NO: 132 - protein, 4 aa, Synthetic construct
GGGS

SEQ ID NO: 133 - protein, 6 aa, Synthetic construct
GGSGGS

SEQ ID NO: 134 - protein, 11 aa, Synthetic construct
GGSGGGSGGSG

SEQ ID NO: 135 - protein, 10 aa, Synthetic construct
GGSGGGSGSG

SEQ ID NO: 136 - protein, 8 aa, Synthetic construct
GGSGGGSG

SEQ ID NO: 137 - protein, 5 aa, Synthetic construct
  REPEAT 1..5: note = (EAAAK) can be repeated n times, wherein n is an integer of 1, 2, 3, 4, 5, 6, 7, 8, 9 or more
EAAAK

SEQ ID NO: 138 - DNA, 114 bp, Synthetic construct
  source 1..114: mol_type = other DNA
acattttgacacccccataatatttttccagaattaacagtataaattgcatctcttgttcaagagttccctatcactctctttaatcactactcacagtaacctcaactcctg